(19) United States
(12) Patent Application Publication
(10) Pub. No.: US 2007/0254917 A1
(43) Pub. Date: Nov. 1, 2007

(54) INTRACELLULAR RECEPTOR MODULATOR COMPOUNDS AND METHODS

(76) Inventors: Robert I. Higuchi, Solana Beach, CA (US); Steven L. Roach, San Diego, CA (US); Lin Zhi, San Diego, CA (US); Mark E. Adams, San Diego, CA (US); Yan Liu, San Diego, CA (US); Donald S. Karanewsky, Escondido, CA (US); Andrew Hudson, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/622,997

(22) Filed: Jan. 12, 2007

Related U.S. Application Data
(63) Continuation of application No. PCT/US05/24625, filed on Jul. 12, 2005.
(60) Provisional application No. 60/587,816, filed on Jul. 14, 2004.

(51) Int. Cl. C07D 215/00; A61K 31/47; A61P 1/00; A61P 11/00; A61P 17/00; A61P 25/00; G01N 33/569; C07D 215/20; A61P 3/00; A61P 35/00; A61P 37/00; A61P 9/00

(52) U.S. Cl. 514/312; 435/7.21; 514/311; 544/333; 546/152; 546/153

(57) ABSTRACT

This invention relates to compounds that bind to intracellular receptors and/or modulate activity of intracellular receptors, and to methods for making and using such compounds.

### RELATED APPLICATIONS

This application is a continuation of co-pending Application No. PCT/US2005/024625, filed Jul. 12, 2005, which is a non-provisional of 60/587,816, filed Jul. 14, 2004, both of which are incorporated herein by reference in their entireties.

### BACKGROUND OF THE INVENTION

1. Field of the Invention

This invention relates to compounds that bind to intracellular receptors and/or modulate activity of intracellular receptors, and to methods for making and using such compounds.

2. Description of the Related Art

Certain intracellular receptors (IRs) have been shown to regulate transcription of certain genes. See e.g. R. M. Evans, Science, 240, 889 (1988). Certain of such IRs are steroid receptors, such as androgen receptors, glucocorticoid receptors, estrogen receptors, mineralocorticoid receptors, and progesterone receptors. Gene regulation by such receptors typically involves binding of an IR by a ligand.

In certain instances, a ligand binds to an IR, forming a receptor/ligand complex. That receptor/ligand complex may then translocate to the nucleus of a cell, where it may bind to the DNA of one or more gene regulatory regions. Once bound to the DNA of a particular gene regulatory region, a receptor/ligand complex may modulate the production of the protein encoded by that particular gene. In certain instances, a receptor/ligand complex regulates expression of certain proteins. In certain instances, a receptor/ligand complex may interact directly with the DNA of a particular gene regulatory region. In certain instances, a receptor/ligand complex may interact with other transcription factors, such as activator protein-1 (AP-1) or nuclear factor κB (NFκB). In certain instances, such interactions result in modulation of transcriptional activation.

### SUMMARY OF THE INVENTION

In certain embodiments, the present invention provides a compound of Formula I, II, or III: 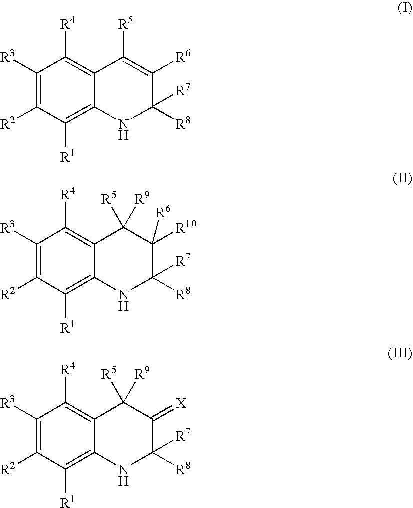
or a pharmaceutically acceptable salt, ester, amide, or prodrug thereof,
wherein:

R¹ and R² are each independently selected from the group consisting of hydrogen, a halogen, —CN, —OR¹⁶, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;

R³ is selected from the group consisting of (a), (b), (c), (d), (e), (f), (g), (h), (i), (j), (k), (l), (m), and (n):

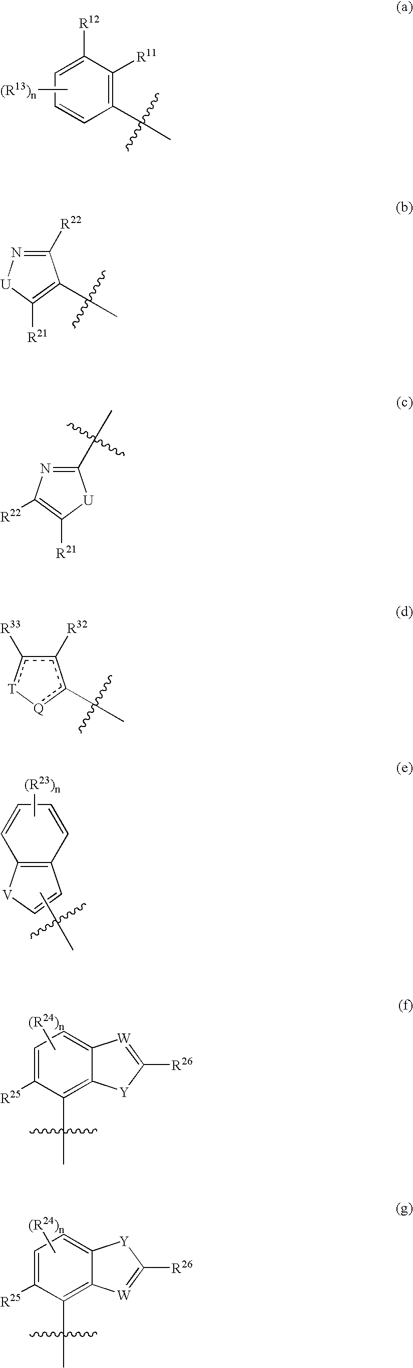

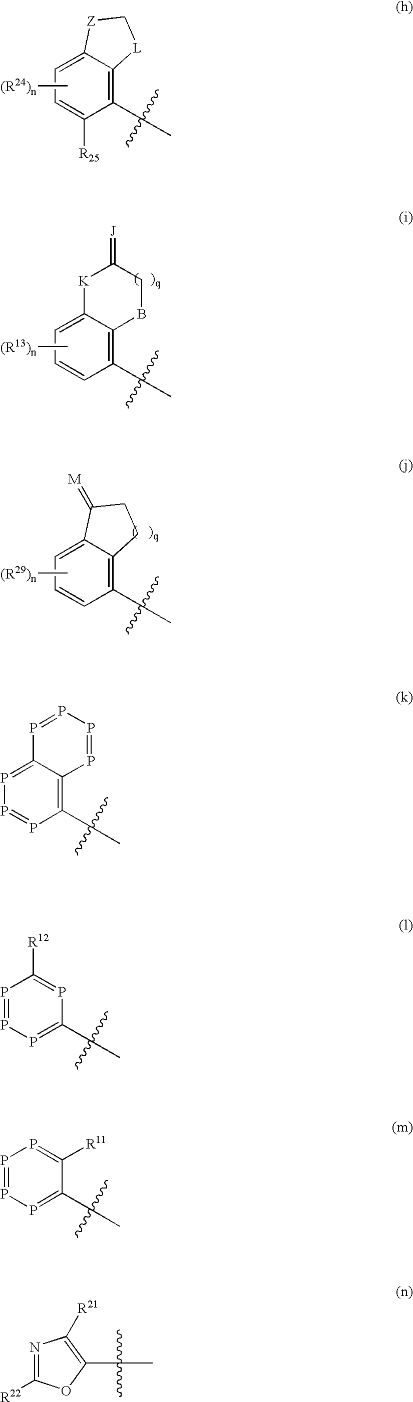

wherein,
R¹¹ is selected from the group consisting of hydrogen, a halogen, —CN, —OR¹⁶, —NR¹⁷R¹⁸, —CH₂R¹⁶, —COR²⁰, —CO₂R²⁰, —CONR²⁰R³⁷, —SOR², —SO₂R²⁰, —NO₂, NR¹⁷(OR¹⁶), an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R¹² is selected from the group consisting of hydrogen, a halogen, —CN, —COR²⁰, —CO₂R²⁰, —CONR²⁰R³⁷, NR¹⁷SO₂R²—NR¹⁷CO₂R²⁰, —NO₂, —OR¹⁶, —NR¹⁷R¹⁸, NR¹⁷(OR¹⁶), an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl or R¹² taken together with R¹¹ form a 3-7 membered ring;
each R¹³ is independently selected from the group consisting of hydrogen, a halogen, CN, —NO₂, OR¹⁶, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl or R¹³ taken together with R¹² form a 3-7 membered ring;
R²¹ is selected from the group consisting of hydrogen, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R²² is selected from the group consisting of hydrogen, a halogen, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R³² and R³³ are each independently selected from the group consisting of hydrogen, a halogen, —OR¹⁶, —CN, COR²⁰, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
each R²³ is independently selected from the group consisting of hydrogen, a halogen, OR¹⁶, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
each R²⁴ is independently selected from the group consisting of hydrogen, a halogen, and —OR¹⁶;
R²⁵ is selected from the group consisting of hydrogen, a halogen, —OR¹⁶, —CN, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R²⁶ is selected from the group consisting of hydrogen, a halogen, —OR¹⁶, —CN, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
each R²⁹ is independently selected from the group consisting of hydrogen, a halogen, and —OR¹⁶;
U is selected from the group consisting of oxygen, sulfur, and —NR¹⁷;
Q and T are each selected from the group consisting of S, O, and CR³⁴ wherein
   either Q is —CR³⁴ and T is selected from the group consisting of S, O, and —NR¹⁷,
   or T is CR³⁴ and Q is selected from the group consisting of S, O, and —NR¹⁷;
V is selected from the group consisting of O, S, and —NR¹⁷;
W is selected from the group consisting of —CR²⁷ and N;
Y is selected from the group consisting of —NR³⁶, S, and O;
Z and L are each selected from the group consisting of CH₂, —NR²⁸, and O, wherein
   either Z is CH₂ and L is selected from the group consisting of —NR²⁸ and O,
   or L is CH₂ and Z is selected from the group consisting of —NR²⁸ and O;
K is selected from the group consisting of O and —NR³⁵;
J is selected from the group consisting of O and S;
B is selected from the group consisting of O and CR²⁷;
M is selected from the group consisting of O and —NOR³⁰;
each P is independently selected from the group consisting of N and CR³¹, provided that no more than two of the Ps are N;
n is selected from 0, 1, 2, 3, and 4; and
q is selected from 0, 1, and 2;

R⁴ is selected from the group consisting of hydrogen, a halogen, NO₂, OR¹⁶, NR¹⁷R¹⁸CN, C═N(OR¹⁶), CO₂R²⁰, CONR²⁰R³⁷, NR¹⁷(OR¹⁶), CR³(OR¹⁶), an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;

R⁵ is selected from the group consisting of hydrogen, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;

R⁶ is selected from the group consisting of hydrogen and OR¹⁶;

R⁷ and R⁸ are each independently selected from the group consisting of hydrogen, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;

R⁹ is selected from the group consisting of hydrogen, OR¹⁶, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;

R¹⁰ is selected from the group consisting of hydrogen and OR¹⁶; and

X is selected from the group consisting of O, S, and NOR¹⁶;

wherein:

R¹⁶ is selected from the group consisting of hydrogen, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;

R¹⁷ and R¹⁸ are each independently selected from the group consisting of hydrogen, COR²⁰, CO₂R²⁰, SO₂R²⁰, S(O)R²⁰, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl; or R17 and R18 are linked to form a 3 to 7 membered ring;

R²⁰ and R³⁷ are each independently selected from the group consisting of hydrogen, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl; or R³⁷ and R²⁰ are linked to form a 3-7 membered ring;

R³⁴ is selected from the group consisting of hydrogen, a halogen, —NO₂, —OR¹⁶, —NR¹⁷R¹⁸, —CN, —COR²⁰, NR¹⁷(OR¹⁶), an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;

R³⁶ is selected from the group consisting of hydrogen, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;

R²⁷ is selected from the group consisting of hydrogen, a halogen, CO₂R²⁰, COR²⁰, CONR²⁰R³⁷, C═N(OR¹⁶), an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl or R²⁷ taken together with R²⁶ form a 3-7 membered ring;

R²⁸ is selected from the group consisting of hydrogen, —COR²⁰, —CO₂R²⁰, —CONR²⁰R³⁷, and SO₂R²⁰, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;

R³⁵ is selected from the group consisting of hydrogen, —COR²⁰, —CO₂R²⁰, CONR²⁰R³⁷, and SO₂R²⁰, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;

R³⁰ is selected from the group consisting of hydrogen an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl; and

R³¹ is selected from the group consisting of hydrogen, a halogen, and —OR¹⁶;

wherein,

at least one of R¹, R² and R⁴ is not hydrogen; and

at least one of R¹¹, R¹², and one R¹³ is not hydrogen.

In certain embodiments, the invention provides a selective glucocorticoid receptor modulator. In certain embodiments, the invention provides a selective glucocorticoid receptor agonist. In certain embodiments, the invention provides a selective glucocorticoid receptor antagonist. In certain embodiments, the invention provides a selective glucocorticoid receptor partial agonist. In certain embodiments, the invention provides a selective glucocorticoid receptor binding compound.

In certain embodiments, the invention provides a selective mineralocorticoid receptor modulator. In certain embodiments, the invention provides a selective mineralocorticoid receptor agonist. In certain embodiments, the invention provides a selective mineralocorticoid receptor antagonist. In certain embodiments, the invention provides a selective mineralocorticoid receptor partial agonist. In certain embodiments, the invention provides a selective mineralocorticoid receptor binding compound.

In certain embodiments, the invention provides a selective glucocorticoid/mineralocorticoid receptor modulator. In certain embodiments, the invention provides a selective glucocorticoid/mineralocorticoid receptor agonist. In certain embodiments, the invention provides a selective glucocorticoid/mineralocorticoid receptor antagonist. In certain embodiments, the invention provides a selective glucocorticoid/mineralocorticoid receptor partial agonist. In certain embodiments, the invention provides a selective glucocorticoid/mineralocorticoid receptor binding compound.

In certain embodiments, the invention provides a pharmaceutical agent comprising a physiologically acceptable carrier, diluent, and/or excipient; and one or more compound of the present invention.

In certain embodiments, the invention provides a compound for treating a patient. In certain embodiments, the invention provides a compound for the treatment of a condition selected from the group consisting of, inflammation, transplant rejection, psoriasis, dermatitis, autoimmune disorder, malignancy, adrenal insufficiency, congenital adrenal hyperplasia, rheumatic fever, granulomatous disease, immune proliferation/apoptosis, conditions of the HPA axis, hypercortisolemia, cytokine imbalance, kidney disease, liver disease, stroke, spinal cord injury, hypercalcemia, hyperglycemia, cerebral edema, thrombocytopenia, Little's syndrome, Addison's disease, cystic fibrosis, myasthenia gravis, autoimmune hemolytic anemia, uveitis, pemphigus vulgaris, multiple sclerosis, nasal polyps, sepsis, infections, type II diabetes, obesity, metabolic syndrome, depression, schizophrenia, mood disorders, Cushing's syndrome, anxiety, sleep disorders, poor memory, glaucoma, wasting, heart disease, fibrosis, hypertension, hyperaldosteronism, and sodium and/or potassium imbalance.

In certain embodiments, the invention provides a method for modulating activity of a glucocorticoid receptor. Certain such methods comprise contacting a glucocorticoid receptor with one or more compounds of the present invention.

In certain embodiments, the invention provides a method for modulating activity of a mineralocorticoid receptor. Certain such methods comprise contacting a mineralocorticoid receptor with one or more compounds of the present invention.

In certain embodiments, the invention provides a method for modulating both the activity of a glucocorticoid receptor and the activity of a mineralocorticoid receptor. Certain such methods comprise contacting a mineralocorticoid receptor and a glucocorticoid receptor with one or more compounds of the present invention.

In certain embodiments, the invention provides a method for identifying a compound that is capable of modulating activity of a glucocorticoid receptor and/or a mineralocorticoid receptor comprising contacting a cell expressing a glucocorticoid receptor and/or a mineralocorticoid receptor with a compound of the present invention and monitoring an effect on the cell. In certain such embodiments, the compound is a quinoline. In certain such embodiments, the compound is derived from a quinoline. In certain embodiments, the compound is a 6-arylquinoline.

In certain embodiments, the invention provides methods of treating a patient comprising administering to the patient a compound of the present invention. In certain embodiments, the invention provides a method of treating a condition selected from the group consisting of inflammation, transplant rejection, psoriasis, dermatitis, autoimmune disorder, malignancy, adrenal insufficiency, congenital adrenal hyperplasia, rheumatic fever, granulomatous disease, immune proliferation/apoptosis, conditions of the HPA axis, hypercortisolemia, cytokine imbalance, kidney disease, liver disease, stroke, spinal cord injury, hypercalcemia, hyperglycemia, cerebral edema, thrombocytopenia, Little's syndrome, Addison's disease, cystic fibrosis, myasthenia gravis, autoimmune hemolytic anemia, uveitis, pemphigus vulgaris, multiple sclerosis, nasal polyps, sepsis, infections, type II diabetes, obesity, metabolic syndrome, depression, schizophrenia, mood disorders, Cushing's syndrome, anxiety, sleep disorders, poor memory, glaucoma, wasting, heart disease, fibrosis, hypertension, hyperaldosteronism, and sodium and/or potassium imbalance.

### DETAILED DESCRIPTION

It is to be understood that both the foregoing general description and the following detailed description are exemplary and explanatory only and are not restrictive of the invention claimed. In this application, the use of the singular includes the plural unless specifically stated otherwise. In this application, the use of “or” means “and/or” unless stated otherwise. Furthermore, use of the term “including” as well as other forms, such as “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

Definitions

Unless specific definitions are provided, the nomenclatures utilized in connection with, and the laboratory procedures and techniques of, analytical chemistry, synthetic organic chemistry, and medicinal and pharmaceutical chemistry described herein are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, pharmaceutical preparation, formulation, and delivery, and treatment of patients. Standard techniques may be used for recombinant DNA, oligonucleotide synthesis, and tissue culture and transformation (e.g. electroporation, lipofection). Reactions and purification techniques may be performed e.g. using kits according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g. Sambrook et al. Molecular Cloning: A Laboratory Manual (2d ed., Cold Spring Harbor Laboratory Press, Cold Spring Harbor, N.Y. (1989)), which is incorporated herein by reference for any purpose.

As used herein, the following terms are defined with the following meanings, unless expressly stated otherwise.

The term “selective binding compound” refers to a compound that selectively binds to any portion of one or more target receptors.

The term “selective glucocorticoid receptor binding compound” refers to a compound that selectively binds to any portion of a glucocorticoid receptor.

The term “selective mineralocorticoid receptor binding compound” refers to a compound that selectively binds to any portion of a mineralocorticoid receptor.

The term “selective glucocorticoid/mineralocorticoid receptor binding compound” refers to a compound that selectively binds to any portion of a glucocorticoid receptor and that also binds to any portion of a mineralocorticoid receptor.

The term “selectively binds” refers to the ability of a selective binding compound to bind to a target receptor with greater affinity than it binds to a non-target receptor. In certain embodiments, selective binding refers to binding to a target with an affinity that is at least 10, 50, 100, 250, 500, or 1000 times greater than the affinity for a non-target.

The term “target receptor” refers to a receptor or a portion of a receptor capable of being bound by a selective binding compound. In certain embodiments, a target receptor is a glucocorticoid receptor. In certain embodiments, a target receptor is a mineralocorticoid receptor. In certain embodiments, glucocorticoid receptors and mineralocorticoid receptors are both target receptors.

The term “modulator” refers to a compound that alters an activity of a molecule. For example, a modulator may cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

The term “selective modulator” refers to a compound that selectively modulates a target activity.

The term “selective glucocorticoid receptor modulator” refers to a compound that selectively modulates at least one activity associated with a glucocorticoid receptor.

The term “selective mineralocorticoid receptor modulator” refers to a compound that selectively modulates at least one activity associated with a mineralocorticoid receptor.

The term “selective glucocorticoid/mineralocorticoid receptor modulator” refers to a compound that selectively modulates at least one activity associated with a glucocorticoid receptor and at least one activity associated with a mineralocorticoid receptor.

The term “selectively modulates” refers to the ability of a selective modulator to modulate a target activity to a greater extent than it modulates a non-target activity.

The term “target activity” refers to a biological activity capable of being modulated by a selective modulator. Certain exemplary target activities include, but are not limited to, binding affinity, signal transduction, enzymatic activity, tumor growth, and inflammation or inflammation-related processes.

The term “receptor mediated activity” refers to any biological activity that results, either directly or indirectly, from binding of a ligand to a receptor.

The term “agonist” refers to a compound, the presence of which results in a biological activity of a receptor that is the same as the biological activity resulting from the presence of a naturally occurring ligand for the receptor.

The term “partial agonist” refers to a compound the presence of which results in a biological activity of a receptor that is of the same type as that resulting from the presence of a naturally occurring ligand for the receptor, but of a lower magnitude.

The term “antagonist” refers to a compound, the presence of which results in a decrease in the magnitude of a biological activity of a receptor. In certain embodiments, the presence of an antagonist results in complete inhibition of a biological activity of a receptor.

The term “alkyl” refers to an aliphatic hydrocarbon group. An alkyl may be a “saturated alkyl,” which means that it does not contain any alkene or alkyne groups. An alkyl group may be an “unsaturated alkyl,” which means that it comprises at least one alkene or alkyne group. An alkyl, whether saturated or unsaturated, may be branched or straight chain. Alkyls may be substituted or unsubstituted. Alkyls include, but are not limited to, methyl, ethyl, propyl, isopropyl, butyl, isobutyl, tertiary butyl, pentyl, hexyl, ethenyl, propenyl, butenyl, ethynyl, butynyl, propynyl, cyclopropyl, cyclobutyl, cyclopentyl, cyclohexyl, and the like, each of which may be optionally substituted.

In certain embodiments, an alkyl comprises 1 to 20 carbon atoms (whenever it appears herein, a numerical range such as “1 to 20” refers to each integer in the given range; e.g. “1 to 20 carbon atoms” means that an alkyl group may comprise only 1 carbon atom, 2 carbon atoms, 3 carbon atoms, etc., up to and including 20 carbon atoms, although the term “alkyl” also includes instances where no numerical range of carbon atoms is designated).

The term “lower alkyl” refers to an alkyl comprising 1 to 5 carbon atoms. The term “medium alkyl” refers to an alkyl comprising 5 to 10 carbon atoms. An alkyl may be designated as “C₁-C₄ alkyl” or similar designations. By way of example only, “C₁-C₄ alkyl” indicates an alkyl having one, two, three, or four carbon atoms (e.g., methyl, ethyl, propyl, iso-propyl, n-butyl, iso-butyl, sec-butyl, t-butyl, ethenyl, propenyl, butenyl, ethynyl, propynyl, and butynyl).

The term “alkenyl” refers to an alkyl group comprising at least one carbon-carbon double bond.

The term “alkynyl” refers to an alkyl group comprising at least one carbon-carbon triple bond.

The term “haloalkyl” refers to an alkyl in which at least one hydrogen atom is replaced with a halogen atom. In certain of the embodiments in which two or more hydrogen atom are replaced with halogen atoms, the halogen atoms are all the same as one another. In certain of such embodiments, the halogen atoms are not all the same as one another.

The term “heteroalkyl” refers to a group comprising an alkyl and one or more heteroatoms. Certain heteroalkyls are acylalkyls, in which the one or more heteroatoms are within an alkyl chain. Certain other heteroalkyls are acylalkyls, in which the heteroatom is not within the alkyl chain. Examples of heteroalkyls include, but are not limited to, CH₃C(═O)CH₂—, CH₃C(═O)CH₂CH₂—, CH₃CH₂C(═O)CH₂CH₂—, CH₃C(═O)CH₂CH₂CH₂—, CH₃OCH₂CH₂—, CH₃NHCH₂—, and the like.

The term “heterohaloalkyl” refers to a heteroalkyl in which at least one hydrogen atom is replaced with a halogen atom.

The term “carbocycle” refers to a group comprising a covalently closed ring, wherein each of the atoms forming the ring is a carbon atom. Carbocylic rings may be formed by three, four, five, six, seven, eight, nine, or more than nine carbon atoms. Carbocycles may be optionally substituted.

The term “heterocycle” refers to a group comprising a covalently closed ring wherein at least one atom forming the ring is a heteroatom. Heterocyclic rings may be formed by three, four, five, six, seven, eight, nine, or more than nine atoms. Any number of those atoms may be heteroatoms (i.e., a heterocyclic ring may comprise one, two, three, four, five, six, seven, eight, nine, or more than nine heteroatoms). In heterocyclic rings comprising two or more heteroatoms, those two or more heteroatoms may be the same or different from one another. Heterocycles may be optionally substituted. Binding to a heterocycle can be at a heteroatom or via a carbon atom. For example, binding for benzo-fused derivatives, may be via a carbon of the benzenoid ring. Examples of heterocycles include, but are not limited to the following: 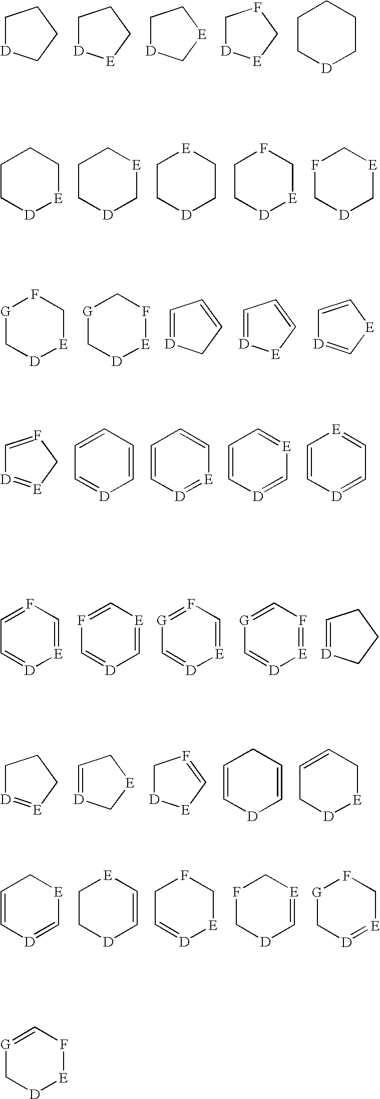
wherein D, E, F, and G independently represent a heteroatom. Each of D, E, F, and G may be the same or different from one another.

The term “heteroatom” refers to an atom other than carbon or hydrogen. Heteroatoms are typically independently selected from the group consisting of oxygen, sulfur, nitrogen, and phosphorus, but are not limited to those atoms. In embodiments in which two or more heteroatoms are present, the two or more heteroatoms may all be the same as one another, or some or all of the two or more heteroatoms may each be different from the others.

The term “aromatic” refers to a group comprising a covalently closed ring having a delocalized π-electron system. Aromatic rings may be formed by five, six, seven, eight, nine, or more than nine atoms. Aromatics may be optionally substituted. Examples of aromatic groups include, but are not limited to phenyl, naphthalenyl, phenanthrenyl, anthracenyl, tetralinyl, fluorenyl, indenyl, and indanyl. The term aromatic includes, for example, benzenoid groups, connected via one of the ring-forming carbon atoms, and optionally carrying one or more substituents selected from the group consisting of an aryl, a heteroaryl, a cycloalkyl, a non-aromatic heterocycle, a halo, a hydroxy, an amino, a cyano, a nitro, an alkylamido, an acyl, a C₁₋₆ alkoxy, a C₁₋₆ alkyl, a C₁₋₆ hydroxyalkyl, a C₁₋₆ aminoalkyl, a C₁₋₆ alkylamino, an alkylsulfenyl, an alkylsulfinyl, an alkylsulfonyl, an sulfamoyl, or a trifluoromethyl. In certain embodiments, an aromatic group is substituted at one or more of the para, meta, and/or ortho positions. Examples of aromatic groups comprising substitutions include, but are not limited to, phenyl, 3-halophenyl, 4-halophenyl, 3-hydroxyphenyl, 4-hydroxyphenyl, 3-aminophenyl, 4-aminophenyl, 3-methylphenyl, 4-methylphenyl, 3-methoxyphenyl, 4-methoxyphenyl, 4-trifluoromethoxyphenyl, 3-cyanophenyl, 4-cyanophenyl, dimethylphenyl, naphthyl, hydroxynaphthyl, hydroxymethylphenyl, (trifluoromethyl)phenyl, alkoxyphenyl, 4-morpholin-4-ylphenyl, 4-pyrrolidin-1-ylphenyl, 4-pyrazolylphenyl, 4-triazolylphenyl, and 4-(2-oxopyrrolidin-1-yl)phenyl.

The term “aryl” refers to an aromatic group wherein each of the atoms forming the ring is a carbon atom. Aryl rings may be formed by five, six, seven, eight, nine, or more than nine carbon atoms. Aryl groups may be optionally substituted.

The term “heteroaryl” refers to an aromatic group wherein at least one atom forming the aromatic ring is a heteroatom. Heteroaryl rings may be formed by three, four, five, six, seven, eight, nine, or more than nine atoms. Heteroaryl groups may be optionally substituted. Examples of heteroaryl groups include, but are not limited to, aromatic C₃₋₈ heterocyclic groups comprising one oxygen or sulfur atom or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl groups are optionally substituted with one or more substituents, independently selected from the group consisting of halo, hydroxy, amino, cyano, nitro, alkylamido, acyl, C₁₋₆-alkoxy, C₁₋₆-alkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl. Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline. In some embodiments, the substituents are halo, hydroxy, cyano, O—C₁₋₆-alkyl, C₁₋₆-alkyl, hydroxy-C₁₋₆-alkyl, and amino-C₁₋₆-alkyl.

The term “non-aromatic ring” refers to a group comprising a covalently closed ring that does not have a delocalized π-electron system.

The term “cycloalkyl” refers to a group comprising a non-aromatic ring wherein each of the atoms forming the ring is a carbon atom. Cycloalkyl rings may be formed by three, four, five, six, seven, eight, nine, or more than nine carbon atoms. Cycloalkyls may be optionally substituted. In certain embodiments, a cycloalkyl comprises one or more unsaturated bonds. Examples of cycloalkyls include, but are not limited to, cyclopropane, cyclobutane, cyclopentane, cyclopentene, cyclopentadiene, cyclohexane, cyclohexene, 1,3-cyclohexadiene, 1,4-cyclohexadiene, cycloheptane, and cycloheptene.

The term “non-aromatic heterocycle” refers to a group comprising a non-aromatic ring wherein one or more atoms forming the ring is a heteroatom. Non-aromatic heterocyclic rings may be formed by three, four, five, six, seven, eight, nine, or more than nine atoms. Non-aromatic heterocycles may be optionally substituted. In certain embodiments, non-aromatic heterocycles comprise one or more carbonyl or thiocarbonyl groups such as, for example, oxo- and thio-containing groups. Examples of non-aromatic heterocycles include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane.

The term “arylalkyl” refers to a group comprising an aryl group bound to an alkyl group.

The term “carbocycloalkyl” refers to a group comprising a carbocyclic cycloalkyl ring. Carbocycloalkyl rings may be formed by three, four, five, six, seven, eight, nine, or more than nine carbon atoms. Carbocycloalkyl groups may be optionally substituted.

The term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g. cycloalkyls and non-aromatic heterocycles). Rings may be optionally substituted. Rings may form part of a ring system.

The term “ring system” refers to two or more rings, wherein two or more of the rings are fused. The term “fused” refers to structures in which two or more rings share one or more bonds.

The term “linked to form a ring” and similar terms refer to instances where two atoms that are bound either to a single atom or to atoms that are bonded or linked through a linking group, are each bound to a linking group, such that the resulting structure forms a ring. That resulting ring includes the two atoms that are linked to form a ring, the atom (or atoms) that previously linked those atoms and the linker. For example, if A and B below are “linked to form a ring” 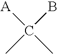
the resulting ring includes A, B, C and a linking group. Unless otherwise indicated, that linking group may be of any length and may be optionally substituted. Referring to the above example, resulting structures include, but are not limited to: 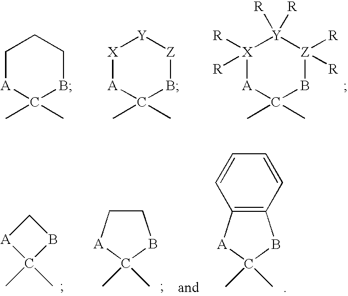
In certain embodiments, the two substituents that together form a ring are not immediately bound to the same atom. For example, if A and B, below, are linked to form a ring: 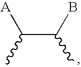
the resulting ring includes A, B, the two atoms that already link A and B and a linking group. Examples of resulting structures include, but are not limited to: 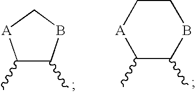
and the like.
In certain embodiments, the atoms that together form a ring are separated by three or more atoms. For example, if A and B, below, are linked to form a ring: 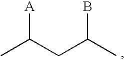
the resulting ring includes A, B, the 3 atoms that already link A and B and a linking group. Examples of resulting structures include, but are not limited to:

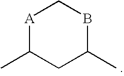

The substituent “R” appearing by itself and without a number designation refers to a substituent selected from the group consisting of hydrogen, alkyl, cycloalkyl, aryl, heteroaryl (bonded through a ring carbon) and non-aromatic heterocycle (bonded through a ring carbon).

The term “O-carboxy” refers to a group of formula RC(═O)O—.

The term “C-carboxy” refers to a group of formula —C(═O)OR.

The term “acetyl” refers to a group of formula —C(═O)CH₃.

The term “trihalomethanesulfonyl” refers to a group of formula X₃CS(═O)₂— where X is a halogen.

The term “cyano” refers to a group of formula —CN.

The term “isocyanato” refers to a group of formula —NCO.

The term “thiocyanato” refers to a group of formula —CNS.

The term “isothiocyanato” refers to a group of formula —NCS.

The term “sulfonyl” refers to a group of formula —S(═O)—R.

The term “S-sulfonamido” refers to a group of formula —S(═O)₂NR.

The term “N-sulfonamido” refers to a group of formula RS(═O)₂NH—.

The term “trihalomethanesulfonamido” refers to a group of formula X₃CS(═O)₂NR—.

The term “O-carbamyl” refers to a group of formula —OC(═O)—NR.

The term “N-carbamyl” refers to a group of formula ROC(═O)NH—.

The term “O-thiocarbamyl” refers to a group of formula —OC(═S)—NR.

The term “N-thiocarbamyl” refers to a group of formula ROC(═S)NH—.

The term “C-amido” refers to a group of formula —C(═O)—NR₂.

The term “N-amido” refers to a group of formula RC(═O)NH—.

The term “ester” refers to a chemical moiety with formula —(R)ₙ—COOR′, where R and R′ are independently selected from the group consisting of alkyl, cycloalkyl, aryl, heteroaryl (bonded through a ring carbon) and non-aromatic heterocycle (bonded through a ring carbon), where n is 0 or 1.

The term “amide” refers to a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), where n is 0 or 1. In certain embodiments, an amide may be an amino acid or a peptide.

The terms “amine,” “hydroxy,” and “carboxyl” include such groups that have been esterified or amidified. Procedures and specific groups used to achieve esterification and amidification are known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

Unless otherwise indicated, the term “optionally substituted,” refers to a group in which none, one, or more than one of the hydrogen atoms has been replaced with one or more group(s) individually and independently selected from the group consisting of: alkyl, heteroalkyl, haloalkyl, heteroholoalkyl, cycloalkyl, aryl, arylalkyl, heteroaryl, non-aromatic heterocycle, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives of amino groups. Such protective derivatives (and protecting groups that may form such protective derivatives) are known to those of skill in the art and may be found in references such as Greene and Wuts, above. In embodiments in which two or more hydrogen atoms have been substituted, the substituent groups may together form a ring.

The term “carrier” refers to a compound that facilitates the incorporation of another compound into cells or tissues. For example, dimethyl sulfoxide (DMSO) is a commonly used carrier for improving incorporation of certain organic compounds into cells or tissues.

The term “pharmaceutical agent” refers to a chemical compound or composition capable of inducing a desired therapeutic effect in a patient. In certain embodiments, a pharmaceutical agent comprises an active agent, which is the agent that induces the desired therapeutic effect. In certain embodiments, a pharmaceutical agent comprises a prodrug. In certain embodiments, a pharmaceutical agent comprises inactive ingredients such as carriers, excipients, and the like.

The term “therapeutically effective amount” refers to an amount of a pharmaceutical agent sufficient to achieve a desired therapeutic effect.

The term “prodrug” refers to an pharmaceutical agent that is converted from a less active form into a corresponding more active form in vivo.

The term “pharmaceutically acceptable” refers to a formulation of a compound that does not significantly abrogate the biological activity, a pharmacological activity and/or other properties of the compound when the formulated compound is administered to a patient. In certain embodiments, a pharmaceutically acceptable formulation does not cause significant irritation to a patient.

The term “co-administer” refers to administering more than one pharmaceutical agent to a patient. In certain embodiments, co-administered pharmaceutical agents are administered together in a single dosage unit. In certain embodiments, co-administered pharmaceutical agents are administered separately. In certain embodiments, co-administered pharmaceutical agents are administered at the same time. In certain embodiments, co-administered pharmaceutical agents are administered at different times.

The term “patient” includes human and animal subjects.

The term “substantially pure” means an object species (e.g., compound) is the predominant species present (i.e., on a molar basis it is more abundant than any other individual species in the composition). In certain embodiments, a substantially purified fraction is a composition wherein the object species comprises at least about 50 percent (on a molar basis) of all species present. In certain embodiments, a substantially pure composition will comprise more than about 80%, 85%, 90%, 95%, or 99% of all species present in the composition. In certain embodiments, the object species is purified to essential homogeneity (contaminant species cannot be detected in the composition by conventional detection methods) wherein the composition consists essentially of a single species.

The term “tissue-selective” refers to the ability of a compound to modulate a biological activity in one tissue to a greater or lesser degree than it modulates a biological activity in another tissue. The biological activities in the different tissues may be the same or they may be different. The biological activities in the different tissues may be mediated by the same type of target receptor. For example, in certain embodiments, a tissue-selective compound may modulate receptor mediated biological activity in one tissue and fail to modulate, or modulate to a lesser degree, receptor mediated biological activity in another tissue type.

The term “monitoring” refers to observing an effect or absence of any effect. In certain embodiments, one monitors cells after contacting those cells with a compound of the present invention. Examples of effects that may be monitored include, but are not limited to, changes in cell phenotype, cell proliferation, receptor activity, or the interaction between a receptor and a compound known to bind to the receptor.

The term “cell phenotype” refers to physical or biological characteristics. Examples of characteristics that constitute phenotype included, but are not limited to, cell size, cell proliferation, cell differentiation, cell survival, apoptosis (cell death), or the utilization of a metabolic nutrient (e.g., glucose uptake). Certain changes or the absence of changes in cell phenotype are readily monitored using techniques known in the art.

The term “cell proliferation” refers to the rate at which cells divide. The number of cells growing in a vessel can be quantified by a person skilled in the art (e.g., by counting cells in a defined area using a light microscope, or by using laboratory apparatus that measure the density of cells in an appropriate medium). One skilled in that art can calculate cell proliferation by determining the number of cells at two or more times.

The term “contacting” refers to bringing two or more materials into close enough proximity that they may interact. In certain embodiments, contacting can be accomplished in a vessel such as a test tube, a petri dish, or the like. In certain embodiments, contacting may be performed in the presence of additional materials. In certain embodiments, contacting may be performed in the presence of cells. In certain of such embodiments, one or more of the materials that are being contacted may be inside a cell. Cells may be alive or may dead. Cells may or may not be intact.

Certain Compounds

Certain compounds that bind to glucocorticoid receptors and/or mineralocorticoid receptors and/or certain compounds that modulate an activity of such receptors play a role in health (e.g. normal growth, development, and/or absence of disease). In certain embodiments, compounds of the present invention are useful for treating any of a variety of diseases or conditions.

Certain compounds have been previously described as receptor modulators or as possible receptor modulators. See e.g. U.S. Pat. Nos. 6,462,038, 5,693,646; 6,380,207; 6,506,766; 5,688,810; 5,696,133; 6,569,896, 6,673,799; 4,636,505; 4,097,578; 3,847,988; U.S. application Ser. No. 10/209,461 (Pub. No. US 2003/0055094); WO 01/27086; WO 02/22585; Zhi, et al. Bioorganic & Medicinal Chemistry Letters 2000, 10, 415-418; Pooley, et. al., J. Med. Chem. 1998, 41, 3461; Hamann, et al. J. Med. Chem. 1998, 41 (4), 623; and Yin, et al., Molecular Pharmacology, 2003, 63 (1), 211-223 the entire disclosures of which are incorporated in their entirety.

In certain embodiments, the present invention provides selective glucocorticoid and/or mineralocorticoid receptor modulators. In certain embodiments, the invention provides selective glucocorticoid and/or mineralocorticoid receptor binding agents. In certain embodiments, the invention provides methods of making and methods of using selective glucocorticoid and/or mineralocorticoid receptor modulators and/or selective glucocorticoid and/or mineralocorticoid binding agents. In certain embodiments, selective glucocorticoid and/or mineralocorticoid modulators are agonists, partial agonists, and/or antagonists for the glucocorticoid and/or mineralocorticoid receptor.

In certain embodiments, the present invention relates to compounds of Formula I, II, or III: 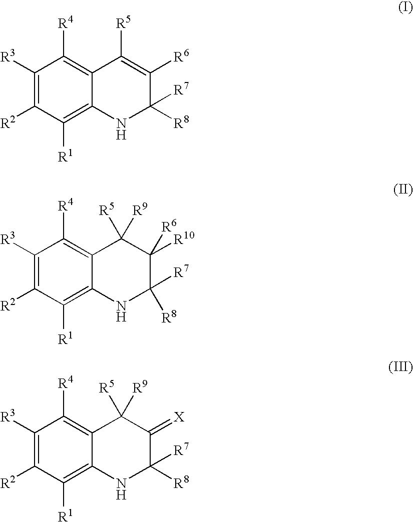
or a pharmaceutically acceptable salt, ester, amide, or prodrug thereof.

In certain embodiments, R¹ is selected from the group consisting of hydrogen, a halogen, —CN, —OR¹⁶, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R¹ is an optionally substituted C₁-C₈ alkyl or an optionally substituted C₃-C₈ cycloalkyl that is fully saturated. In certain embodiments, R¹ is an optionally substituted C₁-C₈ alkyl or an optionally substituted C₃-C₈ cycloalkyl that is not fully saturated. In certain such embodiments, R¹ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R¹ is selected from the group consisting of an optionally substituted methyl, ethyl propyl isopropyl, butyl, sec-butyl, and tert-butyl. In certain embodiments, R¹ is methyl. In certain embodiments, R¹ is trifluoromethyl. In certain of the embodiments where R¹ is a halogen, R¹ is F or Cl.

In certain embodiments, R² is selected from the group consisting of hydrogen, a halogen, —CN, —OR¹⁶, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R² is an optionally substituted C₁-C₈ alkyl or an optionally substituted C₃-C₈ cycloalkyl that is fully saturated. In certain embodiments, R² is an optionally substituted C₁-C₈ alkyl or an optionally substituted C₃-C₈ cycloalkyl that is not fully saturated. In certain such embodiments, R² is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R² is selected from the group consisting of an optionally substituted methyl, ethyl propyl isopropyl, butyl, sec-butyl, and tert-butyl. In certain embodiments, R² is methyl. In certain embodiments, R² is trifluoromethyl. In certain of the embodiments where R² is a halogen, R² is F or Cl.

In certain embodiments, R³ is selected from the group consisting of (a), (b), (c), (d), (e), (f), (g), (h), (i), (j), (k), (l), (m), and (n):

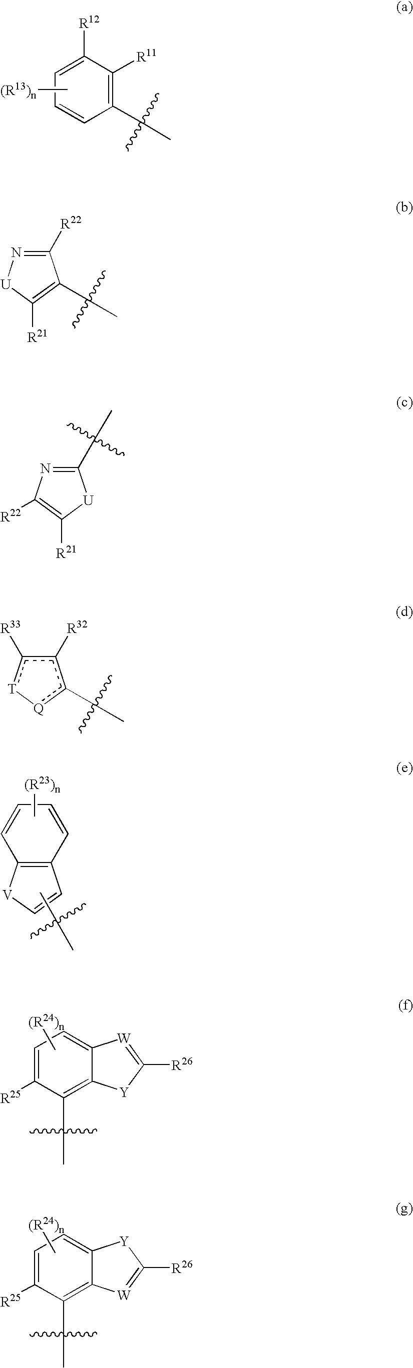

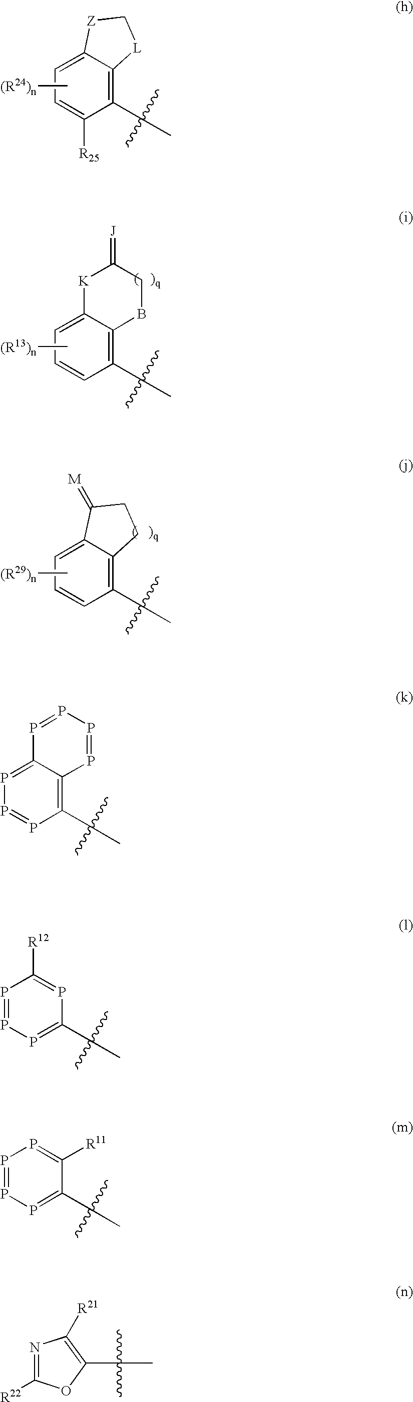

In certain embodiments, R³ is selected from the group consisting of an optionally substituted 2-indolyl, an optionally substituted 3-indolyl, an optionally substituted 4-indolyl, an optionally substituted 6-indolyl, an optionally substituted 7-indolyl, and an optionally substituted 7-indolinyl. In certain embodiments, R³ is a pyridyl, optionally substituted with a C₁-C₆ alkyl, where that alkyl is selected from the group consisting of methyl, ethyl, propyl, isopropyl, butyl, sec-butyl, and tert-butyl. In certain embodiments, R³ is 3-methylpyrid-2-yl. In certain embodiments, R³ is an optionally substituted dibenzofuranyl. In certain embodiments, R³ is 2,3-dihydro-1,4-benzodioxin-6-yl. In certain embodiments, R³ is

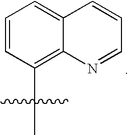

In certain embodiments, R⁴ is selected from the group consisting of hydrogen, a halogen, NO₂, OR⁹, NR¹⁰R¹¹, CN, C═N(OR¹⁶), CO₂R², CONR²⁰R³⁷, NR¹⁷(OR¹⁶), CR³(OR¹⁶), an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R⁴ is an optionally substituted C₁-C₈ alkyl or an optionally substituted C₃-C₈ cycloalkyl that is fully saturated. In certain embodiments, R⁴ is an optionally substituted C₁-C₈ alkyl or an optionally substituted C₃-C₈ cycloalkyl that is not fully saturated. In certain such embodiments, R⁴ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R⁴ is selected from the group consisting of an optionally substituted methyl, ethyl propyl isopropyl, butyl, sec-butyl, and tert-butyl. In certain embodiments, R⁴ is methyl. In certain embodiments, R⁴ is trifluoromethyl. In certain of the embodiments where R⁴ is a halogen, R⁴ is F or Cl.

In certain embodiments at least one of R¹, R² and R⁴ is not hydrogen. In certain embodiments at least two of R¹, R² and R⁴ are not hydrogen. In certain embodiments, at least one of R¹, R² and R⁴ is not methyl. In certain embodiments, if one of R¹, R² and R⁴ is hydrogen, then at least one of the other two of those groups is not methyl.

In certain embodiments, R⁵ is selected from the group consisting of hydrogen, a halogen, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R⁵ is an optionally substituted C₁-C₈ alkyl or an optionally substituted C₃-C₈ cycloalkyl that is fully saturated. In certain embodiments, R⁵ is an optionally substituted C₁-C₈ alkyl or an optionally substituted C₃-C₈ cycloalkyl that is not fully saturated. In certain such embodiments, R⁵ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R⁵ is selected from the group consisting of an optionally substituted methyl, ethyl propyl isopropyl, butyl, sec-butyl, and tert-butyl. In certain embodiments, R⁵ is methyl. In certain embodiments, R⁵ is trifluoromethyl. In certain of the embodiments where R⁵ is a halogen, R⁵ is F or Cl.

In certain embodiments in which R⁵ is a heteroalkyl, the heteroatom of that heteroalkyl is not sulfur or oxygen. In certain of the embodiments where R⁵ is an optionally substituted alkyl, that optionally substituted alkyl is optionally substituted with one or more substituents selected from the group consisting of an aryl, a heteroaryl, a cycloalkyl, and a heterocycle. In certain such embodiments, the optionally substituted alkyl is optionally substituted phenyl. In certain of the embodiments where R⁵ is an optionally substituted alkenyl, that optionally substituted alkenyl is selected from the group consisting of optionally substituted ethenyl, propenyl, butenyl, and pentenyl each of which is optionally substituted with one or more substituents selected from the group consisting of alkyl, aryl, heteroaryl, cycloalkyl, and heterocycle. In certain embodiments, R⁵ is selected from the group consisting of hydrogen, methyl, benzyl, 3-methyl-2-butenyl, and 2-propenyl.

In certain embodiments, R⁶ is selected from the group consisting of hydrogen and OR¹⁶. In certain embodiments, R⁶ is hydroxy.

In certain embodiments, each of R⁷ and R⁸ is independently selected from the group consisting of hydrogen, a halogen, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R⁷ and/or R⁸ is an optionally substituted C₁-C₈ alkyl or an optionally substituted C₃-C₈ cycloalkyl that is fully saturated. In certain embodiments, R⁷ and/or R⁸ is an optionally substituted C₁-C₈ alkyl or an optionally substituted C₃-C₈ cycloalkyl that is not fully saturated. In certain such embodiments, R⁷ and/or R⁸ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R⁷ and/or R⁸ is selected from the group consisting of an optionally substituted methyl, ethyl propyl isopropyl, butyl, sec-butyl, and tert-butyl. In certain embodiments, R⁷ and/or R⁸ is methyl. In certain embodiments, R⁷ and/or R⁸ is trifluoromethyl. In certain of the embodiments where R⁷ and/or R⁸ is a halogen, R⁷ and/or R⁸ is F or Cl. In certain embodiments, R⁷ is methyl. In certain embodiments R⁸ is methyl. In certain embodiments, R⁷ is methyl and R⁸ is methyl. In certain embodiments, at least one of R⁷ and R⁸ is not methyl. In certain embodiments, at least one of R⁷ and R⁸ is not hydrogen. In certain embodiments, if R⁷ is hydrogen, then R⁸ is not methyl.

In certain embodiments, R⁹ is selected from the group consisting of hydrogen, OR¹⁶, a halogen, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R⁹ is an optionally substituted C₁-C₈ alkyl or an optionally substituted C₃-C₈ cycloalkyl that is fully saturated. In certain embodiments, R⁹ is an optionally substituted C₁-C₈ alkyl or an optionally substituted C₃-C₈ cycloalkyl that is not fully saturated. In certain such embodiments, R⁹ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R⁹ is selected from the group consisting of an optionally substituted methyl, ethyl propyl isopropyl, butyl, sec-butyl, and tert-butyl. In certain embodiments, R⁹ is methyl. In certain embodiments, R⁹ is trifluoromethyl. In certain of the embodiments where R⁹ is a halogen, R⁹ is F or Cl. In certain embodiments, R⁹ is selected from the group consisting of hydrogen, methyl, and hydroxy.

In certain embodiments, R¹⁰ is selected from the group consisting of hydrogen and OR¹⁶. In certain embodiments, R¹⁰ is hydroxy.

In certain embodiments, R¹¹ is selected from the group consisting of hydrogen, a halogen, —CN, —OR¹⁶, —NR¹⁷R¹⁸, —CH₂R¹⁶, COR²⁰, CO₂R²⁰, CONR²⁰R³⁷, —SOR²⁰, —SO₂R²⁰, —NO₂, NR¹⁷(OR¹⁶), an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R¹¹ is an optionally substituted C₁-C₈ alkyl or an optionally substituted C₃-C₈ cycloalkyl that is fully saturated. In certain embodiments, R¹¹ is an optionally substituted C₁-C₈ alkyl or an optionally substituted C₃-C₈ cycloalkyl that is not fully saturated. In certain such embodiments, R¹¹ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R¹¹ is selected from the group consisting of an optionally substituted methyl, ethyl propyl isopropyl, butyl, sec-butyl, and tert-butyl. In certain embodiments, R¹¹ is methyl. In certain embodiments, R¹¹ is trifluoromethyl. In certain of the embodiments where R¹¹ is a halogen, R¹¹ is F or Cl. In certain embodiments, where R¹¹ is an optionally substituted alkenyl, that optionally substituted alkenyl is selected from the group consisting of optionally substituted ethenyl, propenyl, butenyl, and pentenyl. In certain embodiments where R¹¹ is an optionally substituted alkenyl, that optionally substituted alkenyl is optionally substituted with one or more substituents, independently selected from the group consisting of an alkyl, an aryl, a heteroaryl, a cycloalkyl, and a heterocycle. In certain embodiments, R¹¹ is a perfluoroalkyl. In certain such embodiments, R¹¹ is trifluoromethyl. In certain embodiments, R¹¹ is an aryl. In certain such embodiments, R¹¹ is phenyl. In certain embodiments, R¹¹ is selected from the group consisting of methyl, hydroxy, methoxy, benzyloxy, phenyl, fluoro, chloro, trifluoromethyl, trifluoromethoxy, —NH₂, —NO₂, —C(O)CH₃, and 2-methyl-2-butenyl.

In certain embodiments, R¹² is selected from the group consisting of hydrogen, a halogen, —CN, —NR¹⁷SO₂R²⁰, —COR²⁰, —CO₂R²⁰, —CONR²⁰R²⁰, NR¹⁷CO₂R²⁰, —NO₂, —OR¹⁶, —CN, —NH₂, —NHC(O)OCH₃, —NHC(O)O^{t}Bu, —NHSO₂CH₃, —NR¹⁷R¹⁸, NR¹⁷(OR¹⁶) an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R¹² is an optionally substituted C₁-C₈ alkyl or an optionally substituted C₃-C₈ cycloalkyl that is fully saturated. In certain embodiments, R¹² is an optionally substituted C₁-C₈ alkyl or an optionally substituted C₃-C₈ cycloalkyl that is not fully saturated. In certain such embodiments, R¹² is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R¹² is selected from the group consisting of an optionally substituted methyl, ethyl propyl isopropyl, butyl, sec-butyl, and tert-butyl. In certain embodiments, R¹² is methyl. In certain embodiments, R¹² is trifluoromethyl. In certain of the embodiments where R¹² is a halogen, R¹² is F or Cl. In certain of the embodiments where R¹² is an optionally substituted haloalkyl, that optionally substituted haloalkyl is an optionally substituted fluoroalkyl. In certain embodiments, R¹¹ and R¹² are linked together to form a 3-7 membered ring. In one embodiment, the 3-7 membered ring is a phenyl group.

In certain embodiments, each R¹³ is independently selected from the group consisting of hydrogen, a halogen, CN, —NO₂, —OCH₃, OR¹⁶, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R¹³ is an optionally substituted C₁-C₈ alkyl or an optionally substituted C₃-C₈ cycloalkyl that is fully saturated. In certain embodiments, R¹³ is an optionally substituted C₁-C₈ alkyl or an optionally substituted C₃-C₈ cycloalkyl that is not fully saturated. In certain such embodiments, R¹³ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R¹³ is selected from the group consisting of an optionally substituted methyl, ethyl propyl isopropyl, butyl, sec-butyl, and tert-butyl. In certain embodiments, R¹³ is methyl. In certain embodiments, R¹³ is trifluoromethyl. In certain of the embodiments where R¹³ is a halogen, R¹³ is F or Cl. In certain embodiments, R¹² and P R¹³ are linked together to form a 3-7 membered ring. In one embodiment, the 3-7 membered ring is a phenyl group.

In certain embodiments, at least one of R¹¹, R¹², and one R¹³ is not hydrogen. In certain embodiments, at least two of R¹¹, R¹², and one R¹³ are not hydrogen. In certain embodiments, if any of R¹¹, R¹², or one R¹³ is hydrogen, then at least one of the other two of those groups is not methyl.

In certain embodiments, each R¹⁶ is independently selected from the group consisting of hydrogen, a halogen, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R¹⁶ is an optionally substituted C₁-C₈ alkyl or an optionally substituted C₃-C₈ cycloalkyl that is fully saturated. In certain embodiments, R¹⁶ is an optionally substituted C₁-C₈ alkyl or an optionally substituted C₃-C₈ cycloalkyl that is not fully saturated. In certain such embodiments, R¹⁶ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R¹⁶ is selected from the group consisting of an optionally substituted methyl, ethyl propyl isopropyl, butyl, sec-butyl, and tert-butyl. In certain embodiments, R¹⁶ is methyl. In certain embodiments, R¹⁶ is trifluoromethyl. In certain of the embodiments where R¹⁶ is a halogen, R¹⁶ is F or Cl. In certain such embodiments, those optionally substituted methyl, ethyl, isopropyl, butyl, sec-butyl, and tert-butyl groups are optionally substituted with one or more substituents independently selected from the group consisting of optionally substituted alkyl, aryl, heteroaryl, cycloalkyl, and heterocycle. In certain embodiments, R¹⁶ is a perfluoroalkyl.

In certain embodiments, each R¹⁷ is independently selected from the group consisting of hydrogen, a halogen, COR²⁰, CO₂R²⁰, SO₂R²⁰, and S(O)R²⁰, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R¹⁷ is an optionally substituted C₁-C₈ alkyl or an optionally substituted C₃-C₈ cycloalkyl that is fully saturated. In certain embodiments, R¹⁷ is an optionally substituted C₁-C₈ alkyl or an optionally substituted C₃-C₈ cycloalkyl that is not fully saturated. In certain such embodiments, R¹⁷ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R¹⁷ is selected from the group consisting of an optionally substituted methyl, ethyl propyl isopropyl, butyl, sec-butyl, and tert-butyl. In certain embodiments, R¹⁷ is methyl. In certain embodiments, R¹⁷ is trifluoromethyl. In certain of the embodiments where R¹⁷ is a halogen, R¹¹ is F or Cl.

In certain embodiments, each R¹⁸ is independently selected from the group consisting of hydrogen, a halogen, COR²⁰, CO₂R²⁰, SO₂R²⁰, and S(O)R²⁰, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R¹⁸ is an optionally substituted C₁-C₈ alkyl or an optionally substituted C₃-C₈ cycloalkyl that is fully saturated. In certain embodiments, R¹⁸ is an optionally substituted C₁-C₈ alkyl or an optionally substituted C₃-C₈ cycloalkyl that is not fully saturated. In certain such embodiments, R¹⁸ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R¹⁸ is selected from the group consisting of an optionally substituted methyl, ethyl propyl isopropyl, butyl, sec-butyl, and tert-butyl. In certain embodiments, R¹⁸ is methyl. In certain embodiments, R¹⁸ is trifluoromethyl. In certain of the embodiments where R¹⁸ is a halogen, R¹⁸ is F or Cl.

In certain embodiments, R¹⁷ and R¹⁸ are linked to form a ring. In certain such embodiments, the ring has 3-7 members. In certain embodiments, the ring is aromatic. In certain embodiments, the ring is non-aromatic.

In certain embodiments, each R²⁰ is independently selected from the group consisting of hydrogen, a halogen, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R²⁰ is an optionally substituted C₁-C₈ alkyl or an optionally substituted C₃-C₈ cycloalkyl that is fully saturated. In certain embodiments, R²⁰ is an optionally substituted C₁-C₈ alkyl or an optionally substituted C₃-C₈ cycloalkyl that is not fully saturated. In certain such embodiments, R²⁰ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R²⁰ is selected from the group consisting of an optionally substituted methyl, ethyl propyl isopropyl, butyl, sec-butyl, and tert-butyl. In certain embodiments, R²⁰ is methyl. In certain embodiments, R²⁰ is trifluoromethyl. In certain of the embodiments where R²⁰ is a halogen, R²⁰ is F or Cl.

In certain embodiments, each R³⁷ is independently selected from the group consisting of hydrogen, a halogen, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R³⁷ is an optionally substituted C₁-C₈ alkyl or an optionally substituted C₃-C₈ cycloalkyl that is fully saturated. In certain embodiments, R³⁷ is an optionally substituted C₁-C₈ alkyl or an optionally substituted C₃-C₈ cycloalkyl that is not fully saturated. In certain such embodiments, R³⁷ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R³⁷ is selected from the group consisting of an optionally substituted methyl, ethyl propyl isopropyl, butyl, sec-butyl, and tert-butyl. In certain embodiments, R³⁷ is methyl. In certain embodiments, R³⁷ is trifluoromethyl. In certain of the embodiments where R³⁷ is a halogen, R³⁷ is F or Cl.

In certain embodiments, R²⁰ and R³⁷ are linked to form a ring. In certain such embodiments, the ring has 3-7 members. In certain embodiments, the ring is aromatic. In certain embodiments, the ring is non-aromatic.

In certain embodiments, R²¹ is selected from the group consisting of hydrogen, a halogen, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R²¹ is an optionally substituted C₁-C₈ alkyl or an optionally substituted C₃-C₈ cycloalkyl that is fully saturated. In certain embodiments, R²¹ is an optionally substituted C₁-C₈ alkyl or an optionally substituted C₃-C₈ cycloalkyl that is not fully saturated. In certain such embodiments, R²¹ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R²¹ is selected from the group consisting of an optionally substituted methyl, ethyl propyl isopropyl, butyl, sec-butyl, and tert-butyl. In certain embodiments, R²¹ is methyl. In certain embodiments, R²¹ is trifluoromethyl. In certain of the embodiments where R²¹ is a halogen, R²¹ is F or Cl.

In certain embodiments, R²² is selected from the group consisting of hydrogen, a halogen, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R²² is an optionally substituted C₁-C₈ alkyl or an optionally substituted C₃-C₈ cycloalkyl that is fully saturated. In certain embodiments, R²² is an optionally substituted C₁-C₈ alkyl or an optionally substituted C₃-C₈ cycloalkyl that is not fully saturated. In certain such embodiments, R²² is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R²² is selected from the group consisting of an optionally substituted methyl, ethyl propyl isopropyl, butyl, sec-butyl, and tert-butyl. In certain embodiments, R²² is methyl. In certain embodiments, R²² is trifluoromethyl. In certain of the embodiments where R²² is a halogen, R²² is F or Cl.

In certain embodiments, each R²³ is independently and selected from the group consisting of hydrogen, a halogen, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R²³ is an optionally substituted C₁-C₈ alkyl or an optionally substituted C₃-C₈ cycloalkyl that is fully saturated. In certain embodiments, R²³ is an optionally substituted C₁-C₈ alkyl or an optionally substituted C₃-C₈ cycloalkyl that is not fully saturated. In certain such embodiments, R²³ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R²³ is selected from the group consisting of an optionally substituted methyl, ethyl propyl isopropyl, butyl, sec-butyl, and tert-butyl. In certain embodiments, R²³ is methyl. In certain embodiments, R²³ is trifluoromethyl. In certain of the embodiments where R²³ is a halogen, R²³ is F or Cl.

In certain embodiments, R²⁴ is selected from the group consisting of hydrogen, a halogen, —OR¹⁶, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R²⁴ is an optionally substituted C₁-C₈ alkyl or an optionally substituted C₃-C₈ cycloalkyl that is fully saturated. In certain embodiments, R²⁴ is an optionally substituted C₁-C₈ alkyl or an optionally substituted C₃-C₈ cycloalkyl that is not fully saturated. In certain such embodiments, R²⁴ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R²⁴ is selected from the group consisting of an optionally substituted methyl, ethyl propyl isopropyl, butyl, sec-butyl, and tert-butyl. In certain embodiments, R²⁴ is methyl. In certain embodiments, R²⁴ is trifluoromethyl. In certain of the embodiments where R²⁴ is a halogen, R²⁴ is F or Cl. In certain embodiments, R²⁴ is methoxy.

In certain embodiments, R²⁵ is selected from the group consisting of hydrogen, a halogen, —OR¹⁶, —CN, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R²⁵ is an optionally substituted C₁-C₈ alkyl or an optionally substituted C₃-C₈ cycloalkyl that is fully saturated. In certain embodiments, R²⁵ is an optionally substituted C₁-C₈ alkyl or an optionally substituted C₃-C₈ cycloalkyl that is not fully saturated. In certain such embodiments, R²⁵ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R²⁵ is selected from the group consisting of an optionally substituted methyl, ethyl propyl isopropyl, butyl, sec-butyl, and tert-butyl. In certain embodiments, R²⁵ is methyl. In certain embodiments, R²⁵ is trifluoromethyl. In certain of the embodiments where R²⁵ is a halogen, R²⁵ is F or Cl. In certain embodiments, R²⁵ is methoxy.

In certain embodiments, R²⁶ is selected from the group consisting of hydrogen, a halogen, CO₂R²⁰, COR²⁰, CONR²⁰R³⁷, C═N(OR¹⁶), an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R²⁶ is an optionally substituted C₁-C₈ alkyl or an optionally substituted C₃-C₈ cycloalkyl that is fully saturated. In certain embodiments, R²⁶ is an optionally substituted C₁-C₈ alkyl or an optionally substituted C₃-C₈ cycloalkyl that is not fully saturated. In certain such embodiments, R²⁶ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R²⁶ is selected from the group consisting of an optionally substituted methyl, ethyl propyl isopropyl, butyl, sec-butyl, and tert-butyl. In certain embodiments, R²⁶ is methyl. In certain embodiments, R²⁶ is trifluoromethyl. In certain of the embodiments where R²⁶ is a halogen, R²⁶ is F or Cl.

In certain embodiments, R²⁷ is selected from the group consisting of hydrogen, a halogen, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R²⁷ is an optionally substituted C₁-C₈ alkyl or an optionally substituted C₃-C₈ cycloalkyl that is fully saturated. In certain embodiments, R²⁷ is an optionally substituted C₁-C₈ alkyl or an optionally substituted C₃-C₈ cycloalkyl that is not fully saturated. In certain such embodiments, R²⁷ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R²⁷ is selected from the group consisting of an optionally substituted methyl, ethyl propyl isopropyl, butyl, sec-butyl, and tert-butyl. In certain embodiments, R²⁷ is methyl. In certain embodiments, R¹¹ is trifluoromethyl. In certain of the embodiments where R²⁷ is a halogen, R²⁷ is F. Br, or Cl. In certain embodiments R²⁷ is —CH₂CH₂C(O)CH₃. In certain embodiments, R²⁶ and R²⁷ are linked together to form a 3-7 membered ring. In one embodiment, the 3-7 membered ring is a phenyl group.

In certain embodiments, R²⁸ is selected from the group consisting of hydrogen, a halogen, —COR²⁰, —CO₂R²⁰, —CONR²⁰, —CONR²⁰R³⁷, SO₂R²⁰, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R²⁸ is an optionally substituted C₁-C₈ alkyl or an optionally substituted C₃-C₈ cycloalkyl that is fully saturated. In certain embodiments, R²⁸ is an optionally substituted C₁-C₈ alkyl or an optionally substituted C₃-C₈ cycloalkyl that is not fully saturated. In certain such embodiments, R²⁸ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R²⁸ is selected from the group consisting of an optionally substituted methyl, ethyl propyl isopropyl, butyl, sec-butyl, and tert-butyl. In certain embodiments, R²⁸ is methyl. In certain embodiments, R²⁸ is trifluoromethyl. In certain of the embodiments where R²⁸ is a halogen, R²⁸ is F or Cl.

In certain embodiments, R²⁹ is selected from the group consisting of hydrogen, a halogen, —OR¹⁶, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R²⁹ is an optionally substituted C₁-C₈ alkyl or an optionally substituted C₃-C₈ cycloalkyl that is fully saturated. In certain embodiments, R²⁹ is an optionally substituted C₁-C₈ alkyl or an optionally substituted C₃-C₈ cycloalkyl that is not fully saturated. In certain such embodiments, R²⁹ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R²⁹ is selected from the group consisting of an optionally substituted methyl, ethyl propyl isopropyl, butyl, sec-butyl, and tert-butyl. In certain embodiments, R²⁹ is methyl. In certain embodiments, R²⁹ is trifluoromethyl. In certain of the embodiments where R²⁹ is a halogen, R²⁹ is F or Cl.

In certain embodiments, R³⁰ is selected from the group consisting of hydrogen, a halogen, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R³⁰ is an optionally substituted C₁-C₈ alkyl or an optionally substituted C₃-C₈ cycloalkyl that is fully saturated. In certain embodiments, R³⁰ is an optionally substituted C₁-C₈ alkyl or an optionally substituted C₃-C₈ cycloalkyl that is not fully saturated. In certain such embodiments, R³⁰ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R³⁰ is selected from the group consisting of an optionally substituted methyl, ethyl propyl isopropyl, butyl, sec-butyl, and tert-butyl. In certain embodiments, R³⁰ is methyl. In certain embodiments, R³⁰ is trifluoromethyl. In certain of the embodiments where R³⁰ is a halogen, R³⁰ is F or Cl.

In certain embodiments, R³¹ is selected from the group consisting of hydrogen, a halogen, —OR¹⁶, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R³¹ is an optionally substituted C₁-C₈ alkyl or an optionally substituted C₃-C₈ cycloalkyl that is fully saturated. In certain embodiments, R³¹ is an optionally substituted C₁-C₈ alkyl or an optionally substituted C₃-C₈ cycloalkyl that is not fully saturated. In certain such embodiments, R³¹ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R³¹ is selected from the group consisting of an optionally substituted methyl, ethyl propyl isopropyl, butyl, sec-butyl, and tert-butyl. In certain embodiments, R³¹ is methyl. In certain embodiments, R³¹ is trifluoromethyl. In certain of the embodiments where R³¹ is a halogen, R³¹ is F or Cl.

In certain embodiments, R³² is selected from the group consisting of hydrogen, a halogen, —OR¹⁶, —CN, —COR²⁰, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R³² is an optionally substituted C₁-C₈ alkyl or an optionally substituted C₃-C₈ cycloalkyl that is fully saturated. In certain embodiments, R³² is an optionally substituted C₁-C₈ alkyl or an optionally substituted C₃-C₈ cycloalkyl that is not fully saturated. In certain such embodiments, R³² is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R³² is selected from the group consisting of an optionally substituted methyl, ethyl propyl isopropyl, butyl, sec-butyl, and tert-butyl. In certain embodiments, R³² is methyl. In certain embodiments, R³² is trifluoromethyl. In certain of the embodiments where R³² is a halogen, R³² is F or Cl.

In certain embodiments, R³³ is selected from the group consisting of hydrogen, a halogen, —OR¹⁶, —CN, —COR²⁰, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R³³ is an optionally substituted C₁-C₈ alkyl or an optionally substituted C₃-C₈ cycloalkyl that is fully saturated. In certain embodiments, R³³ is an optionally substituted C₁-C₈ alkyl or an optionally substituted C₃-C₈ cycloalkyl that is not fully saturated. In certain such embodiments, R³³ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R³³ is selected from the group consisting of an optionally substituted methyl, ethyl propyl isopropyl, butyl, sec-butyl, and tert-butyl. In certain embodiments, R³³ is methyl. In certain embodiments, R³³ is trifluoromethyl. In certain of the embodiments where R³³ is a halogen, R³³ is F or Cl.

In certain embodiments, R³⁴ is selected from the group consisting of hydrogen, a halogen, —NO₂, —OR¹⁶, —NR¹⁷R¹⁸, —CN, —COR²⁰, NR¹⁷(OR¹⁶) an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R³⁴ is an optionally substituted C₁-C₈ alkyl or an optionally substituted C₃-C₈ cycloalkyl that is fully saturated. In certain embodiments, R³⁴ is an optionally substituted C₁-C₈ alkyl or an optionally substituted C₃-C₈ cycloalkyl that is not fully saturated. In certain such embodiments, R³⁴ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R³⁴ is selected from the group consisting of an optionally substituted methyl, ethyl propyl isopropyl, butyl, sec-butyl, and tert-butyl. In certain embodiments, R³⁴ is methyl. In certain embodiments, R³⁴ is trifluoromethyl. In certain of the embodiments where R³³ is a halogen, R³⁴ is F or Cl.

In certain embodiments, R³⁵ is selected from the group consisting of hydrogen, a halogen, —COR²⁰, —CO₂R²⁰, —CONR²⁰, —CONR²⁰R³⁷, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R³⁵ is an optionally substituted C₁-C₈ alkyl or an optionally substituted C₃-C₈ cycloalkyl that is fully saturated. In certain embodiments, R³⁵ is an optionally substituted C₁-C₈ alkyl or an optionally substituted C₃-C₈ cycloalkyl that is not fully saturated. In certain such embodiments, R³⁵ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R³⁵ is selected from the group consisting of an optionally substituted methyl, ethyl propyl isopropyl, butyl, sec-butyl, and tert-butyl. In certain embodiments, R³⁵ is methyl. In certain embodiments, R³⁵ is trifluoromethyl. In certain of the embodiments where R³⁵ is a halogen, R³⁵ is F or Cl.

In certain embodiments, R³⁶ is selected from the group consisting of hydrogen, a halogen, an optionally substituted C₁-C₆ alkyl, an optionally substituted C₁-C₆ heteroalkyl, an optionally substituted C₁-C₆ haloalkyl, an optionally substituted C₁-C₆ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl. In certain embodiments, R³⁶ is an optionally substituted C₁-C₈ alkyl or an optionally substituted C₃-C₈ cycloalkyl that is fully saturated. In certain embodiments, R³⁶ is an optionally substituted C₁-C₈ alkyl or an optionally substituted C₃-C₈ cycloalkyl that is not fully saturated. In certain such embodiments, R³⁶ is selected from the group consisting of an optionally substituted C₂-C₈ alkenyl, an optionally substituted C₂-C₈ alkynyl, an optionally substituted C₃-C₈ cycloalkenyl, and an optionally substituted C₃-C₈ cycloalkynyl. In certain of the embodiments, R³⁶ is selected from the group consisting of an optionally substituted methyl, ethyl propyl isopropyl, butyl, sec-butyl, and tert-butyl. In certain embodiments, R³⁶ is methyl. In certain embodiments, R²⁶ is trifluoromethyl. In certain of the embodiments where R³⁶ is a halogen, R³⁶ is F or Cl.

In certain embodiments, U is selected from the group consisting of oxygen, sulfur, nitrogen, and —NR¹⁷.

In certain embodiments, Q is selected from the group consisting of nitrogen, phosphorous, sulfur, oxygen, —NR¹⁷, and —CR³⁴. In certain embodiments, T selected from the group consisting of nitrogen, phosphorous, sulfur, oxygen, —NR¹⁷, and —CR³⁴. In certain embodiments, Q is —CR³⁴ and T is selected from the group consisting of sulfur, oxygen, and —NR¹⁷. In certain embodiments, T is CR³⁴ and Q is selected from the group consisting of sulfur, oxygen, and —NR¹⁷. In certain embodiments, either one of Q or T is —CR³⁴ and the other is selected from the group consisting of sulfur, oxygen, and —NR¹⁷.

In certain embodiments, V is selected from the group consisting of nitrogen, phosphorous, oxygen, sulfur, and —NR¹⁷.

In certain embodiments, n is selected from the group consisting of 0, 1, 2, 3, and 4. In certain embodiments, q is selected from the group consisting of 0, 1, and 2.

In certain embodiments, W is selected from the group consisting of —CR²⁷ and nitrogen;

In certain embodiments, Y is selected from the group consisting of —NR³⁶, sulfur, and oxygen.

In certain embodiments, Z is selected from the group consisting of CH₂, —NR²⁸, and oxygen. In certain embodiments, L is selected from the group consisting of CH₂, —NR²⁸, and oxygen. In certain embodiments, Z is CH₂ and L is —NR²⁸ or oxygen. In certain embodiments, L is CH₂, and Z is —NR²⁸ or oxygen. In certain embodiments, either one of L or Z is CH₂ and the other is selected from the group consisting of —NR²⁸ and oxygen.

In certain embodiments, K is oxygen or —NR³⁵.

In certain embodiments, K is oxygen or sulfur.

In certain embodiments, B is selected from the group consisting of oxygen, or CR²⁷, CH₂ and C(R²⁷)₂.

In certain embodiments, M is oxygen or —NOR³⁰.

In certain embodiments, P is nitrogen or —CR³¹. In certain embodiments, at least five P are —CR³¹.

In certain embodiments, X is selected from the group consisting of oxygen, sulfur, and NOR¹⁶.

In embodiments in which two or more of a particular group are present, the identities of those two or more particular groups are selected independently and, thus, may be the same or different from one another. For example, certain compounds of the invention comprise two or more R¹⁶ groups. The identities of those two or more R¹⁶ groups are each selected independently. Thus, in certain embodiments, those R¹⁶ groups are all the same as one another; in certain embodiments, those R¹⁶ groups are all different from one another; and in certain embodiments, some of those R¹⁶ groups are the same as one another and some are different from one another. This independent selection applies to any group that is present in a compound more than once.

In certain embodiments, a compound of Formula I, Formula II, or Formula III is a selective glucocorticoid receptor modulator. In certain embodiments, a compound of Formula I, Formula II, or Formula III is a selective glucocorticoid receptor agonist. In certain embodiments, a compound of Formula I, Formula II, or Formula III is a selective glucocorticoid receptor antagonist. In certain embodiments, a compound of Formula I, Formula II, or Formula III is a selective glucocorticoid receptor partial agonist. In certain embodiments, a compound of Formula I, Formula II, or Formula III is a tissue-specific selective glucocorticoid receptor modulator. In certain embodiments, a compound of Formula I, Formula II, or Formula III is a gene-specific selective glucocorticoid receptor modulator. In certain embodiments, a compound of Formula I, Formula II, or Formula III is a selective glucocorticoid receptor binding compound.

In certain embodiments, a compound of Formula I, Formula II, or Formula III is a selective mineralocorticoid receptor modulator. In certain embodiments, a compound of Formula I, Formula II, or Formula III is a selective mineralocorticoid receptor agonist. In certain embodiments, a compound of Formula I, Formula II, or Formula III is a selective mineralocorticoid receptor antagonist. In certain embodiments, a compound of Formula I, Formula II, or Formula III is a selective mineralocorticoid receptor partial agonist. In certain embodiments, a compound of Formula I, Formula II, or Formula III is a tissue-specific selective mineralocorticoid receptor modulator. In certain embodiments, a compound of Formula I, Formula II, or Formula III is a gene-specific selective mineralocorticoid receptor modulator. In certain embodiments, a compound of Formula I, Formula II, or Formula III is a selective mineralocorticoid receptor binding compound.

In certain embodiments, a compound of Formula I, Formula II, or Formula III is a selective glucocorticoid/mineralocorticoid receptor modulator. In certain embodiments, a compound of Formula I, Formula II, or Formula III is a selective glucocorticoid/mineralocorticoid receptor agonist. In certain embodiments, a compound of Formula I, Formula II, or Formula III is a selective glucocorticoid/mineralocorticoid receptor antagonist. In certain embodiments, a compound of Formula I, Formula II, or Formula III is a selective glucocorticoid/mineralocorticoid receptor partial agonist. In certain embodiments, a compound of Formula I, Formula II, or Formula III is a tissue-specific selective glucocorticoid/mineralocorticoid receptor modulator. In certain embodiments, a compound of Formula I, Formula II, or Formula III is a gene-specific selective glucocorticoid/mineralocorticoid receptor modulator. In certain embodiments, a compound of Formula I, Formula II, or Formula III is a selective glucocorticoid/mineralocorticoid receptor binding compound.

In certain embodiments, the invention provides compounds selected from the group consisting of:
(±)-5-Chloro-1,2,3,4-tetrahydro-2,2,4,8-tetramethyl-6-(thiazol-2-yl)quinoline (compound 101),
(±)-6-(4-Acetylthiophen-2-yl)-5-chloro-1,2,3,4-tetrahydro-2,2,4,8-tetramethylquinoline (compound 102),
(±)-5-Chloro-1,2,3,4-tetrahydro-6-(indol-2-yl)-2,2,4,8-tetramethylquinoline (compound 103),
(±)-5-Chloro-6-(2,6-dimethoxyphenyl)-1,2,3,4-tetrahydro-2,2,4,8-tetramethylquinoline (compound 104),
(±)-5-Chloro-6-(3-cyano-2-methoxyphenyl)-1,2,3,4-tetrahydro-2,2,4,8-tetramethylquinoline (compound 105),
(+)-5-Chloro-6-(3-cyano-2-methoxyphenyl)-1,2,3,4-tetrahydro-2,2,4,8-tetramethylquinoline (compound 105A),
(−)-5-Chloro-6-(3-cyano-2-methoxyphenyl)-1,2,3,4-tetrahydro-2,2,4,8-tetramethylquinoline (compound 105B),
(±)-6-(3-Amino-5-methylisoxazol-4-yl)-5-chloro-1,2,3,4-tetrahydro-2,2,4,8-tetramethylquinoline (compound 106),
(±)-5-Chloro-1,2,3,4-tetrahydro-6-(2-methoxyphenyl)-2,2,4,8-tetramethylquinoline (compound 107),
(±)-5-Chloro-1,2,3,4-tetrahydro-2,2,4,8-tetramethyl-6-(quinolin-8-yl)quinoline (compound 108),
(±)-6-(Benzothiophen-3-yl)-5-chloro-1,2,3,4-tetrahydro-2,2,4,8-tetramethylquinoline (compound 109),
(±)-5-Chloro-1,2,3,4-tetrahydro-2,2,4,8-tetramethyl-6-(5-methyl-3-phenylisoxazol-4-yl)quinoline (compound 110),
(±)-5-Chloro-1,2,3,4-tetrahydro-2,2,4,8-tetramethyl-6-(1,3,5-trimethylpyrazol-4-yl)quinoline (compound III),
(±)-5-Chloro-6-(2,4-dimethoxyphenyl)-1,2,3,4-tetrahydro-2,2,4,8-tetramethylquinoline (compound 112),
(±)-6-(2-Aminophenyl)-5-chloro-1,2,3,4-tetrahydro-2,2,4,8-tetramethylquinoline (compound 113),
(±)-5-Chloro-6-(3,5-dimethylisoxazol-4-yl)-1,2,3,4-tetrahydro-2,2,4,8-tetramethylquinoline (compound 114),
(−)-5-Chloro-6-(3,5-dimethylisoxazol-4-yl)-1,2,3,4-tetrahydro-2,2,4,8-tetramethylquinoline (compound 114B),
(+)-5-Chloro-6-(3,5-dimethylisoxazol-4-yl)-1,2,3,4-tetrahydro-2,2,4,8-tetramethylquinoline (compound 114A),
(±)-6-(5-Acetylthiophen-2-yl)-5-chloro-1,2,3,4-tetrahydro-2,2,4,8-tetramethylquinoline (compound 115),
(±)-6-(Benzothiophen-2-yl)-5-chloro-1,2,3,4-tetrahydro-2,2,4,8-tetramethylquinoline (compound 116),
(±)-5-Chloro-6-(2-fluorophenyl)-1,2,3,4-tetrahydro-2,2,4,8-tetramethylquinoline (compound 117),
(±)-5-Chloro-6-(2-chlorophenyl)-1,2,3,4-tetrahydro-2,2,4,8-tetramethylquinoline (compound 118),
(±)-6-(2-Acetylphenyl)-5-chloro-1,2,3,4-tetrahydro-2,2,4,8-tetramethylquinoline (compound 119),
(±)-5-Chloro-1,2,3,4-tetrahydro-6-(indol-4-yl)-2,2,4,8-tetramethylquinoline (compound 120),
(±)-5-Chloro-6-(5-chloro-2-methoxyphenyl)-1,2,3,4-tetrahydro-2,2,4,8-tetramethylquinoline (compound 121),
(±)-5-Chloro-1,2,3,4-tetrahydro-2,2,4,8-tetramethyl-6-(2-nitrophenyl)quinoline (compound 122),
(±)-5-Chloro-6-(2,3-dichlorophenyl)-1,2,3,4-tetrahydro-2,2,4,8-tetramethylquinoline (compound 123),
(±)-5-Chloro-1,2,3,4-tetrahydro-2,2,4,8-tetramethyl-6-[2-(trifluoromethyl)phenyl]quinoline (compound 124),
(±)-5-Chloro-1,2,3,4-tetrahydro-2,2,4,8-tetramethyl-6-(2-methyl-3-nitrophenyl)quinoline (compound 125),
(±)-6-(2-Biphenyl)-5-chloro-1,2,3,4-tetrahydro-2,2,4,8-tetramethylquinoline (compound 126),
(±)-5-Chloro-6-(dibenzofuran-1-yl)-1,2,3,4-tetrahydro-2,2,4,8-tetramethylquinoline (compound 127),
(±)-5-Chloro-1,2,3,4-tetrahydro-6-(indol-6-yl)-2,2,4,8-tetramethylquinoline (compound 128),
(±)-5-Chloro-6-(2,3-dihydro-1,4-benzodioxin-6-yl)-1,2,3,4-tetrahydro-2,2,4,8-tetramethylquinoline (compound 129),
(±)-5-Chloro-6-[2-fluoro-3-(trifluoromethyl)phenyl]-1,2,3,4-tetrahydro-2,2,4,8-tetramethylquinoline (compound 130),
(±)-5-Chloro-1,2,3,4-tetrahydro-2,2,4,8-tetramethyl-6-[2-(trifluoromethoxy)phenyl]quinoline (compound 131),
(±)-5-Chloro-6-(5-cyano-2-methoxyphenyl)-1,2,3,4-tetrahydro-2,2,4,8-tetramethylquinoline (compound 132),
(±)-6-(1-Acetyl-3,5-dimethylpyrazol-4-yl)-5-chloro-1,2,3,4-tetrahydro-2,2,4,8-tetramethylquinoline (compound 133),
(±)-5-Chloro-1,2,3,4-tetrahydro-6-(indol-3-yl)-2,2,4,8-tetramethylquinoline (compound 134),
(±)-5-Chloro-1,2,3,4-tetrahydro-2,2,4,8-tetramethyl-6-(naphthal-1-yl)quinoline (compound 135),
(±)-5-Chloro-1,2,3,4-tetrahydro-2,2,4,8-tetramethyl-6-(3-methylpyrid-2-yl)quinoline (compound 136),
(±)-5-Chloro-6-(5-fluoroindol-7-yl)-1,2,3,4-tetrahydro-2,2,4,8-tetramethylquinoline (compound 137),
(±)-5-Chloro-1,2,3,4-tetrahydro-2,2,4,8-tetramethyl-6-(2-methylindol-7-yl)quinoline (compound 138),
(±)-5-Chloro-1,2,3,4-tetrahydro-2,2,4,8-tetramethyl-6-(3-methylindol-7-yl)quinoline (compound 139),
(±)-5-Chloro-6-(5-chloroindol-7-yl)-1,2,3,4-tetrahydro-2,2,4,8-tetramethylquinoline (compound 140),
(±)-5-Chloro-6-(4-fluoroindol-7-yl)-1,2,3,4-tetrahydro-2,2,4,8-tetramethylquinoline (compound 141),
(±)-5-Chloro-6-(4-chloroindol-7-yl)-1,2,3,4-tetrahydro-2,2,4,8-tetramethylquinoline (compound 142),
(±)-5-Chloro-6-(4,5-difluoroindol-7-yl)-1,2,3,4-tetrahydro-2,2,4,8-tetramethylquinoline (compound 143),
(±)-5-Chloro-1,2,3,4-tetrahydro-6-(4-methoxyindol-7-yl)-2,2,4,8-tetramethylquinoline (compound 144),
(±)-5-Chloro-6-(4-chloro-3-methylindol-7-yl)-1,2,3,4-tetrahydro-2,2,4,8-tetramethylquinoline (compound 145),
(±)-5-Chloro-6-(2,3-dimethylindol-7-yl)-1,2,3,4-tetrahydro-2,2,4,8-tetramethylquinoline (compound 146),
(±)-5-Chloro-6-(4-fluoro-3-methylindol-7-yl)-1,2,3,4-tetrahydro-2,2,4,8-tetramethylquinoline (compound 147),
(±)-5-Chloro-1,2,3,4-tetrahydro-2,2,4,8-tetramethyl-6-(1-methylindol-7-yl)quinoline (compound 148),
(±)-5-Chloro-1,2,3,4-tetrahydro-6-(indol-7-yl)-2,2,4,8-tetramethylquinoline (compound 149),
(−)-5-Chloro-1,2,3,4-tetrahydro-6-(indol-7-yl)-2,2,4,8-tetramethylquinoline (compound 149B),
(+)-5-Chloro-1,2,3,4-tetrahydro-6-(indol-7-yl)-2,2,4,8-tetramethylquinoline (compound 149A),
(±)-5-Chloro-6-(3-cyano-2,6-dimethoxyphenyl)-1,2,3,4-tetrahydro-2,2,4,8-tetramethylquinoline (compound 150),
(±)-5-Chloro-1,2,3,4-tetrahydro-6-(3-hydroxy-2-methoxyphenyl)-2,2,4,8-tetramethylquinoline (compound 151),
(±)-5-Chloro-6-(1-tetralon-5-yl)-1,2,3,4-tetrahydro-2,2,4,8-tetramethylquinoline (compound 152),
(±)-5-Chloro-6-(1-indanon-4-yl)-1,2,3,4-tetrahydro-2,2,4,8-tetramethylquinoline (compound 153),
(±)-5-Chloro-6-(1-hydroxyiminoindan-4-yl)-1,2,3,4-tetrahydro-2,2,4,8-tetramethylquinoline (compound 154),
(±)-5-Chloro-6-(3-cyano-2-methylphenyl)-1,2,3,4-tetrahydro-2,2,4,8-tetramethylquinoline (compound 155),
(±)-5-Chloro-1,2,3,4-tetrahydro-6-(2-methoxy-3-nitrophenyl)-2,2,4,8-tetramethylquinoline (compound 156),
(±)-5-Chloro-1,2,3,4-tetrahydro-6-(2-methoxy-6-nitrophenyl)-2,2,4,8-tetramethylquinoline (compound 157),
(±)-6-(2-Benzyloxy-3-nitrophenyl)-5-chloro-1,2,3,4-tetrahydro-2,2,4,8-tetramethylquinoline (compound 158),
(±)-6-(Benzothiophen-3-yl)-5-chloro-1,2,3,4-tetrahydro-3β-hydroxy-2,2,4α,8-tetramethylquinoline (compound 159),
(±)-5-Chloro-1,2,3,4-tetrahydro-3β-hydroxy-2,2,4α,8-tetramethyl-6-(thiophen-3-yl)quinoline (compound 160),
(±)-5-Chloro-1,2,3,4-tetrahydro-3β-hydroxy-6-(indol-7-yl)-2,2,4α,8-tetramethylquinoline (compound 161),
(+)-5-Chloro-1,2,3,4-tetrahydro-3β-hydroxy-6-(indol-7-yl)-2,2,4α,8-tetramethylquinoline (compound 161A),
(−)-5-Chloro-1,2,3,4-tetrahydro-3β-hydroxy-6-(indol-7-yl)-2,2,4α,8-tetramethylquinoline (compound 161B),
(±)-5-Chloro-1,2,3,4-tetrahydro-3β-hydroxy-2,2,4α,8-tetramethyl-6-(naphthal-1-yl)quinoline (compound 162),
(±)-5-Chloro-6-(4-fluoroindol-7-yl)-1,2,3,4-tetrahydro-3β-hydroxy-2,2,4α,8-tetramethylquinoline (compound 163),
(±)-5-Chloro-6-(3,5-dimethylisoxazol-4-yl)-1,2,3,4-tetrahydro-3β-hydroxy-2,2,4α,8-tetramethylquinoline (compound 164),
(±)-5-Chloro-6-(3-cyano-2-methoxyphenyl)-1,2,3,4-tetrahydro-3β-hydroxy-2,2,4α,8-tetramethylquinoline (compound 165),
(±)-5-Chloro-1,2,3,4-tetrahydro-3β-hydroxy-6-(4-fluoro-3-methylindol-7-yl)-2,2,4α,8-tetramethylquinoline (compound 166),
(±)-5-Chloro-1,2,3,4-tetrahydro-3β-hydroxy-6-(5-fluoroindol-7-yl)-2,2,4α,8-tetramethylquinoline (compound 167),
(±)-5-Chloro-1,2,3,4-tetrahydro-3β-hydroxy-6-(3-methylindol-7-yl)-2,2,4α,8-tetramethylquinoline (compound 168),
(±)-7-Chloro-6-(3-cyano-2-methoxyphenyl)-1,2,3,4-tetrahydro-2,2,4,8-tetramethylquinoline (compound 169),
(±)-7-Chloro-6-(3-cyanophenyl)-1,2,3,4-tetrahydro-2,2,4,8-tetramethylquinoline (compound 170),
(±)-7-Chloro-1,2,3,4-tetrahydro-6-(indol-7-yl)-2,2,4,8-tetramethylquinoline (compound 171),
(±)-7-Chloro-6-(3,5-dimethylisoxazol-4-yl)-1,2,3,4-tetrahydro-3β-hydroxy-2,2,4a,8-tetramethylquinoline (compound 172),
(±)-7-Chloro-1,2,3,4-tetrahydro-3β-hydroxy-6-(indol-7-yl)-2,2,4α,8-tetramethylquinoline (compound 173),
5-Chloro-6-(3-cyano-2-methoxyphenyl)-1,2-dihydro-2,2,4-trimethylquinoline (compound 174),
7-Chloro-6-(3-cyano-2-methoxyphenyl)-1,2-dihydro-2,2,4-trimethylquinoline (compound 175),
(±)-5-Chloro-6-(3-cyano-2-methoxyphenyl)-1,2,3,4-tetrahydro-2,2,4-trimethylquinoline (compound 176),
(±)-7-Chloro-6-(3-cyano-2-methoxyphenyl)-1,2,3,4-tetrahydro-2,2,4-trimethylquinoline (compound 177),
5-Chloro-6-(3,5-dimethylisoxazol-4-yl)-1,2-dihydro-2,2,4,8-tetramethylquinoline (compound 178),
(±)-5-Chloro-6-(3-cyano-2-methoxyphenyl)-1,4-dihydro-2,2,4,8-tetramethyl-2H-quinolin-3-one (compound 179),
(±)-4-Benzyl-5-chloro-6-(3-cyano-2-methoxyphenyl)-1,4-dihydro-2,2,4,8-tetramethyl-2H-quinolin-3-one (compound 180),
5-Chloro-6-(3-cyano-2-methoxyphenyl)-1,4-dihydro-2,2,4,4,8-pentamethyl-2H-quinolin-3-one (compound 181),
(±)-5-Chloro-6-(3,5-dimethylisoxazol-4-yl)-1,4-dihydro-2,2,4,8-tetramethyl-2H-quinolin-3-one (compound 182),
5-Chloro-6-(3,5-dimethylisoxazol-4-yl)-1,4-dihydro-2,2,4,4,8-pentamethyl-2H-quinolin-3-one (compound 183),
(±)-4-Benzyl-5-chloro-6-(3,5-dimethylisoxazol-4-yl)-1,4-dihydro-2,2,4,8-tetramethyl-2H-quinolin-3-one (compound 184),
(±)-5-Chloro-4-(3,3-dimethylallyl)-6-(3,5-dimethylisoxazol-4-yl)-1,4-dihydro-2,2,4,8-tetramethyl-2H-quinolin-3-one (compound 185),
(±)-5-Chloro-1,4-dihydro-6-(indol-7-yl)-2,2,4,8-tetramethyl-2H-quinolin-3-one (compound 186),
5-Chloro-1,4-dihydro-6-(indol-7-yl)-2,2,4,4,8-pentamethyl-2H-quinolin-3-one (compound 187),
(±)-4-Benzyl-5-chloro-1,4-dihydro-6-(indol-7-yl)-2,2,4,8-tetramethyl-2H-quinolin-3-one (compound 188),
(±)-5-Chloro-4-(3,3-dimethylallyl)-1,4-dihydro-6-(indol-7-yl)-2,2,4,8-tetramethyl-2H-quinolin-3-one (compound 189),
(±)-4-Allyl-5-chloro-1,4-dihydro-6-(indol-7-yl)-2,2,4,8-tetramethyl-2H-quinolin-3-one (compound 190),
(±)-5-Chloro-6-(3-cyano-2-methoxyphenyl)-1,2,3,4-tetrahydro-3α-hydroxy-2,2,4α,8-tetramethylquinoline (compound 191),
(±)-5-Chloro-6-(3,5-dimethylisoxazol-4-yl)-1,2,3,4-tetrahydro-3α-hydroxy-2,2,4α,8-tetramethylquinoline (compound 192),
(±)-5-Chloro-1,2,3,4-tetrahydro-3α-hydroxy-6-(indol-7-yl)-2,2,4α,8-tetramethylquinoline (compound 193),
(±)-6-(Benzothiophen-3-yl)-5-chloro-1,2,3,4-tetrahydro-3α-hydroxy-2,2,4α,8-tetramethylquinoline (compound 194),
(±)-5-Chloro-1,2,3,4-tetrahydro-3α-hydroxy-2,2,4α,8-tetramethyl-6-(naphthal-1-yl)quinoline (compound 195),
(±)-5-Chloro-1,2,3,4-tetrahydro-3-hydroxy-6-(indol-7-yl)-2,2,4,4,8-pentamethylquinoline (compound 196),
(±)-5-Chloro-6-(3,5-dimethylisoxazol-4-yl)-1,2,3,4-tetrahydro-3-hydroxy-2,2,4,4,8-pentamethylquinoline (compound 197),
(±)-6-(3-Amino-2-methoxyphenyl)-5-chloro-1,2,3,4-tetrahydro-2,2,4,8-tetramethylquinoline (compound 198),
(±)-5-Chloro-1,2,3,4-tetrahydro-6-[2-methoxy-3-(methoxycarbonylamino)phenyl]-2,2,4,8-tetramethylquinoline (compound 199),
(±)-5-Chloro-1,2,3,4-tetrahydro-6-[3-(tert-butoxycarbonylamino)-2-methoxyphenyl]-2,2,4,8-tetramethylquinoline (compound 200),
(±)-5-Chloro-1,2,3,4-tetrahydro-6-[2-methoxy-3-(methylsulfonamido)phenyl]-2,2,4,8-tetramethylquinoline (compound 201),
(±)-5-Chloro-1,2,3,4-tetrahydro-6-(2-hydroxy-3-nitrophenyl)-2,2,4,8-tetramethylquinoline (compound 202),
(±)-5-Chloro-1,2,3,4-tetrahydro-2,2,4,8-tetramethyl-6-[2-(methylbut-2-enyloxy)-3-nitrophenyl]quinoline (compound 203),
(±)-6-(2H-1,4-Benzoxazin-3(4H)-on-8-yl)-5-chloro-1,2,3,4-tetrahydro-2,2,4,8-tetramethylquinoline (compound 204),
(±)-5-Chloro-1,2,3,4-tetrahydro-2,2,4,8-tetramethyl-6-(4-methyl-2H-1,4-benzoxazin-3(4H)-on-8-yl)quinoline (compound 205),
(±)-6-(2-Benzoxazolinon-7-yl)-5-chloro-1,2,3,4-tetrahydro-2,2,4,8-tetramethylquinoline (compound 206),
(±)-6-(3-Amino-2-hydroxyphenyl)-5-chloro-1,2,3,4-tetrahydro-2,2,4,8-tetramethylquinoline (compound 207),
(±)-6-(2-Amino-6-methoxyphenyl)-5-chloro-1,2,3,4-tetrahydro-2,2,4,8-tetramethylquinoline (compound 208),
(±)-5-Chloro-1,2,3,4-tetrahydro-6-(6-methoxyindol-7-yl)-2,2,4,8-tetramethylquinoline (compound 209),
(±)-5-Chloro-1,2,3,4-tetrahydro-6-(indolin-7-yl)-2,2,4,8-tetramethylquinoline (compound 210),
(±)-6-(3-Bromoindol-7-yl)-5-chloro-1,2,3,4-tetrahydro-2,2,4,8-tetramethylquinoline (compound 211),
(±)-5-Chloro-1,2,3,4-tetrahydro-2,2,4,8-tetramethyl-6-(2-oxindol-7-yl)quinoline (compound 212),
(±)-5-Chloro-1,2,3,4-tetrahydro-4-hydroxy-6-(indol-2-yl)-2,2,4,8-tetramethylquinoline (compound 213),
5-Chloro-1,2-dihydro-6-(indol-2-yl)-2,2,4,8-tetramethylquinoline (compound 214),
(±)-5-Chloro-1,2,3,4-tetrahydro-4-hydroxy-2,2,4,8-tetramethyl-6-(naphthal-1-yl)quinoline (compound 215),
(±)-1,2,3,4-Tetrahydro-3β-hydroxy-6-(indol-7-yl)-2,2,4α,5,8-pentamethylquinoline (compound 216),
(±)-6-(3,5-Dimethylisoxazol-4-yl)-1,2,3,4-tetrahydro-3β-hydroxy-2,2,4α,5,8-pentamethylquinoline (compound 217),
(±)-5-Fluoro-1,2,3,4-tetrahydro-3β-hydroxy-2,2,4α,8-tetramethyl-6-(naphthal-1-yl)quinoline (compound 218),
(±)-6-(3,5-Dimethylisoxazol-4-yl)-5-fluoro-1,2,3,4-tetrahydro-3β-hydroxy-2,2,4α,8-tetramethylquinoline (compound 219),
(±)-5-Fluoro-1,2,3,4-tetrahydro-3β-hydroxy-6-(indol-7-yl)-2,2,4α,8-tetramethylquinoline (compound 220),
(±)-5-Chloro-1,2,3,4-tetrahydro-3β-hydroxy-6-(indolin-7-yl)-2,2,4α,8-tetramethylquinoline (compound 221),
(±)-5-Fluoro-1,2,3,4-tetrahydro-3β-hydroxy-6-(indolin-7-yl)-2,2,4α,8-tetramethylquinoline (compound 222),
(±)-5-Chloro-1,2,3,4-tetrahydro-3β-hydroxy-6-[3-(butan-3-on-1-yl)indol-7-yl]-2,2,4α,8-tetramethylquinoline (compound 223);
5-Chloro-6-(3-cyanophenyl)-1,2-dihydro-2,2,4-trimethylquinoline (compound 224);
(±)-5-Chloro-6-(3-cyanophenyl)-1,2,3,4-tetrahydro-2,2,4-trimethylquinoline (compound 225);
(+)-5-Chloro-6-(3-cyanophenyl)-1,2,3,4-tetrahydro-2,2,4-trimethylquinoline (compound 225A);
(−)-5-Chloro-6-(3-cyanophenyl)-1,2,3,4-tetrahydro-2,2,4-trimethylquinoline (compound 225B);
5-Chloro-6-(3-cyanophenyl)-1,2-dihydro-1,2,2,4-tetramethylquinoline (compound 226);
5-Chloro-8-fluoro-1,2-dihydro-2,2,4-trimethyl-6-(3-nitrophenyl)quinoline (compound 227);
5-Chloro-1,2-dihydro-2,2,4,8-tetramethyl-6-(3-nitrophenyl)quinoline (compound 228);
6-[3,5-Bis(trifluoromethyl)phenyl]-5-chloro-1,2-dihydro-2,2,4-trimethylquinoline (compound 229);
5-Chloro-1,2-dihydro-2,2,4-trimethyl-6-[3-(trifluoromethyl)phenyl]quinoline (compound 230);
5-Chloro-6-(3-cyanophenyl)-1,2-dihydro-2,2,4,8-tetramethylquinoline (compound 231);
5-Chloro-6-(3-cyano-4-fluorophenyl)-1,2-dihydro-2,2,4,8-tetramethylquinoline (compound 232);
6-(3-Acetylphenyl)-5-chloro-1,2-dihydro-2,2,4,8-tetramethylquinoline (compound 233);
5-Chloro-1,2-dihydro-2,2,4,8-tetramethyl-6-(3-methylphenyl)quinoline (compound 234);
5-Chloro-6-[4-chloro-3-(trifluoromethyl)phenyl]-1,2-dihydro-2,2,4,8-tetramethylquinoline (compound 235);
5-Chloro-6-(3-cyano-2-methylphenyl)-1,2-dihydro-2,2,4,8-tetramethylquinoline (compound 236);
5-Chloro-6-(3-fluoro-2-methylphenyl)-1,2-dihydro-2,2,4,8-tetramethylquinoline (compound 237);
5-Chloro-1,2-dihydro-2,2,4,8-tetramethyl-6-[3-(propionyl)phenyl]quinoline (compound 238);
6-(3-Carbamoylphenyl)-5-chloro-1,2-dihydro-2,2,4-trimethylquinoline (compound 239);
6-(3-Carboxymethylphenyl)-5-chloro-1,2-dihydro-2,2,4,8-tetramethylquinoline (compound 240);
5-Chloro-6-(5-cyanothiophen-3-yl)-1,2-dihydro-2,2,4,8-tetramethylquinoline (compound 241);
5-Chloro-6-(5-cyanopyrid-3-yl)-1,2-dihydro-2,2,4,8-tetramethylquinoline (compound 242);
(±)-6-(3-Acetylphenyl)-5-chloro-1,2,3,4-tetrahydro-2,2,4,8-tetramethylquinoline (compound 243);
(+)-6-(3-Acetylphenyl)-5-chloro-1,2,3,4-tetrahydro-2,2,4,8-tetramethylquinoline (compound 243A);
(−)-6-(3-Acetylphenyl)-5-chloro-1,2,3,4-tetrahydro-2,2,4,8-tetramethylquinoline (compound 243B);
(±)-5-Chloro-6-(5-cyanothiophen-3-yl)-1,2,3,4-tetrahydro-2,2,4,8-tetramethylquinoline (compound 244);
(±)-5-Acetoxy-6-(3-cyanophenyl)-1,2,3,4-tetrahydro-2,2,4,8-tetramethylquinoline (compound 245);
6-[3-(N-Methoxy-N-methylcarbamoyl)phenyl]-5-chloro-1,2-dihydro-2,2,4-trimethylquinoline (compound 246);
5-Chloro-1,2-dihydro-2,2,4,8-tetramethyl-6-[3-(2-methylpropionyl)phenyl]quinoline (compound 247);
(±)-5-Chloro-6-(3-cyano-2-hydroxyphenyl)-1,2,3,4-tetrahydro-2,2,4,8-tetramethylquinoline (compound 248);
(±)-6-(3-Cyanophenyl)-1,2,3,4-tetrahydro-5-hydroxy-2,2,4,8-tetramethylquinoline (compound 249);
(±)-6-(3-Cyanophenyl)-1,2,3,4-tetrahydro-5-methoxy-2,2,4,8-tetramethylquinoline (compound 250);
(±)-6-(5-Carbamoylpyrid-3-yl)-5-chloro-1,2,3,4-tetrahydro-2,2,4,8-tetramethylquinoline (compound 251);
(±)-5-Chloro-6-(2-cyanothiophen-3-yl)-1,2,3,4-tetrahydro-2,2,4,8-tetramethylquinoline (compound 252);
(±)-5-Chloro-6-[3-(cyanomethyl)phenyl]-1,2,3,4-tetrahydro-2,2,4,8-tetramethylquinoline (compound 253);
(±)-6-(3-Cyanophenyl)-5-(2,2-dimethylpropionyloxy)-1,2,3,4-tetrahydro-2,2,4,8-tetramethylquinoline (compound 254);
(±)-5-Chloro-1,2,3,4-tetrahydro-2,2,4,8-tetramethyl-6-(5-nitrothiophen-2-yl)quinoline (compound 255);
(±)-5-Chloro-1,2,3,4-tetrahydro-2,2,4,8-tetramethyl-6-(pyrimidin-5-yl)quinoline (compound 256);
6-(3-Acetylphenyl)-5,7-dichloro-1,2-dihydro-2,2,4-trimethylquinoline (compound 257);
(±)-1,2,3,4-Tetrahydro-3β-hydroxy-6-(indol-7-yl)-2,2,4α,8-tetramethylquinoline (compound 258);
(±)-6-(3,5-Dimethylisoxazol-4-yl)-1,2,3,4-tetrahydro-3β-hydroxy-2,2,4α,8-tetramethylquinoline (compound 259);
(±)-1,2,3,4-Tetrahydro-3β-hydroxy-2,2,4α,8-tetramethyl-6-(quinolin-8-yl)quinoline (compound 260);
(±)-5-Chloro-1,2,3,4-tetrahydro-6-(indol-7-yl)-2,2,4,8-tetramethylquinoline (compound 261);
(±)-5-Chloro-1,2,3,4-tetrahydro-3β-hydroxy-2,2,4α,8-tetramethyl-6-(6-fluoro-2-nitrophenyl)-quinoline (compound 262);
(±)-5-Chloro-1,2,3,4-tetrahydro-3β-hydroxy-6-(6-fluoroindol-7-yl)-2,2,4α,8-tetramethylquinoline (compound 263);
(±)-5-Chloro-1,2,3,4-tetrahydro-3β-hydroxy-2,2,4α,8-tetramethyl-6-(4,6-difluoro-2-nitrophenyl)quinoline (compound 264);
(±)-5-Chloro-1,2,3,4-tetrahydro-3β-hydroxy-6-(4,6-difluoroindol-7-yl)-2,2,4α,8-tetramethylquinoline (compound 265);
(±)-5-Chloro-1,2,3,4-tetrahydro-3β-hydroxy-6-(5-fluoroindol-7-yl)-2,2,4α,8-tetramethylquinoline (compound 266);
(±)-1,2,3,4-Tetrahydro-3β-hydroxy-2,2,4α,8-tetramethyl-6-(6-methoxy-2-nitrophenyl)-quinoline (compound 267);
(±)-1,2,3,4-Tetrahydro-3β-hydroxy-6-(6-methoxy-indol-7-yl)-2,2,4α,8-tetramethylquinoline (compound 268);
(±)-7-Fluoro-1,2,3,4-tetrahydro-3β-hydroxy-6-(indol-7-yl)-2,2,4α,8-tetramethylquinoline (compound 269);
(±)-6-(3,5-Dimethylisoxazol-4-yl)-1,2,3,4-tetrahydro-3β-hydroxy-5-methoxy-2,2,4α,8-tetramethylquinoline (compound 270);
(±)-1,2,3,4-tetrahydro-3β-hydroxy-5-methoxy-2,2,4α,8-tetramethyl-6-(naphth-1-yl)quinoline (compound 271);
(±)-1,2,3,4-tetrahydro-3β-hydroxy-6-(indol-7-yl)-5-methoxy-2,2,4α,8-tetramethylquinoline (compound 272);
(±)-5-Chloro-6-(2-fluoropyrid-3-yl)-1,2,3,4-tetrahydro-3β-hydroxy-2,2,4α,8-tetramethylquinoline (compound 273);
(±)-5-Chloro-1,2,3,4-tetrahydro-3β-hydroxy-6-(2-methoxypyrid-3-yl)-2,2,4α,8-tetramethylquinoline (compound 274);
(±)-5-Chloro-1,2,3,4-tetrahydro-8-fluoro-3β-hydroxy-6-(indol-7-yl)-2,2,4α-trimethylquinoline (compound 275);
(±)-5-Cyano-1,2,3,4-tetrahydro-3β-hydroxy-6-(indol-7-yl)-2,2,4α,8-tetramethylquinoline (compound 276);
(±)-5-Ethynyl-1,2,3,4-tetrahydro-3β-hydroxy-6-(indol-7-yl)-2,2,4α,8-tetramethylquinoline (compound 277);
(±)-1,2,3,4-Tetrahydro-3β-hydroxy-6-(indol-7-yl)-2,2,4α,8-tetramethyl-E-(2-phenylethenyl)quinoline (compound 278);
(±)-5-Carbomethoxy-1,2,3,4-tetrahydro-6-(indol-7-yl)-2,2,4,8-tetramethylquinoline (compound 279);
(±)-5-Carboxy-1,2,3,4-tetrahydro-6-(indol-7-yl)-2,2,4,8-tetramethylquinoline (compound 280);
(±)-5-Chloro-1,2,3,4-tetrahydro-6-(6-methoxy-3-methylindol-7-yl)-2,2,4,8-tetramethylquinoline (compound 281);
(±)-5-Chloro-1,2,3,4-tetrahydro-3β-hydroxy-2,2,4α,8-tetramethyl-6-(oxazol-5-yl)quinoline (compound 282);
(±)-5-Chloro-1,2,3,4-tetrahydro-3β-hydroxy-6-(5-methoxyindol-7-yl)-2,2,4α,8-tetramethylquinoline (compound 283);
(±)-5-Chloro-1,2,3,4-tetrahydro-3β-hydroxy-2,2,4α,8-tetramethyl-6-(pyrid-4-yl)quinoline (compound 284);
(±)-5-Cyano-1,2,3,4-tetrahydro-3β-hydroxy-6-(indolin-7-yl)-2,2,4α,8-tetramethylquinoline (compound 285);
(±)-5-Chloro-1,2,3,4-tetrahydro-3α-methoxy-2,2,4α,8-tetramethyl-6-(naphthal-1-yl)quinoline (compound 286);
(±)-1,2,3,4-Tetrahydro-3β-hydroxy-6-(indolin-7-yl)-5-(methoxyimino)-2,2,4α,8-tetramethylquinoline (compound 287);
(±)-1,2,3,4-Tetrahydro-5-(hydroxymethyl)-6-(indol-7-yl)-2,2,4,8-tetramethylquinoline (compound 288);
(±)-5-(3-(2-Fluoroethoxy)benzyloxymethyl)-1,2,3,4-tetrahydro-6-(indol-7-yl)-2,2,4,8-tetramethylquinoline (compound 289);
(±)-5-((6-Fluoro-4H-benzo[1,3]dioxin-8-yl)methoxymethyl)-1,2,3,4-tetrahydro-6-(indol-7-yl)-2,2,4,8-tetramethylquinoline (compound 290);
(±)-5-(2-Fluoro-3-methylbenzyloxymethyl)-1,2,3,4-tetrahydro-6-(indol-7-yl)-2,2,4,8-tetramethylquinoline (compound 291);

and pharmaceutically acceptable salts, esters, amides, or prodrugs of any of those compounds.

Certain compounds of the present inventions may exist as stereoisomers including optical isomers. The present disclosure is intended to include all stereoisomers and both the racemic mixtures of such stereoisomers as well as the individual enantiomers that may be separated according to methods that are known in the art or that may be excluded by synthesis schemes known in the art designed to yield predominantly one enantomer relative to another.

Certain exemplary compounds of Formula I, II or III are set forth below.

| | |
|---|---|
| | (I) |
| 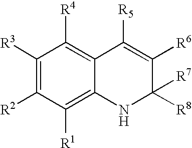 | |

| Cmpd | | | | | | | | |
|---|---|---|---|---|---|---|---|---|
| # | R¹ | R² | R³ | R⁴ | R⁵ | R⁶ | R⁷ | R⁸ |
| 174 | H | H | 3-cyano-2-methoxy- | Cl | CH₃ | H | CH₃ | CH₃ |
| | | | phenyl | | | | | |
| 175 | H | Cl | 3-cyano-2-methoxy- | H | CH₃ | H | CH₃ | CH₃ |
| | | | phenyl | | | | | |
| 178 | CH₃ | H | 3,5-dimethyl- | Cl | CH₃ | H | CH₃ | CH₃ |
| | | | isoxazol-4-yl | | | | | |
| 214 | CH₃ | H | indol-2-yl | Cl | CH₃ | H | CH₃ | CH₃ |
| 227 | F | H | 3-nitrophenyl | Cl | CH₃ | H | CH₃ | CH₃ |
| 233 | CH₃ | H | 3-acetylphenyl | Cl | CH₃ | H | CH₃ | CH₃ |

| | |
|---|---|
| | (II) |
| 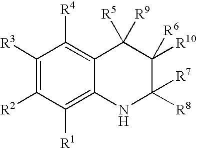 | |

| Cmpd | | | | | | | | | | |
|---|---|---|---|---|---|---|---|---|---|---|
| # | R¹ | R² | R³ | R⁴ | R⁵ | R⁶ | R⁷ | R⁸ | R⁹ | R¹⁰ |
| 101 | CH₃ | H | thiazol-2-yl | Cl | CH₃ | H | CH₃ | CH₃ | H | H |
| 102 | CH₃ | H | 4-acetylthiophen-2-yl | Cl | CH₃ | H | CH₃ | CH₃ | H | H |
| 103 | CH₃ | H | indol-2-yl | Cl | CH₃ | H | CH₃ | CH₃ | H | H |
| 104 | CH₃ | H | 2,6-dimethoxyphenyl | Cl | CH₃ | H | CH₃ | CH₃ | H | H |
| 105 | CH₃ | H | 3-cyano-2-methoxyphenyl | Cl | CH₃ | H | CH₃ | CH₃ | H | H |
| 106 | CH₃ | H | 3-amino-5-methylisoxazol-4-yl | Cl | CH₃ | H | CH₃ | CH₃ | H | H |
| 107 | CH₃ | H | 2-methoxyphenyl | Cl | CH₃ | H | CH₃ | CH₃ | H | H |
| 108 | CH₃ | H | quinolin-8-yl | Cl | CH₃ | H | CH₃ | CH₃ | H | H |
| 109 | CH₃ | H | benzothiophen-3-yl | Cl | CH₃ | H | CH₃ | CH₃ | H | H |
| 110 | CH₃ | H | 5-methyl-3-phenylisoxazol-4-yl | Cl | CH₃ | H | CH₃ | CH₃ | H | H |
| 111 | CH₃ | H | 1,3,5-trimethylpyrazol-4-yl | Cl | CH₃ | H | CH₃ | CH₃ | H | H |
| 112 | CH₃ | H | 2,4-dimethoxyphenyl | Cl | CH₃ | H | CH₃ | CH₃ | H | H |
| 113 | CH₃ | H | 2-aminophenyl | Cl | CH₃ | H | CH₃ | CH₃ | H | H |
| 114 | CH₃ | H | 3,5-dimethylisoxazol-4-yl | Cl | CH₃ | H | CH₃ | CH₃ | H | H |
| 115 | CH₃ | H | 5-acetylthiophen-2-yl | Cl | CH₃ | H | CH₃ | CH₃ | H | H |
| 116 | CH₃ | H | benzothiophen-2-yl | Cl | CH₃ | H | CH₃ | CH₃ | H | H |
| 117 | CH₃ | H | 2-fluorophenyl | Cl | CH₃ | H | CH₃ | CH₃ | H | H |
| 118 | CH₃ | H | 2-chlorophenyl | Cl | CH₃ | H | CH₃ | CH₃ | H | H |
| 119 | CH₃ | H | 2-acetylphenyl | Cl | CH₃ | H | CH₃ | CH₃ | H | H |
| 120 | CH₃ | H | indol-4-yl | Cl | CH₃ | H | CH₃ | CH₃ | H | H |
| 121 | CH₃ | H | 5-chloro-2-methoxyphenyl | Cl | CH₃ | H | CH₃ | CH₃ | H | H |
| 122 | CH₃ | H | 2-nitrophenyl | Cl | CH₃ | H | CH₃ | CH₃ | H | H |
| 123 | CH₃ | H | 2,3-dichlorophenyl | Cl | CH₃ | H | CH₃ | CH₃ | H | H |
| 124 | CH₃ | H | 2-(trifluoromethyl)phenyl | Cl | CH₃ | H | CH₃ | CH₃ | H | H |
| 125 | CH₃ | H | 2-methyl-3-nitrophenyl | Cl | CH₃ | H | CH₃ | CH₃ | H | H |
| 126 | CH₃ | H | 2-biphenyl | Cl | CH₃ | H | CH₃ | CH₃ | H | H |
| 127 | CH₃ | H | dibenzofuran-1-yl | Cl | CH₃ | H | CH₃ | CH₃ | H | H |
| 128 | CH₃ | H | indol-6-yl | Cl | CH₃ | H | CH₃ | CH₃ | H | H |
| 129 | CH₃ | H | 2,3-dihydro-1,4-benzodioxin-6-yl | Cl | CH₃ | H | CH₃ | CH₃ | H | H |
| 130 | CH₃ | H | 2-fluoro-3-(trifluoromethyl)phenyl | Cl | CH₃ | H | CH₃ | CH₃ | H | H |
| 131 | CH₃ | H | 2-(trifluoromethoxy)phenyl | Cl | CH₃ | H | CH₃ | CH₃ | H | H |
| 132 | CH₃ | H | 5-cyano-2-methoxyphenyl | Cl | CH₃ | H | CH₃ | CH₃ | H | H |
| 133 | CH₃ | H | 1-acetyl-3,5-dimethylpyrazol-4-yl | Cl | CH₃ | H | CH₃ | CH₃ | H | H |
| 134 | CH₃ | H | indol-3-yl | Cl | CH₃ | H | CH₃ | CH₃ | H | H |
| 135 | CH₃ | H | naphthal-1-yl | Cl | CH₃ | H | CH₃ | CH₃ | H | H |
| 136 | CH₃ | H | 3-methylpyrid-2-yl | Cl | CH₃ | H | CH₃ | CH₃ | H | H |
| 137 | CH₃ | H | 5-fluoroindol-7-yl | Cl | CH₃ | H | CH₃ | CH₃ | H | H |
| 138 | CH₃ | H | 2-methylindol-7-yl | Cl | CH₃ | H | CH₃ | CH₃ | H | H |
| 139 | CH₃ | H | 3-methylindol-7-yl | Cl | CH₃ | H | CH₃ | CH₃ | H | H |
| 140 | CH₃ | H | 5-chloroindol-7-yl | Cl | CH₃ | H | CH₃ | CH₃ | H | H |
| 141 | CH₃ | H | 4-fluoroindol-7-yl | Cl | CH₃ | H | CH₃ | CH₃ | H | H |
| 142 | CH₃ | H | 4-chloroindol-7-yl | Cl | CH₃ | H | CH₃ | CH₃ | H | H |
| 143 | CH₃ | H | 4,5-difluoroindol-7-yl | Cl | CH₃ | H | CH₃ | CH₃ | H | H |
| 144 | CH₃ | H | 4-methoxyindol-7-yl | Cl | CH₃ | H | CH₃ | CH₃ | H | H |
| 145 | CH₃ | H | 4-chloro-3-methylindol-7-yl | Cl | CH₃ | H | CH₃ | CH₃ | H | H |
| 146 | CH₃ | H | 2,3-dimethylindol-7-yl | Cl | CH₃ | H | CH₃ | CH₃ | H | H |
| 147 | CH₃ | H | 4-fluoro-3-methylindol-7-yl | Cl | CH₃ | H | CH₃ | CH₃ | H | H |
| 148 | CH₃ | H | 1-methylindol-7-yl | Cl | CH₃ | H | CH₃ | CH₃ | H | H |
| 149 | CH₃ | H | indol-7-yl | Cl | CH₃ | H | CH₃ | CH₃ | H | H |
| 150 | CH₃ | H | 3-cyano-2,6-dimethoxyphenyl | Cl | CH₃ | H | CH₃ | CH₃ | H | H |
| 151 | CH₃ | H | 3-hydroxy-2-methoxyphenyl | Cl | CH₃ | H | CH₃ | CH₃ | H | H |
| 152 | CH₃ | H | 1-tetralon-5-yl | Cl | CH₃ | H | CH₃ | CH₃ | H | H |
| 153 | CH₃ | H | 1-indanon-4-yl | Cl | CH₃ | H | CH₃ | CH₃ | H | H |
| 154 | CH₃ | H | 1-hydroxyimino-indan-4-yl | Cl | CH₃ | H | CH₃ | CH₃ | H | H |
| 155 | CH₃ | H | 3-cyano-2-methylphenyl | Cl | CH₃ | H | CH₃ | CH₃ | H | H |
| 156 | CH₃ | H | 2-methoxy-3-nitrophenyl | Cl | CH₃ | H | CH₃ | CH₃ | H | H |
| 157 | CH₃ | H | 2-methoxy-6-nitrophenyl | Cl | CH₃ | H | CH₃ | CH₃ | H | H |
| 158 | CH₃ | H | 2-benzyloxy-3-nitrophenyl | Cl | CH₃ | H | CH₃ | CH₃ | H | H |
| 159 | CH₃ | H | benzothiophen-3yl | Cl | CH₃ | OH | CH₃ | CH₃ | H | H |
| 160 | CH₃ | H | thiophen-3-yl | Cl | CH₃ | OH | CH₃ | CH₃ | H | H |
| 161 | CH₃ | H | indol-7-yl | Cl | CH₃ | OH | CH₃ | CH₃ | H | H |
| 162 | CH₃ | H | naphthal-1-yl | Cl | CH₃ | OH | CH₃ | CH₃ | H | H |
| 163 | CH₃ | H | 4-fluoroindol-7-yl | Cl | CH₃ | OH | CH₃ | CH₃ | H | H |
| 164 | CH₃ | H | 3,5-dimethylisoxazol-4-yl | Cl | CH₃ | OH | CH₃ | CH₃ | H | H |
| 165 | CH₃ | H | 3-cyano-2-methoxyphenyl | Cl | CH₃ | OH | CH₃ | CH₃ | H | H |
| 166 | CH₃ | H | 4-fluoro-3-methylindol-7-yl | Cl | CH₃ | OH | CH₃ | CH₃ | H | H |
| 167 | CH₃ | H | 4-fluoro-3-methylindol-7-yl | Cl | CH₃ | OH | CH₃ | CH₃ | H | H |
| 168 | CH₃ | H | 3-methylindol-7-yl | Cl | CH₃ | OH | CH₃ | CH₃ | H | H |
| 169 | CH₃ | Cl | 3-cyano-2-methoxyphenyl | H | CH₃ | H | CH₃ | CH₃ | H | H |
| 170 | CH₃ | Cl | 3-cyanophenyl | H | CH₃ | H | CH₃ | CH₃ | H | H |
| 171 | CH₃ | Cl | indol-7-yl | H | CH₃ | H | CH₃ | CH₃ | H | H |
| 172 | CH₃ | Cl | 3,5-dimethylisoxazol-4-yl | H | CH₃ | OH | CH₃ | CH₃ | H | H |
| 173 | CH₃ | Cl | indol-7-yl | H | CH₃ | OH | CH₃ | CH₃ | H | H |
| 176 | H | H | 3-cyano-2-methoxyphenyl | Cl | CH₃ | H | CH₃ | CH₃ | H | H |
| 177 | H | Cl | 3-cyano-2-methoxyphenyl | H | CH₃ | H | CH₃ | CH₃ | H | H |
| 191 | CH₃ | H | 3-cyano-2-methoxyphenyl | Cl | CH₃ | H | CH₃ | CH₃ | H | OH |
| 192 | CH₃ | H | 3,5-dimethylisoxazol-4-yl | Cl | CH₃ | H | CH₃ | CH₃ | H | OH |
| 193 | CH₃ | H | indol-7-yl | Cl | CH₃ | H | CH₃ | CH₃ | H | OH |
| 194 | CH₃ | H | benzothiophen-3-yl | Cl | CH₃ | H | CH₃ | CH₃ | H | OH |
| 195 | CH₃ | H | naphthal-1-yl | Cl | CH₃ | H | CH₃ | CH₃ | H | OH |
| 196 | CH₃ | H | indol-7-yl | Cl | CH₃ | OH | CH₃ | CH₃ | CH₃ | H |
| 197 | CH₃ | H | 3,5-dimethylisoxazol-4-yl | Cl | CH₃ | OH | CH₃ | CH₃ | CH₃ | H |
| 198 | CH₃ | H | 3-amino-2-methoxyphenyl | Cl | CH₃ | H | CH₃ | CH₃ | H | H |
| 199 | CH₃ | H | 2-methoxy-3-(methoxycarbonyl- | Cl | CH₃ | H | CH₃ | CH₃ | H | H |
| | | | amino)phenyl | | | | | | | |
| 200 | CH₃ | H | 3-(tert-butoxy-carbonyl- | Cl | CH₃ | H | CH₃ | CH₃ | H | H |
| | | | amino)-2-methoxyphenyl | | | | | | | |
| 201 | CH₃ | H | 2-methoxy-3-(methyl- | Cl | CH₃ | H | CH₃ | CH₃ | H | H |
| | | | sulfonamido)phenyl | | | | | | | |
| 202 | CH₃ | H | 2-hydroxy-3-nitrophenyl | Cl | CH₃ | H | CH₃ | CH₃ | H | H |
| 203 | CH₃ | H | 2-(methylbut-2-enyloxy)-3- | Cl | CH₃ | H | CH₃ | CH₃ | H | H |
| | | | nitrophenyl | | | | | | | |
| 204 | CH₃ | H | 2H-1,4-benzoxazin-3(4H)-on-8-yl | Cl | CH₃ | H | CH₃ | CH₃ | H | H |
| 205 | CH₃ | H | 4-methyl-2H-1,4-benzoxazin-3- | Cl | CH₃ | H | CH₃ | CH₃ | H | H |
| | | | (4H)-on-8-yl | | | | | | | |
| 206 | CH₃ | H | 2-benzoxazolinon-7-yl | Cl | CH₃ | H | CH₃ | CH₃ | H | H |
| 207 | CH₃ | H | 3-amino-2-hydroxyphenyl | Cl | CH₃ | H | CH₃ | CH₃ | H | H |
| 208 | CH₃ | H | 2-amino-6-methoxyphenyl | Cl | CH₃ | H | CH₃ | CH₃ | H | H |
| 209 | CH₃ | H | 6-methoxyindol-7-yl | Cl | CH₃ | H | CH₃ | CH₃ | H | H |
| 210 | CH₃ | H | indolin-7-yl | Cl | CH₃ | H | CH₃ | CH₃ | H | H |
| 211 | CH₃ | H | 3-bromoindol-7-yl | Cl | CH₃ | H | CH₃ | CH₃ | H | H |
| 212 | CH₃ | H | 2-oxindol-7-yl | Cl | CH₃ | H | CH₃ | CH₃ | H | H |
| 213 | CH₃ | H | indol-2-yl | Cl | CH₃ | H | CH₃ | CH₃ | OH | H |
| 215 | CH₃ | H | naphthal-1-yl | Cl | CH₃ | H | CH₃ | CH₃ | OH | H |
| 216 | CH₃ | H | indol-7-yl | CH₃ | CH₃ | OH | CH₃ | CH₃ | H | H |
| 217 | CH₃ | H | 3,5-dimethylisoxazol-4-yl | CH₃ | CH₃ | OH | CH₃ | CH₃ | H | H |
| 218 | CH₃ | H | naphthal-1-yl | F | CH₃ | OH | CH₃ | CH₃ | H | H |
| 219 | CH₃ | H | 3,5-dimethylisoxazol-4-yl | F | CH₃ | OH | CH₃ | CH₃ | H | H |
| 220 | CH₃ | H | indol-7-yl | F | CH₃ | OH | CH₃ | CH₃ | H | H |
| 221 | CH₃ | H | indolin-7-yl | Cl | CH₃ | OH | CH₃ | CH₃ | H | H |
| 222 | CH₃ | H | indolin-7-yl | F | CH₃ | OH | CH₃ | CH₃ | H | H |
| 223 | CH₃ | H | 3-(butan-3-on-1-yl)indol-7-yl | Cl | CH₃ | OH | CH₃ | CH₃ | H | H |
| 245 | CH₃ | H | 3-cyanophenyl | acetoxy | CH₃ | H | CH₃ | CH₃ | H | H |

| | |
|---|---|
| | (III) |
| 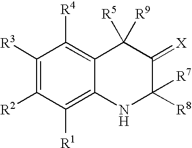 | |

| Cmpd | | | | | | | | |
|---|---|---|---|---|---|---|---|---|
| # | R¹ | R² | R³ | R⁴ | R⁵ | R⁷ | R⁸ | R⁹ |
| 179 | CH₃ | H | 3-cyano-2-methoxyphenyl | Cl | CH₃ | CH₃ | CH₃ | H |
| 180 | CH₃ | H | 3-cyano-2-methoxyphenyl | Cl | CH₃ | CH₃ | CH₃ | benzyl |
| 181 | CH₃ | H | 3-cyano-2-methoxyphenyl | Cl | CH₃ | CH₃ | CH₃ | CH₃ |
| 182 | CH₃ | H | 3,5-dimethylisoxazol-4-yl | Cl | CH₃ | CH₃ | CH₃ | H |
| 183 | CH₃ | H | 3,5-dimethylisoxazol-4-yl | Cl | CH₃ | CH₃ | CH₃ | CH₃ |
| 184 | CH₃ | H | 3,5-dimethylisoxazol-4-yl | Cl | CH₃ | CH₃ | CH₃ | benzyl |
| 185 | CH₃ | H | 3,5-dimethylisoxazol-4-yl | Cl | CH₃ | CH₃ | CH₃ | 3,3-dimethylallyl |
| 186 | CH₃ | H | indol-7-yl | Cl | CH₃ | CH₃ | CH₃ | H |
| 187 | CH₃ | H | indol-7-yl | Cl | CH₃ | CH₃ | CH₃ | CH₃ |
| 188 | CH₃ | H | indol-7-yl | Cl | CH₃ | CH₃ | CH₃ | benzyl |
| 189 | CH₃ | H | indol-7-yl | Cl | CH₃ | CH₃ | CH₃ | 3,3-dimethylallyl |
| 190 | CH₃ | H | indol-7-yl | Cl | CH₃ | CH₃ | CH₃ | allyl |

Certain Synthesis Methods

Certain synthesis schemes are now provided. The synthesis schemes are provide only to illustrate possible ways to make certain compounds of the invention and do not limit the invention in any way. One of skill in the art will recognize that compounds of the present invention may be synthesized through any of a variety of schemes using a variety of different starting materials.

In certain embodiments, synthesis of 6-aryl- and 6-heteroaryl 1,2,3,4-tetrahydroquinoline compounds (e.g. Structures 6, (+)-6, and (−)-6) is accomplished using Scheme I.

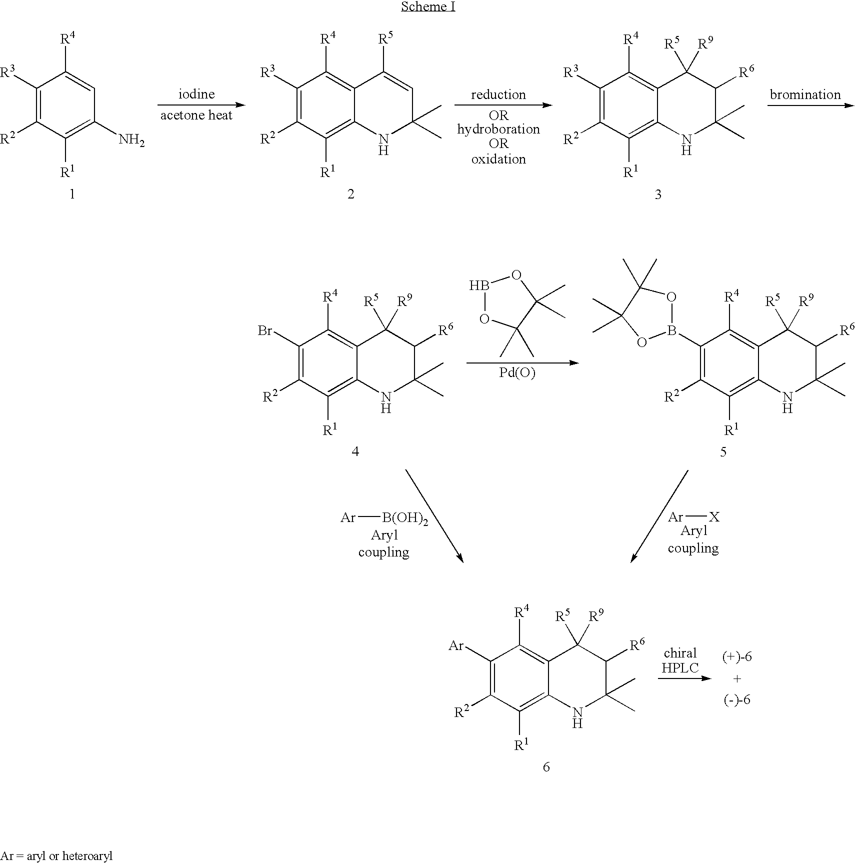

The process of Scheme I begins with Skraup quinoline synthesis of an aniline (Structure 1), with a ketone, for example, acetone in the presence of iodine heated in a sealed tube at elevated temperatures to afford a dihydroquinoline (Structure 2). See Pooley, C. L. F., et al, J. Med. Chem. 41:3461 (1998), which is incorporated herein by reference in its entirety. The olefin of the dihydroquinoline can be functionalized in a number of ways. For example, the quinoline can be reduced by treatment with a reducing agent, for example, triethylsilane, in the presence of an acid, for example trifluoroacetic acid, to afford a tetrahydroquinoline (Structure 3, R⁶, R⁹=H). Alternatively, the dihydroquinoline can be hydrated by, for example, treatment with a hydroborating agent, for example diborane, and subsequently treated with an oxidant, such as hydrogen peroxide, in the presence of a base, for example, sodium hydroxide to afford either a 4-hydroxy-1,2,3,4-tetrahydroquinoline (Structure 3, R⁶=H, R⁹=OH), or a 4α-alkyl-3β-hydroxy-1,2,3,4-tetrahydroquinoline (Structure 3, R⁶=OH, R⁹=H). Alternatively, the dihydroquinoline can be oxidized by treatment with an oxidant, for example, osmium tetraoxide, to afford a 3,4-dihydroxy-1,2,3,4-tetrahydroquinoline (Structure 3, R⁶, R⁹=OH). Structure 3 can be halogenated at the 6-position by treatment with a brominating agent, for example, N-bromosuccinimide, to afford a compound of Structure 4. Treatment of Structure 4 with an organometallic reagent, for example, an aryl boronic acid, in the presence of a transition metal catalyst, for example, [1,1′-bis(diphenylphosphino)ferrocene]dichloropalladium(II), in the presence of a base, for example, aqueous sodium carbonate, to afford a compound of Structure 6.

A compound of Structure 4 can be metallated to a compound of Structure 5 by treatment with a boronating agent, for example, 4,4,5,5-tetramethyl-1,3,2-dioxaborolane, in the presence of a transition metal catalyst, for example, [1,1′-bis(diphenylphosphino)ferrocene]dichloropalladium(II), in the presence of a base, for example, triethylamine, to afford a compound of Structure 5. Treatment of Structure 4 with halide, for example, an aryl bromide, in the presence of a transition metal catalyst, for example, [1,1′-bis(diphenylphosphino)ferrocene]dichloropalladium(II), in the presence of a base, for example, aqueous sodium carbonate, affords a compound of Structure 6. Tetrahydroquinoline compounds of Structure 6 (or any chiral synthetic precursor of Structure 6) can be separated into their corresponding enantiomers, (+)-6 and (−)-6 by chiral HPLC, with, for example, a preparative Chiracel OJ column eluted with hexanes:isopropanol. Alternatively, the enantiomers (+)-6 and (−)-6 could be prepared in enantiomerically enriched form via an enantiospecific synthesis of a synthetic precursor of Structure 6, for example, by asymmetric hydroboration of Structure 2 to afford a compound of Structure 3 in enantiomerically enriched form.

In certain embodiments, synthesis of 6-aryl- or 6-heteroaryl-1,2,3,4-tetrahydroquinolines is accomplished using Scheme II.

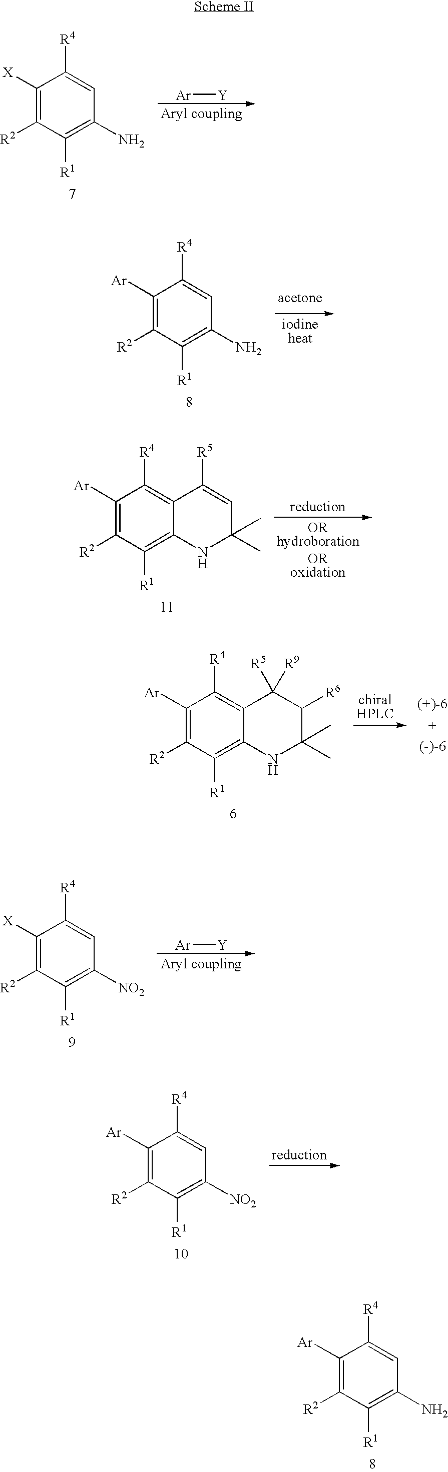

The process of Scheme II begins with an aryl cross-coupling, with, for example, an aryl halide (Structure 7), such as a 4-bromoaniline, with an aryl boronic acid, in the presence of a transition metal catalyst, for example, [1,1′-bis(diphenylphosphino)ferrocene]dichloropalladium(II), in the presence of a base, for example, aqueous sodium carbonate, to afford a compound of Structure 8. An alternate synthesis of Structure 8 begins with a halonitrobenzene, for example 4-bromonitrobenzene, and an aryl boronic acid to afford a compound of Structure 10. Treatment of Structure 10 with a reducing agent, for example, zinc metal, affords Structure 8. A compound of Structure 8 can be converted to a dihydroquinoline by treatment with, for example, iodine, in acetone heated in a sealed tube at elevated temperatures to afford a compound of Structure 11. The quinoline can be reduced by treatment with a reducing agent, for example, triethylsilane, in the presence of an acid, for example trifluoroacetic acid, to afford a tetrahydroquinoline (Structure 6, R⁶, R⁹=H). Alternatively, the dihydroquinoline can be hydrated by, for example, treatment with a hydroborating agent, for example diborane, and subsequently treated with an oxidant, such as hydrogen peroxide, in the presence of a base, for example, sodium hydroxide to afford either a 4-hydroxy-1,2,3,4-tetrahydroquinoline (Structure 6, R⁶=H, R⁹=OH), or a 4α-alkyl-3β-hydroxy-1,2,3,4-tetrahydroquinoline (Structure 6, R⁶=OH, R⁹=H). Tetrahydroquinoline compounds of Structure 6 (or any chiral synthetic precursor of Structure 6) can be separated into their corresponding enantiomers, (+)-6 and (−)-6 by chiral HPLC, with, for example, a preparative Chiracel OJ column eluted with hexanes:isopropanol. Alternatively, the enantiomers (+)-6 and (−)-6 could be prepared in enantiomerically enriched form via an enantiospecific synthesis of Structure 6, for example, by asymmetric hydroboration of Structure 11 to afford a compound of Structure 6 in enantiomerically enriched form.

In certain embodiments, synthesis of 4α-alkyl-3α-hydroxy-1,2,3,4-tetrahydroquinoline compounds (e.g. Structure 14), 1,4-dihydro-2H-quinolin-3-one compounds (e.g. Structures 13 and 15), and 4,4-dialkyl-3-hydroxy-1,2,3,4-tetrahydroquinoline compounds (e.g. Structure 16) is accomplished using Scheme III.

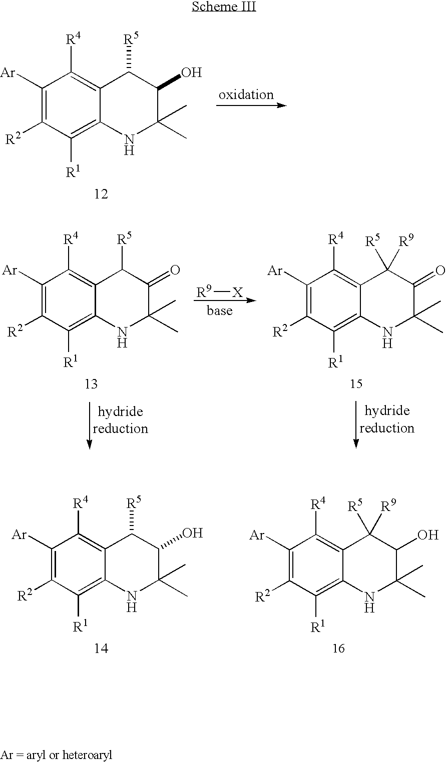

The process of Scheme III begins with the treatment of a 4α-alkyl-3β-hydroxy-1,2,3,4-tetrahydroquinoline, for example, 5-chloro-6-(3,5-dimethyl-isoxazol-4-yl)-1,2,3,4-tetrahydro-3β-hydroxy-2,2,4α,8-tetramethylquinoline, with an oxidizing agent, for example, sulfur trioxide/pyridine to afford a 1,4-dihydro-2H-quinolin-3-one of Structure 13. Treatment of the 2H-quinolin-3-one with a hydride reducing agent, for example, sodium borohydride, affords a 4α-alkyl-3α-hydroxy-1,2,3,4-tetrahydroquinoline compound of Structure 14. Alternatively, treatment of Structure 13 with alkylating agent, for example, allyl bromide, and a base, for example sodium hydride, affords a compound of Structure 15. Treatment of Structure 15 with a reducing agent, for example sodium borohydride, affords a compound of Structure 16. Compounds of Structure 13, 14, 15, or 16 can be separated into their corresponding enantiomers, by chiral HPLC, with, for example, a preparative Chiracel OJ column eluted with hexanes:isopropanol

In certain embodiments, synthesis of 4α-alkyl-3α-hydroxy-1,2,3,4-tetrahydroquinoline compounds (e.g. Structure 22) is accomplished using Scheme IV.

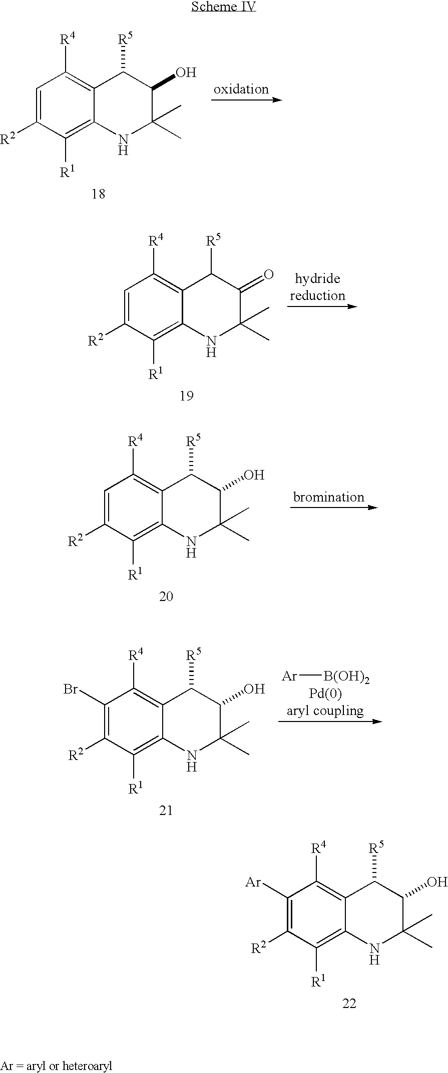

Treatment of a 4α-alkyl-3β-hydroxy-1,2,3,4-tetrahydroquinoline (Structure 18) with an oxidizing agent, for example sulfur trioxide/pyridine, affords a compound of Structure 19. Treatment of Structure 19 with a hydride reducing agent, for example, sodium borohydride, affords a compound of Structure 20. Treatment of Structure 20 with a brominating agent, for example N-bromosuccinimide, affords a compound of Structure 21. Treatment of Structure 21 with an aryl boronic acid or aryl boronate, in the presence of a transition metal catalyst, for example, [1,1′-bis(diphenylphosphino)ferrocene]dichloropalladium(II), and a base, for example, sodium carbonate, affords a compound of Structure 22. Tetrahydroquinoline compounds of Structure 22 (or any chiral synthetic precursor of Structure 22) can be separated into their corresponding enantiomers, (+)-22 and (−)-22 by chiral HPLC, with, for example, a preparative Chiracel OJ column eluted with hexanes:isopropanol.

In certain embodiments, synthesis of 4α-alkyl-3α-hydroxy-1,2,3,4-tetrahydroquinoline compounds (e.g. Structure 26), 1,4-dihydro-2H-quinolin-3-one compounds (Structures 24A and 28), and 4,4-dialkyl-3-hydroxy-1,2,3,4-tetrahydroquinoline compounds (Structure 29) is accomplished using Scheme V.

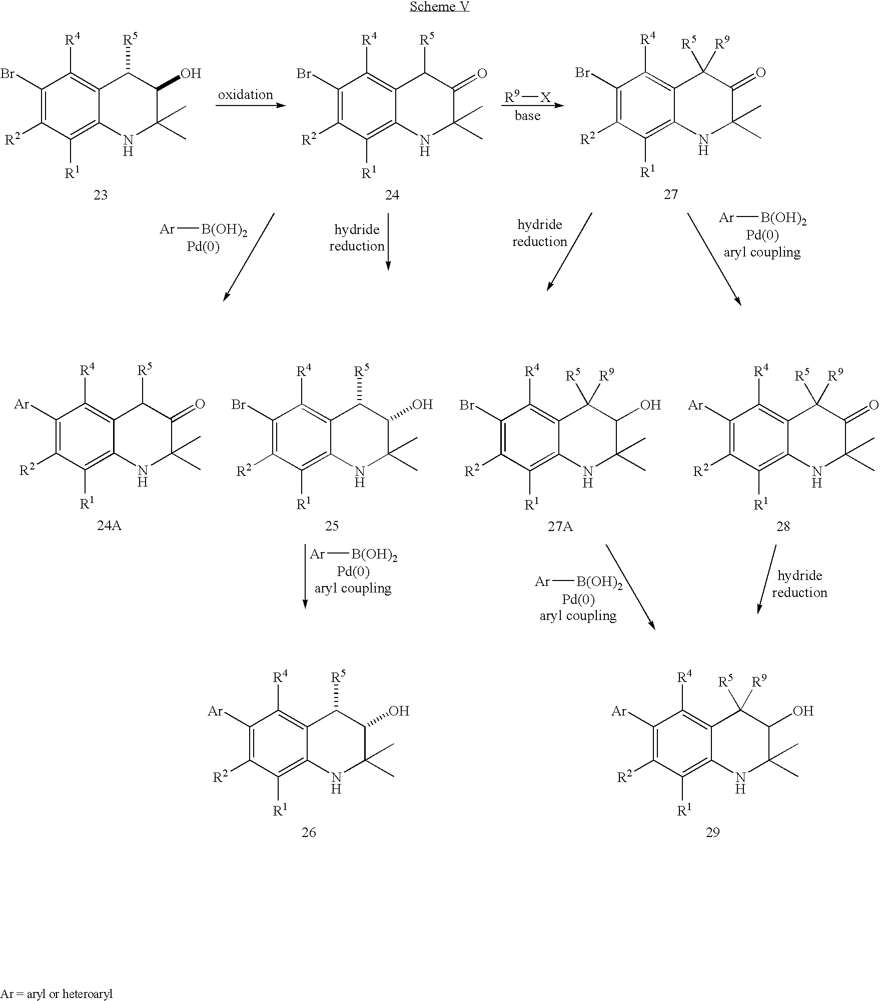

A 4α-alkyl-6-bromo-3β-hydroxy-1,2,3,4-tetrahydroquinoline (Structure 23) is treated with an oxidant, for example, sulfur trioxide/pyridine, to afford a compound of Structure 24. Treatment of a compound of Structure 24 with a hydride reducing agent, for example, sodium borohydride, affords a compound of Structure 25. Treatment of Structure 25 with an aryl boronic acid or aryl boronate, in the presence of a transition metal catalyst, for example, [1,1′-bis(diphenylphosphino)ferrocene]dichloropalladium(II), and a base, for example, sodium carbonate, affords a compound of Structure 26. Alternatively, a compound of Structure 24 may be treated with an alkylating agent, for example methyl iodide, and a base, for example sodium hydride, to afford a compound of Structure 27. Treatment of Structure 27 with an aryl boronic acid or aryl boronate, in the presence of a transition metal catalyst, for example, [1,1′-bis(diphenylphosphino)ferrocene]dichloropalladium(II), and a base, for example, sodium carbonate, affords a compound of Structure 28. Treatment of Structure 28 with a reducing agent, for example sodium borohydride, affords a compound of Structure 29. Alternatively a compound of Structure 27 can be treated with a reducing agent, for example sodium borohydride, to afford a compound of Structure 27A. Then treatment of Structure 27A with an aryl boronic acid or aryl boronate, in the presence of a transition metal catalyst, for example, [1,1′-bis(diphenylphosphino)ferrocene]dichloropalladium(II), and a base, for example, sodium carbonate, affords a compound of Structure 29. Alternatively, treatment of a compound of Structure 24 with an aryl boronic acid or aryl boronate, in the presence of a transition metal catalyst, for example, [1,1′-bis(diphenylphosphino)ferrocene]dichloropalladium(II), and a base, for example, sodium carbonate, affords a compound of Structure 24A.

In certain embodiments, synthesis of 6-aryl- and 6-heteroaryl bromides, boronic acids, and boronate esters is accomplished using Schemes VI-X. The process of Scheme VI begins with the treatment of a phenol, for example 2-cyanophenol, with a brominating agent, for example N-bromosuccinimide, in the presence of a base, for example diisopropylamine, to afford an o-bromophenol (Structure 31). Structure 31 can be alkylated by treatment with an alkyl halide, for example, methyl iodide, in the presence of a base, for example potassium carbonate, to afford a compound of Structure 32. A compound of Structure 32 can be converted to a compound of Structure 33 by treatment with a boronating agent, for example, 4,4,5,5-tetramethyl-1,3,2-dioxaborolane, in the presence of a transition metal catalyst, for example, Pd₂dba₃, and a phosphorus ligand, for example, 2-(dicyclohexylphosphino)biphenyl, in the presence of a base, for example, triethylamine, to afford a compound of Structure 33.

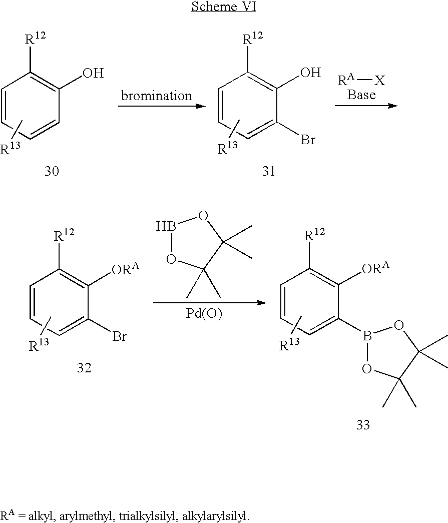

The process of Scheme VII begins with a metallation of a 1,3-dimethoxybenzene of Structure 34, for example, 2,4-dimethoxybenzonitrile, with a base, for example lithium tetramethylpiperidide, and a silylating agent, for example, chlorotrimethylsilane, to afford a compound of Structure 35. Compound 35 is converted to the corresponding bromide by treatment with a brominating agent, for example, N-bromosuccimide, to afford a compound of Structure 36.

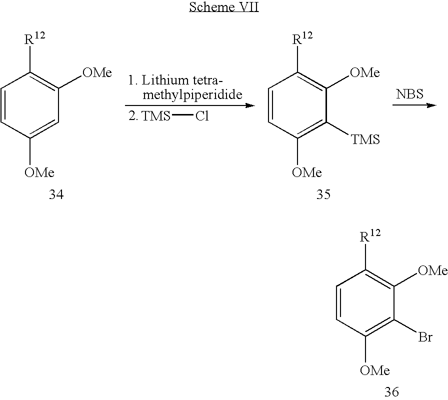

The process of Scheme VIII is the treatment of Structure 37, for example, 1-indanone, with bromine in the presence of a Lewis Acid, for example, aluminum chloride, to afford a compound of Structure 38.

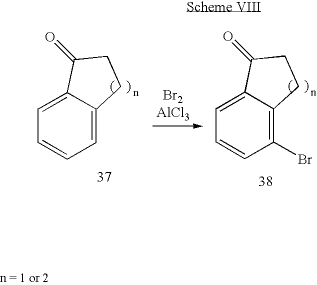

The process of Scheme IX is the treatment of a 2-nitrohalobenzene (Structure 39), for example, 1-bromo-2-nitrobenzene, with a vinyl Grignard reagent, for example vinylmagnesium bromide, to afford a compound of Structure 40.

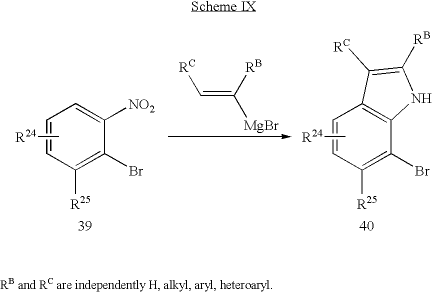

The process of Scheme X is the treatment of Structure 41 with an acid, for example, hydrochloric acid in acetic acid, to afford a compound of Structure 42.

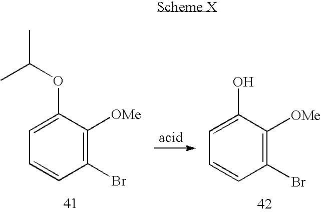

The synthesis of compounds of Structure 44 and 45 is depicted in Scheme XI and begins with the treatment of Structure 43 with a reducing agent, for example zinc dust, to afford the corresponding amino compound of Structure 44. Structure 44 can be alkylated, acylated, or sulfonylated be treatment, for example, with methyl iodide, methyl chloroformate, or methansulfonyl chloride, respectively, to afford compounds of Structure 45.

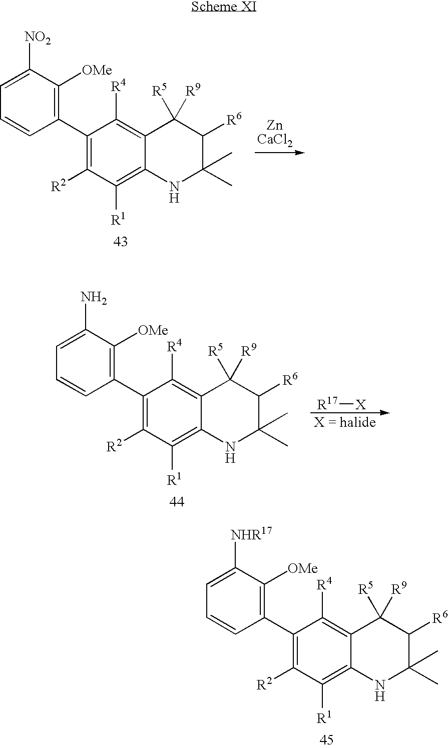

The synthesis of compounds of Structure 48 and 49 is depicted in Scheme XII. Deprotection of an ether of Structure 46 can be accomplished by treatment with an acid, for example methanesulfonic acid to afford a phenol of Structure 47. Treatment of Structure 47 with a haloformate or haloacetate, for example, ethyl bromoacetate, followed by reduction with, for example zinc dust, affords a compound of Structure 48. Treatment of Structure 48 with an alkylating agent, for example, methyl iodide, in the presence of a base, for example, sodium hydride, affords a compound of Structure 49. Alternatively, treatment of Structure 47 with a reducing agent, for example, zinc dust, affords a compound of Structure 47A. Alternatively, treatment of Structure 47 with an alkylating agent, for example, allyl bromide, in the presence of a base, for example potassium carbonate, affords a compound of Structure 47B.

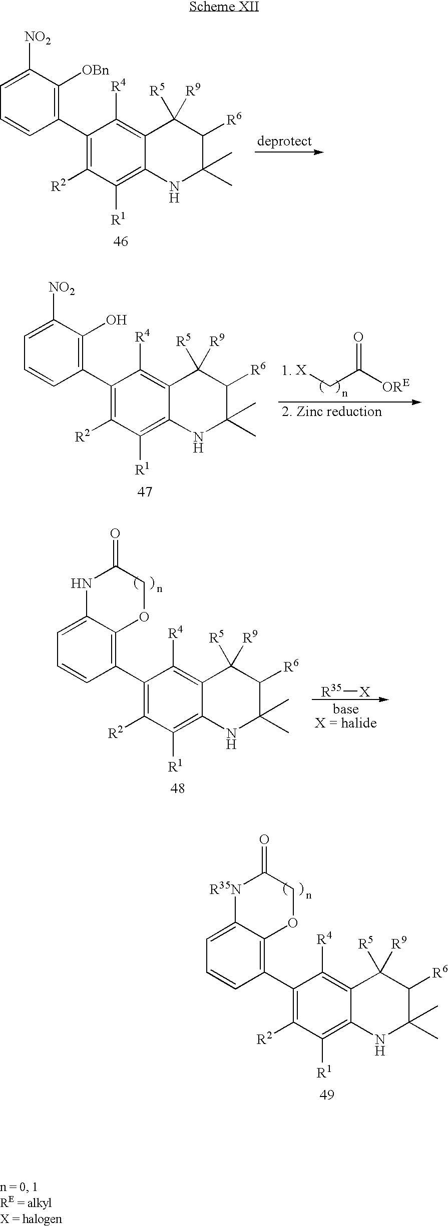

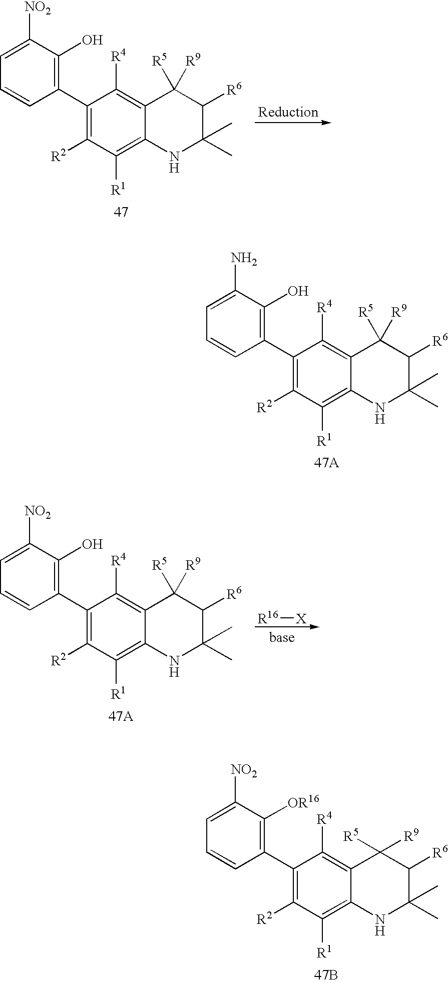

The synthesis of compounds of Structure 51 is depicted in Scheme XIII. A nitro derivative of Structure 50 is treated with an ethenyl magnesium halide, for example, vinyl magnesium bromide, to afford a compound of Structure 51. Treatment of Structure 50 with a reducing agent, for example, zinc metal, affords a Compound of Structure 51B.

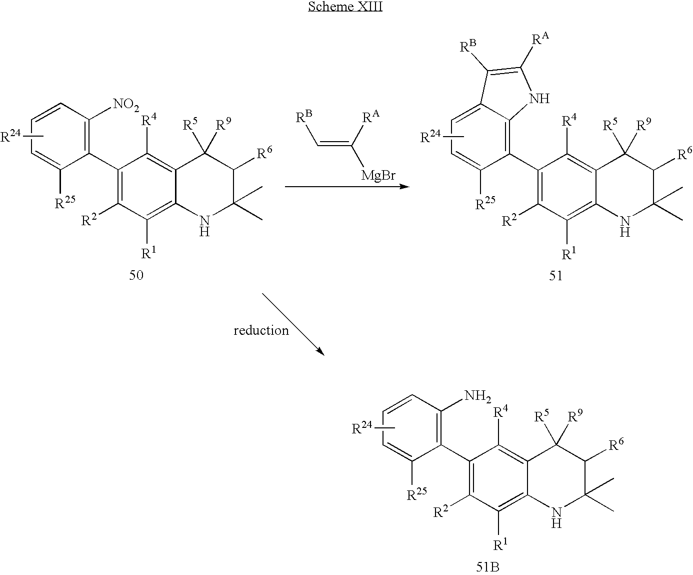

The synthesis of compounds of Structure 53 and 54 is depicted in Scheme XIV. An indole compound of Structure 52 can be alkylated at the 3-position of the indole by treatment with an ethenyl ketone, for example, methyl vinyl ketone, in the presence of a Lewis acid, for example, indium trichloride, to afford a compound of Structure 53.

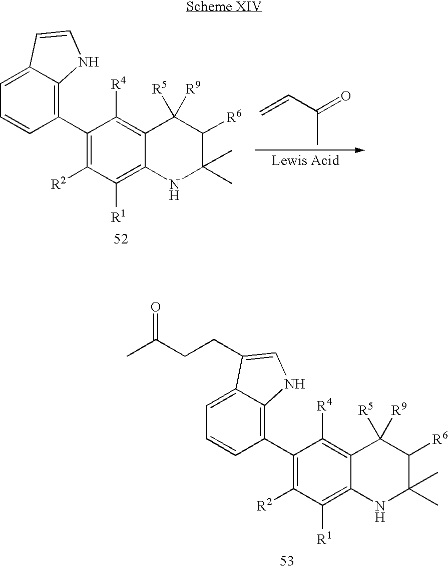

The synthesis of compounds of Structure 55, 56 and 57 is depicted in Scheme XIV. An indole of Structure 54 is treated with a brominating agent, for example, N-bromosuccinimide, in the presence of water, to afford a mixture of compounds of Structure 55 and 56. Structure 56 may be treated with an organometallic reagent, for example, an aryl boronic acid, in the presence of a transition metal catalyst, for example, [1,1′-bis(diphenylphosphino)ferrocene]dichloropalladium(II), in the presence of a base, for example, aqueous sodium carbonate, to afford a compound of Structure 57. Alternatively, a compound of Structure 54 can be treated with a reducing agent, for example, sodium cyanoborohydride, in the presence of an acid, for example, acetic acid, to afford a compound of Structure 58.

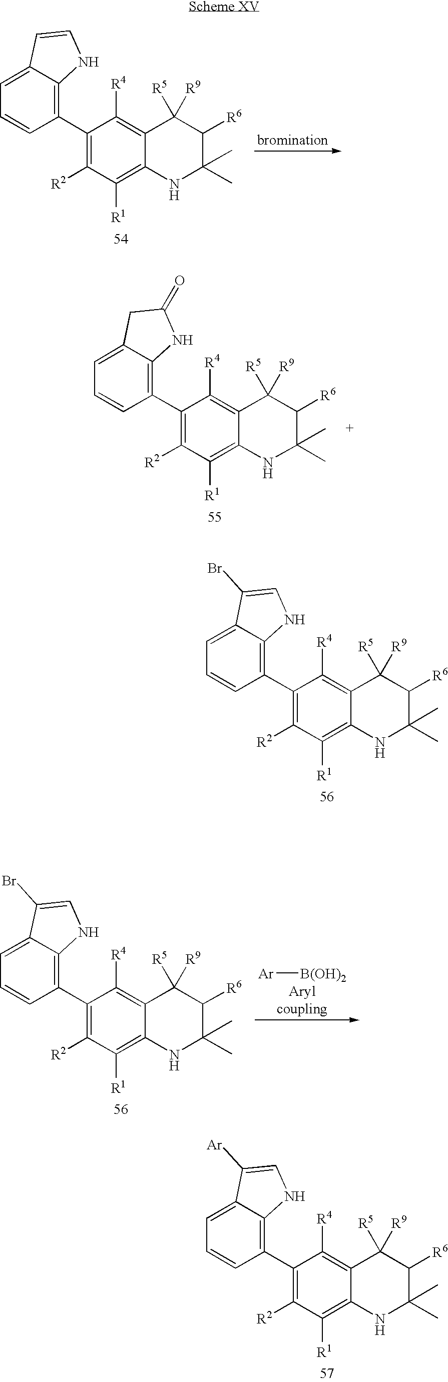

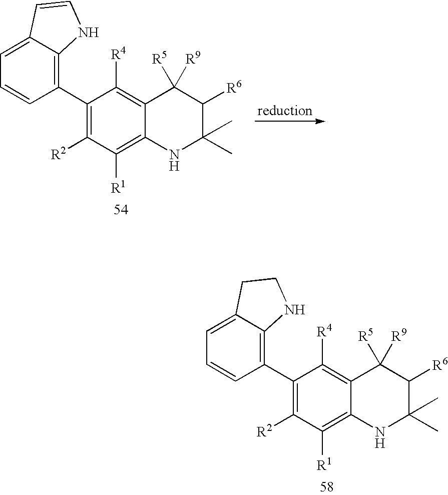

The synthesis of compounds of Structure 60 is depicted in Scheme XVI. A compound of Structure 59 is treated with hydroxylamine hydrochloride or an alkoxy amine hydrochloride to afford a compound of Structure 60.

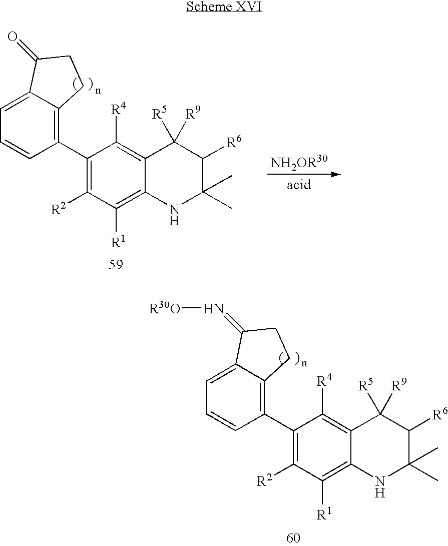

The synthesis of compounds of Structure 63 is depicted in Scheme XVII. Treatment a 4-hydroxy-1,2,3,4-tetrahydroquinoline (Structure 61) with an acid, for example, trifluoroacetic acid, affords a compound of Structure 62. Treatment of Structure 62 with an organometallic reagent, for example, an aryl boronic acid, in the presence of a transition metal catalyst, for example, [1,1′-bis(diphenylphosphino)ferrocene]dichloropalladium(II), in the presence of a base, for example, aqueous sodium carbonate, affords a compound of Structure 63. Alternatively, a compound of Structure 63 can be obtained by treatment of a 4-hydroxy-1,2,3,4-tetrahydroquinoline of Structure 64 with an acid, for example, trifluoroacetic acid.

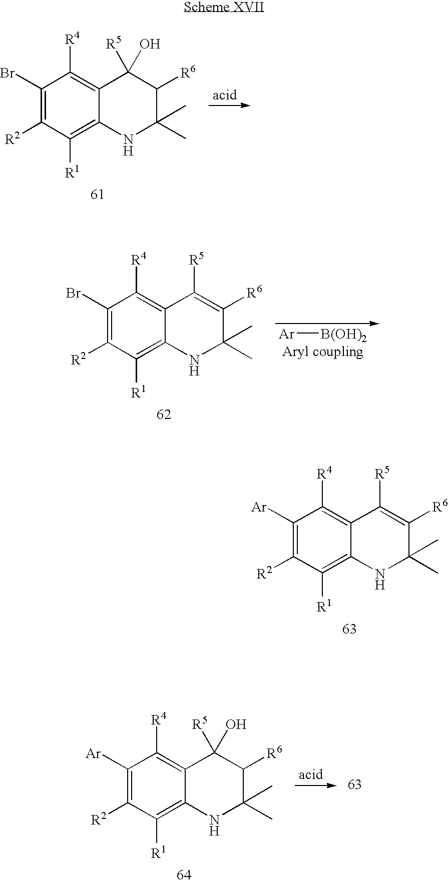

Synthesis of compounds of Structure 68 is may be accomplished as set forth in Scheme XVIII. Treatment of a 3,4-dihydroquinoline with a cyanating agent, for example, zinc cyamide, in the presence of a metal catalyst, for example Pd₂dba₃, affords a compound of Structure 65. The olefin of Structure 65 can be treated in a number of ways. For example, the dihydroquinoline can be hydrated by treatment with a hydroborating agent, such as diborane, and subsequently treated with an oxidant, such as hydrogen peroxide, in the presence of a base, for example, sodium hydroxide, to afford either a 4-hydroxy-1,2,3,4-tetrahydroquinoline (Structure 66, R⁶=H, R9=OH) or a 4α-alkyl-3β-hydroxy-1,2,3,4-tetrahydroquinoline. (Structure 66, R6=OH, R9=H). Structure 66 may be converted to the desired products as described in Scheme I, starting from Structure 3.

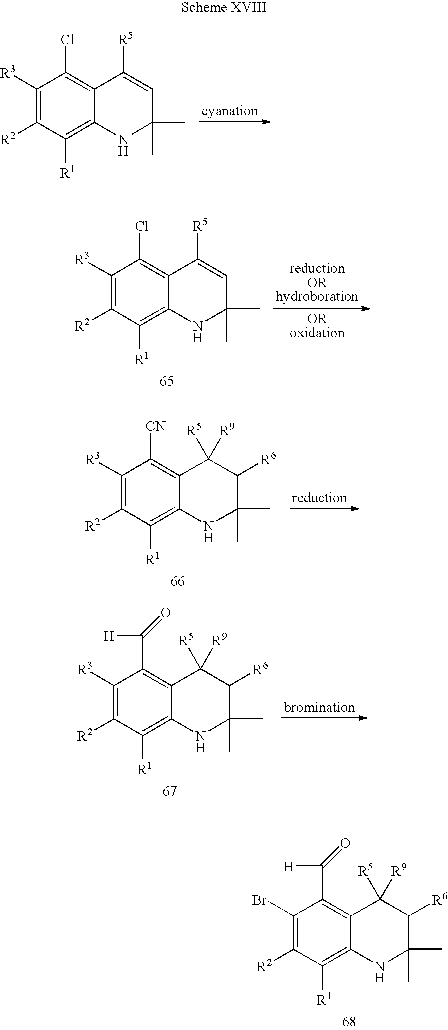

Alternatively, the cyano derivative of Structure 66 can be partially reduced to the corresponding aldehyde with a reducing agent, for example, diisobutylaluminum hydride, to afford a compound of Structure 67. Structure 67 can be halogenated at the 6-position by treatment with a halogenating agent, for example, N-bromosuccinimide, to afford a compound of Structure 68.

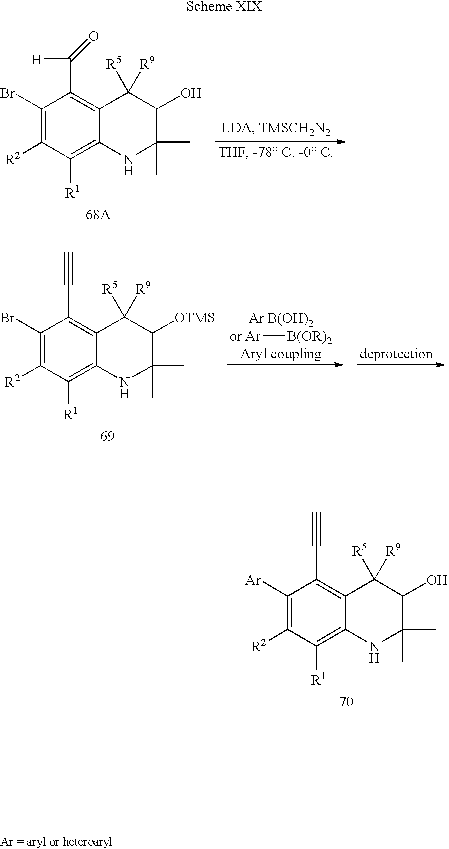

An example of how a compound of Structure 68 can be elaborated is illustrated in Scheme XIX. A compound of Structure 68A can be converted to the corresponding acetylene by treatment with a base, for example, lithium diisopropylamine, and (trimethylsilyl)diazomethane, to afford a compound of Structure 69. Treatment of Structure 69 with an organometallic agent, for example, an aryl boronic acid, in the presence of a transition metal catalyst, for example, [1,1′-bis(diphenylphosphino)ferrocene]dichloropalladium(II), in the presence of a base, for example, sodium carbonate, affords the corresponding arylated product. Deprotection of the silyl ether is effected by treatment with a fluoride source, for example, TBAF, to afford a compound of Structure 70.

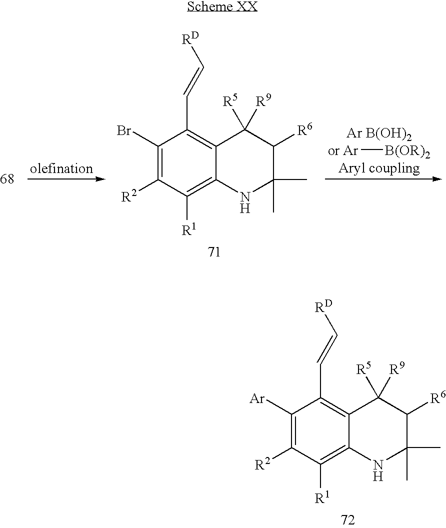

In certain embodiments, the synthesis of tetrahydroquinoline compounds of Structure 72 is accomplished using Scheme XX. Treatment of a compound of Structure 68 with an olefinating reagent, for example, diethyl benzylphosphonate, and a base, for example, sodium hydride, affords a compound of Structure 71. Treatment of Structure 71 with an organometallic agent, for example, an aryl boronic acid, in the presence of a transition metal catalyst, for example, [1,1′-bis(diphenylphosphino)ferrocene]dichloropalladium(II), in the presence of a base, for example, sodium carbonate, affords a compound of Structure 72.

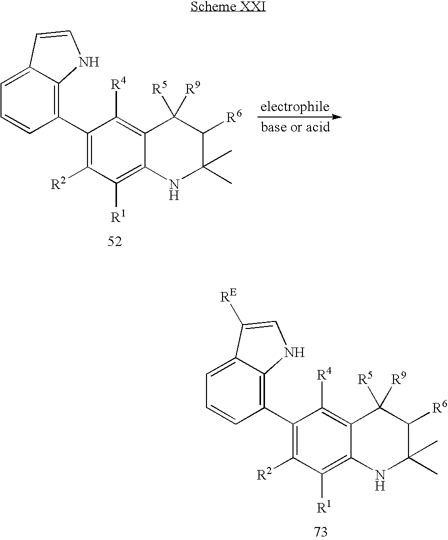

In certain embodiments, the synthesis of compounds of Structure 73 is depicted in Scheme XXI. A compound of Structure 52 is treated with an electrophile, for example, allyl bromide, in the presence of an agent to promote the reaction, for example, sodium hydride, to afford a compound of Structure 73.

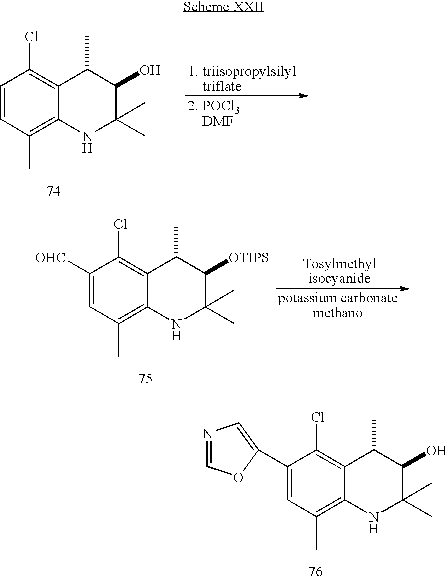

In certain embodiments, synthesis of 6-heteroaryl 1,2,3,4-tetrahydroquinoline compounds of Structure 76 is depicted in Scheme XXII. A compound of Structure 74 is treated with a silylating agent, for example, triisopropylsilyl triflate, followed by treatment with an acylating agent, generated from, for example, POCl₃ and DMF, to afford Structure 75. Structure 75 is then treated to form a heterocycle, for example, an oxazole, by treatment with, for example, tosylmethyl isocyamide, to afford a compound of Structure 76.

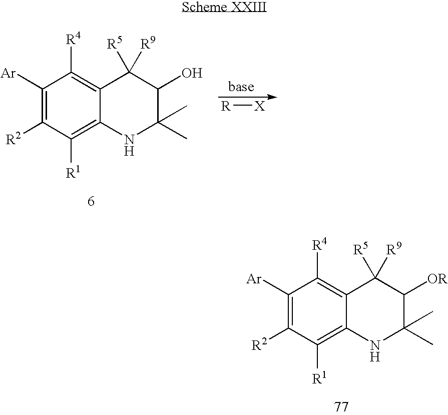

In certain embodiments, synthesis of 6-heteroaryl 1,2,3,4-tetrahydroquinoline compounds of Structure 76 is depicted in Scheme XXIII. A compound of Structure 6 may be alkylated with a base, for example, sodium bis(trimethylsilyl)amide, and an alkylating agent, for example, iodomethane, to afford a compound of Structure 77.

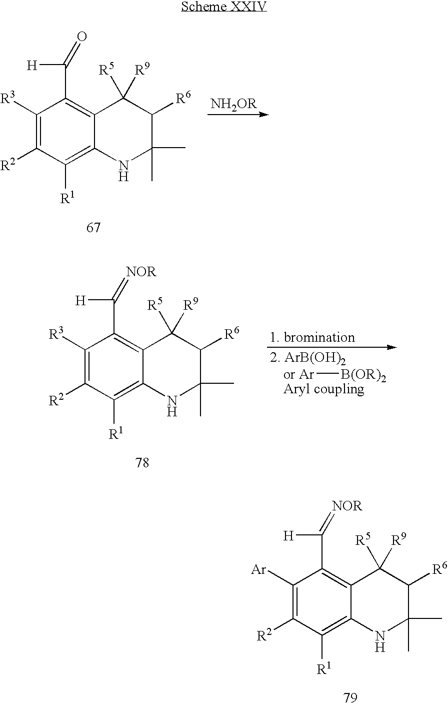

In certain embodiments, synthesis of 6-aryl and 6-heteroaryl 1,2,3,4-tetrahydroquinoline compounds of Structure 79 is depicted in Scheme XXIV. Structure 67 is converted to the corresponding oxime by treatment an alkoxyamine hydrochloride, for example, methoxyamine hydrochloride, to afford a compound of Structure 78. Structure 78 can be halogenated at the 6-position by treatment with a halogenating agent, for example, N-bromosuccinimide, followed by treatment with an organometallic agent, for example, an aryl boronic acid, in the presence of a transition metal catalyst, for example, [1,1′-bis(diphenylphosphino)ferrocene]dichloropalladium(II), in the presence of a base, for example, sodium carbonate, to afford a compound of Structure 79.

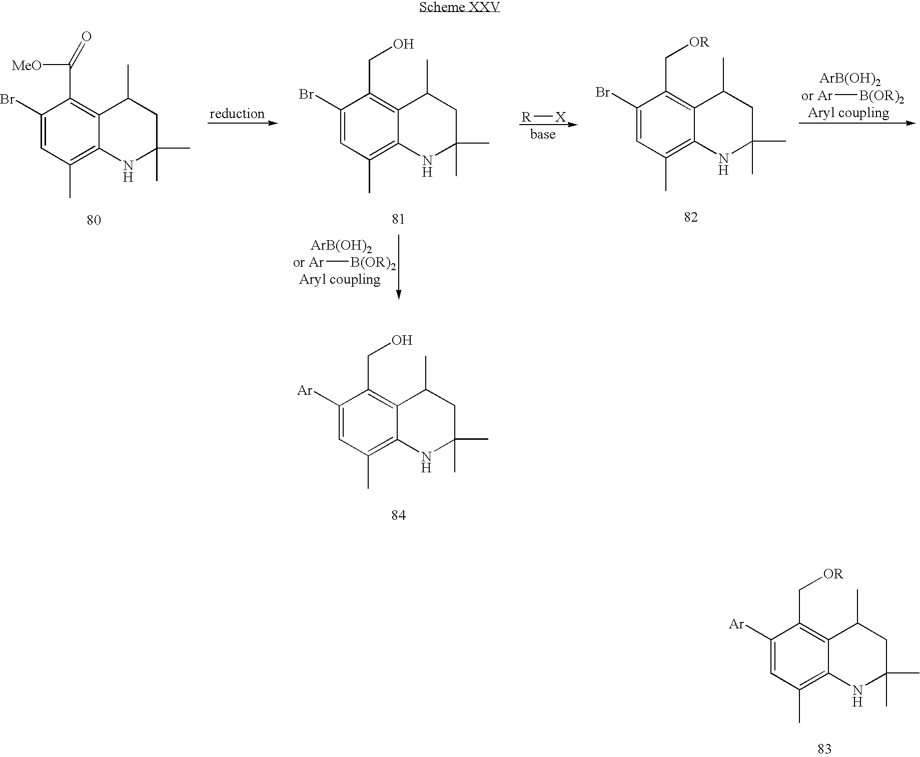

In certain embodiments, synthesis of 6-aryl and 6-heteroaryl 1,2,3,4-tetrahydroquinoline compounds of Structure 83 and 84 is conducted as depicted in Scheme XXV. A compound of Structure 80 is converted to Structure 81 by treatment with a reducing agent, for example, lithium aluminum hydride, to afford a compound of Structure 81. Treatment of Structure 81 with an organometallic agent, for example, an aryl boronic acid, in the presence of a transition metal catalyst, for example, [1,1′-bis(diphenylphosphino)ferrocene]dichloropalladium(II), in the presence of a base, for example, sodium carbonate, affords a compound of Structure 84. Alternatively, a compound of Structure 81 can be alkylated at the oxygen by treatment with an alkyl halide, for example benzyl bromide, in the presence of a base, for example, sodium hydride, to afford a compound of Structure 82. Treatment of Structure 82 with an organometallic agent, for example, an aryl boronic acid, in the presence of a transition metal catalyst, for example, [1,1′-bis(diphenylphosphino)ferrocene]dichloropalladium(II), in the presence of a base, for example, sodium carbonate, affords a compound of Structure 83.

In certain embodiments, the invention provides a salt corresponding to any of the compounds provided herein. In certain embodiments, the invention provides a salt corresponding to a selective glucocorticoid receptor modulator, a selective mineralocorticoid receptor modulator and/or a selective glucocoroticoid/mineralocorticoid receptor modulator. In certain embodiments, the invention provides a salt corresponding to a selective glucocorticoid receptor binding agent, a selective mineralocorticoid receptor binding agent and/or a selective glucocoroticoid/mineralocorticoid receptor binding agent. In certain embodiments, a salt is obtained by reacting a compound with an inorganic acid, such as hydrochloric acid, hydrobromic acid, sulfuric acid, nitric acid, phosphoric acid, methanesulfonic acid, ethanesulfonic acid, p-toluenesulfonic acid, salicylic acid, and the like. In certain embodiments, a salt is obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

In certain embodiments, one or more carbon atoms of a compound of the present invention is replaced with silicon. See e.g. WO 03/037905A1; Tacke and Zilch, Endeavour, New Series, 10, 191-197 (1986); Bains and Tacke, Curr. Opin. Drug Discov Devel. July:6(4):526-43 (2003). In certain embodiments, compounds of the present invention comprising one or more silicon atoms possess certain desired properties, including, but not limited to, greater stability and/or longer half-life in a patient, when compared to the same compound in which none of the carbon atoms have been replaced with a silicon atom.

Certain Assays

In certain embodiments, compounds of the present invention are capable of modulating activity of glucocorticoid and/or mineralocorticoid receptors in a “co-transfection” assay (also called a “cis-trans” assay), which has been discussed previously. See e.g. Evans et al., Science, 240:889-95 (1988); U.S. Pat. Nos. 4,981,784 and 5,071,773; Pathirana et al., “Nonsteroidal Human Progesterone Receptor Modulators from the Marie Alga Cymopolia Barbata,” Mol. Pharm. 47:630-35 (1995)). Modulating activity in a co-transfection assay has been shown to correlate with in vivo modulating activity. Thus, in certain embodiments, such assays are predictive of in vivo activity. See, e.g. Berger et al., J. Steroid Biochem. Molec. Biol. 41:773 (1992).

In certain co-transfection assays, two different co-transfection plasmids are prepared. In the first co-transfection plasmid, cloned cDNA encoding an intracellular receptor (e.g., glucocorticoid or mineralocoticoid receptor) is operatively linked to a constitutive promoter (e.g., the SV 40 promoter). In the second co-transfection plasmid, cDNA encoding a reporter protein, such as firefly luciferase (LUC), is operatively linked to a promoter that is activated by a receptor-dependant activation factor. Both co-transfection plasmids are co-transfected into the same cells. Expression of the first co-transfection plasmid results in production of the intracellular receptor protein. Activation of that intracellular receptor protein (e.g. by binding of an agonist) results in production of a receptor-dependant activation factor for the promoter of the second co-transfection plasmid. That receptor-dependant activation factor in turn results in expression of the reporter protein encoded on the second co-transfection plasmid. Thus, reporter protein expression is linked to activation of the receptor. Typically, that reporter activity can be conveniently measured (e.g. as increased luciferase production).

Certain co-transfection assays can be used to identify agonists, partial agonists, and/or antagonists of intracellular receptors. In certain embodiments, to identify agonists, co-transfected cells are exposed to a test compound. If the test compound is an agonist or partial agonist, reporter activity is expected to be higher compared to co-transfected cells in the absence of the test compound. In certain embodiments, to identify antagonists, the cells are exposed to a known agonist (e.g., the natural ligand for the receptor) in the presence and absence of a test compound. If the test compound is an antagonist, reporter activity is expected to be lower than that of cells exposed only to the known agonist.

In certain embodiments, compounds of the invention are used to detect the presence, quantity and/or state of receptors in a sample. In certain of such embodiments, samples are obtained from a patient. In certain embodiments, compounds are radio- or isotopically-labeled. For example, compounds of the present invention that selectively bind glucocorticoid and or mineralocorticoid receptors may be used to determine the presence or amount of such receptors in a sample, such as cell homogenates and lysates.

Certain Pharmaceutical Agents

In certain embodiments, at least one selective glucocoroticoid receptor modulator, or pharmaceutically acceptable salt, ester, amide, and/or prodrug thereof, either alone or combined with one or more pharmaceutically acceptable carriers, forms a pharmaceutical agent. In certain embodiments, at least one selective mineralocorticoid receptor modulator, or pharmaceutically acceptable salt, ester, amide, and/or prodrug thereof, either alone or combined with one or more pharmaceutically acceptable carriers, forms a pharmaceutical agent. In certain embodiments, at least one selective glucocoroticoid/mineralocorticoid receptor modulator, or pharmaceutically acceptable salt, ester, amide, and/or prodrug thereof, either alone or combined with one or more pharmaceutically acceptable carriers, forms a pharmaceutical agent. In certain embodiments, the pharmaceutical agent comprises at least one compound of Formula I, II, or III, as defined and described herein. Techniques for formulation and administration of compounds of the present invention may be found for example, in “Remington's Pharmaceutical Sciences,” Mack Publishing Co., Easton, Pa., 18th edition, 1990, which is incorporated herein by reference in its entirety.

In certain embodiments, a pharmaceutical agent comprising one or more compounds of the present invention, such as a compound of Formula I, II, or III, is prepared using known techniques, including, but not limited to mixing, dissolving, granulating, dragee-making, levigating, emulsifying, encapsulating, entrapping or tabletting processes.

In certain embodiments, a pharmaceutical agent comprising one or more compounds of the present invention is a liquid (e.g. a suspension, elixir and/or solution). In certain of such embodiments, a liquid pharmaceutical agent comprising one or more compounds of the present invention is prepared using ingredients known in the art, including, but not limited to, water, glycols, oils, alcohols, flavoring agents, preservatives, and coloring agents.

In certain embodiments, a pharmaceutical agent comprising one or more compounds of the present invention is a solid (e.g. a powder, tablet, and/or capsule). In certain of such embodiments, a solid pharmaceutical agent comprising one or more compounds of the present invention is prepared using ingredients known in the art, including, but not limited to, starches, sugars, diluents, granulating agents, lubricants, binders, and disintegrating agents.

In certain embodiments, a pharmaceutical agent comprising one or more compounds of the present invention is formulated as a depot preparation. Certain of such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical agent comprising one or more compounds of the present invention comprises a delivery system. Examples of delivery systems include, but are not limited to, liposomes and emulsions. Certain delivery systems are useful for preparing certain pharmaceutical agents including those comprising hydrophobic compounds. In certain embodiments, certain organic solvents such as dimethylsulfoxide are used.

In certain embodiments, a pharmaceutical agent comprising one or more compounds of the present invention comprises one or more tissue-specific delivery molecules designed to deliver the pharmaceutical agent to specific tissues or cell types. For example, in certain embodiments, pharmaceutical agents include liposomes coated with a tissue-specific antibody.

In certain embodiments, a pharmaceutical agent comprising one or more compounds of the present invention comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™, and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g. polyvinyl pyrrolidone; and other sugars or polysaccharides may substitute for dextrose.

In certain embodiments, a pharmaceutical agent comprising one or more compounds of the present invention comprises a sustained-release system. A non-limiting example of such a sustained-release system is a semi-permeable matrix of solid hydrophobic polymers. In certain embodiments, sustained-release systems may, depending on their chemical nature, release compounds over a period of hours, days, weeks or months.

Certain compounds used in pharmaceutical agent of the present invention may be provided as pharmaceutically acceptable salts with pharmaceutically compatible counterions. Pharmaceutically compatible salts may be formed with many acids, including but not limited to hydrochloric, sulfuric, acetic, lactic, tartaric, malic, succinic, etc.

In certain embodiments, a pharmaceutical agent comprising one or more compounds of the present invention comprises an active ingredient in a therapeutically effective amount. In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, a pharmaceutical agent comprising one or more compounds of the present invention is formulated as a prodrug. In certain embodiments, prodrugs are useful because they are easier to administer than the corresponding active form. For example, in certain instances, a prodrug may be more bioavailable (e.g. through oral administration) than is the corresponding active form. In certain instances, a prodrug may have improved solubility compared to the corresponding active form. In certain embodiments, a prodrug is an ester. In certain embodiments, such prodrugs are less water soluble than the corresponding active form. In certain instances, such prodrugs possess superior transmittal across cell membranes, where water solubility is detrimental to mobility. In certain embodiments, the ester in such prodrugs is metabolically hydrolyzed to carboxylic acid. In certain instances the carboxylic acid containing compound is the corresponding active form. In certain embodiments, a prodrug comprises a short peptide (polyaminoacid) bound to an acid group. In certain of such embodiments, the peptide is metabolized to form the corresponding active form.

In certain embodiments, a pharmaceutical agent comprising one or more compounds of the present invention is useful for treating a conditions or disorder in a mammalian, and particularly in a human patient. Suitable administration routes include, but are not limited to, oral, rectal, transmucosal, intestinal, enteral, topical, suppository, through inhalation, intrathecal, intraventricular, intraperitoneal, intranasal, intraocular and parenteral (e.g., intravenous, intramuscular, intramedullary, and subcutaneous). In certain embodiments, pharmaceutical intrathecals are administered to achieve local rather than systemic exposures. For example, pharmaceutical agents may be injected directly in the area of desired effect (e.g., in the renal or cardiac area).

In certain embodiments, a pharmaceutical agent comprising one or more compounds of the present invention is administered in the form of a dosage unit (e.g. tablet, capsule, bolus, etc.). In certain embodiments, such dosage units comprise a selective glucocorticoid and/or minerlaocorticoid receptor modulator in a dose from about 1 μg/kg of body weight to about 50 mg/kg of body weight. In certain embodiments, such dosage units comprise a selective glucocorticoid and/or minerlaocorticoid receptor modulator in a dose from about 2 μg/kg of body weight to about 25 mg/kg of body weight. In certain embodiments, such dosage units comprise a selective glucocorticoid and/or minerlaocorticoid receptor modulator in a dose from about 10 μg/kg of body weight to about 5 mg/kg of body weight. In certain embodiments, pharmaceutical agents are administered as needed, once per day, twice per day, three times per day, or four or more times per day. It is recognized by those skilled in the art that the particular dose, frequency, and duration of administration depends on a number of factors, including, without limitation, the biological activity desired, the condition of the patient, and tolerance for the pharmaceutical agent.

In certain embodiments, a pharmaceutical agent comprising a compound of the present invention is prepared for oral administration. In certain of such embodiments, a pharmaceutical agent is formulated by combining one or more compounds of the present invention with one or more pharmaceutically acceptable carriers. Certain of such carriers enable compounds of the invention to be formulated as tablets, pills, dragees, capsules, liquids, gels, syrups, slurries, suspensions and the like, for oral ingestion by a patient. In certain embodiments, pharmaceutical agents for oral use are obtained by mixing one or more compounds of the present invention and one or more solid excipient. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical agents are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain of such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical agents for oral administration are push-fit capsules made of gelatin. Certain of such push-fit capsules comprise one or more compounds of the present invention in admixture with one or more filler such as lactose, binders such as starches, and/or lubricants such as talc or magnesium stearate and, optionally, stabilizers. In certain embodiments, pharmaceutical agents for oral administration are soft, sealed capsules made of gelatin and a plasticizer, such as glycerol or sorbitol. In certain soft capsules, one or more compounds of the present invention are be dissolved or suspended in suitable liquids, such as fatty oils, liquid paraffin, or liquid polyethylene glycols. In addition, stabilizers may be added.

In certain embodiments, pharmaceutical agents are prepared for buccal administration. Certain of such pharmaceutical agents are tablets or lozenges formulated in conventional manner.

In certain embodiments, a pharmaceutical agent is prepared for administration by injection (e.g., intravenous, subcutaneous, intramuscular, etc.). In certain of such embodiments, a pharmaceutical agent comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical agents for injection are presented in unit dosage form, e.g. in ampoules or in multi-dose containers. Certain pharmaceutical agents for injection are suspensions, solutions or emulsions in oily or aqueous vehicles, and may contain formulatory agents such as suspending, stabilizing and/or dispersing agents. Certain solvents suitable for use in pharmaceutical agents for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the compounds to allow for the preparation of highly concentrated solutions.

In certain embodiments, a pharmaceutical agent is prepared for transmucosal administration. In certain of such embodiments penetrants appropriate to the barrier to be permeated are used in the formulation. Such penetrants are generally known in the art.

In certain embodiments, a pharmaceutical agent is prepared for administration by inhalation. Certain of such pharmaceutical agents for inhalation are prepared in the form of an aerosol spray in a pressurized pack or a nebulizer. Certain of such pharmaceutical agents comprise a propellant, e.g. dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a compound of the invention and a suitable powder base such as lactose or starch.

In certain embodiments, a pharmaceutical agent is prepared for rectal administration, such as a suppositories or retention enema. Certain of such pharmaceutical agents comprise known ingredients, such as cocoa butter and/or other glycerides.

In certain embodiments, a pharmaceutical agent is prepared for topical administration. Certain of such pharmaceutical agents comprise bland moisturizing bases, such as ointments or creams. Exemplary suitable ointment bases include, but are not limited to, petrolatum, petrolatum plus volatile silicones, lanolin and water in oil emulsions such as Eucerin™, available from Beiersdorf (Cincinnati, Ohio). Exemplary suitable cream bases include, but are not limited to, Nivea™ Cream, available from Beiersdorf (Cincinnati, Ohio), cold cream (USP), Purpose Cream™, available from Johnson & Johnson (New Brunswick, N.J.), hydrophilic ointment (USP) and Lubriderm™, available from Pfizer (Morris Plains, N.J.).

In certain embodiments, the formulation, route of administration and dosage for a pharmaceutical agent of the present invention can be chosen in view of a particular patient's condition. (See e.g. Fingl et al. 1975, in “The Pharmacological Basis of Therapeutics”, Ch. 1 p. 1). In certain embodiments, a pharmaceutical agent is administered as a single dose. In certain embodiments, a pharmaceutical agent is administered as a series of two or more doses administered over one or more days.

In certain embodiments, a pharmaceutical agent of the present invention is administered to a patient between about 0.1% and 500%, 5% and 200%, 10% and 100%, 15% and 85%, 25% and 75%, or 40% and 60% of an established human dosage. Where no human dosage is established, a suitable human dosage may be inferred from ED₅₀ or ID₅₀ values, or other appropriate values derived from in vitro or in vivo studies.

In certain embodiments, a daily dosage regimen for a patient comprises an oral dose of between 0.1 mg and 2000 mg, 5 mg and 1500 mg, 10 mg and 1000 mg, 20 mg and 500 mg, 30 mg and 200 mg, or 40 mg and 100 mg of a compound of the present invention. In certain embodiments, a daily dosage regimen is administered as a single daily dose. In certain embodiments, a daily dosage regimen is administered as two, three, four, or more than four doses.

In certain embodiments, a pharmaceutical agent of the present invention is administered by continuous intravenous infusion. In certain of such embodiments, from 0.1 mg to 500 mg of a composition of the present invention is administered per day.

In certain embodiments, a pharmaceutical agent of the invention is administered for a period of continuous therapy. For example, a pharmaceutical agent of the present invention may be administered over a period of days, weeks, months, or years.

Dosage amount, interval between doses, and duration of treatment may be adjusted to achieve a desired effect. In certain embodiments, dosage amount and interval between doses are adjusted to maintain a desired concentration on compound in a patient. For example, in certain embodiments, dosage amount and interval between doses are adjusted to provide plasma concentration of a compound of the present invention at an amount sufficient to achieve a desired effect. In certain of such embodiments the plasma concentration is maintained above the minimal effective concentration (MEC). In certain embodiments, pharmaceutical agents of the present invention are administered with a dosage regimen designed to maintain a concentration above the MEC for 10-90% of the time, between 30-90% of the time, or between 50-90% of the time.

In certain embodiments in which a pharmaceutical agent is administered locally, the dosage regimen is adjusted to achieve a desired local concentration of a compound of the present invention.

In certain embodiments, a pharmaceutical agent may be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

In certain embodiments, a pharmaceutical agent is in powder form for constitution with a suitable vehicle, e.g., sterile pyrogen-free water, before use.

Certain Combination Therapies

In certain embodiments, one or more pharmaceutical agents of the present invention are co-administered with one or more other pharmaceutical agents. In certain embodiments, such one or more other pharmaceutical agents are designed to treat the same disease or condition as the one or more pharmaceutical agents of the present invention. In certain embodiments, such one or more other pharmaceutical agents are designed to treat a different disease or condition as the one or more pharmaceutical agents of the present invention. In certain embodiments, such one or more other pharmaceutical agents are designed to treat an undesired effect of one or more pharmaceutical agents of the present invention. In certain embodiments, one or more pharmaceutical agents of the present invention is co-administered with another pharmaceutical agent to treat an undesired effect of that other pharmaceutical agent. In certain embodiments, one or more pharmaceutical agents of the present invention and one or more other pharmaceutical agents are administered at the same time. In certain embodiments, one or more pharmaceutical agents of the present invention and one or more other pharmaceutical agents are administered at the different times. In certain embodiments, one or more pharmaceutical agents of the present invention and one or more other pharmaceutical agents are prepared together in a single formulation. In certain embodiments, one or more pharmaceutical agents of the present invention and one or more other pharmaceutical agents are prepared separately.

Examples of pharmaceutical agents that may be co-administered with a pharmaceutical agent of the present invention include, but are not limited to, analgesics (e.g., acetaminophen); anti-inflammatory agents, including, but not limited to non-steroidal anti-inflammatory drugs (e.g. ibuprofen, COX-1 inhibitors, and COX-2, inhibitors); salicylates; antibiotics; antivirals; antifungal agents; antidiabetic agents (e.g. biguanides, glucosidase inhibitors, insulins, sulfonylureas, and thiazolidenediones); adrenergic modifiers; diuretics; hormones (e.g. anabolic steroids, androgen, estrogen, calcitonin, progestin, somatostan, and thyroid hormones); immunomodulators; muscle relaxants; antihistamines; osteoporosis agents (e.g., biphosphonates, calcitonin, and estrogens); prostaglandins, antineoplastic agents; psychotherapeutic agents; sedatives; poison oak or poison sumac products; antibodies; and vaccines.

Certain Indications

In certain embodiments, the invention provides methods of treating a patient comprising administering one or more compounds of the present invention. In certain embodiments, such patient suffers from a glucocorticoid receptor mediated condition. In certain embodiments, such patient suffers from a mineralocorticoid receptor mediated condition. In certain embodiments, such patient suffers from a glucocorticoid/minerlaocorticoid receptor mediated condition. In certain embodiments, a patient is treated prophylactically to reduce or prevent the occurrence of a condition.

In certain embodiments, one or more compounds of the present invention is used to treat inflammation, including, but not limited to, rheumatoid arthritis, asthma (acute or chronic), chronic obstructive pulmonary disease, lupus, osteoarthritis, rhinosinusitis, allergic rhinitis, inflammatory bowel disease, polyarteritis nodosa, Wegener's granulomatosis, giant cell arteritis, urticaria, angiodema, tendonitis, bursitis, autoimmune chronic hepatitis, and cirrhosis; transplant rejection; psoriasis; dermatitis; an autoimmune disorder; malignancy, including, but not limited to, leukemia, myeomas, and lymphomas; adrenal insufficiency; congenital adrenal hyperplasia; rheumatic fever; granulomatous disease; immune proliferation/apoptosis; conditions of the HPA axis; hypercortisolemia; cytokine imbalance, including, but not limited to Th/1/Th2 cytokine imbalance; kidney disease; liver disease; stroke; spinal cord injury; hypercalcemia; hyperglycemia; cerebral edema; thrombocytopenia; Little's syndrome; Addison's disease; cystic fibrosis; myasthenia gravis; autoimmune hemolytic anemia; uveitis; pemphigus vulgaris; multiple sclerosis; nasal polyps; sepsis; infections, including, but not limited to, bacterial, viral, rickettsial, and parasitic; type II diabetes; obesity; metabolic syndrome; schizophrenia; mood disorders, including, but not limited to depression; Cushing's syndrome; anxiety; sleep disorders; poor memory; glaucoma; wasting; heart disease; fibrosis; hypertension; hyperaldosteronism; and sodium and/or potassium imbalance.

### EXAMPLES

The following examples, including experiments and results achieved, are provided for illustrative purposes only and are not to be construed as limiting the present invention.

### Example 1

General Methods

General Method 1: Skraup cyclization of an aniline to a 1,2-dihydro-2,2,4-trimethylquinoline. A solution of an aniline (1.0 equiv), iodine (0.2-0.4 equiv), N,O-bis(trimethylsilyl)acetamide (2 equiv) in acetone (0.1-0.2 M) is heated in a sealed tube (110-130° C.) for 16-24 h. After heating, the solution is then processed by either a non-aqueous workup or by an aqueous work-up. In the non-aquesuous workup, the solution is evaporated under reduced pressure and chromatographed using silica gel and EtOAc:hexanes to afford the desired product as an oil. In the aqueous workup, the solution is mixed with an aqueous solution of sodium thiosulfate and a first organic layer of a 1:1 mixture of EtOAc:hexanes. The first organic layer is collected. The aqueous layer is then extracted a second time with a second layer of EtOAc:hexanes (1:1). The first and second organic layers are combined and that combined organic solution is washed with brine, dried over magnesium sulfate, filtered, and concentrated under reduced pressure. Flash chromatography (using silica gel) of the product of that process affords the desired compound.

General Method 2: Reduction of a 1,2-dihydroquinoline to a 1,2,3,4-tetrahydroquinoline. A solution of a 1,2-dihydroquinoline (1 equiv), triethylsilane (5 equiv) and trifluoroacetic acid (5 equiv) in 1,2-dichloroethane (0.5 M) is heated at reflux for 12-18 h, resulting in a dark brown solution. That dark brown solution is mixed with EtOAc and saturated sodium bicarbonate, resulting in an aqueous layer and a first organic layer. The first organic layer is collected and the aqueous layer is extracted with a second organic layer of EtOAc. The first organic layer and the second organic layer are combined and that combined organic layer is washed with brine, dried over magnesium sulfate, filtered, and concentrated under reduced pressure. Flash chromatography (silica gel) affords the desired compound as an oil.

General Method 3: Aromatic bromination of a 1,2,3,4-tetrahydroquinoline. To a solution of a 1,2,3,4-tetrahydroquinoline (1 equiv) in chloroform (0.2 M) at −10° C. is added N-bromosuccinimide (1.03 equiv) in portions over 15 minutes. After 1.5 hours, the mixture is washed with water, resulting in an aqueous layer and a first organic layer. The first organic layer is collected and the aqueous layer is extracted with a second organic layer of dichloromethane. The first and second organic layers are combined and that combined organic layer is washed with water, dried over magnesium sulfate, filtered, and concentrated under reduced pressure. Flash chromatography (silica gel) affords the desired 6-bromo-1,2,3,4-tetrahydroquinoline.

General Method 4. Palladium-catalyzed conversion of an aryl bromide to an aryl pinacol boronate. In a Schlenck reaction flask, a mixture of an aryl bromide (1 equiv) and [1,1′-bis(diphenylphosphino)ferrocene]dichloropalladium(II) complex with dichloromethane (3-10 mol %) is placed under vacuum, and back-filled with nitrogen. Dioxane (0.1-0.2 M) is added, followed by triethylamine (3-5 equiv), and pinacolborane (2-4 equiv). The solution is heated to reflux for 18 hours. Additional triethylamine and pinacolborane is added as need to complete the reaction. The mixture is poured into cold saturated ammonium chloride, resulting in an aqueous layer and a first organic layer. The aqueous layer is extracted with EtOAc, and the organic layer from that extraction is combined with the first organic layer. That combined organic layer is washed with brine, dried over magnesium sulfate, filtered and concentrated under reduced pressure. Flash chromatography (silica gel) affords the desired compound.

General Method 5. Palladium-catalyzed Suzuki cross-coupling of an aryl halide and an aryl boronic acid or aryl pinacol boronate. In a Schlenck reaction flask, a mixture of an aryl bromide (1 equiv); an aryl boronic acid or aryl pinacol boronate (1.0-1.3 equiv); and [1,1′-bis(diphenylphosphino)ferrocene]dichloropalladium(II) complex with dichloromethane (3-10 mol %) is placed under vacuum, and back-filled with nitrogen. Dioxane (0.1-0.2 M) and 2M sodium carbonate (2 equiv) are introduced sequentially. The mixture is heated (95-100° C.) for 16-24 h. The mixture is partitioned between saturated ammonium chloride and EtOAc, resulting in a first organic layer and an aqueous layer. The first organic layer is collected and the aqueous layer is extracted with EtOAc. The organic layer from that extraction is combined with the first collected organic layer and that combined organic layer is washed with brine, dried over magnesium sulfate, filtered, and concentrated under reduced pressure. Flash chromatography (silica gel, EtOAc/hexanes or other specified solvent), preparative thin-layer chromatography (prep TLC, EtOAc/hexanes or other specified solvents), preparative HPLC and/or recrystallization affords the desired compound.

General Method 6. Resolution of racemic compounds to their corresponding enantiomers (+)-6 and (−)-6 via chiral HPLC. A preparative chiral HPLC column (20×250 mm OR 10×250 mm) on a Beckman Gold HPLC is equilibrated with an eluent of hexanes:isopropanol. A solution of a racemic compound in MeOH, EtOH, or iPrOH is prepared and injections are monitored to insure that baseline separation is achieved. Compound elution is monitored by absorbance detection at 254 nM. The solvents of the separated enantiomers are removed in vacuo.

### 5-Chloro-1,2-dihydro-2,2,4,8-tetramethylquinoline (Structure 2 of Scheme 1, where R¹=Me, R²=H, R³=H, R⁴=Cl, R⁵=Me)

This compound was prepared using General Method 1 from 5-chloro-2-methylaniline (6.4 g, 45 mmol), iodine (3.8 g, 15 mmol), N,O-bis(trimethylsilyl)acetamide (18 g, 90 mmol) in 300 mL acetone heated at 130° C. for 18 h to afford, after an aqueous workup, 3.99 g (40%) of 5-chloro-1,2-dihydro-2,2,4,8-tetramethylquinoline, an amber oil, after flash chromatography (5:1 hexanes:EtOAc). ¹H NMR (500 MHz, CDCl₃) δ 6.80 (d, J=8.3, 1H), 6.62 (d, J=8.3, 1H), 5.45 (d, J=1.5, 1H), 3.73 (broad s, 1H), 2.31 (d, J=1.5, 1H), 2.08 (s, 3H), 1.26 (s, 6H).

### (±)-5-Chloro-1,2,3,4-tetrahydro-2,2,4,8-tetramethylquinoline (Structure 3 of Scheme 1, where R¹=Me, R²=H, R³=H, R⁴=Cl, R⁵=Me, R⁶=H, R⁹=H)

This compound was prepared using General Method 2 from 5-chloro-1,2-dihydro-2,2,4,8-tetramethylquinoline (4.5 g, 20.4 mmol) heated for 16 h to afford 2.7 g (59%) of (±)-5-chloro-1,2,3,4-tetrahydro-2,2,4,8-tetramethylquinoline, a light amber oil. ¹H NMR (500 MHz, CDCl₃) δ 6.80 (d, J=7.8, 1H), 6.62 (d, J=7.8, 1H), 3.47 (broad s, 1H), 3.20-3.30 (m, 1H), 2.05 (s, 3H), 1.93 (dd, J=13.7, 7.3, 1H), 1.74 (dd, J=13.7, 5.2, 1H), 1.40 (d, J=6.8, 3H), 1.34 (s, 3H), 1.19 (s, 3H).

### (±)-6-Bromo-5-chloro-1,2,3,4-tetrahydro-2,2,4,8-tetramethylquinoline (Structure 4 of Scheme 1, where R¹=Me, R²=H, R⁴=Cl, R⁵=Me, R⁶=H, R⁹=H)

This compound was prepared using General Method 3 from (±)-5-chloro-1,2,3,4-tetrahydro-2,2,4,8-tetramethylquinoline (3.13 g, 14 mmol) to afford 2.80 g (66%) of (±)-6-bromo-5-chloro-1,2,3,4-tetrahydro-2,2,4,8-tetramethylquinoline, a brown solid. ¹H NMR (400 MHz, CDCl₃) δ 7.14 (s, 1H), 3.47 (broad s, 1H), 3.25-3.35 (m, 1H), 2.04 (s, 3H), 1.91 (dd, J=13.6, 7.2, 1H), 1.75 (dd, J=13.6, 4.8, 1H), 1.37 (d, J=7.2, 3H), 1.33 (s, 3H), 1.19 (s, 3H).

### (±)-5-Chloro-1,2,3,4-tetrahydro-2,2,4,8-tetramethyl-6-(4,4,5,5-tetramethyl-1,3,2-dioxaborolan-2-yl)quinoline (Structure 5 of Scheme 1, where R¹=Me, R²=H, R⁴=Cl, R⁵=Me, R⁶=H, R⁹=H)

This compound was prepared using General Method 4 from (±)-6-bromo-5-chloro-1,2,3,4-tetrahydro-2,2,4,8-tetramethylquinoline (1.63 g, 5.40 mmol) to afford 1.45 g (77%) of (±)-5-chloro-1,2,3,4-tetrahydro-2,2,4,8-tetramethyl-6-(4,4,5,5-tetramethyl-1,3,2-dioxaborolan-2-yl)quinoline, a brown solid, after flash chromatography (12% EtOAc/hexanes). ¹H NMR (500 MHz, CDCl₃) δ 7.27 (s, 1H), 3.68 (broad s, 1H), 3.28-3.38 (m, 1H), 2.04 (s, 3H), 1.90 (dd, J=13.5, 7.0, 1H), 1.76 (dd, J=13.6, 4.5, 1H), 1.38 (d, J=7.2, 3H), 1.34 (s, 12H), 1.34 (s, 3H), 1.19 (s, 3H).

### (±)-5-Chloro-1,2,3,4-tetrahydro-2,2,4,8-tetramethyl-6-(thiazol-2-yl)quinoline (Compound 101, Structure 6 of Scheme I, where R¹=Me, R²=H, R⁴=Cl, R⁵=Me, R⁶=H, R⁹=H, Ar=thiazol-2-yl)

This compound was prepared using General Method 5 from (±)-5-chloro-1,2,3,4-tetrahydro-2,2,4,8-tetramethyl-6-(4,4,5,5-tetramethyl-1,3,2-dioxaborolan-2-yl)quinoline (50 mg, 0.15 mmol) and 2-bromothiazole (61 mg, 0.37 mmol) to afford 31 mg (69%) of Compound 101 after purification by prep TLC (25% EtOAc/hexanes). ¹H NMR (400 MHz, CDCl₃) δ 7.84 (d, J=3.3, 1H), 7.76 (s, 1H), 7.32 (d, J=3.2, 1H), 3.76 (broad s, 1H), 3.35-3.45 (m, 1H), 2.12 (s, 3H), 1.96 (dd, J=13.6, 6.8, 1H), 1.82 (dd, J=13.6, 4.2, 1H) 1.42 (d, J=7.1, 3H), 1.38 (s, 3H), 1.24 (s, 3H).

### Example 2

### (±)-6-(4-Acetylthiophen-2-yl)-5-chloro-1,2,3,4-tetrahydro-2,2,4,8-tetramethylquinoline (Compound 102, Structure 6 of Scheme I, where R¹=Me, R²=H, R⁴=Cl, R⁵=Me, R⁶=H, R⁹=H, Ar=4-acetylthiophen-2-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (123 mg, 0.36 mmol) and 4-acetyl-2-bromothiophene (90 mg, 0.44 mmol) to afford 59 mg (46%) of Compound 102, after prep TLC (25% EtOAc/hexanes). ¹H NMR (400 MHz, CDCl₃) δ 7.98 (d, J=1.5, 1H), 7.55 (d, J=1.5, 1H), 7.25 (s, 1H), 3.65 (broad s, 1H), 3.34-3.38 (m, 1H), 2.53 (s, 3H), 2.21 (s, 3H), 1.95 (dd, J=13.7, 7.0, 1H), 1.81 (dd, J=13.7, 4.3, 1H), 1.42 (d, J=7.1, 3H), 1.37 (s, 3H), 1.24 (s, 3H).

### Example 3

### (±)-5-Chloro-1,2,3,4-tetrahydro-6-(indol-2-yl)-2,2,4,8-tetramethylquinoline (Compound 103, Structure 6 of Scheme I, where R¹=Me, R²=H, R⁴=Cl, R⁵=Me, R⁶=H, R⁹=H, Ar=indol-2-yl)

To prepare this compound, (±)-6-Bromo-5-chloro-1,2,3,4-tetrahydro-2,2,4,8-tetramethylquinoline and 1-(t-butoxycarbonyl)indole-2-boronic acid were treated as described in General Method 5 (EXAMPLE 1) to afford (±)-5-chloro-1,2,3,4-tetrahydro-6-[(1-t-butoxycarbonyl)indol-2-yl]-2,2,4,8-tetramethylquinoline. That compound was combined with trifluoroacetic acid and the mixture was stirred at room temperature, quenched with water and neutralized with potassium carbonate. That quenched, neutralized mixture was extracted with EtOAc, and the resulting organic layer was dried over magnesium sulfate, filtered, and concentrated. Flash chromatography (10% EtOAc/hexanes) affords Compound 103. ¹H NMR (500 MHz, CDCl₃) δ 8.56 (br s, 1H), 7.62 (d, J=7.8 Hz, 1H), 7.39 (d, J=7.9, 1H), 7.38 (s, 1H), 7.17 (t, J=7.4 Hz, 1H), 7.10 (t, J=7.2, 1H), 6.66 (s, ½H), 6.65 (s, ½H), 3.32-3.44 (m, 2H), 2.12 (s, 3H), 1.98 (dd, J=7.0, 13.5 Hz, 1H), 1.82 (dd, J=4.3, 13.5 Hz), 1.44 (d, J=7.2, 3H), 1.39 (s, 3H), 1.25 (s, 3H).

### Example 4

### (±)-5-Chloro-6-(2,6-dimethoxyphenyl)-1,2,3,4-tetrahydro-2,2,4,8-tetramethylquinoline (Compound 104, Structure 6 of Scheme I, where R¹=Me, R²=H, R⁴=Cl, R⁵=Me, R⁶=H, R⁹=H, Ar=2,6-dimethoxyphenyl)

To prepare this compound, (±)-6-Bromo-5-chloro-1,2,3,4-tetrahydro-2,2,4,8-tetramethylquinoline (71 mg, 0.23 mol); 2,6-dimethoxyphenylboronic acid (64 mg, 0.35 mmol); palladium acetate (2.6 mg, 0.012 mmol); 2-(di-t-butylphosphino)biphenyl (10 mg, 0.029 mmol); and potassium fluoride (41 mg, 0.70 mmol) were placed in a Schlenck flask, evacuated and back-filled with nitrogen twice. THF (2.3 mL) was added, and the resulting suspension was heated at 70° C. for 20 h. After heating, the suspension was partitioned between EtOAc and saturated ammonium chloride, and the resulting organic layer washed with brine, dried over magnesium sulfate, filtered, and concentrated. Flash chromatography (12% EtOAc/hexanes) affords 11 mg (13%) of Compound 104. ¹H NMR (500 MHz, CDCl₃) δ 7.29 (t, J=8.2, 1H), 6.78 (s, 1H), 6.64 (d, J=8.2, 2H), 3.75 (s, 3H), 3.74 (s, 3H), 3.50 (broad s, 1H), 3.3-3.4 (m, 1H), 2.08 (s, 3H), 1.97 (dd, J=13.5, 7.0, 1H), 1.78 (dd, J=13.7, 4.0, 1H), 1.44 (d, J=7.0, 3H), 1.37 (s, 3H), 1.25 (s, 3H).

### Example 5

### (±)-5-Chloro-6-(3-cyano-2-methoxyphenyl)-1,2,3,4-tetrahydro-2,2,4,8-tetramethylquinoline (Compound 105, Structure 6 of Scheme I, where R¹=Me, R²=H, R⁴=Cl, R⁵=Me, R⁶=H, R⁹=H, Ar=3-cyano-2-methoxyphenyl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (279 mg, 0.80 mmol) and 3-bromo-2-methoxybenzonitrile (254 mg, 1.20 mmol) to afford 220 mg (78%) of Compound 105, after flash chromatography (80% dichloromethane/hexanes). ¹H NMR (500 MHz, CDCl₃) δ 7.56 (dd, J=7.6, 1.5, 1H), 7.40-7.50 (m, 1H), 7.16 (dd, J=7.6, 7.6, 1H), 6.82 (s, 1H), 3.68 (broad s, 3H), 3.61 (broad s, 1H), 3.30-3.40 (m, 1H), 2.10 (s, 3H), 1.98 (dd, J=13.6, 7.3, 1H), 1.81 (dd, J=13.6, 4.4, 1H), 1.43 (d, J=7.0, 3H), 1.39 (s, 3H), 1.26 (s, 3H).

### Example 5A

### (+)-5-Chloro-6-(3-cyano-2-methoxyphenyl)-1,2,3,4-tetrahydro-2,2,4,8-tetramethylquinoline (Compound 105A, Structure (+)-6 of Scheme I, where R¹=Me, R²=H, R⁴=Cl, R⁵=Me, R⁶=H, R⁹=H, Ar=3-cyano-2-methoxyphenyl), and (−)-5-Chloro-6-(3-cyano-2-methoxyphenyl)-1,2,3,4-tetrahydro-2,2,4,8-tetramethylquinoline (Compound 105B, Structure

(−)-6 of Scheme I, where R¹=Me, R²=H, R⁴=Cl, R⁵=Me, R⁶=H, R⁹=H, Ar=3-cyano-2-methoxyphenyl)

These compounds were isolated from the racemic compound of Example 5 using General Method 6 (EXAMPLE 1) on a Chiracel AD column (20×250 mm, 5% isopropanol/hexanes, 6 ml/min, to afford Compounds 105A and 105B. Data for Compound 105A: HPLC (Chiralcel AD, 5% isopropanol/hexanes, 6 ml/min) t_{R} 13.0 min; [α]_{D}=+10.5. Data for Compound 105B: HPLC (Chiralcel AD, 5% isopropanol/hexanes, 6 ml/min) t_{R} 13.9 min; [α]_{D}=−10.1.

### Example 6

### (±)-6-(3-Amino-5-methylisoxazol-4-yl)-5-chloro-1,2,3,4-tetrahydro-2,2,4,8-tetramethylquinoline (Compound 106, Structure 6 of Scheme I, where R¹=Me, R²=H, R⁴=Cl, R⁵=Me, R⁶=H, R⁹=H, Ar=3-amino-5-methylisoxazol-4-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (38 mg, 0.11 mmol) and 3-amino-4-bromo-5-methylisoxazole (34 mg, 0.15 mmol) to afford 4 mg (11%) of Compound 106, after flash chromatography (50% EtOAc/hexanes). ¹H NMR (400 MHz, CDCl₃) δ 6.79 (s, ½H), 6.78 (s, ½H), 3.86 (broad s, 1H), 3.83 (broad s, 1H), 3.62 (broad s, 1H), 3.28-3.38 (m, 1H), 2.23 (s, 3/2H), 2.21 (s, 3/2H), 2.08 (s, 3H), 1.90-2.00 (m, 1H), 1.80 (dd, J=13.6, 4.2, 1H), 1.42 (d, J=7.1, 3H), 1.38 (s, 3H), 1.25 (s, 3/2H), 1.24 (s, 3/2H).

### Example 7

### (±)-5-Chloro-1,2,3,4-tetrahydro-6-(2-methoxyphenyl)-2,2,4,8-tetramethylquinoline (Compound 107, Structure 6 of Scheme I, where R¹=Me, R²=H, R⁴=Cl, R⁵=Me, R⁶=H, R⁹=H, Ar=2-methoxyphenyl)

This compound was prepared using General Method 5 (EXAMPLE 1) from (±)-6-bromo-5-chloro-1,2,3,4-tetrahydro-2,2,4,8-tetramethylquinoline (30 mg, 0.10 mol), and 2-methoxyphenylboronic acid (20 mg, 0.13 mmol) to afford Compound 107 after flash chromatography (20% EtOAc/hexanes). ¹H NMR (400 MHz, CDCl₃) δ 7.30-7.38 (m, 1H), 7.15-7.25 (m, 1H), 6.95-7.05 (m, 2H), 6.84 (s, 1H), 3.78 (broad s, 3H), 3.51 (broad s, 1H), 3.30-3.40 (m, 1H), 2.08 (s, 3H), 1.96 (dd, J=13.4, 7.0, 1H), 1.78 (dd, J=13.4, 3.9, 1H), 1.45 (d, J=6.8, 3/2H), 1.42 (d, J=6.9, 3/2H), 1.37 (s, 3H), 1.24 (s, 3H).

### Example 8

### (±)-5-Chloro-1,2,3,4-tetrahydro-2,2,4,8-tetramethyl-6-(quinolin-8-yl)quinoline (Compound 108, Structure 6 of Scheme I, where R¹=Me, R²=H, R⁴=Cl, R⁵=Me, R⁶=H, R⁹=H, Ar=quinolin-8-yl)

This compound was prepared using General Method 5 (EXAMPLE 1) from (±)-6-bromo-5-chloro-1,2,3,4-tetrahydro-2,2,4,8-tetramethylquinoline (30 mg, 0.10 mmol) and 8-quinolineboronic acid (22 mg, 0.13 mmol) to afford 22 mg (63%) of Compound 108 after flash chromatography (40% EtOAc/hexanes). ¹H NMR (400 MHz, CDCl₃) δ 8.91-8.95 (m, 1H), 8.18 (d, J=8.2, 1H), 7.82 (d, J=8.0, 1H), 7.50-7.70 (m, 2H), 7.30-7.40 (m, 1H), 6.97 (s, 1H), 3.60 (broad s, 1H), 3.32-3.42 (m, 1H), 2.10 (s, 3H), 1.98 (dd, J=13.4, 6.9, 1H), 1.81 (broad d, J=13.4, 1H), 1.48 (d, J=7.2, 3/2H), 1.45 (d, J=7.1, 3/2H), 1.28 (s, 3H).

### Example 9

### (±)-6-(Benzothiophen-3-yl)-5-chloro-1,2,3,4-tetrahydro-2,2,4,8-tetramethylquinoline (Compound 109, Structure 6 of Scheme I, where R¹=Me, R²=H, R⁴=Cl, R⁵=Me, R⁶=H, R⁹=H, Ar=benzothiophen-3-yl)

This compound was prepared using General Method 5 (EXAMPLE 1) from (±)-6-bromo-5-chloro-1,2,3,4-tetrahydro-2,2,4,8-tetramethylquinoline (30 mg, 0.10 mmol) and thianaphthene-3-boronic acid (23 mg, 0.13 mmol) to afford 12 mg (33%) of Compound 109, after flash chromatography (50% dichloromethane/hexanes). ¹H NMR (400 MHz, CDCl₃) δ 7.85-7.90 (m, 1H), 7.52-7.58 (m, 1H), 7.30-7.40 (m, 3H), 6.95 (s, 1H), 3.60 (broad s, 1H), 3.32-3.42 (m, 1H), 2.10 (s, 3H), 1.99 (dd, J=13.5, 7.1, 1H), 1.82 (dd, J=13.5, 4.4, 1H), 1.46 (d, J=7.1, 3H), 1.40 (s, 3H), 1.27 (s, 3H).

### Example 10

### (±)-5-Chloro-1,2,3,4-tetrahydro-2,2,4,8-tetramethyl-6-(5-methyl-3-phenylisoxazol-3-yl)quinoline (Compound 110, Structure 6 of Scheme I, where R¹=Me, R²=H, R⁴=Cl, R⁵=Me, R⁶=H, R⁹=H, Ar=5-methyl-3-phenylisoxazol-3-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (42 mg, 0.12 mmol) and 4-iodo-5-methyl-3-phenylisoxazole (41 mg, 0.14 mmol) to afford 13 mg (28%) of Compound 110. ¹H NMR (400 MHz, CDCl₃) δ 7.43-7.53 (m, 2H), 7.22-7.38 (m, 3H), 6.74 (s, ½H), 6.69 (s, ½H), 3.58 (broad s, 1H), 3.20-3.33 (m, 1H), 2.33 (s, 3/2H), 2.32 (s, 3/2H), 2.06 (s, 3/2H), 2.02 (s, 3/2H), 1.93-2.01 (m, 1H), 1.74-1.82 (m, 1H), 1.41 (d, J=3/2H), 1.38 (s, 3/2H), 1.37 (s, 3/2H), 1.30 (d, J=7.0, 3/2H), 1.26 (s, 3/2H), 1.24 (s, 3/2H).

### Example 11

### (±)-5-Chloro-1,2,3,4-tetrahydro-2,2,4,8-tetramethyl-6-(1,3,5-trimethylpyrazol-4-yl)quinoline (Compound III, Structure 6 of Scheme I, where R¹=Me, R²=H, R⁴=Cl, R⁵=Me, R⁶=H, R⁹=H, Ar=1,3,5-trimethylpyrazol-4-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (29 mg, 0.083 mmol) and 4-bromo-1,3,5-trimethylpyrazole (21 mg, 0.11 mmol) to afford 4.5 mg (16%) of Compound III after flash chromatography (50% EtOAc/hexanes) and preparative HPLC (HiChrom C18, 10×250 mm, 80% MeOH/water, 2.5 mL/min). ¹H NMR (400 MHz, CDCl₃) δ 6.72 (s, ½H), 6.71 (s, ½H), 3.77 (s, 3/2H), 3.76 (s, 3/2H), 3.52 (broad s, 1H), 3.28-3.38 (m, 1H), 2.07-2.12 (m, 6H), 2.08 (s, 3H), 1.90-2.00 (m, 1H), 1.75-1.82 (m, 1H), 1.43 (d, J=7.2, 3/2H), 1.42 (d, J=7.2, 3/2H), 1.24 (s, 6H).

### Example 12

### (±)-5-Chloro-6-(2,4-dimethoxyphenyl)-1,2,3,4-tetrahydro-2,2,4,8-tetramethylquinoline (Compound 112, Structure 6 of Scheme I, where R¹=Me, R²=H, R⁴=Cl, R⁵=Me, R⁶=H, R⁹=H, Ar=2,4-dimethoxyphenyl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (34 mg, 0.098 mmol) and 1-bromo-2,4-dimethoxybenzene (28 mg, 0.13 mmol) to afford 16 mg (46%) of Compound 112 after flash chromatography (15% EtOAc/hexanes) and preparative HPLC (Beckman Ultrasphere ODS, 10×250 mm, 80% MeOH/water). ¹H NMR (400 MHz, CDCl₃) δ 7.05-7.15 (m, 1H), 6.82 (s, 1H), 6.50-6.56 (m, 2H), 3.84 (s, 3H), 3.76 (s, 3H), 3.52 (broad s, 1H), 3.30-3.40 (m, 1H), 2.07 (s, 3H), 1.95 (dd, J=13.5, 7.0, 1H), 1.78 (dd, J=13.5, 4.1, 1H), 1.40-1.50 (m, 3H), 1.37 (s, 3H), 1.24 (s, 3H).

### Example 13

### (±)-6-(2-Aminophenyl)-5-chloro-1,2,3,4-tetrahydro-2,2,4,8-tetramethylquinoline (Compound 113, Structure 6 of Scheme I, where R¹=Me, R²=H, R⁴=Cl, R⁵=Me, R⁶=H, R⁹=H, Ar=2-aminophenyl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (87 mg, 0.25 mmol) and 2-bromoaniline (52 mg, 0.30 mmol) to afford 40 mg (51%) of Compound 113, after flash chromatography (25% EtOAc/hexanes). ¹H NMR (500 MHz, CDCl₃) δ 7.13-7.18 (m, 1H), 7.07 (dd, J=7.3, 1.5, ½H), 7.03 (dd, J=7.3, 1.5, ½H), 6.86 (s, 1H), 6.70-6.83 (m, 2H), 3.56 (broad s, 3H), 3.30-3.40 (m, 1H), 2.08 (s, 3H), 1.94-2.00 (m, 1H), 1.77-1.83 (m, 1H), 1.43 (d, J=7.3, 3/2H), 1.42 (d, J=7.3, 3/2H), 1.39 (s, 3/2H), 1.38 (s, 3/2H), 1.25 (s, 3/2H), 1.24 (s, 3/2).

### Example 14

### (±)-5-Chloro-6-(3,5-dimethylisoxazol-4-yl)-1,2,3,4-tetrahydro-2,2,4,8-tetramethylquinoline (Compound 114, Structure 6 of Scheme I, where R¹=Me, R²=H, R⁴=Cl, R⁵=Me, R⁶=H, R⁹=H, Ar=3,5-dimethylisoxazol-4-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (68 mg, 0.20 mmol) and 4-bromo-3,5-dimethylisoxazole (48 mg, 0.27 mmol) to afford 36 mg (58%) of Compound 114 after flash chromatography (100% dichloromethane to 2% EtOAc/dichloromethane, gradient elution). ¹H NMR (400 MHz, CDCl₃) δ 6.70 (s, 1H), 3.59 (broad s, 1H), 3.30-3.40 (m, 1H), 2.27 (s, 3/2H), 2.25 (s, 3/2H), 2.15 (s, 3/2H), 2.13 (s, 3/2H), 2.08 (s, 3H), 1.93-2.00 (m, 1H), 1.78-1.82 (m, 1H), 1.42 (d, J=7.3, 3/2H), 1.41 (d, J=7.3, 3/2H), 1.38 (s, 3H), 1.26 (s, 3/2H), 1.25 (s, 3/2H).

### Example 14A

### (+)-5-Chloro-6-(3,5-dimethylisoxazol-4-yl)-1,2,3,4-tetrahydro-2,2,4,8-tetramethylquinoline (Compound 114A, Structure (+)-6 of Scheme I, where R¹=Me, R²=H, R⁴=Cl, R⁵=Me, R⁶═H, R⁹=H, Ar=3,5-dimethylisoxazol-4-yl), and (−)-5-Chloro-6-(3,5-dimethylisoxazol-4-yl)-1,2,3,4-tetrahydro-2,2,4,8-tetramethylquinoline (Compound 114B, Structure (−)-6 of Scheme L where R¹=Me, R²=H, R⁴=Cl, R⁵=Me, R⁶=H, R⁹=H, Ar=3,5-dimethylisoxazol-4-yl)

These compounds were isolated from the racemic compound of Example 14 using General Method 6 (EXAMPLE 1) on a Chiracel OJ column (20×250 mm, 10% isopropanol/hexanes, 6 ml/min, to afford Compounds 114A and 114B. Data for Compound 114A: HPLC (Chiralcel OJ, 10% EtOH/hexanes, 6 ml/min) t_{R} 17.9 min; [α]_{D}=+2.9. Data for Compound 114B: HPLC (Chiralcel OJ, 10% EtOH/hexanes, 6 ml/min) t_{R} 16.0 min; [α]_{D}=−3.0.

### Example 15

### (±)-6-(5-Acetylthiophen-2-yl)-5-chloro-1,2,3,4-tetrahydro-2,2,4,8-tetramethylquinoline (Compound 115, Structure 6 of Scheme I, where R¹=Me, R²=H, R⁴=Cl, R⁵=Me, R⁶=H, R⁹=H, Ar=5-acetylthiophen-2-yl)

This compound was prepared using General Method 5 (EXAMPLE 1) from (±)-6-bromo-5-chloro-1,2,3,4-tetrahydro-2,2,4,8-tetramethylquinoline (60 mg, 0.20 mmol) and 5-acetyl-2-thiopheneboronic acid (41 mg, 0.20 mmol) to afford 27 mg (39%) of Compound 115 after flash chromatography (65% dichloromethane/hexanes to 80% dichloromethane/hexanes, gradient elution). ¹H NMR (500 MHz, CDCl₃) δ 7.64 (d, J=3.9, 1H), 7.24 (d, J=3.9, 1H), 7.10 (s, 1H), 3.71 (broad s, 1H), 3.35-3.42 (m, 1H), 2.56 (s, 3H), 2.09 (s, 3H), 1.94 (dd, J=13.7, 6.8, 1H), 1.82 (dd, J=13.7, 3.9, 1H), 1.42 (d, J=7.3, 3H), 1.38 (s, 3H), 1.25 (s, 3H).

### Example 16

### (±)-6-(Benzothiophen-2-yl)-5-chloro-1,2,3,4-tetrahydro-2,2,4,8-tetramethylquinoline (Compound 116, Structure 6 of Scheme I, where R¹=Me, R²=H, R⁴=Cl, R⁵=Me, R⁶=H, R⁹⁼H, Ar=2-benzothiophen-2-yl)

This compound was prepared using General Method 5 (EXAMPLE 1) from (±)-6-bromo-5-chloro-1,2,3,4-tetrahydro-2,2,4,8-tetramethylquinoline (30 mg, 0.10 mmol) and thianaphthene2-boronic acid (21 mg, 0.12 mmol) to afford 30 mg (84%) of Compound 116 after flash chromatography (20% dichloromethane/hexanes). ¹H NMR (500 MHz, CDCl₃) δ 7.82 (d, J=7.8 Hz, 1H), 7.77 (d, J=7.8 Hz, 1H), 7.40 (s, 1H), 7.34-7.36 (m, 1H), 7.28-7.32 (m, 1H), 7.14 (s, 1H), 3.66 (s, 1H), 3.39-3.41 (m, 1H), 2.12 (s, 3H), 1.97 (dd, J=6.8, 13.2 Hz, 1H), 1.82 (dd, J=4.4, 13.7 Hz, 1H), 1.45 (d, J=6.8 Hz, 3H), 1.39 (s, 3H), 1.26 (s, 3H).

### Example 17

### (±)-5-Chloro-6-(2-fluorophenyl)-1,2,3,4-tetrahydro-2,2,4,8-tetramethylquinoline (Compound 117, Structure 6 of Scheme I, where R¹=Me, R²=H, R⁴=Cl, R⁵=Me, R⁶=H, R⁹=H, Ar=2-fluorophenyl)

This compound was prepared using General Method 5 (EXAMPLE 1) from (±)-6-bromo-5-chloro-1,2,3,4-tetrahydro-2,2,4,8-tetramethylquinoline (30 mg, 0.10 mmol) and 2-fluorophenylboronic acid (17 mg, 0.12 mmol) to afford 17 mg (53%) of Compound 117 after flash chromatography (10% EtOAc/hexanes). ¹H NMR (500 MHz, CDCl₃) δ 7.26-7.29 (m, 2H), 7.09-7.17 (m, 2H), 6.87 (s, 1H), 3.57 (s, 1H), 3.35-3.38 (m, 1H), 2.09 (s, 3H), 1.97 (dd, J=3.8, 13.2 Hz, 1H), 1.80 (dd, J=4.4, 13.7 Hz, 1H), 1.44 (d, J=7.3 Hz, 3H), 1.39 (s, 3H), 1.25 (s, 3H).

### Example 18

### (±)-5-Chloro-6-(2-chlorophenyl)-1,2,3,4-tetrahydro-2,2,4,8-tetramethylquinoline (Compound 118, Structure 6 of Scheme I, where R¹=Me, R²=H, R⁴=Cl, R⁵=Me, R⁶=H, R⁹=H, Ar=2-chlorophenyl)

This compound was prepared using General Method 5 (EXAMPLE 1) from (±)-6-bromo-5-chloro-1,2,3,4-tetrahydro-2,2,4,8-tetramethylquinoline (30 mg, 0.10 mmol) and 2-chlorophenylboronic acid (19 mg, 0.12 mmol) to afford 8 mg (24%) of Compound 118 after flash chromatography (10% EtOAc/hexanes). ¹H NMR (500 MHz, CDCl₃) δ 7.32-7.35 (m, 1H), 7.24-7.29 (m, 3H), 6.80 (s, ½H), 6.79 (s, ½H), 3.56 (s, 1H), 3.24-3.38 (m, 1H), 2.09 (s, 3H), 1.94-1.97 (m, 1H), 1.80-1.82 (m, 1H), 1.44 (d, J=6.8 Hz, 3/2H), 1.42 (d, J=6.8 Hz, 3/2H), 1.39 (s, 3/2H), 1.38 (s, 3/2H), 1.26 (s, 3/2H), 1.25 (s, 3/2H).

### Example 19

### (±)-6-(2-Acetylphenyl)-5-chloro-1,2,3,4-tetrahydro-2,2,4,8-tetramethylquinoline (Compound 119, Structure 6 of Scheme I, where R¹=Me, R²=H, R⁴=Cl, R⁵=Me, R⁶=H, R⁹=H, Ar=2-acetylphenyl)

This compound was prepared using General Method 5 (EXAMPLE 1) from (±)-6-bromo-5-chloro-1,2,3,4-tetrahydro-2,2,4,8-tetramethylquinoline (30 mg, 0.10 mmol) and 2-acetylphenylboronic acid (20 mg, 0.12 mmol) to afford 12 mg (35%) of Compound 119 after flash chromatography (10% EtOAc/hexanes). ¹H NMR (500 MHz, CDCl₃) δ 7.65 (t, J=6.8 Hz, 1H), 7.47-7.49 (m, 1H), 7.39 (t, J=7.3 Hz, 1H), 7.34 (d, J=7.3 Hz, ½H), 7.28 (d, J=7.8 Hz, ½H), 6.78 (s, ½H), 6.76 (s, ½H), 3.58 (s, 1H), 3.32-3.34 (m, 1H), 2.17 (s, 3/2H), 2.08 (s, 3/2H), 2.07 (s, 3/2H), 2.05 (s, 3/2H), 1.95-2.01 (m, 1H), 1.77-1.82 (m, 1H), 1.43 (d, J=7.3 Hz, 3/2H), 1.40 (d, J=6.8 Hz, 3/2H), 1.38 (s, 3/2H), 1.37 (s, 3/2H), 1.24 (s, 3/2H), 1.23 (s, 3/2H).

### Example 20

### (±)-5-Chloro-1,2,3,4-tetrahydro-6-(indol-4-yl)-2,2,4,8-tetramethylquinoline (Compound 120, Structure 6 of Scheme I, where R¹=Me, R²=H, R⁴=Cl, R⁵=Me, R⁶=H, R⁹=H, Ar=indol-4-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (62 mg, 0.18 mol) and 4-bromoindole (30 mg, 0.15 mmol) to afford 23 mg (38%) of Compound 120 after flash chromatography (10% EtOAc/hexanes). ¹H NMR (500 MHz, CDCl₃) δ 8.17 (br s, 1H), 7.37 (d, J=8.3 Hz, 1H), 7.24 (t, J=7.3 Hz, 1H), 7.18-7.20 (m, 1H), 7.04-7.15 (m, 1H), 7.01 (s, 1H), 6.39 (s, 1H), 3.71 (s, 1H), 3.37-3.42 (m, 1H), 2.12 (s, 3H), 2.01 (dd, J=7.3, 13.7 Hz, 1H), 1.82 (dd, J=4.4, 13.7 Hz, 1H), 1.48 (d, J=6.8 Hz, 3H), 1.41 (s, 3H), 1.27 (s, 3H).

### Example 21

### (±)-5-Chloro-6-(5-chloro-2-methoxyphenyl)-1,2,3,4-tetrahydro-2,2,4,8-tetramethylquinoline (Compound 121, Structure 6 of Scheme I, where R¹=Me, R²=H, R⁴=Cl, R⁵=Me, R⁶=H, R⁹=H, Ar=5-chloro-2-methoxyphenyl)

This compound was prepared using General Method 5 (EXAMPLE 1) from (±)-6-bromo-5-chloro-1,2,3,4-tetrahydro-2,2,4,8-tetramethylquinoline (30 mg, 0.10 mmol) and 5-chloro-2-methoxyphenylboronic acid (22 mg, 0.12 mmol) to afford 11 mg (32%) of Compound 121 after flash chromatography (10% EtOAc/hexanes). ¹H NMR (500 MHz, CDCl₃) δ 7.25-7.29 (m, 1H), 7.19 (d, J=2.4 Hz, 1H), 6.89 (d, J=8.8 Hz, ½H), 6.86 (d, J=8.8 Hz, ½H), 6.80 (s, 1H), 3.76 (s, 3H), 3.56 (s, 1H), 3.32-3.35 (m, 1H), 2.07 (s, 3H), 1.91-1.95 (m, 1H), 1.78-1.82 (m, 1H), 1.40-1.43 (m, 3H), 1.37 (s, 3H), 1.24 (s, 3H).

### Example 22

### (±)-5-Chloro-1,2,3,4-tetrahydro-2,2,4,8-tetramethyl-6-(2-nitrophenyl)quinoline (Compound 122, Structure 6 of Scheme I, where R¹=Me, R²=H, R⁴=Cl, R⁵=Me, R⁶=H, R⁹=H, Ar=2-nitrophenyl)

This compound was prepared using General Method 5 (EXAMPLE 1) from (±)-6-bromo-5-chloro-1,2,3,4-tetrahydro-2,2,4,8-tetramethylquinoline (30 mg, 0.10 mmol) and 2-nitrobenzeneboronic acid (20 mg, 0.12 mmol) to afford 12 mg (35%) of Compound 122 after flash chromatography (10% EtOAc/hexanes). ¹H NMR (500 MHz, CDCl₃) δ 7.95-7.99 (m, 1H), 7.57-7.60 (m, 1H), 7.46 (t, J=7.8 Hz, 1H), 7.40 (dd, J=1.0, 7.3 Hz, ½H), 7.34 (dd, J=1.0, 7.8 Hz, ½H), 6.81 (s, ½H), 6.80 (s, ½H), 3.59 (s, ½H), 3.58 (s, ½H), 3.22-3.38 (m, 1H), 2.09 (s, 3/2H), 2.08 (s, 3/2H), 1.93-1.98 (m, 1H), 1.76-1.81 (m, 1H), 1.37-1.43 (m, 6H), 1.25 (s, 3/2H), 1.24 (s, 3/2H).

### Example 23

### 5-Chloro-6-(2,3-dichlorophenyl)-1,2,3,4-tetrahydro-2,2,4,8-tetramethylquinoline (Compound 123, Structure 6 of Scheme I, where R¹=Me, R²=H, R⁴=Cl, R⁵=Me, R⁶=H, R⁹=H, Ar=2,3-dichlorophenyl)

This compound was prepared using General Method 5 (EXAMPLE 1) from (±)-6-bromo-5-chloro-1,2,3,4-tetrahydro-2,2,4,8-tetramethylquinoline (30 mg, 0.10 mmol) and 2,3-dichlorobenzeneboronic acid (23 mg, 0.12 mmol) to afford 14 mg (38%) of Compound 123 after flash chromatography (10% EtOAc/hexanes). Low resolution MS (EI) m/e 367, 369.

### Example 24

### 5-Chloro-1,2,3,4-tetrahydro-2,2,4,8-tetramethyl-6-[2-(trifluoromethyl)phenyl]quinoline (Compound 124, Structure 6 of Scheme I, where R¹=Me, R²=H, R⁴=Cl, R⁵=Me, R⁶=H, R⁹=H, Ar=2-(trifluoromethyl)phenyl)

This compound was prepared using General Method 5 (EXAMPLE 1) from (±)-6-bromo-5-chloro-1,2,3,4-tetrahydro-2,2,4,8-tetramethylquinoline (30 mg, 0.10 mmol) and 2-(trifluoromethyl)phenylboronic acid (23 mg, 0.12 mmol) to afford 13 mg of Compound 124 after flash chromatography (10% EtOAc/hexanes). Low resolution MS (EI) m/e 367.

### Example 25

### (±)-5-Chloro-1,2,3,4-tetrahydro-2,2,4,8-tetramethyl-6-(2-methyl-3-nitrophenyl)quinoline (Compound 125, Structure 6 of Scheme I, where R¹=Me, R²=H, R⁴=Cl, R⁵=Me, R⁶=H, R⁹=H, Ar=2-methyl-3-nitrophenyl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (52 mg, 0.15 mmol) and 2-bromo-6-nitrotoluene (36 mg, 0.16 mmol) to afford 43 mg (80%) of Compound 125 after flash chromatography (20% EtOAc/hexanes). ¹H NMR (500 MHz, CDCl₃) δ 7.79-7.83 (m, 1H), 7.42 (dd, J=7.8, 1.5, ½H), 7.37 (dd, J=7.3, 1.5, ½H), 7.28-7.34 (m, 1H), 6.72 (s, 1H), 3.59 (broad s, 1H), 3.28-3.38 (m, 1H), 2.30 (s, 3/2H), 2.25 (s, 3/2H), 2.09 (s, 3H), 1.94-2.01 (m, 1H), 1.78-1.84 (m, 1H), 1.43 (d, J=6.8, 3/2H), 1.42 (d, J=6.8, 3/2H), 1.39 (s, 3H), 1.27 (s, 3/2H), 1.25 (s, 3/2H).

### Example 26

### (±)-6-(2-Biphenyl)-5-chloro-1,2,3,4-tetrahydro-2,2,4,8-tetramethylquinoline (Compound 126, Structure 6 of Scheme I, where R¹=Me, R²=H, R⁴=Cl, R⁵=Me, R⁶=H, R⁹=H, Ar=2-biphenyl)

This compound was prepared using General Method 5 (EXAMPLE 1) from (±)-6-bromo-5-chloro-1,2,3,4-tetrahydro-2,2,4,8-tetramethylquinoline (30 mg, 0.10 mmol) and 2-biphenylboronic acid (24 mg, 0.12 mmol) to afford 8 mg of Compound 126. Low resolution MS (EI) m/e 375.

### Example 27

### (±)-5-Chloro-6-(dibenzofuran-4-yl)-1,2,3,4-tetrahydro-2,2,4,8-tetramethylquinoline (Compound 127, Structure 6 of Scheme I, where R¹=Me, R²=H, R⁴=Cl, R⁵=Me, R⁶=H, R⁹=H, Ar=dibenzofuran-4-yl)

This compound was prepared using General Method 5 (EXAMPLE 1) from (±)-6-bromo-5-chloro-1,2,3,4-tetrahydro-2,2,4,8-tetramethylquinoline (30 mg, 0.10 mmol) and 4-dibenzofuranboronic acid (25 mg, 0.12 mmol) to afford 10 mg of Compound 127 after flash chromatography (10% EtOAc/hexanes). ¹H NMR (500 MHz, CDCl₃) δ 7.98 (d, J=7.3 Hz, 1H), 7.93 (d, J=7.3 Hz, 1H), 7.55 (d, J=8.3 Hz, 1H), 7.32-7.44 (m, 3H), 7.26 (s, 1H), 7.07 (s, 1H), 3.64 (s, 1H), 3.40-3.43 (m, 1H), 2.14 (s, 3H), 2.00 (dd, J=6.8, 13.7 Hz, 1H), 1.84 (dd, J=3.9, 13.7 Hz, 1H), 1.48 (d, J=7.3 Hz, 3H), 1.42 (s, 3H), 1.29 (s, 3H).

### Example 28

### 5-Chloro-1,2,3,4-tetrahydro-6-(indol-6-yl)-2,2,4,8-tetramethylquinoline (Compound 128 Structure 6 of Scheme I, where R¹=Me, R²=H, R⁴=Cl, R⁵=Me, R⁶=H, R⁹=H, Ar=indol-6-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (52 mg, 0.15 mmol) and 6-bromoindole (22 mg (0.11 mmol) to afford 6 mg of Compound 128 after flash chromatography (10% EtOAc/hexanes). ¹H NMR (500 MHz, CDCl₃) δ 8.16 (br s, 1H), 7.64 (d, J=8.3 Hz, 1H), 7.42 (s, 1H), 7.21-7.23 (m, 1H), 6.97 (s, 1H), 6.56-6.57 (m, 1H), 3.52 (s, 1H), 3.38-3.40 (m, 1H), 2.11 (s, 3H), 1.98 (dd, J=7.3, 13.7 Hz, 1H), 1.81 (dd, J=4.4, 13.7 Hz, 1H), 1.45 (d, J=7.3 Hz, 3H), 1.39 (s, 3H), 1.25 (s, 3H).

### Example 29

### (±)-5-Chloro-6-(2,3-dihydro-1,4-benzodioxin-6-yl)-1,2,3,4-tetrahydro-2,2,4,8-tetramethylquinoline (Compound 129, Structure 6 of Scheme I, where R¹=Me, R²=H, R⁴=Cl, R⁵=Me, R⁶=H, R⁹=H, Ar=2,3-dihydro-1,4-benzodioxin-6-yl)

This compound was prepared using General Method 5 (EXAMPLE 1) from (±)-6-bromo-5-chloro-1,2,3,4-tetrahydro-2,2,4,8-tetramethylquinoline (30 mg, 0.10 mmol) and 2,3-dihydro-1,4-benzodioxin-6-ylboronic acid. ¹H NMR (500 MHz, CDCl₃) δ 6.97 (s, 1H), 6.83-6.91 (m, 3H), 4.29 (s, 4H), 3.66 (s, 1H), 3.36-3.41 (m, 1H), 2.09 (s, 3H), 1.93 (dd, 1H), 1.79 (dd, 1H), 1.42 (dd, J=6.8 Hz, 3H), 1.37 (s, 1H), 1.24 (s, 3H).

### Example 30

### (±)-5-Chloro-6-[2-fluoro-3-(trifluoromethyl)phenyl]-1,2,3,4-tetrahydro-2,2,4,8-tetramethylquinoline (Compound 130, Structure 6 of Scheme I, where R¹=Me, R²=H, R⁴=Cl, R⁵=Me, R⁶=H, R⁹=H, Ar=2-fluoro-3-(trifluoromethyl)phenyl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (50 mg, 0.14 mmol) and 3-bromo-2-fluorobenzotrifluoride (24 mg, 0.1 mmol) to afford 14 mg of Compound 130 after flash chromatography (10% EtOAc/hexanes. ¹H NMR (500 MHz, CDCl₃) δ 7.43-7.58 (m, 2H), 7.21-7.24 (m, 1H), 6.83 (s, 1H), 3.48 (s, 1H), 3.35-3.38 (m, 1H), 2.09 (s, 3H), 1.96-1.99 (m, 1H), 1.78-1.81 (m, 1H), 1.42 (d, J=6.8 Hz, 3H), 1.38 (s, 3H), 1.27 (s, 3H).

### Example 31

### (±)-5-Chloro-1,2,3,4-tetrahydro-2,2,4,8-tetramethyl-6-[2-(trifluoromethoxy)phenyl]quinoline (Compound 131, Structure 6 of Scheme I, where R¹=Me, R²=H, R⁴=Cl, R⁵=Me, R⁶=H, R⁹=H, Ar=2-(trifluoromethoxy)phenyl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (50 mg, 0.14 mmol) and 1-iodo-2-(trifluoromethoxy)benzene (29 mg, 0.10 mmol) to afford Compound 131 after flash chromatography (15% EtOAc/hexanes). ¹H NMR (500 MHz, CDCl₃) δ 7.26-7.36 (m, 4H), 6.80 (s, 1H), 3.55 (br s, 1H), 3.34-3.38 (m, 1H), 2.09 (s, 3H), 1.92-2.02 (m, 1H), 1.79 (dd, J=4.4, 13.7 Hz, 1H), 1.42 (d, J=6.8, 3H), 1.38 (s, 3H), 1.24 (s, 3H).

### Example 32

### (±)-5-Chloro-6-(5-cyano-2-methoxyphenyl)-1,2,3,4-tetrahydro-2,2,4,8-tetramethylquinoline (Compound 132, Structure 6 of Scheme I, where R¹=Me, R²=H, R⁴=Cl, R⁵=Me, R⁶=H, R⁹=H, Ar=5-cyano-2-methoxyphenyl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (70 mg, 0.21 mmol) and 3-bromo-4-methoxybenzonitrile (53 mg, 0.25 mmol) to afford 27 mg of Compound 132 after flash chromatography. ¹H NMR (400 MHz, CDCl₃) δ 7.61 (dd, J=2.1, J=8.5, 1H), 7.45 (broad s, 1H), 6.97 (d, J=8.6, 1H), 6.77 (s, 1H), 3.83 (s, 3H), 3.61 (s, 1H), 3.35-3.37 (m, 1H), 2.07 (s, 3H), 1.80-1.96 (m, 2H), 1.42 (broad s, 3H), 1.37 (s, 3H), 1.24 (s, 3H).

### Example 33

### (±)-6-(1-Acetyl-3,5-dimethylpyrazol-4-yl)-5-chloro-1,2,3,4-tetrahydro-2,2,4,8-tetramethylquinoline (Compound 133, Structure 6 of Scheme I, where R¹=Me, R²=H, R⁴=Cl, R⁵=Me, R⁶=H, R⁹=H, Ar=1-acetyl-3,5-dimethylpyrazol-4-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (49 mg, 0.14 mmol) and 1-acetyl-4-bromo-3,5-dimethylpyrazole (46 mg, 0.21 mmol) to afford Compound 133 after flash chromatography (15% EtOAc/hexanes) and preparative HPLC (Beckman Ultrasphere ODS, 10×250 mm, 75% MeOH/water with 0.1% TFA). ¹H NMR (500 MHz, CDCl₃) δ 6.68 (s, 1H), 3.58 (broad s, 1H), 3.30-3.40 (m, 1H), 2.71 (s, 3H), 2.40 (s, 3/2H), 2.38 (s, 3/2H), 2.14 (s, 3H), 2.12 (s, 3/2H), 2.09 (s, 3H), 1.94-2.00 (m, 1H), 1.77-1.83 (m, 1H), 1.43 (d, J=7.0, 3/2H), 1.42 (d, J=7.0, 3/2H), 1.38 (s, 3H), 1.26 (s, 3/2H), 1.25 (s, 3/2H).

### Example 34

### (±)-5-Chloro-1,2,3,4-tetrahydro-6-(indol-3-yl)-2,2,4,8-tetramethylquinoline (Compound 134, Structure 6 of Scheme I, where R¹=Me, R²=H, R⁴=Cl, R⁵=Me, R⁶=H, R⁹=H, Ar=indol-3-yl)

To prepare this compound, first (±)-5-chloro-1,2,3,4-tetrahydro-6-[(triisopropylsilyl)indol-3-yl]-2,2,4,8-tetramethylquinoline was prepared using General Method 5 (EXAMPLE 1) from (±)-5-Chloro-1,2,3,4-tetrahydro-2,2,4,8-tetramethyl-6-(4,4,5,5-tetramethyl-1,3,2-dioxaborolan-2-yl)quinoline (70 mg, 0.20 mmol) and 3-bromo-1-(triisopropylsilyl)indole (74 mg, 0.21 mmol) to afford 23 mg of (±)-5-chloro-1,2,3,4-tetrahydro-6-[(triisopropylsilyl)indol-3-yl]-2,2,4,8-tetramethylquinoline after flash chromatography (10% EtOAc/hexanes). That compound was dissolved in 1 mL THF, cooled to 0° C., and treated with tetrabutylammonium fluoride (TBAF, 1M in THF, 0.05 mL). That solution was allowed to warm to room temperature, stirred for 4 hours, then partioned between EtOAc and saturated ammonium chloride. The organic layer washed with brine, dried over magnesium sulfate, filtered, and concentrated. Compound 138 (3 mg, 4% overall) was isolated after flash chromatography (25% EtOAc/hexanes) and preparative HPLC (Beckman Ultrasphere ODS, 10×250 mm, 90% MeOH/water). ¹H NMR (500 MHz, CDCl₃) δ 8.18 (broad s, 1H), 7.61 (d, J=7.8, 1H), 7.41 (d, J=8.3, 1H), 7.30 (d, J=2.4, 1H), 7.19-7.24 (m, 1H), 7.11-7.16 (m, 1H), 7.08 (s, 1H), 3.53 (broad s, 1H), 3.35-3.45 (m, 1H), 2.12 (s, 3H), 1.99 (dd, J=13.4, 7.1, 1H), 1.81 (dd, J=13.2, 4.4, 1H), 1.47 (d, J=7.3, 3H), 1.39 (s, 3H), 1.26 (s, 3H).

### Example 35

### (±)-5-Chloro-1,2,3,4-tetrahydro-2,2,4,8-tetramethyl-6-(naphthal-1-yl)quinoline (Compound 135, Structure 6 of Scheme I, where R¹=Me, R²=H, R⁴=Cl, R⁵=Me, R⁶=H, R⁹=H, Ar=naphthal-1-yl)

This compound was prepared using General Method 5 (EXAMPLE 1) from (±)-6-bromo-5-chloro-1,2,3,4-tetrahydro-2,2,4,8-tetramethylquinoline (30 mg, 0.10 mmol) and 1-naphthaleneboronic acid (22 mg, 0.13 mmol) to afford 20 g (57%) of Compound 135 after flash chromatography (30% dichloromethane/hexanes). ¹H NMR (500 MHz, CDCl₃) δ 7.87 (d, J=8.3, 1H), 7.84 (d, J=8.3, 1H), 7.65 (d, J=8.3, ½H), 7.56 (d, J=8.3, ½H), 7.33-7.53 (m, 4H), 6.89 (s, 1H), 3.59 (s, ½H), 3.58 (s, ½H), 3.30-3.40 (m, 1H), 2.11 (s, 3H), 1.97-2.06 (m, 1H), 1.80-1.86 (m, 1H), 1.48 (d, J=6.8, 3/2H), 1.45 (d, J=6.8, 3/2H), 1.42 (s, 3/2H), 1.41 (s, 3/2H), 1.30 (s, 3/2H), 1.27 (s, 3/2H).

### Example 36

### (±)-5-Chloro-1,2,3,4-tetrahydro-2,2,4,8-tetramethyl-6-(3-methylpyrid-2-yl)quinoline (Compound 136, Structure 6 of Scheme I, where R¹=Me, R²=H, R⁴=Cl, R⁵=Me, R⁶=H, R⁹=H, Ar=3-methylpyrid-2-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (50 mg, 0.14) and 2-bromo-3-methylpyridine (21 mg, 0.12 mmol) to afford 7 mg (16%) of Compound 136 after flash chromatography (30% EtOAc/hexanes). ¹H NMR (500 MHz, CDCl₃) δ 8.52 (br s, 1H), 7.59 (t, J=6.3 Hz, 1H), 7.21 (dd, J=4.9. 7.3 Hz, 1H), 6.89 (s, ½H), 6.86 (s, ½H), 3.57 (s, ½H), 3.61 (s, ½H) 3.28-3.41 (m, 1H), 2.26 (s, 3/2H), 2.20 (s, 3/2H), 2.12 (s, 3H), 1.96-2.06 (m, 1H), 1.78-1.86 (m, 1H), 1.46 (d, J=6.8 Hz, 3/2H), 1.44 (d, J=7.3 Hz, 3/2H), 1.40 (s, 3H), 1.29 (s, 3/2H), 1.22 (s, 3/2H).

### Example 37

### (±)-5-Chloro-6-(5-fluoroindol-7-yl)-1,2,3,4-tetrahydro-2,2,4,8-tetramethylquinoline (Compound 137, Structure 6 of Scheme I, where R¹=Me, R²=H, R⁴=Cl, R⁵=Me, R⁶=H, R⁹=H, Ar=5-fluoroindol-7-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (35 mg, 0.10 mmol) and 7-bromo-5-fluoroindole (32 mg, 0.15 mmol) to afford 29 mg (80%) of Compound 137 after flash chromatography (20% EtOAc/hexanes). ¹H NMR (500 MHz, CDCl₃) δ 8.02 (broad s, 1H), 7.18-7.30 (m, 2H), 6.97 (s, 1H), 6.86-6.94 (m, 1H), 6.55 (s, 1H), 3.64 (broad s, 1H), 3.35-3.42 (m, 1H), 2.11 (s, 3H), 1.99 (dd, J=13.6, 6.8, 1H), 1.80-1.88 (m, 1H), 1.47 (d, J=6.8, 3/2H), 1.45 (d, J=7.3, 3/2H), 1.41 (s, 3H), 1.28 (s, 3/2H), 1.27 (s, 3/2H).

### Example 38

### (±)-5-Chloro-1,2,3,4-tetrahydro-2,2,4,8-tetramethyl-6-(2-methylindol-7-yl)quinoline (Compound 138, Structure 6 of Scheme I, where R¹=Me, R²=H, R⁴=Cl, R⁵=Me, R⁶=H, R⁹=H, Ar=2-methylindol-7-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (35 mg, 0.10 mmol) and 7-bromo-2-methylindole (30 mg, 0.14 mmol) to afford 20 mg (57%) of Compound 138 after flash chromatography (15% EtOAc/hexanes). ¹H NMR (500 MHz, CDCl₃) δ 7.74 (broad s, ½H), 7.72 (broad s, ½H), 7.48 (d, J=7.8, 1H), 7.08-7.14 (m, 1H), 6.98-7.06 (m, 1H), 6.99 (s, 1H), 6.25 (s, 1H), 3.61 (broad s, ½H), 3.59 (broad s, ½H), 3.35-3.45 (m, 1H), 2.42 (s, 3/2H), 2.40 (s, 3/2H), 2.12 (s, 3H), 1.96-2.02 (m, 1H), 1.80-1.86 (m, 1H), 1.48 (d, J=7.4, 3/2H), 1.45 (d, J=7.4, 3/2H), 1.41 (s, 3H), 1.30 (s, 3/2H), 1.27 (s, 3/2H). ¹H NMR (500 MHz, DMSO-d₆, 50° C.) δ 10.3 (broad s, 1H), 7.35 (d, J=7.8, 1H), 6.96 (t, J=7.3, 1H), 6.90 (s, 1H), 6.81 (d, J=7.3, 1H), 6.16 (s, 1H), 4.81 (broad s, 1H), 3.26-3.34 (m, 1H), 2.37 (s, 3H), 2.13 (s, 3H), 1.93 (dd, J=13.7, 6.8, 1H), 1.83 (dd, J=13.7, 3.6, 1H), 1.43 (d, J=7.3, 3H), 1.41 (s, 3H), 1.29 (s, 3H).

### Example 39

### (±)-5-Chloro-1,2,3,4-tetrahydro-2,2,4,8-tetramethyl-6-(3-methylindol-7-yl)quinoline (Compound 139, Structure 6 of Scheme I, where R¹=Me, R²=H, R⁴=Cl, R⁵=Me, R⁶=H, R⁹=H, Ar=3-methylindol-7-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (35 mg, 0.10 mmol) and 7-bromo-3-methylindole (30 mg, 0.14 mmol) to afford 14 mg (40%) of Compound 139 after flash chromatography (15% EtOAc/hexanes). ¹H NMR (500 MHz, CDCl₃) δ 7.79 (broad s, ½H), 7.78 (broad s, ½H), 7.57 (d, J=7.8, 1H), 7.18 (dd, J=7.8, 7.3, 1H), 7.14 (d, J=7.3, ½H), 7.10 (d, J=6.8, ½H), 6.99 (s, 1H), 6.97 (s, ½H), 6.94 (s, ½H), 3.60 (broad s, 1H), 3.37-3.43 (m, 1H), 2.37 (s, 3H), 2.11 (s, 3H), 1.99 (dd, J=13.5, 7.1, 1H), 1.80-1.88 (m, 1H), 1.48 (d, J=6.8, 3/2H), 1.46 (d, J=6.8, 3/2H), 1.41 (s, 3H), 1.29 (s, 3/2H), 1.27 (s, 3/2H).

### Example 40

### (±)-5-Chloro-6-(5-chloroindol-7-yl)-1,2,3,4-tetrahydro-2,2,4,8-tetramethylquinoline (Compound 140, Structure 6 of Scheme I, where R¹=Me, R²=H, R⁴=Cl, R⁵=Me, R⁶=H, R⁹=H, Ar=5-chloroindol-7-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (70 mg, 0.20 mmol) and 7-bromo-5-chloroindole (55 mg, 0.24 mmol) to afford 8 mg (11%) of Compound 140 after flash chromatography (25% EtOAc/hexanes) and preparative HPLC (HiChrom C18, 10×250 mm, 80% MeOH/water, 3 mL/min). ¹H NMR (500 MHz, CDCl₃) δ 8.04 (broad s, 1H), 7.58 (d, J=2.0, 1H), 7.21 (s, ½H), 7.19 (s, ½H), 7.12 (s, ½H), 7.08 (s, ½H), 6.96 (s, 1H), 6.53 (s, 1H), 3.63 (broad s, 1H), 3.35-3.43 (m, 1H), 2.10 (s, 3H), 1.98 (dd, J=13.5, 7.1, 1H), 1.80-1.88 (m, 1H), 1.47 (d, J=7.3, 3/2H), 1.44 (d, J=7.3, 3/2H), 1.41 (s, 3H), 1.28 (s, 3/2H), 1.27 (s, 3/2H).

### Example 41

### (±)-5-Chloro-6-(4-fluoroindol-7-yl)-1,2,3,4-tetrahydro-2,2,4,8-tetramethylquinoline (Compound 141, Structure 6 of Scheme I, where R¹=Me, R²=H, R⁴=Cl, R⁵=Me, R⁶=H, R⁹=H, Ar=4-fluoroindol-7-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (136 mg, 0.39 mmol) and 7-bromo-4-fluoroindole (75 mg, 0.35 mol) to afford 59 mg (44%) of Compound 141 after flash chromatography (10% EtOAc/hexanes). ¹H NMR (500 MHz, CDCl₃) δ 8.14 (s, ½H), 8.12 (s, ½H) 7.14 (d, J=14.1 Hz, 1H), 6.88-7.14 (m, 1H), 6.95 (s, 1H), 6.83 (t, J=8.3 Hz, 1H), 6.67 (s, 1H), 3.62 (broad s, 1H), 3.32-3.42 (m, 1H), 2.11 (s, 3H), 1.99 (dd, J=6.8, 13.4 Hz, 1H), 1.79-1.84 (m, 1H), 1.48 (d, J=6.8 Hz, 3/2H), 1.45 (d, J=7.3 Hz, 3/2H), 1.41 (s, 3H), 1.29 (s, 3/2H) 1.27 (s, 3/2H).

### Example 42

### (±)-5-Chloro-6-(4-chloroindol-7-yl)-1,2,3,4-tetrahydro-2,2,4,8-tetramethylquinoline (Compound 142, Structure 6 of Scheme I, where R¹=Me, R²=H, R⁴=Cl, R⁵=Me, R⁶=H, R⁹=H, Ar=4-chloroindol-7-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (126 mg, 0.36 mmol) and 7-bromo-4-chloroindole (75 mg, 0.33 mol) to afford 100 mg (75%) of Compound 142 after flash chromography (10% EtOAc/hexanes). ¹H NMR (500 MHz, CDCl₃) δ 8.20 (br s, 1H), 7.27 (s, ½H), 7.24 (s, ½H), 7.20 (d, J=7.8 Hz, 1H), 7.10 (d, J=7.1 Hz, ½H), 7.06 (d, J=7.1 Hz, ½H), 6.72 (s, 1H), 3.67 (br s, 1H), 3.37-3.44 (m, 1H), 2.15 (s, 3H), 2.02 (dd, J=6.6, 13.4 Hz, 1H), 1.82-1.91 (m, 1H), 1.52 (d, J=6.8 Hz, 3/2H), 1.48 (d, J=7.3, 3/2H), 1.45 (s, 3H), 1.32 (s, 3/2H), 1.31 (s, 3/2H).

### Example 43

### (±)-5-Chloro-6-(4,5-difluoroindol-7-yl)-1,2,3,4-tetrahydro-2,2,4,8-tetramethylquinoline (Compound 143, Structure 6 of Scheme I, where R¹=Me, R²=H, R⁴=Cl, R⁵=Me, R⁶=H, R⁹=H, Ar=4,5-difluoroindol-7-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (126 mg, 0.36 mmol) and 7-bromo-4,5-difluoroindole (70 mg, 0.30 mmol) to afford 100 mg (89%) of Compound 143 after flash chromatography (10% EtOAc/hexanes). ¹H NMR (500 MHz, CDCl₃) δ 8.07 (br s, 1H), 7.12-7.19 (m, 1H), 6.93-7.01 (m, 1H), 6.92 (s, 1H), 6.66 (s, 1H), 3.64 (br s, 1H), 3.32-3.41 (m, 1H), 2.10 (s, 3H), 1.97 (dd, J=6.8, 13.7 Hz, 1H), 1.79-1.88 (m, 1H), 1.42-1.48 (m, 3H), 1.40 (s, 3H), 1.27 (s, 3H).

### Example 44

### (±)-5-Chloro-1,2,3,4-tetrahydro-6-(4-methoxyindol-7-yl)-2,2,4,8-tetramethylquinoline (Compound 144, Structure 6 of Scheme I, where R¹=Me, R²=H, R⁴=Cl, R⁵=Me, R⁶=H, R⁹=H, Ar=4-methoxyindol-7-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (85 mg, 0.24 mmol) and 7-bromo-4-methoxyindole (50 mg, 0.22 mmol) to afford 40 mg (49%) of Compound 144 after flash chromatography (10% EtOAc/hexanes). ¹H NMR (500 MHz, CDCl₃) δ 8.42 (br s, 1H), 7.11-7.18 (m, 2H), 7.02 (s, 1H), 6.73 (s, 1H), 6.64 (s, ½H), 6.62 (s, ½H), 4.04 (s, 3H), 3.62 (broad s, 1H), 3.38-3.46 (m, 1H), 2.15 (s, 3H), 2.03 (dd, J=6.3, 10.5 Hz, 1H), 1.82-1.91 (m, 1H), 1.52 (d, J=6.8 Hz, 3/2H), 1.49 (d, J=6.8, 3/2H), 1.45 (s, 3H), 1.32 (s, 3/2H), 1.31 (s, 3/2H).

### Example 45

### (±)-5-Chloro-6-(4-chloro-3-methylindol-7-yl)-1,2,3,4-tetrahydro-2,2,4,8-tetramethylquinoline (Compound 145, Structure 6 of Scheme I, where R¹=Me, R²=H, R⁴=Cl, R⁵=Me, R⁶=H, R⁹=H, Ar=4-chloro-3-methylindol-7-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (76 mg, 0.22 mol) and 7-bromo-4-chloro-3-methylindole (50 mg, 0.20 mmol) to afford 30 mg of Compound 145 after flash chromatography (10% EtOAc/hexanes). ¹H NMR (500 MHz, CDCl₃) δ 7.82 (broad s, ½H), 7.83 (broad s, ½H), 7.12 (d, J=2.2 Hz, 1H), 7.02 (d, J=7.8 Hz, 1H), 6.98 (d, J=5.4 Hz, 1H), 6.96 (s, 1H), 3.65 (broad s, ½H), 3.64 (broad s, ½H), 3.38-3.42 (m, 1H), 2.61 (s, 3H), 2.14 (s, 3H), 2.02 (dd, J=7.3, 13.8 Hz, 1H), 1.81-1.90 (m, 1H), 1.50 (d, J=7.3 Hz, 3/2H), 1.48 (d, J=7.3, 3/2H), 1.44 (s, 3H), 1.32 (s, 3/2H), 1.30 (s, 3/2H).

### Example 46

### (±)-5-Chloro-6-(2,3-dimethylindol-7-yl)-1,2,3,4-tetrahydro-2,2,4,8-tetramethylquinoline (Compound 146, Structure 6 of Scheme I, where R¹=Me, R²=H, R⁴=Cl, R⁵=Me, R⁶=H, R⁹=H, Ar=2,3-dimethylindol-7-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (86 mg, 0.24 mmol) and 7-bromo-2,3-dimethylindole (50 mg, 0.22 mmol) to afford 15 mg of Compound 146 after flash chromatography (10% EtOAc/hexanes). ¹H NMR (500 MHz, CDCl₃) δ 7.63 (s, ½H), 7.59 (s, ½H), 7.49 (d, J=7.8 Hz, 1H), 7.17 (t, J=5.9 Hz, 1H), 7.09 (d, J=6.2 Hz, ½H), 7.06 (d, J=6.2 Hz, ½H), 7.02 (s, 1H), 3.64 (s, 1H), 3.40-3.48 (m, 1H), 2.38 (s, 3/2H), 2.37 (s, 3/2H), 2.29 (s, 3H), 2.15 (s, 3H), 2.01-2.08 (m, 1H), 1.84-1.92 (m, 1H), 1.52 (d, J=6.8 Hz, 3/2H), 1.50 (d, J=6.8 Hz, 3/2H), 1.45 (s, 3H), 1.34 (s, 3/2H), 1.31 (s, 3/2H).

### Example 47

### (±)-5-Chloro-6-(4-fluoro-3-methylindol-7-yl)-1,2,3,4-tetrahydro-2,2,4,8-tetramethylquinoline (Compound 147, Structure 6 of Scheme I, where R¹=Me, R²=H, R⁴=Cl, R⁵=Me, R⁶=H, R⁹=H, Ar=4-fluoro-3-methylindol-7-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (85 mg, 0.24 mmol) and 7-bromo-4-fluoro-3-methylindole (50 mg, 0.22 mmol) to afford 35 mg of Compound 147 after flash chromatography (10% EtOAc/hexanes). ¹H NMR (500 MHz, CDCl₃) δ 7.81 (br s, 1H), 6.98-7.21 (m, 1H), 6.97 (s, 1H), 6.91 (s, ½H), 6.89 (s, ½H), 6.80 (t, J=8.8 Hz, 1H), 3.63 (br s, 1H), 3.38-3.61 (m, 1H), 2.52 (s, 3H), 2.14 (s, 3H), 2.02 (dd, J=6.8, 13.7 Hz, 1H), 1.82-1.90 (m, 1H), 1.51 (d, J=6.8, 3/2H), 1.48 (d, J=6.8 Hz, 3/2H), 1.44 (s, 3H), 1.32 (s, 3/2H), 1.30 (s, 3/2H).

### Example 48

### (±)-5-Chloro-1,2,3,4-tetrahydro-2,2,4,8-tetramethyl-6-(1-methylindol-7-yl)quinoline (Compound 148, Structure 6 of Scheme I, where R¹=Me, R²=H, R⁴=Cl, R⁵=Me, R⁶=H, R⁹=H, Ar=1-methylindol-7-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (38 mg, 0.11 mmol) and 7-bromo-1-methylindole (28 mg, 0.13 mmol) to afford 8 mg (21%) of Compound 148 after flash chromatography (20% EtOAc/hexanes) and preparative HPLC (HiChrom C18, 10×250 mm, 80% MeOH/water, 2.5 mL/min). ¹H NMR (400 MHz, CDCl₃) δ 7.60 (d, J=7.8, 1H), 7.09 (dd, J=7.8, 7.4, 1H), 6.90-7.00 (m, 3H), 6.51 (s, ½H), 6.50 (s, ½H), 3.54 (broad s, 1H), 3.36 (s, 3H), 3.28-3.36 (m, 1H), 2.11 (s, 3H), 1.99 (dd, J=13.5, 7.1, 1H), 1.79 (dd, J=13.5, 5.2, 1H), 1.43 (s, J=6.8, 3H), 1.39 (s, 3H), 1.25 (s, 3H).

### Example 49

### (±)-5-Chloro-1,2,3,4-tetrahydro-6-(indol-7-yl)-2,2,4,8-tetramethylquinoline (Compound 149, Structure 6 of Scheme I, where R¹=Me, R²=H, R⁴=Cl, R⁵=Me, R⁶=H, R⁹=H, Ar=indol-7-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (35 mg, 0.10 mmol) and 7-bromoindole (26 mg, 0.13 mmol) to afford 3 mg (9%) of Compound 149, after flash chromatography (20% EtOAc/hexanes). ¹H NMR (500 MHz, CDCl₃) δ 8.05 (broad s, 1H), 7.63 (d, J=7.8, 1H), 7.10-7.20 (m, 3H), 7.00 (s, 1H), 6.59 (s, 1H), 3.60 (broad s, 1H), 3.35-3.43 (m, 1H), 2.11 (s, 3H), 1.99 (dd, J=13.4, 7.1, 1H), 1.80-1.88 (m, 1H), 1.48 (d, J=6.8, 3/2H), 1.45 (d, J=7.3, 3/2H), 1.41 (s, 3H), 1.29 (s, 3/2H), 1.27 (s, 3/2H).

### Example 49A

### (+)-5-Chloro-1,2,3,4-tetrahydro-6-(indol-7-yl)-2,2,4,8-tetramethylquinoline (Compound 149A, Structure (+)-6 of Scheme I, where R¹=Me, R²=H, R⁴=Cl, R⁵=Me, R⁶=H, R⁹=H, Ar=indol-7-yl), and (−)-5-Chloro-1,2,3,4-tetrahydro-6-(indol-7-yl)-2,2,4,8-tetramethylquinoline (Compound 149B, Structure (−)-6 of Scheme I, where R¹=Me, R²=H, R⁴=Cl, R⁵=Me, R⁶=H, R⁹=H, Ar=indol-7-yl)

These compounds were isolated from the racemic compound of Example 49 using General Method 6 (EXAMPLE 1) on a Chiracel OJ column (20×250 mm, 30% isopropanol/hexanes, 6 ml/min, to afford Compounds 149A and 149B. Data for Compound 149A: HPLC (Chiralcel OJ, 30% EtOH/hexanes, 6 ml/min) t_{R} 47.5 min; [α]_{D}=+19.3. Data for Compound 149B: HPLC (Chiralcel OJ, 30% EtOH/hexanes, 6 ml/min) t_{R} 38.8 min; [α]_{D}=−20.7.

### Example 50

### (±)-5-Chloro-6-(3-cyano-2,6-dimethoxyphenyl)-1,2,3,4-tetrahydro-2,2,4,8-tetramethylquinoline (Compound 150, Structure 6 of Scheme I, where R¹=Me, R²=H, R⁴=Cl, R⁵=Me, R⁶=H, R⁹=H, Ar=3-cyano-2,6-dimethoxyphenyl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (70 mg, 0.20 mmol) and 3-bromo-2,6-dimethoxybenzonitrile (58 mg, 0.24 mmol) to afford 6 mg (8%) of Compound 150, after flash chromatography (30% EtOAc/hexanes) and preparative HPLC (Beckman Ultrasphere ODS, 10×250 mm, 75% MeOH/water, 3 mL/min). ¹H NMR (500 MHz, CDCl₃) δ 7.56 (d, J=8.6, 1H), 6.73-6.76 (m, 2H), 3.81 (s, 3/2H), 3.80 (s, 3/2H), 3.63 (s, 3/2H), 3.60 (s, 3/2H), 3.58 (broad s, 1H), 3.28-3.38 (m, 1H), 2.08 (s, 3H), 1.99 (dd, J=13.4, 7.3, 1H), 1.75-1.82 (m, 1H), 1.44 (d, J=7.3, 3/2H), 1.40 (d, J=7.3, 3/2H), 1.39 (s, 3/2H), 1.38 (s, 3/2H), 1.26 (s, 3/2H), 1.25 (s, 3/2H).

### Example 51

### (±)-5-Chloro-1,2,3,4-tetrahydro-6-(3-hydroxy-2-methoxyphenyl)-2,2,4,8-tetramethylquinoline (Compound 151, Structure 6 of Scheme I, where R¹=Me, R²=H, R⁴=Cl, R⁵=Me, R⁶=H, R⁹=H, Ar=3-hydroxy-2-methoxyphenyl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (21 mg, 0.06 mmol) to afford 12 mg (57%) of Compound 151 after flash chromatography (90% dichloromethane/hexanes to 2% EtOAc/dichloromethane, gradient elution). ¹H NMR (500 MHz, CDCl₃) δ 7.00 (dd, J=7.9, 7.8, 1H), 6.94 (dd, J=8.0, 1.6, 1H), 6.88 (s, 1H), 6.70-6.80 (m, 1H), 5.86 (broad s, 1H), 3.56 (broad s, 1H), 3.45 (broad s, 3H), 3.30-3.40 (m, 1H), 2.10 (s, 3H), 1.98 (dd, J=13.5, 7.2, 1H), 1.80 (dd, J=13.5, 4.5, 1H), 1.43 (d, J=6.8, 3H), 1.38 (s, 3H), 1.24 (s, 3H).

### Example 52

### (±)-5-Chloro-6-(1-tetralon-5-yl)-1,2,3,4-tetrahydro-2,2,4,8-tetramethylquinoline (Compound 152, Structure 6 of Scheme I, where R¹=Me, R²=H, R⁴=Cl, R⁵=Me, R⁶=H, R⁹=H, Ar=1-tetralon-5-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (114 mg, 0.34 mol) and 5-(trifluoromethanesulfonyl)oxy-1-tetralone (100 mg, 0.34 mmol) to afford 45 mg of Compound 152. ¹H NMR (400 MHz, CDCl₃) δ 8.05-8.07 (m, 1H), 7.31-7.36 (m, 2H), 6.74 (s, 1H), 3.57 (s, 1H), 3.32-3.35 (m, 1H), 2.61-2.66 (m, 4H), 2.09 (s, 3H), 1.94-2.05 (m, 3H), 1.80-1.82 (m, 1H), 1.44 (d, J=7.6, 3/2H), 1.42 (d, J=7.7, 3/2H), 1.38 (s, 3H), 1.26 (s, 3/2H), 1.24 (s, 3/2H).

### Example 53

### (±)-5-Chloro-6-(1-indanon-4-yl)-1,2,3,4-tetrahydro-2,2,4,8-tetramethylquinoline (Compound 153, Structure 6 of Scheme I, where R¹=Me, R²=H, R⁴=Cl, R⁵=Me, R⁶=H, R⁹=H, Ar=1-indanon-4-yl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (35 mg, 0.10 mmol) and 4-bromo-indan-1-one (28 mg, 0.13 mmol) to afford 22 mg (63%) of Compound 153 after flash chromatography (20% EtOAc/hexanes). ¹H NMR (400 MHz, CDCl₃) δ 7.75 (d, J=7.4, 1H), 7.40-7.52 (m, 1H), 7.41 (dd, J=7.4, 7.3, 1H), 6.80 (s, 1H), 3.60 (broad s, 1H), 3.30-3.40 (m, 1H), 2.80-3.10 (m, 2H), 2.60-2.70 (m, 2H), 2.10 (s, 3H), 1.98 (dd, J=13.5, 7.0, 1H), 1.82 (broad d, J=13.3, 1H), 1.44 (d, J=7.0, 3H), 1.40 (s, 3H), 1.27 (s, 3H).

### Example 54

### (±)-5-Chloro-6-(1-hydroxyiminoindan-4-yl)-1,2,3,4-tetrahydro-2,2,4,8-tetramethylquinoline (Compound 154, Structure 60 of Scheme XVI, where R¹=Me, R²=H, R⁴=Cl, R⁵=Me, R⁶=H, R⁹=H, R³⁰=H, n=1)

To prepare this compound, a solution of Compound 153 (EXAMPLE 53) (15 mg, 0.042 mmol), hydroxylamine hydrochloride (15 mg, 0.21 mmol), and sodium acetate (17 mg, 0.21 mmol) in 1 mL EtOH was heated at reflux for 2 hours. The solution was then partitioned between EtOAc and water, and the organic layer washed with brine, dried over magnesium sulfate, filtered, and concentrated. Flash chromatography (30% EtOAc/hexanes) afforded Compound 154. ¹H NMR (500 MHz, CDCl₃) δ 7.91 (broad s, 1H), 7.65 (dd, J=7.3, 1.0, 1H), 7.31 (dd, J=7.8, 7.3, 1H), 7.18-7.26 (m, 1H), 6.79 (s, 1H), 3.55 (broad s, 1H), 3.30-3.40 (m, 1H), 2.80-3.10 m, 4H), 2.09 (s, 3H), 1.98 (dd, J=13.4, 7.1, 1H), 1.81 (broad d, J=13.2, 1H), 1.44 (d, J=6.8, 3H), 1.39 (s, 3H), 1.26 (s, 3H).

### Example 55

### (±)-5-Chloro-6-(3-cyano-2-methylphenyl)-1,2,3,4-tetrahydro-2,2,4,8-tetramethylquinoline (Compound 155, Structure 6 of Scheme I, where R¹=Me, R²=H, R⁴=Cl, R⁵=Me, R⁶=H, R⁹=H, Ar=3-cyano-2-methylphenyl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (50 mg, 0.14 mmol) and 3-bromo-2-methylbenzonitrile (20 mg, 0.10 mmol) to afford 12 mg of Compound 155 after flash chromatography (10% EtOAc/hexanes). ¹H NMR (500 MHz, CDCl₃) δ 7.59 (dd, J=1.0, 7.3 Hz, 1H), 7.39 (d, J=1.0, 7.8 Hz, ½H), 7.34 (d, J=1.0, 7.3 Hz, ½H), 7.27-7.30 (m, 1H), 6.70 (s, 1H), 3.58 (broad s, 1H), 3.23-3.33 (m, 1H), 2.36 (s, 3/2H), 2.31 (s, 3/2H), 2.17 (s, 3/2H), 2.09 (s, 3/2H), 1.92-2.01 (m, 1H), 1.78-1.82 (m, 1H), 1.43 (d, J=6.8 Hz, 3/2H), 1.41 (d, J=6.8 Hz, 3/2H), 1.39 (s, 3H), 1.26 (s, 3/2H), 1.25 (s, 3/2H)

### Example 56

### (±)-5-Chloro-1,2,3,4-tetrahydro-6-(2-methoxy-3-nitrophenyl)-2,2,4,8-tetramethylquinoline (Compound 156, Structure 6 of Scheme I, where R¹=Me, R²=H, R⁴=Cl, R⁵=Me, R⁶=H, R⁹=H, Ar=2-methoxy-3-nitrophenyl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (330 mg, 0.95 mmol) and 2-bromo-6-nitroanisole (200 mg, 0.86 mmol) to afford 260 mg (73% of Compound 156 after flash chromatography (30% EtOAc/hexanes). ¹H NMR (500 MHz, CDCl₃) δ 7.72 (d, J=7.8, 1H), 7.45 (d, J=7.5 Hz, 1H), 7.18 (t, J=7.8 Hz, 1H), 6.85 (s, ½H), 6.88 (s, ½H), 3.62 (br s, 1H), 3.56 (s, 3/2H), 3.53 (s, 3/2H), 3.32-3.36 (m, 1H), 2.09 (s, 3H), 1.98 (dd, J=7.3, 13.2 Hz, 1H), 1.81 (dd, J=4.4, 13.7 Hz, 1H), 1.44 (d, J=7.3 Hz, 3H), 1.39 (s, 3H), 1.25 (s, 3H).

### Example 57

### (±)-5-Chloro-1,2,3,4-tetrahydro-6-(2-methoxy-6-nitrophenyl)-2,2,4,8-tetramethylquinoline (Compound 157, Structure 6 of Scheme I, where R¹=Me, R²=H, R⁴=Cl, R⁵=Me, R⁶=H, R⁹=H, Ar=2-methoxy-6-nitrophenyl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (35 mg, 0.1 mmol) and 2-bromo-3-nitroanisole (21 mg, 0.090 mmol) to afford 22 mg (65%) of Compound 157 after flash chromatography (30% EtOAc/hexanes). ¹H NMR (500 MHz, CDCl₃) δ 7.40-7.46 (m, 2H), 7.13-7.16 (m, 1H), 6.68 (s, ½H), 6.67 (s, ½H), 3.81 (s, 3/2H), 3.80 (s, 3/2H), 3.56 (s, ½H), 3.54 (s, ½H), 3.25-3.35 (m, 1H), 2.05 (s, 3/2H), 2.04 (s, 3/2H), 1.93-2.00 (m, 1H), 1.74-1.81 (m, 1H), 1.42 (d, J=7.3, 3/2H), 1.41 (d, J=7.3, 3/2H), 1.37 (s, 3/2H), 1.36 (s, 3/2H), 1.25 (s, 3/2H), 1.23 (s, 3/2H).

### Example 58

### (±)-6-(2-Benzyloxy-3-nitrophenyl)-5-chloro-1,2,3,4-tetrahydro-2,2,4,8-tetramethylquinoline (Compound 158, Structure 6 of Scheme I, where R¹=Me, R²=H, R⁴=Cl, R⁵=Me, R⁶=H, R⁹=H, Ar=2-benzyloxy-3-nitrophenyl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (384 mg, 1.1 mmol) and 2-benzyloxy-1-bromo-3-nitrobenzene (300 mg, 0.97 mmol) to afford 300 mg (60%) of Compound 158 after flash chromatography (5% EtOAc/hexanes). ¹H NMR (500 MHz, CDCl₃) δ 7.75-7.76 (m, 1H), 7.75 (d, J=6.8 Hz, ½H), 7.49 (d, J=6.8 Hz, ½H), 7.21-7.26 (m, 5H), 6.97-7.01 (m, 1H), 6.90 (d, J=12.2 Hz, 1H), 4.68-4.75 (m, 2H), 3.71 (br s, 1H), 3.35-3.38 (m, 1H), 2.07 (s, 3/2H), 2.05 (s, 3/2H), 1.99 (dd, J=7.3, 13.7 Hz, 1H), 1.81-1.84 (m, 1H), 1.45 (d, J=7.3 Hz, 3H), 1.42 (3/2H), 1.41 (s, 3/2H), 1.23 (s, 3H).

### Example 59

### (±)-5-Chloro-1,2,3,4-tetrahydro-3β-hydroxy-2,2,4α,8-tetramethylquinoline (Structure 3 of Scheme 1, where R¹=Me, R²=H, R³=H, R⁴=Cl, R⁵=α-Me, R⁶=β-OH, R⁹=H)

This compound was prepared by the hydroboration of a 4-alkyl-1,2-dihydroquinoline to produce a 4α-alkyl-1,2,3,4-tetrahydro-3β-hydroxyquinoline (trans-isomer), as follows, herein referred to as General Method 7. To a rapidly stirring solution of 5.0 g (22.6 mmol) of 5-chloro-1,2-dihydro-2,2,4,8-tetramethylquinoline in 100 mL of anhydrous tetrahydrofuran (0.23 M) at 0° C. under nitrogen was added dropwise, over 20 minutes, 27.0 mL of a 1.5 M solution of borane in tetrahydrofuran (1.8 equiv). After addition of the borane was complete, the mixture was stirred at for an additional 20 minutes at 0° C. and then at room temperature for 5 hours to produce an intermediate organoborane. That intermediate organoborane was oxidized by adding, successively, 25 mL of a 2.0 N aqueous potassium hydroxide solution (2.2 equiv) and 20 mL of 30% hydrogen peroxide (8.7 equiv) at 0° C. That mixture was then stirred at room temperature for 2 hours and then the mixture was diluted with 80 mL of water, resulting in a first organic layer and an aqueous layer. The first organic layer was collected and the aqueous phase was extracted with ethyl acetate. The organic layer from that extraction was combined with the first organic layer and that combined organic layer washed with brine and dried over sodium sulfate. Filtration and concentration of the filtrate in vacuo gave a brown oil which was chromatographed on silica gel. Elution with hexanes-ethyl acetate (4:1) provided 3.47 g (65%) of (±)-5-chloro-1,2,3,4-tetrahydro-3β-hydroxy-2,2,4α,8-tetramethylquinoline, as a white solid.

### 6-Bromo-5-chloro-1,2,3,4-tetrahydro-3β-hydroxy-2,2,4α,8-tetramethylquinoline (Structure 4 of Scheme 1, where R¹=Me, R²=H, R⁴=Cl, R⁵=α-Me, R⁶=β-OH, R⁹=H)

This compound was prepared using General Method 3 (EXAMPLE 1) from (±)-5-chloro-1,2,3,4-tetrahydro-3β-hydroxy-2,2,4α,8-tetramethylquinoline to afford (±)-6-bromo-5-chloro-1,2,3,4-tetrahydro-3β-hydroxy-2,2,4α,8-tetramethylquinoline after flash chromatography.

### (±)-6-(Benzothiophen-3-yl)-5-chloro-3β-hydroxy-2,2,4α,8-tetramethylquinoline (Compound 159, Structure 6 of Scheme I, where R¹=Me, R²=H, R⁴=Cl, R⁵=α-Me, R⁶=β-OH, R⁹=H, Ar=benzothiophen-3-yl)

This compound was prepared using General Method 5 (EXAMPLE 1) from (±)-6-bromo-5-chloro-1,2,3,4-tetrahydro-3β-hydroxy-2,2,4α,8-tetramethylquinoline and thianaphthene-3-boronic acid to afford Compound 159. ¹H NMR (500 MHz, CD₃OD) δ 7.88-7.81 (m, 1H), 7.42-7.40 (m, 1H), 7.35-7.28 (m, 3H), 6.90 (s, 1H), 3.47 (d, 1H, J=6.8), 2.94 (qn, 1H, J=6.8), 2.15 (s, 3H), 1.50 (d, 3H, J=6.8), 1.35 (s, 3H), 1.06 (s, 3H).

### Example 60

### (±)-5-Chloro-1,2,3,4-tetrahydro-3β-hydroxy-2,2,4α,8-tetramethyl-6-(thiophen-3-yl)quinoline (Compound 160, Structure 6 of Scheme I, where R¹=Me, R²=H, R⁴=Cl, R⁵=α-Me, R⁶=β-OH, R⁹=H, Ar=thiophen-3-yl)

This compound was prepared using General Method 5 (EXAMPLE 1) from (±)-6-bromo-5-chloro-1,2,3,4-tetrahydro-3β-hydroxy-2,2,4α,8-tetramethylquinoline and 3-thiopheneboronic acid to afford Compound 160. ¹H NMR (500 MHz, CD₃OD) δ 7.36-7.35 (m, 1H), 7.29-7.28 (m, 1H), 7.21-7.19 (m, 1H), 6.94 (s, 1H), 3.43 (d, 1H, J=6.8), 2.89 (qn, 1H, J=6.8), 2.12 (s, 3H), 1.45 (d, 3H, J=6.8), 1.32 (s, 3H), 1.01 (s, 3H).

### Example 61

### (±)-5-Chloro-1,2,3,4-tetrahydro-3β-hydroxy-6-(indol-7-yl)-2,2,4α,8-tetramethylquinoline (Compound 161, Structure 6 of Scheme I, where R¹=Me, R²=H, R⁴=Cl, R⁵=α-Me, R⁶=β-OH, R⁹=H, Ar=indol-7-yl)

This compound was prepared using General Method 5 (EXAMPLE 1) from (±)-6-bromo-5-chloro-1,2,3,4-tetrahydro-3β-hydroxy-2,2,4α,8-tetramethylquinoline and 7-(4,4,5,5-tetramethyl-1,3,2-dioxaborolan-2-yl)indole to afford Compound 161. ¹H NMR (500 MHz, CDCl₃) δ 8.03 (br s, 1H), 7.63 (d, 1H, J=7.8), 7.18-7.07 (m, 3H), 7.01 (s, 1H), 6.58 (br s, 1H), 3.63-3.59 (m, 2H), 3.20-3.13 (m, 1H), 2.13 (s, 3H), 1.91 (d, 0.5H, J=7.8), 1.85 (d, 0.5H, J=7.8), 1.57 (d, 1.5H, J=6.8), 1.53 (d, 1.5H, J=6.8), 1.36 (s, 3H), 1.26 (s, 1.5H), 1.22 (s, 1.5H).

### Example 61A

### (+)-5-Chloro-1,2,3,4-tetrahydro-3β-hydroxy-6-(indol-7-yl)-2,2,4α,8-tetramethylquinoline (Compound 161A, Structure (+)-6 of Scheme I, where R¹=Me, R²=H, R⁴=Cl, R⁵=α-Me, R⁶=β-OH, R⁹=H, Ar=indol-7-yl) and (−)-5-Chloro-1,2,3,4-tetrahydro-3β-hydroxy-6-(indol-7-yl)-2,2,4α,8-tetramethylquinoline (Compound 161B, Structure (−)-6 of Scheme I, where R¹=Me, R²=H, R⁴=Cl, R⁵=α-Me, R⁶=β-OH, R⁹=H, Ar=indol-7-yl)

The compounds were isolated from the racemic compound of Example 61 using General Method 6 (EXAMPLE 1) on a Chiracel OJ column (10×250 mm, 35% isopropanol/hexanes, 2.5 ml/min, to afford Compounds 161A and 161B. Data for Compound 161A: HPLC (Chiralcel OJ, 35% EtOH/hexanes, 2.5 ml/min) t_{R} 23.2 min; [α]_{D}=+56 (c=0.1, EtOH). Data for Compound 161B: HPLC (Chiralcel OJ, 35% EtOH/hexanes, 2.5 ml/min) t_{R} 15.3 min; [α]_{D}=−48 (c=0.1, EtOH).

### Example 62

### (±)-5-Chloro-1,2,3,4-tetrahydro-3β-hydroxy-2,2,4α,8-tetramethyl-6-(naphthal-1-yl)quinoline (Compound 162, Structure 6 of Scheme I, where R¹=Me, R²=H, R⁴=Cl, R⁵=α-Me, R⁶=β-OH, R⁹=H, Ar=naphthal-1-yl)

This compound was prepared using General Method 5 (EXAMPLE 1) from (±)-6-bromo-5-chloro-1,2,3,4-tetrahydro-3β-hydroxy-2,2,4α,8-tetramethylquinoline and 1-naphthaleneboronic acid to afford Compound 162. ¹H NMR (500 MHz, CDCl₃) δ 7.88 (dd, 1H, J=1.9, J=7.3), 7.85 (d, 1H, J=7.8), 7.61 (d, 0.5H, J=8.2), 7.52-7.33 (m, 4.5H), 6.92 (s, 1H), 3.64-3.58 (m, 2H), 3.17-3.08 (m, 1H), 2.14 (s, 3H), 1.88 (d, 0.5H, J=8.2), 1.85 (d, 0.5H, J=8.2), 1.57 (d, 1.5H, J=6.8), 1.53 (d, 1.5H, J=6.8), 1.39 (s, 1.5H), 1.38 (s, 1.5H), 1.28 (s, 1.5H), 1.24 (s, 1.5H).

### Example 63

### 5-Chloro-1,2,3,4-tetrahydro-3β-hydroxy-2,2,4α,8-tetramethyl-6-(4,4,5,5-tetramethyl-1,3,2-dioxaborolan-2-yl)quinoline (Structure 5 of Scheme 1, where R¹=Me, R²=H, R⁴=Cl, R⁵=α-Me, R⁶=β-OH, R⁹=H)

This compound was prepared using General Method 4 from (±)-6-bromo-5-chloro-1,2,3,4-tetrahydro-3β-hydroxy-2,2,4α,8-tetramethylquinoline to afford (±)-5-chloro-1,2,3,4-tetrahydro-3β-hydroxy-2,2,4α,8-tetramethyl-6-(4,4,5,5-tetramethyl-1,3,2-dioxaborolan-2-yl)quinoline, after flash chromatography (EtOAc/hexanes).

### (±)-5-Chloro-6-(4-fluoroindol-7-yl)-1,2,3,4-tetrahydro-3β-hydroxy-2,2,4α,8-tetramethylquinoline (Compound 163, Structure 6 of Scheme I, where R¹=Me, R²=H, R⁴=Cl, R⁵=α-Me, R⁶=β-OH, R⁹=H, Ar=4-fluoroindol-7-yl)

This compound was prepared using General Method 5 (EXAMPLE 1) from (±)-5-chloro-1,2,3,4-tetrahydro-3β-hydroxy-2,2,4α,8-tetramethyl-6-(4,4,5,5-tetramethyl-1,3,2-dioxaborolan-2-yl)quinoline and 7-bromo-4-fluoroindole to afford Compound 163. ¹H NMR (500 MHz, CDCl₃) δ 8.17 (br s, 1H), 7.13-7.15 (m, 1H), 6.97-7.02 (m, 2H), 6.80-6.84 (m, 1H), 6.65 (s, 1H), 3.63-3.65 (m, 2H), 3.10-3.18 (m, 1H), 2.13 (s, 3/2H), 2.16 (s, 3/2H), 1.76-2.00 (m, 1H), 1.56 (d, J=6.8 Hz, 3/2H), 1.52 (d, J=6.8 Hz, 3/2H), 1.36 (s, 3H), 1.26 (s, 3/2H), 1.22 (s, 3/2H).

### Example 64

### (±)-5-Chloro-6-(3,5-dimethylisoxazol-4-yl)-1,2,3,4-tetrahydro-3β-hydroxy-2,2,4α,8-tetramethylquinoline (Compound 164, Structure 6 of Scheme I, where R¹=Me, R²=H, R⁴=Cl, R⁵=α-Me, R⁶=β-OH, R⁹=H, Ar=3,5-dimethylisoxazol-4-yl)

This compound was prepared using General Method 5 (EXAMPLE 1) from (±)-6-bromo-5-chloro-1,2,3,4-tetrahydro-3β-hydroxy-2,2,4α,8-tetramethylquinoline and 3,5-dimethyl-4-isoxazolylboronic acid to afford Compound 164. ¹H NMR (500 MHz, CD₃OD) δ 6.77 (s, 1H), 3.37 (d, J=6.8, 1H), 2.91 (qn, 0.5H, J=6.8), 2.89 (qn, 0.5H, J=6.8), 2.23 (s, 1.5H), 2.21 (s, 1.5H), 2.14 (s, 3H), 2.10 (s, 1.5H), 2.06 (s, 1.5H), 1.46 (d, 1.5H, J=6.8), 1.45 (d, 1.5H, J=6.8), 1.33 (s, 3H), 1.03 (s, 3H).

### Example 65

### (±)-5-Chloro-6-(3-cyano-2-methoxyphenyl)-1,2,3,4-tetrahydro-3β-hydroxy-2,2,4α,8-tetramethylquinoline (Compound 165, Structure 6 of Scheme I, where R¹=Me, R²=H, R⁴=Cl, R⁵=α-Me, R⁶=β-OH, R⁹=H, Ar=3-cyano-2-methoxyphenyl)

This compound was prepared using General Method 5 (EXAMPLE 1) from (±)-6-bromo-5-chloro-1,2,3,4-tetrahydro-3β-hydroxy-2,2,4α,8-tetramethylquinoline and 3-(4,4,5,5-tetramethyl-1,3,2-dioxaborolan-2-yl)-2-methoxybenzonitrile to afford of Compound 165. ¹H NMR (500 MHz, CD₃OD) δ 7.62 (dd, J=1.5, J=7.8, 1H), 7.48-7.42 (br m, 1H), 7.25 (t, J=7.8, 1H), 6.83 (s, 1H), 3.62-3.53 (m, 3H), 3.44 (d, J=6.8, 1H), 2.92-2.88 (m, 1H), 2.13 (s, 3H), 1.46 (d, J=6.8, 3H), 1.34 (s, 3H), 1.01 (s, 3H).

### Example 66

### (±)-5-Chloro-1,2,3,4-tetrahydro-3β-hydroxy-6-(4-fluoro-3-methylindol-7-yl)-2,2,4α,8-tetramethylquinoline (Compound 166, Structure 6 of Scheme I, where R¹=Me, R²=H, R⁴=Cl, R⁵=α-Me, R⁶=β-OH, R⁹=H, Ar=4-fluoro-3-methylindol-7-yl)

This compound was prepared using General Method 5 (EXAMPLE 1) from (±)-5-chloro-1,2,3,4-tetrahydro-3β-hydroxy-2,2,4α,8-tetramethyl-6-(4,4,5,5-tetramethyl-1,3,2-dioxaborolan-2-yl)quinoline and 7-bromo-4-fluoro-3-methylindole to afford Compound 166. ¹H NMR (500 MHz, CDCl₃) δ 7.78 (br s, 1H), 6.92-6.98 (m, 2H), 6.87 (s, ½H), 6.85 (s, ½H), 6.78 (d, J=7.8 Hz, ½H), 6.75 (d, J=7.8 Hz, ½H), 3.59-3.61 (m, 2H), 3.10-3.17 (m, 1H), 2.45 (s, 3H), 2.13 (s, 3H), 1.89 (d, J=8.0 Hz, ½H), 1.83 (d, J=7.8 Hz, ½H), 1.56 (d, J=6.8 Hz, 3/2H), 1.52 (d, J=6.8 Hz, 1H), 1.36 (s, 3H), 1.23 (s, 3/2H), 1.26 (s, 3/2H).

### Example 67

### (±)-5-Chloro-1,2,3,4-tetrahydro-3β-hydroxy-6-(5-fluoroindol-7-yl)-2,2,4α,8-tetramethylquinoline (Compound 167, Structure 6 of Scheme I, where R¹=Me, R²=H, R⁴=Cl, R⁵=α-Me, R⁶=β-OH, R⁹=H, Ar=5-fluoroindol-7-yl)

This compound was prepared using General Method 5 (EXAMPLE 1) from (±)-5-chloro-1,2,3,4-tetrahydro-3β-hydroxy-2,2,4α,8-tetramethyl-6-(4,4,5,5-tetramethyl-1,3,2-dioxaborolan-2-yl)quinoline and 7-bromo-5-fluoroindole to afford Compound 167. ¹H NMR (500 MHz, CDCl₃) δ 8.02 (br s, 1H), 7.26 (s, 1H), 7.20-7.23 (m, 1H), 7.00 (s, 1H), 6.87-6.92 (m, 1H), 6.55 (s, 1H), 3.62-3.64 (m, 2H), 3.11-3.16 (m, 1H), 2.18 (s, 3/2H), 2.17 (s, 3/2H), 1.92 (d, J=6.8 Hz, ½H), 1.86 (d, J=7.3 Hz, ½H), 1.52-1.57 (m, 3H), 1.37 (s, 3H), 1.23 (s, 3/2H), 1.27 (s, 3/2H).

### Example 68

### (±)-5-Chloro-1,2,3,4-tetrahydro-3β-hydroxy-6-(3-methylindol-7-yl)-2,2,4α,8-tetramethylquinoline (Compound 168, Structure 6 of Scheme I, where R¹=Me, R²=H, R⁴=Cl, R⁵=α-Me, R⁶=β-OH, R⁹=H, Ar=3-methylindol-7-yl)

This compound was prepared using General Method 5 (EXAMPLE 1) from (±)-5-chloro-1,2,3,4-tetrahydro-3β-hydroxy-2,2,4α,8-tetramethyl-6-(4,4,5,5-tetramethyl-1,3,2-dioxaborolan-2-yl)quinoline and 7-bromo-3-methylindole to afford Compound 168. ¹H NMR (500 MHz, CDCl₃) δ 7.98 (br s, 1H), 7.57 (d, J=7.8 Hz, 1H), 7.13-7.16 (m, 1H), 7.07-7.09 (m, 1H), 7.01 (s, 1H), 6.94 (s, ½H), 6.96 (s, ½H), 3.70-3.72 (m, 2H), 3.13-3.18 (m, 1H), 2.36 (s, 3H), 2.13 (s, 3H), 1.85-1.91 (m, 1H), 1.52-1.57 (m, 3H), 1.37 (s, 3H), 1.23 (s, 3/2H), 1.26 (s, 3/2H).

### Example 69

### 7-Chloro-1,2-dihydro-2,2,4,8-tetramethylquinoline (Structure 2 of Scheme 1, where R¹=Me, R²=Cl, R³=H, R⁴=H, R⁵=Me)

This compound was prepared using General Method 1 (EXAMPLE 1) from 3-chloro-2-methylaniline (9.5 g, 67 mmol), iodine (5.0 g, 20 mmol), N,O-bis(trimethylsilyl)acetamide (26 g, 130 mmol) in 335 mL acetone heated at 130° C. for 18 h to afford, after an aqueous workup, 7.3 g (49%) of 7-chloro-1,2-dihydro-2,2,4,8-tetramethylquinoline, an amber oil, after flash chromatography (12% EtOAc/hexanes). ¹H NMR (500 MHz, CDCl₃) δ 6.87 (d, J=8.3, 1H), 6.66 (d, J=8.3, 1H), 5.31 (d, J=1.5, 1H), 3.68 (broad s, 1H), 2.16 (s, 3H), 1.97 (d, J=1.5, 3H), 1.29 (s, 6H).

### (±)-7-Chloro-1,2,3,4-tetrahydro-2,2,4,8-tetramethylquinoline (Structure 3 of Scheme 1, where R¹=Me, R²=Cl, R³=H, R⁴=H, R⁵=Me, R⁶=H, R⁹=H)

This compound was prepared using General Method 2 (EXAMPLE 1) from 7-chloro-1,2-dihydro-2,2,4,8-tetramethylquinoline (3.9 g, 17 mmol) heated for 3 hours to afford 1.7 g (43%) of 7-chloro-1,2,3,4-tetrahydro-2,2,4,8-tetramethylquinoline, after flash chromatography (10% EtOAc/hexanes). ¹H NMR (400 MHz, CDCl₃) δ 6.97 (d, J=8.3, 1H), 6.69 (d, J=8.3, 1H), 3.55 (broad s, 1H), 2.85-2.95 (m, 1H), 2.15 (s, 3H), 1.70-1.80 (m, 1H), 1.40 (dd, J=12.6, 12.6, 1H), 1.31 (d, J=6.7, 3H), 1.29 (s, 3H), 1.17 (s, 3H).

### (±)-6-Bromo-7-chloro-1,2,3,4-tetrahydro-2,2,4,8-tetramethylquinoline (Structure 4 of Scheme 1, where R¹=Me, R²=Cl, R⁴=H, R⁵=Me, R⁶=H, R⁹=H)

This compound was prepared using General Method 3 from 7-chloro-1,2,3,4-tetrahydro-2,2,4,8-tetramethylquinoline (1.6 g, 7.0 mmol) to afford 1.25 g (59%) of (±)-6-bromo-7-chloro-1,2,3,4-tetrahydro-2,2,4,8-tetramethylquinoline, a brown solid. ¹H NMR (400 MHz, CDCl₃) δ 7.29 (s, 1H), 3.53 (broad s, 1H), 2.82-2.92 (m, 1H), 2.21 (s, 3H), 1.70-1.80 (m, 1H), 1.39 (dd, J=12.6, 12.6, 1H), 1.31 (d, J=6.7, 3H), 1.29 (s, 3H), 1.16 (s, 3H).

### (±)-7-Chloro-6-(3-cyano-2-methoxyphenyl)-1,2,3,4-tetrahydro-2,2,4,8-tetramethylquinoline (Compound 169, Structure 6 of Scheme I, where R¹=Me, R²=Cl, R⁴=H, R⁵=Me, R⁶=H, R⁹=H, Ar=3-cyano-2-methoxyphenyl)

This compound was prepared using General Method 5 (EXAMPLE 1) from (±)-6-bromo-7-chloro-1,2,3,4-tetrahydro-2,2,4,8-tetramethylquinoline (43 mg, 0.14 mmol) and 2-methoxy-3-(4,4,5,5-tetramethyl-1,3,2-dioxaborolan-2-yl)benzonitrile (43 mg, 0.17 mmol) to afford 32 mg (64%) of Compound 169 after flash chromatography (33% EtOAc/hexanes). ¹H NMR (400 MHz, CDCl₃) δ 7.55, (dd, J=7.8, 1.8, 1H), 7.44 (broad d, J=7.7, 1H), 7.16 (dd, J=7.7, 7.7, 1H), 6.97 (s, 1H), 3.68 (s, 3H), 3.65 (broad s, 1H), 2.86-2.96 (m, 1H), 2.22 (s, 3H), 1.78 (dd, J=13.0, 5.5, 1H), 1.40-1.50 (m, 1H), 1.33 (s, 3H), 1.31 (d, J=6.7, 3H), 1.22 (s, 3H).

### Example 70

### (±)-7-Chloro-6-(3-cyanophenyl)-1,2,3,4-tetrahydro-2,2,4,8-tetramethylquinoline (Compound 170, Structure 6 of Scheme I, where R¹=Me, R²=Cl, R⁴=H, R⁵=Me, R⁶=H, R⁹=H, Ar=3-cyanophenyl)

This compound was prepared using General Method 5 (EXAMPLE 1) from (±)-6-bromo-7-chloro-1,2,3,4-tetrahydro-2,2,4,8-tetramethylquinoline (33 mg, 0.11 mmol) and 3-cyanophenylboronic acid (21 mg, 0.14 mmol) to afford 22 mg (61%) of Compound 170 after flash chromatography (20% EtOAc/hexanes). ¹H NMR (400 MHz, CDCl₃) δ 7.70 (s, 1H), 7.62-7.68 (m, 1H), 7.55-7.60 (m, 1H), 7.47 (dd, J=7.7, 7.7, 1H), 6.98 (s, 1H), 3.70 broad s, 1H), 2.90-3.00 (m, 1H), 2.23 (s, 3H), 1.79 (dd, J=12.9, 5.4, 1H), 1.45 (dd, J=12.7, 12.6, 1H), 1.34 (d, 3H), 1.33 (s, 3H), 1.22 (s, 3H).

### Example 71

### 7-(4,4,5,5-tetramethyl-1,3,2-dioxaborolan-2-yl)indole7-(4,4,5,5-tetramethyl-1,32-dioxaborolan-2-yl)indole

This compound was prepared according to General Method 4 (EXAMPLE 1) from 7-bromoindole (0.29 g, 1.5 mmol) to afford 0.20 g (54%) of 7-(4,4,5,5-tetramethyl-1,3,2-dioxaborolan-2-yl)indole after flash chromatography (15% ethyl acetate/hexanes). ¹H NMR (500 MHz, CDCl₃) δ 9.27 (broad s, 1H), 7.80 (d, J=7.8, 1H), 7.69 (dd, J=6.8, 1.0, 1H), 7.29 (t, J=2.4, 1H), 7.16 (d, J=7.8, 1H), 6.58 (dd, J=2.9, 2.4, 1H), 1.42 (s, 12H).

### (±)-7-Chloro-1,2,3,4-tetrahydro-6-(indol-7-yl)-2,2,4,8-tetramethylquinoline (Compound 171, Structure 6 of Scheme I, where R¹=Me, R²=Cl, R⁴=H, R⁵=Me, R⁶=H, R⁹=H, Ar=indol-7-yl)

This compound was prepared using General Method 5 (EXAMPLE 1) from (±)-6-bromo-7-chloro-1,2,3,4-tetrahydro-2,2,4,8-tetramethylquinoline (36 mg, 0.12 mmol) and 7-(4,4,5,5-tetramethyl-1,3,2-dioxaborolan-2-yl)indole (32 mg, 0.13 mmol) to afford 12 mg (29%) of Compound 171, after flash chromatography (25% EtOAc/hexanes). Additional purification by preparative HPLC (Ultrasphere ODS, 10×250 mm, 85% MeOH/water, 3 mL/min) afforded 2 mg (5%) of final compound 171. ¹H NMR (500 MHz, CDCl₃) δ 8.04 (broad s, 1H), 7.64 (d, J=7.8, 1H), 7.14 (s, 1H), 7.04-7.20 (m, 3H), 6.58-6.61 (m, 1H), 3.66 (broad s, 1H), 2.90-3.00 (m, 1H), 2.26 (s, 3H), 1.76-1.82 (m, 1H), 1.47 (dd, J=12.7, 12.7, 1H), 1.34 (s, 3H), 1.32 (d, J=6.4, 3/2H), 1.29 (d, J=6.4, 3/2H), 1.24 (s, 3H).

### Example 72

### 7-Chloro-1,2,3,4-tetrahydro-3β-hydroxy-2,2,4α,8-tetramethylquinoline (Structure 3 of Scheme 1, where R¹=Me, R²=Cl, R³=H, R⁴=H, R⁵=α-Me, R⁶=β-OH, R⁹=H)

This compound was prepared using General Method 7 (EXAMPLE 59) from 7-chloro-1,2-dihydro-2,2,4,8-tetramethylquinoline (1.6 g, 7.4 mmol) to afford 0.90 g (49%) of 7-chloro-1,2,3,4-tetrahydro-3β-hydroxy-2,2,4α,8-tetramethylquinoline after flash chromatography (25% EtOAc/hexanes). ¹H NMR (500 MHz, CDCl₃) δ 6.97 (d, J=8.3, 1H), 6.73 (d, J=8.3, 1H), 3.60 (broad s, 1H), 3.31 (dd, J=9.5, 6.3, 1H), 2.62-2.72 (m, 1H), 2.16 (s, 3H), 1.71 (d, J=6.3, 1H), 1.40 (d, J=6.8, 3H), 1.34 (s, 3H), 1.07 (s, 3H).

### (±)-6-Bromo-7-chloro-1,2,3,4-tetrahydro-3β-hydroxy-2,2,4α,8-tetramethylquinoline (Structure 4 of Scheme 1, where R¹=Me, R²=Cl, R⁴=H, R⁵=α-Me, R⁶=β-OH, R⁹=H)

This compound was prepared using General Method 3 (EXAMPLE 1) from 7-chloro-1,2,3,4-tetrahydro-3β-hydroxy-2,2,4α,8-tetramethylquinoline (0.88 g, 3.7 mol) to afford 0.67 g (57%) of (±)-6-bromo-7-chloro-1,2,3,4-tetrahydro-3β-hydroxy-2,2,4α,8-tetramethylquinoline after flash chromatography (35% EtOAc/hexanes). ¹H NMR (500 MHz, CDCl₃) δ 7.29 (s, 1H), 3.60 (broad s, 1H), 3.29 (dd, J=9.8, 5.9, 1H), 2.62-2.72 (m, 1H), 2.23 (s, 3H), 1.73 (d, J=5.9, 1H), 1.40 (d, J=6.3, 3H), 1.34 (s, 3H), 1.06 (s, 3H).

### (±)-7-Chloro-6-(3,5-dimethylisoxazol-4-yl)-1,2,3,4-tetrahydro-3β-hydroxy-2,2,4α,8-tetramethylquinoline (Compound 172, Structure 6 of Scheme I, where R¹=Me, R²=Cl, R⁴=H, R⁵=α-Me, R⁶=β-OH, R⁹=H, Ar=3,5-dimethylisoxazol-4-yl)

This compound was prepared using a modified General Method 5 (Palladium-catalyzed Suzuki cross-coupling of an aryl halide and an aryl boronic acid or aryl pinacol boronate), as follows. In a Schlenck reaction flask, a mixture of (±)-6-bromo-7-chloro-1,2,3,4-tetrahydro-3β-hydroxy-2,2,4α,8-tetramethylquinoline (45 mg, 0.14 mol); 3,5-dimethyl-4-isoxazolylboronic acid (25 mg, 0.18 mmol); and Pd₂dba₃ (5.5 mg, 0.006 mmol), and DPPF (7.3 mg, 0.013 mmol) was evacuated under vacuum, and back-filled with nitrogen. Dioxane (0.1-0.2 M) and 2M sodium carbonate (2 equiv) were introduced sequentially. The mixture was heated (95-100° C.) for 16-24 hours. The mixture was partitioned between saturated ammonium chloride and EtOAc, and the aqueous layer was extracted with EtOAc. The combined organic layers were washed with brine, dried over magnesium sulfate, filtered, and concentrated under reduced pressure. Flash chromatography (40% EtOAc/hexanes) afforded 32 mg (68%) of Compound 172. ¹H NMR (500 MHz, CDCl₃) δ 6.86 (s, 1H), 3.72 (broad s, 1H), 3.30-3.40 (m, 1H), 2.70-2.78 (m, 1H), 2.27 (s, 3/2H), 2.26 (s, 3/2H), 2.23 (s, 3H), 2.14 (s, 3/2H), 2.13 (s, 3/2H), 1.78 (d, J=5.9, ½H), 1.77 (d, J=5.9, ½H), 1.40 (d, J=6.8, 3H), 1.37 (s, 3H), 1.13 (s, 3H).

### Example 73

### (±)-7-Chloro-1,2,3,4-tetrahydro-3β-hydroxy-6-(indol-7-yl)-2,2,4α,8-tetramethylquinoline (Compound 173, Structure 6 of Scheme I, where R¹=Me, R²=Cl, R⁴=H, R⁵=α-Me, R⁶=β-OH, R⁹=H, Ar)

This compound was prepared using General Method 5 (EXAMPLE 1) from (±)-6-bromo-7-chloro-1,2,3,4-tetrahydro-3β-hydroxy-2,2,4α,8-tetramethylquinoline (71 mg, 0.22 mol) and 7-(4,4,5,5-tetramethyl-1,3,2-dioxaborolan-2-yl)indole (59 mg, 0.24 mmol) (EXAMPLE 71) to afford 56 mg (71%) of Compound 173 after flash chromatography (45% EtOAc/hexanes). ¹H NMR (500 MHz, CDCl₃) δ 8.03 (broad s, 1H), 7.64 (d, J=7.8, 1H), 7.03-7.20 (m, 4H), 6.60 (s, 1H), 3.73 (broad s, 1H), 3.34-3.40 (m, 1H), 2.70-2.80 (m, 1H), 2.28 (s, 3H), 1.78 (d, J=5.9, ½H), 1.75 (d, J=6.3 ½H), 1.41 (d, J=6.8, 3/2H), 1.39 (s, 3H), 1.38 (d, J=6.4, 3/2H), 1.15 (s, 3H).

### Example 74

### 4′-Amino-2′-chloro-2-methoxybiphenyl-3-carbonitrile (Structure 8 of Scheme 2, where R¹=H, R²=H, R⁴=Cl)

This compound was prepared using General Method 5 (EXAMPLE 1) from 3-chloro-4-(4,4,5,5-tetramethyl-1,3,2-dioxaborolan-2-yl)-phenylamine (0.20 g, 0.80 mmol) and 3-bromo-2-methoxybenzonitrile (0.19 g, 0.88 mmol) to afford 0.14 g (68%) of 4′-amino-2′-chloro-2-methoxybiphenyl-3-carbonitrile after flash chromatography (40% EtOAc/hexanes).

### Example 75

### 5-Chloro-6-(3-cyano-2-methoxyphenyl)-1,2-dihydro-2,2,4-trimethylquinoline (Compound 174, Structure 11 of Scheme 2, where R¹=H, R²=H, R⁴=Cl, R⁵=Me, Ar=3-cyano-2-methoxyphenyl) and 7-chloro-6-(3-cyano-2-methoxyphenyl)-1,2-dihydro-2,2,4-trimethylquinoline (Compound 175, Structure 11 of Scheme 2, where R¹=H, R²=Cl, R⁴=H, R⁵=Me, Ar=3-cyano-2-methoxyphenyl)

These compounds were prepared using General Method 1 (EXAMPLE 1) from 4′-amino-2′-chloro-2-methoxybiphenyl-3-carbonitrile (EXAMPLE 74) (0.11 g, 0.42 mmol) to afford 21 mg (15%) of Compound 174 and 61 mg (42%) of Compound 175 after flash chromatography (20% EtOAc/hexanes).

Data for Compound 174: ¹H NMR (500 MHz, CDCl₃) δ 7.57 (dd, J=7.6, 1.5, 1H), 7.44 (dd, J=7.6, 1.5, 1H), 7.17 (dd, J=7.6, 7.6, 1H), 6.87 (d, J=8.2, 1H), 6.50 (d, J=8.2, 1H), 5.51 (d, J=1.3, 1H), 3.98 (broad s, 1H), 3.69 (s, 3H), 2.32 (d, J=1.5, 1H), 1.29 (s, 6H). Data for Compound 175: ¹H NMR (500 MHz, CDCl₃) δ 7.57 (dd, J=7.7, 1.7, 1H), (7.46 (dd, J=7.7, 1.7, 1H), 7.17 (dd, J=7.6, 7.6, 1H), 6.93 (s, 1H), 6.54 (s, 1H), 5.36 (broad s, 1H), 3.87 (broad s, 1H), 3.71 (s, 3H), 1.94 (d, J=1.5, 3H), 1.32 (s, 6H).

### Example 76

### (±)-5-Chloro-6-(3-cyano-2-methoxyphenyl)-1,2,3,4-tetrahydro-2,2,4,8-tetramethylquinoline (Compound 176, Structure 6 of Scheme II, where R¹=H, R²=H, R⁴=Cl, R⁵=Me, R⁶=H, R⁹=H, Ar=3-cyano-2-methoxyphenyl)

This compound was prepared using General Method 2 (EXAMPLE 1) from Compound 174 (EXAMPLE 75) (14 mg, 0.041 mmol) to afford 12 mg (86%) of Compound 176 after flash chromatography (20% EtOAc/hexanes). ¹H NMR (500 MHz, CDCl₃) δ 7.56 (dd, J=7.6, 1.8, 1H), 7.38-7.50 (m, 1H), 7.16 (dd, J=7.6, 7.6, 1H), 6.88 (d, J=8.2, 1H), 6.43 (d, J=8.2, 1H), 3.79 (broad s, 1H), 3.68 (broad s, 3H), 3.28-3.38 (m, 1H), 1.98 (dd, J=13.4, 7.0, 1H), 1.79 (dd, J=13.4, 4.3, 1H), 1.43 (d, J=7.0, 3H), 1.36 (s, 3H), 1.24 (s, 3H).

### Example 77

### (±)-7-Chloro-6-(3-cyano-2-methoxyphenyl)-1,2,3,4-tetrahydro-2,2,4-trimethylquinoline (Compound 177, Structure 6 of Scheme II, where R¹=H, R²=Cl, R⁴=H, R⁵=Me, R⁶=H, R⁹=H, Ar=3-cyano-2-methoxyphenyl)

This compound was prepared using General Method 2 (EXAMPLE 1) from Compound 175 (EXAMPLE 75) (27 mg, 0.080 mmol) to afford 19 mg (70%) of Compound 177 after flash chromatography (20% EtOAc/hexanes). ¹H NMR (500 MHz, CDCl₃) δ 7.56 (dd, J=7.7, 1.6, 1H), 7.47 (dd, J=7.7, 1.6, 1H), 7.17 (dd, J=7.7, 7.7, 1H), 7.06 (s, ½H), 7.05 (s, ½H), 6.55 (s, 1H), 3.83 (broad s, 1H), (3.70 (s, 3H), 2.88-2.95 (m, 1H), 1.77 (dd, J=12.8, 5.5, 1H), 1.45 (dd, J=12.8, 12.5, 1H), 1.31 (d, J=6.7, 3H), 1.28 (s, 3H), 1.23 (s, 3H).

### Example 78

### 5-Chloro-6-(3,5-dimethylisoxazol-4-yl)-1,2-dihydro-2,2,4,8-tetramethylquinoline (Compound 178, Structure 11 of Scheme 2, where R¹=Me, R²=H, R¹=H, R⁵=Me, Ar=3,5-dimethylisoxazol-4-yl)

This compound was prepared using General Method 1 (EXAMPLE 1) from 2-methyl-4-(3,5-dimethylisoxazol-4-yl)phenylamine (96 mg, 0.41 mmol) to afford 22 mg (17%) of Compound 178. ¹H NMR (400 MHz, CDCl₃) δ 6.71 (s, 1H), 5.50 (broad s, 1H), 3.84 (broad s, 1H), 2.32 (s, 3H), 2.26 (s, 3H), 2.14 (s, 3H), 2.10 (s, 3H), 1.30 (s, 6H).

### Example 79

General Method 8: PCC oxidation of an alcohol to a ketone. To a solution of an alcohol (1 equiv) in anhydrous dichloromethane (0.025 M) at room temperature, pyridinium chlorochromate (3.5 equiv) is added. After stirring for 4.5 hours, the reaction mixture is diluted with ether and stirred vigorously for 10 minutes. The suspension is filtered through a pad of Celite-silica gel and washed successively with ether. The filtrate is concentrated in vacuo and chromatographed on silica gel. Elution with hexanes-ethyl acetate affords the desired ketone.

### (±)-5-Chloro-6-(3-cyano-2-methoxyphenyl)-1,4-dihydro-2,2,4,8-tetramethyl-2H-quinolin-3-one (Compound 179, Structure 13 of Scheme III, where R¹=Me, R²=H, R⁴=Cl, R⁵=Me, Ar=3-cyano-2-methoxyphenyl)

This compound was prepared using General Method 8 from Compound 165 (EXAMPLE 65) to afford Compound 179. ¹H NMR (500 MHz, CDCl₃) δ 7.59 (dd, J=1.9, 7.8, 1H), 7.49-7.44 (m, 1H), 7.20 (t, J=7.3, 1H), 6.94 (s, 1H), 4.25 (q, J=7.3, 1H), 3.73 (s, 1H), 3.68 (br s, 3H), 2.20 (s, 3H), 1.53 (s, 3H), 1.42 (d, J=7.3, 3H), 1.22 (s, 3H).

### Example 80

General Method 10. Alkylation of a 2H-quinolin-3-one with an alkyl halide, in the presence of potassium tert-butoxide. To a solution of a ketone (0.22 g, 0.69 mmol, 1 equiv) in anhydrous tetrahydrofuran (8 mL) at room temperature under nitrogen, 0.31 g (2.78 mmol, 4 equiv) of potassium tert-butoxide is added. After stirring for 20 minutes, an alkyl halide (5 equiv) is added dropwise over 3 minutes. The reaction is stirred for an additional 1 hour, quenched by the addition of 5 mL of a saturated aqueous ammonium chloride solution, and diluted with water and ethyl acetate, resulting in an aqueous layer and a first organic layer. The first organic layer is collected and the aqueous layer is extracted with a second organic layer of ethyl acetate. The first and second organic layers are combined and that combined organic layer is dried over anhydrous magnesium sulfate, filtered, and concentrated in vacuo to give a yellow solid. The crude product is chromatographed on 6 g silica gel. Elution with hexanes-ethyl acetate affords the desired ketone.

### (±)-4-Benzyl-5-chloro-6-(3-cyano-2-methoxyphenyl)-1,4-dihydro-2,2,4,8-tetramethyl-2H-quinolin-3-one (Compound 180, Structure 15 of Scheme III, where R¹=Me, R²=H, R⁴=Cl R⁵=Me, R⁹=benzyl Ar=3-cyano-2-methoxyphenyl)

This compound was prepared using General Method 10 from Compound 179 (EXAMPLE 79) and benzyl bromide to afford Compound 180. ¹H NMR (500 MHz, CD₃OD) δ 7.65 (dd, J=1.9, 7.8, 1H), 7.46 (dd, J=1.9, 7.8, 1H), 7.27 (t, J=7.8, 1H), 6.96 (s, 1H), 3.63 (s, 3H), 2.21 (s, 3H), 1.69 (s, 3H), 1.68 (s, 3H), 1.37 (s, 3H), 1.36 (s, 3H).

### Example 81

### 5-Chloro-6-(3-cyano-2-methoxyphenyl)-1,4-dihydro-2,2,4,4,8-pentamethyl-2H-quinolin-3-one (Compound 181, Structure 15 of Scheme III, where R¹=Me, R²=H, R⁴=Cl, R⁵=Me, R⁹-methyl Ar=3-cyano-2-methoxyphenyl)

This compound was prepared using General Method 10 (EXAMPLE 80) from Compound 179 (EXAMPLE 79) and iodomethane to afford Compound 181. ¹H NMR (500 MHz, CD₃OD) δ 7.65 (dd, J=1.9 7.8, 1H), 7.46 (dd, J=1.9, 7.8, 1H), 7.27 (t, J=7.8, 1H), 6.96 (s, 1H), 3.63 (s, 3H), 2.21 (s, 3H), 1.69 (s, 3H), 1.68 (s, 3H), 1.37 (s, 3H), 1.36 (s, 3H).

### Example 82

General Method 9: Swern oxidation of an alcohol to a ketone. A solution of anhydrous dimethyl sulfoxide (10 equiv) dissolved in anhydrous dichloromethane (0.6 M) at −78° C. under nitrogen is treated dropwise with a solution of oxalyl chloride (5 equiv) in dichloromethane (2 M). After stirring at −78° C. for 20 min, the alcohol (1 equiv), dissolved in anhydrous dichloromethane (0.4 M), is added dropwise over 5 minutes. The reaction is stirred at −78° C. for 20 minutes, then warmed to −40° C. and stirred for 20 minutes. After cooling back to −78° C., anhydrous triethylamine (10 equiv) is added dropwise over 3 minutes. The reaction is allowed to warm to 0° C. over 1.5 hours and is poured into a saturated aqueous sodium bicarbonate solution. The aqueous layer is extracted with dichloromethane, and the combined organic layers are dried over sodium sulfate, filtered, and concentrated in vacuo. The crude residue is chromatographed on silica gel. Elution with hexanes-ethyl acetate affords the desired ketone.

### (±)-5-Chloro-6-(3,5-dimethylisoxazol-4-yl)-1,4-dihydro-2,2,4,8-tetramethyl-2H-quinolin-3-one (Compound 182, Structure 13 of Scheme III, where R¹=Me, R²=H, R⁴=Cl, R⁵=Me, Ar=3,5-dimethylisoxazol-4-yl)

This compound was prepared using General Method 9 from Compound 164 (EXAMPLE 64) to afford Compound 182. ¹H NMR (500 MHz, CDCl₃) δ 6.82 (s, 1H), 3.99 (q, J=7.3, 0.5H), 3.89 (q, J=7.3, 0.5H), 3.71 (br s, 1H), 2.29 (s, 1.5H), 2.25 (s, 1.5H), 2.19 (s, 3H), 2.16 (s, 1.5H), 2.13 (s, 1.5H), 1.52 (s, 3H), 1.42 (d, J=1.4, 1.5H), 1.40 (d, J=1.4, 1.5H), 1.22 (s, 3H).

### Example 83

### 5-Chloro-6-(3,5-dimethylisoxazol-4-yl)-1,4-dihydro-2,2,4,4,8-pentamethyl-2H-quinolin-3-one (Compound 183, Structure 15 of Scheme III, where R¹=Me, R²=H, R⁴=Cl, R⁵=Me, R⁹=Me Ar=3,5-dimethylisoxazol-4-yl)

This compound was prepared using General Method 10 (EXAMPLE 80) from Compound 182 (EXAMPLE 82) and iodomethane to afford Compound 183. ¹H NMR (500 MHz, CDCl₃) δ 6.82 (s, 1H), 3.72 (brs, 1H), 2.26 (s, 3H), 2.16 (s, 3H), 2.13 (s, 3H), 1.73 (s, 3H), 1.71 (s, 3H), 1.39 (s, 6H).

### Example 84

### (±)-4-Benzyl-5-chloro-6-(3,5-dimethylisoxazol-4-yl)-1,4-dihydro-2,2,4,8-tetramethyl-2H-quinolin-3-one (Compound 184, Structure 15 of Scheme III, where R¹=Me, R²=H, R⁴=Cl R⁵=Me, R⁹=benzyl Ar=3,5-dimethylisoxazol-4-yl)

This compound was prepared using General Method 10 (EXAMPLE 80) from Compound 182 (EXAMPLE 82) and benzyl bromide to afford Compound 184. ¹H NMR (500 MHz, CD₃OD) δ 7.00-6.97 (m, 1H), 6.95-6.92 (m, 2H), 6.83 (d, J=3.4, 1H), 6.67-6.62 (m, 2H), 3.61 (d, J=12.7, 0.5H), 3.58 (d, J=12.7, 0.5H), 3.49 (d, J=13.1, 0.5H), 3.47 (d, J=13.1, 0.5H), 2.30 (s, 1.5H), 2.24 (s, 1.5H), 2.17 (s, 1.5H), 2.10 (s, 1.5H), 1.95-1.93 (m, 6H), 1.24 (s, 3H), 1.23-1.19 (m, 6H).

### Example 85

### (±)-5-Chloro-4-(3,3-dimethylallyl)-6-(3,5-dimethylisoxazol-4-yl)-1,4-dihydro-2,2,4,8-tetramethyl-2H-quinolin-3-one (Compound 185, Structure 15 of Scheme III, where R¹=Me. R²=H, R⁴=Cl, R⁵=Me, R⁹=3,3-dimethylallyl Ar=3,5-dimethylisoxazol-4-yl)

This compound was prepared using General Method 10 (EXAMPLE 80) from Compound 182 (EXAMPLE 82) and 3,3-dimethylallyl bromide to afford Compound 185. ¹H NMR (500 MHz, CDCl₃) δ 6.81 (s, 1H), 4.68 (t, 0.5H), 4.65 (t, 0.5H), 3.69 (br s, 1H), 3.14-3.09 (m, 1H), 2.89 (dd, J=7.3, 14.6, 0.5H), 2.86 (dd, J=7.3, 14.6, 0.5H), 2.27 (s, 1.5H), 2.24 (s, 1.5H), 2.15 (br s, 3H), 2.14 (s, 1.5), 2.11 (s, 1.5H), 1.79 (s, 1.5H), 1.77 (s, 1.5H), 1.49-1.47 (m, 3H), 1.41 (s, 1.5H), 1.38 (s, 1.5H), 1.36 (s, 1.5H), 1.34 (s, 1.5H), 1.33 (s, 1.5H), 1.33 (s, 1.5H).

### Example 86

### (±)-6-Bromo-5-chloro-1,4-dihydro-2,2,4,8-tetramethyl-2H-quinolin-3-one (Structure 24 of Scheme V, where R¹=Me, R²=H, R⁴=Cl, R⁵=Me)

This compound was prepared using General Method 9 (EXAMPLE 82) from (±)-6-bromo-5-chloro-1,2,3,4-tetrahydro-3β-hydroxy-2,2,4α,8-tetramethylquinoline (EXAMPLE 59) to afford 0.78 g (79%) of (±)-6-bromo-5-chloro-1,4-dihydro-2,2,4,8-tetramethyl-2H-quinolin-3-one after flash chromatography (20% EtOAc/hexanes).

### (±)-5-Chloro-1,4-dihydro-6-(indol-7-yl)-2,2,4α,8-tetramethyl-2H-quinolin-3-one (Compound 186, Structure 24 of Scheme V, where R¹=Me, R²=H, R⁴=Cl, R⁵=Me, Ar=indol-7-yl)

This compound was prepared using General Method 5 (EXAMPLE 1) from (±)-6-bromo-5-chloro-1,4-dihydro-2,2,4,8-tetramethyl-2H-quinolin-3-one and 7-(4,4,5,5-tetramethyl-1,3,2-dioxaborolan-2-yl)indole to afford Compound 186. ¹H NMR (500 MHz, CD₃OD) δ 7.75-7.73 (m, 1H), 7.18-7.14 (m 1H), 7.09-7.01 (m 2H), 6.99-6.92 (m, 1H), 6.45 (br s, 1H), 4.03-3.88 (bm, 1H), 2.25 (s, 3H), 1.52 (s, 3H), 1.41 (br s, 3H), 1.13 (br s, 1H).

### Example 87

### 6-Bromo-5-chloro-1,4-dihydro-2,2,4,4,8-pentamethyl-2H-quinolin-3-one (Structure 27 of Scheme V, where R¹=Me, R²=H, R⁴=Cl, R⁵=Me, R⁹=Me)

This compound was prepared using General Method 10 (EXAMPLE 80) from (±)-6-bromo-5-chloro-1,4-dihydro-2,2,4,8-tetramethyl-2H-quinolin-3-one and iodomethane to afford 6-bromo-5-chloro-1,4-dihydro-2,2,4,4,8-pentamethyl-2H-quinolin-3-one.

### 5-Chloro-1,4-dihydro-6-(indol-7-yl)-2,2,4,4,8-pentamethyl-2H-quinolin-3-one (Compound 187, Structure 28 of Scheme V, where R¹=Me, R²=H, R⁴=Cl, R⁵=Me, R⁹=Me, Ar=indol-7-yl)

This compound was prepared using General Method 5 (EXAMPLE 1) from 6-bromo-5-chloro-1,4-dihydro-2,2,4,4,8-pentamethyl-2H-quinolin-3-one and 7-(4,4,5,5-tetramethyl-1,3,2-dioxaborolan-2-yl)indole to afford Compound 187. ¹H NMR (500 MHz, CD₃OD) δ 7.55 (dd, 1H, J=1.5, J=8.3), 7.19 (d, 1H, J=3.4), 7.19-7.05 (m, 2H), 6.95 (dd, 1H, J=1.5, J=8.3), 6.50 (d, 1H, J=3.4), 2.26 (s, 3H), 1.79 (s, 3H), 1.73 (s, 3H), 1.43 (s, 6H).

### Example 88

### (±)-4-Benzyl-6-bromo-5-chloro-1,4-dihydro-2,2,4,8-tetramethyl-2H-quinolin-3-one (Structure 27 of Scheme V, where R¹=Me, R²=H, R⁴=Cl, R⁵=Me, R⁹=benzyl)

This compound was prepared using General Method 10 (EXAMPLE 80) from (±)-6-bromo-5-chloro-1,4-dihydro-2,2,4,8-tetramethyl-2H-quinolin-3-one and benzyl bromide to afford (±)-4-benzyl-6-bromo-5-chloro-1,4-dihydro-2,2,4,8-tetramethyl-2H-quinolin-3-one.

### (±)-4-Benzyl-5-chloro-1,4-dihydro-6-(indol-7-yl)-2,2,4,8-tetramethyl-2H-quinolin-3-one (Compound 188, Structure 28 of Scheme V, where R¹=Me, R²=H, R⁴=Cl, R⁵=Me, R⁹-benzyl Ar=indol-7-yl)

This compound was prepared using General Method 5 (EXAMPLE 1) from (±)-4-benzyl-6-bromo-5-chloro-1,4-dihydro-2,2,4,8-tetramethyl-2H-quinolin-3-one and 7-(4,4,5,5-tetramethyl-1,3,2-dioxaborolan-2-yl)indole to afford Compound 188. ¹H NMR (500 MHz, CDCl₃) δ 8.05 (br s, 0.5H), 7.68 (d, 0.5H, J=8.2), 7.64 (d, 0.5H, J=7.8), 7.57 (br s, 0.5H), 7.24-6.98 (m, 7H), 6.84 (dd, 1H, J=1.4, J=7.8), 6.68 (dd, 1H, J=1.4, J=7.8), 6.63-6.60 (m, 1H), 3.77 (d, 0.5H, J=13.6), 3.67 (d, 0.5H, J=13.2), 3.58 (d, 0.5H, J=13.6), 3.52 (br s, 0.5H), 3.47 (d, 0.5H, J=13.2), 3.24 (br s, 0.5H), 2.01 (s, 3H), 1.97 (s, 1.5H), 1.93 (s, 1.5H), 1.37 (s, 1.5H), 1.36 (s, 1.5H), 1.29 (s, 1.5H), 1.20 (s, 1.5H).

### Example 89

### (±)-6-Bromo-5-chloro-4-(3,3-dimethylallyl)-1,4-dihydro-2,2,4,8-tetramethyl-2H-quinolin-3-one (Structure 27 of Scheme V, 4-(3,3-dimethylallyl)- where R¹=Me, R²=H, R⁴=Cl, R⁵=Me, R⁹=3,3-dimethylallyl)

This compound was prepared using General Method 10 (EXAMPLE 80) from (±)-6-bromo-5-chloro-1,4-dihydro-2,2,4,8-tetramethyl-2H-quinolin-3-one and 3,3-dimethylallyl bromide to afford (±)-6-bromo-5-chloro-4-(3,3-dimethylallyl)-1,4-dihydro-2,2,4,8-tetramethyl-2H-quinolin-3-one.

### 5-Chloro-4-(3,3-dimethylallyl)-1,4-dihydro-6-(indol-7-yl)-2,2,4,8-tetramethyl-2H-quinolin-3-one (Compound 189, Structure 28 of Scheme V, where R¹=Me, R³=H, R⁴=Cl, R⁵=Me, R⁹=3,3-dimethylallyl Ar=indol-7-yl)

This compound was prepared using General Method 5 (EXAMPLE 1) from (±)-6-bromo-5-chloro-4-(3,3-dimethylallyl)-1,4-dihydro-2,2,4,8-tetramethyl-2H-quinolin-3-one and 7-(4,4,5,5-tetramethyl-1,3,2-dioxaborolan-2-yl)indole to afford Compound 189. ¹H NMR (500 MHz, CD₃OD) δ 10.0 (br s, 0.5H), 9.78 (br s, 0.5H), 7.55 (d, 1H, J=7.8), 7.21-7.18 (m, 1H), 7.11-7.05 (m, 2H), 6.98 (dd, 0.5H, J=0.9, J=7.3), 6.93 (dd, J=0.9, J=7.3), 6.52-6.49 (m, 1H), 4.90-4.81 (m 0.5H), 4.78-4.75 (m, 0.5H), 3.22-3.14 (m, 1H), 2.96-2.91 (m, 0.5H), 2.86-2.81 (m, 0.5H), 2.26 (s, 1.5H), 2.23 (s, 1.5H), 1.88 (s, 1.5H), 1.81 (s, 1.5H), 1.58 (s, 1.5H), 1.54 (s, 1.5H), 1.52 (s, 1.5H), 1.44 (s, 1.5H), 1.40 (s, 1.5H), 1.37 (s, 1.5H), 1.36 (s, 3H).

### Example 90

### (±)-4-Allyl-6-bromo-5-chloro-1,4-dihydro-2,2,4,8-tetramethyl-2H-quinolin-3-one (Structure 27 of Scheme V, where R¹=Me, R²=H, R⁴=Cl, R⁵=Me, R⁹=allyl)

This compound was prepared using General Method 10 (EXAMPLE 80) from (±)-6-bromo-5-chloro-1,4-dihydro-2,2,4,8-tetramethyl-2H-quinolin-3-one and allyl bromide to afford (±)-4-allyl-6-bromo-5-chloro-1,4-dihydro-2,2,4,8-tetramethyl-2H-quinolin-3-one.

### (±)-4-Allyl-5-chloro-1,4-dihydro-6-(indol-7-yl)-2,2,4,8-tetramethyl-2H-quinolin-3-one (Compound 190, Structure 28 of Scheme V, where R¹=Me, R²=H, R⁴=Cl, R⁵=Me, R⁹-allyl Ar=indol-7-yl)

This compound was prepared using General Method 5 (EXAMPLE 1) from (±)-4-allyl-6-bromo-5-chloro-1,4-dihydro-2,2,4,8-tetramethyl-2H-quinolin-3-one and 7-(4,4,5,5-tetramethyl-1,3,2-dioxaborolan-2-yl)indole to afford Compound 190. ¹H NMR (500 MHz, CDCl₃) δ 8.06 (br s, 0.5H), 7.91 (br s, 0.5H), 7.28 (d, 1H, J=7.8), 7.24-7.20 (m, 2H), 7.18-7.10 (m, 2H), 6.65-6.63 (m, 1H), 5.50-5.37 (m, 1H), 5.05-4.83 (m, 2H), 3.78-3.74 (m, 1H), 3.36-3.29 (m, 1H), 3.15-3.09 (m, 0.5H), 3.00-2.96 (m, 0.5H), 2.20 (s, 3H), 1.88 (s, 1.5H), 1.84 (s, 1.5H), 1.45 (s, 1.5H), 1.44 (s, 1.5H), 1.42 (s, 1.5H).

### Example 91

General Method 11. Reduction of a 2H-quinolin-3-one with sodium borohydride to afford an alcohol. To a solution of a 2H-quinolin-3-one (1.0 equiv) in 0.17 M anhydrous methanol at room temperature under nitrogen, sodium borohydride (2.0 equiv) is added. The reaction is stirred for 40 minutes then poured into a saturated solution of sodium bicarbonate (10 mL/mmol). The aqueous phase is extracted with ethyl acetate (3×10 mL/mmol), dried over sodium sulfate, filtered, and concentrated in vacuo. The residue is chromatographed over silica gel to afford the desired alcohol.

### (±)-6-Bromo-5-chloro-1,2,3,4-tetrahydro-3α-hydroxy-2,2,4α,8-tetramethylquinoline (Structure 25 of Scheme V, where R¹=Me, R²=H, R⁴=Cl, R⁵=Me)

This compound was prepared using General Method 11 from (±)-6-bromo-5-chloro-1,4-dihydro-2,2,4,8-tetramethyl-2H-quinolin-3-one (0.27 g, 0.85 mmol) to afford 0.23 g (86%) of (±)-6-bromo-5-chloro-1,2,3,4-tetrahydro-3α-hydroxy-2,2,4α,8-tetramethylquinoline after flash chromatography (20% EtOAc/hexanes).

### (±)-5-Chloro-6-(3-cyano-2-methoxyphenyl)-1,2,3,4-tetrahydro-3α-hydroxy-2,2,4α,8-tetramethylquinoline (Compound 191, Structure 26 of Scheme V, where R¹=Me, R²=H, R⁴=Cl, R⁵=Me, Ar=3-cyano-2-methoxyphenyl)

This compound was prepared using General Method 5 (EXAMPLE 1) from (±)-6-bromo-5-chloro-1,2,3,4-tetrahydro-3α-hydroxy-2,2,4α,8-tetramethylquinoline and 2-methoxy-3-(4,4,5,5-tetramethyl-1,3,2-dioxaborolan-2-yl)benzonitrile to afford Compound 191. ¹H NMR (500 MHz, CD₃OD) δ 7.60 (dd, 1H, J=1.4, J=7.3), 7.49-7.41 (br m, 1H), 7.22 (t, 1H, J=7.3), 6.80 (s, 1H), 3.80-3.74 (br s, 1H), 3.58 (br s, 3H), 3.42-3.37 (m, 1H), 2.11 (s, 3H), 1.37 (d, 3H, J=6.8), 1.32 (s, 6H).

### Example 92

### (±)-5-Chloro-6-(3,5-dimethylisoxazol-4-yl)-1,2,3,4-tetrahydro-3α-hydroxy-2,2,4α,8-tetramethylquinoline (Compound 192, Structure 26 of Scheme V, where R¹=Me, R²=H, R⁴=Cl, R⁵=Me, Ar=3,5-dimethylisoxazol-4-yl)

This compound was prepared using General Method 5 (EXAMPLE 1) from (±)-6-bromo-5-chloro-1,2,3,4-tetrahydro-3α-hydroxy-2,2,4α,8-tetramethylquinoline and 3,5-dimethyl-4-isoxazolylboronic acid to afford Compound 192. ¹H NMR (500 MHz, CD₃OD) δ 6.74 (s, 1H), 3.77 (d, 0.5H, J=6.4), 3.69 (d, 0.5H, J=6.4), 3.41-3.34 (m, 1H), 2.23 (s, 1.5H), 2.21 (s, 1.5H), 2.10 (s, 1.5H), 2.09 (s, 3H), 2.06 (s, 1.5H), 1.36 (d, 1.5H, J=7.3), 1.35 (d, 1.5H, J=7.3), 1.31 (s, 6H).

### Example 93

### (±)-5-Chloro-1,2,3,4-tetrahydro-3α-hydroxy-6-(indol-7-yl)-2,2,4α,8-tetramethylquinoline (Compound 193, Structure 26 of Scheme V, where R¹=Me, R²=H, R⁴=Cl, R⁵=Me, Ar=indol-7-yl)

This compound was prepared using General Method 5 (EXAMPLE 1) from (±)-6-bromo-5-chloro-1,2,3,4-tetrahydro-3α-hydroxy-2,2,4α,8-tetramethylquinoline and 7-(4,4,5,5-tetramethyl-1,3,2-dioxaborolan-2-yl)indole to afford Compound 193. ¹H NMR (500 MHz, CD₃OD) δ 7.49 (d, 1H, J=7.8), 7.14 (br s, 1H), 7.01 (t, 1H, J=7.3), 6.95-6.90 (m, 2H), 6.44 (d, 1H, J=2.4), 3.81 (d, 1H, J=5.9), 3.51-3.40 (br m, 1H), 2.11 (s, 3H), 1.45-1.41 (m, 3H), 1.38 (s, 3H), 1.37 (s, 3H).

### Example 94

### (±)-6-(Benzothiophen-3-yl)-5-chloro-1,2,3,4-tetrahydro-3α-hydroxy-2,2,4α,8-tetramethylquinoline (Compound 194, Structure 26 of Scheme V, where R¹=Me, R²=H, R⁴=Cl, R⁵=Me, Ar=benzothiophen-3-yl)

This compound was prepared using General Method 5 (EXAMPLE 1) from (±)-6-bromo-5-chloro-1,4-dihydro-2,2,4,8-tetramethyl-2H-quinolin-3-one and thianaphthene-3-boronic acid to afford Compound 194. ¹H NMR (500 MHz, CD₃OD) δ 7.88-7.86 (m, 1H), 7.44-7.41 (m, 1H), 7.34-7.30 (m, 3H), 6.88 (br s, 1H), 3.82 (d, 1H, J=6.3), 3.44-3.41 (m, 1H), 2.11 (s, 3H), 1.41 (d, 3H, J=6.8), 1.34 (s, 3H), 1.33 (s, 3H).

### Example 95

### (±)-5-Chloro-1,2,3,4-tetrahydro-3α-hydroxy-2,2,4α,8-tetramethyl-6-(naphthal-1-yl)quinoline (Compound 195, Structure 26 of Scheme V, where R¹=Me, R²=H, R⁴=Cl, R⁵=Me Ar=naphthal-1-yl)

This compound was prepared using General Method 5 (EXAMPLE 1) from (±)-6-bromo-5-chloro-1,2,3,4-tetrahydro-3α-hydroxy-2,2,4α,8-tetramethylquinoline and 1-naphthaleneboronic acid to afford Compound 195. ¹H NMR (500 MHz, CDCl₃) δ 7.88 (d, 1H, J=8.2), 7.85 (d, 1H, J=8.2), 7.62 (d, 0.5H, J=8.2), 7.53-7.32 (m, 4.5H), 6.91 (d, 1H, J=2.4), 3.83 (dd, 0.5H, J=5.9, J=7.8), 3.77 (dd, 0.5H, J=5.9, J=7.8), 3.57 (br s, 0.5H), 3.55 (br s, 0.5H), 3.43 (dq, 0.5H, J=5.9, J=6.8), 3.34 (dq, 0.5H, J=5.9, J=6.8), 2.14 (s, 1.5H), 2.13 (s, 1.5H), 1.97 (d, 0.5H, J=7.8), 1.93 (d, 0.5H, J=7.8), 1.53 (d, 1.5H, J=6.8), 1.47 (d, 1.5H, J=6.8), 1.40 (s, 1.5H), 1.39 (s, 1.5H), 1.32 (s, 1.5H), 1.28 (s, 1.5H).

### Example 96

### (±)-5-Chloro-1,2,3,4-tetrahydro-3-hydroxy-6-(indol-7-yl)-2,2,4,4,8-pentamethylquinoline (Compound 196, Structure 29 of Scheme V, where R¹=Me, R²=H, R⁴=Cl, R⁵=Me, R⁹=Me, Ar=indol-7-yl)

This compound was prepared using General Method 11 (EXAMPLE 91) from 5-chloro-1,4-dihydro-6-(indol-7-yl)-2,2,4,4,8-pentamethyl-2H-quinolin-3-one (Compound 187) to afford Compound 196. ¹H NMR (500 MHz, CDCl₃) δ 8.02-7.92 (br m, 1H), 7.63 (d, 1H, J=7.8), 7.19-7.18 (m, 1H), 7.16 (t, 1H, J=7.3), 7.08 (d, 1H, J=6.8), 6.99 (s, 1H), 6.59 (m, 1H), 3.61-3.60 (br m, 1H), 3.53 (d, 0.5H, J=8.2), 3.49 (d, 0.5H, J=8.2), 2.11 (s, 3H), 2.09 (d, 0.5H, J=8.2), 2.03 (d, 0.5H, J=8.2), 1.73 (s, 1.5H), 1.68 (s, 1.5H), 1.67 (s, 1.5H), 1.64 (s, 1.5H), 1.36 (s, 1.5H), 1.35 (s, 1.5H), 1.32 (s, 1.5H), 1.30 (s, 1.5H).

### Example 97

### 6-Bromo-5-chloro-1,2,3,4-tetrahydro-3-hydroxy-2,2,4,4,8-pentamethylquinoline (Structure 27A of Scheme V, where R¹=Me, R²=H, R⁴=Cl, R⁵=Me, R⁹=Me)

This compound was prepared using General Method 11 (EXAMPLE 91) from 6-bromo-5-chloro-1,4-dihydro-2,2,4,4,8-pentamethyl-2H-quinolin-3-one to afford (±)-6-bromo-5-chloro-1,2,3,4-tetrahydro-3-hydroxy-2,2,4,4,8-pentamethylquinoline.

### (±)-5-Chloro-6-(3,5-dimethylisoxazol-4-yl)-1,2,3,4-tetrahydro-3-hydroxy-2,2,4,4,8-pentamethylquinoline (Compound 197, Structure 29 of Scheme V, where R¹=Me, R²=H, R⁴=Cl, R⁵=Me, R⁹=Me, Ar=3,5-dimethylisoxazol-4-yl)

This compound was prepared using General Method 5 (EXAMPLE 1) from (±)-6-bromo-5-chloro-1,2,3,4-tetrahydro-3-hydroxy-2,2,4,4,8-pentamethylquinoline and 3,5-dimethyl-4-isoxazolylboronic acid to afford Compound 197. ¹H NMR (500 MHz, CD₃OD) δ 6.76 (s, 1H), 3.45 (s, 1H), 2.22 (s, 1.5H), 2.21 (s, 1.5H), 2.09 (s, 3H), 2.07 (s, 1.5H), 2.06 (s, 1.5H), 1.61 (s, 1.5H), 1.60 (s, 1.5H), 1.59 (s, 1.5H), 1.58 (s, 1.5H), 1.32 (s, 3H), 1.17 (s, 3H).

### Example 98

### (±)-6-(3-Amino-2-methoxyphenyl)-5-chloro-1,2,3,4-tetrahydro-2,2,4,8-tetramethylquinoline (Compound 198, Structure 44 of Scheme XI, where R¹=Me, R²=H, R⁴=Cl, R⁵=Me, R⁶=H, R⁹=H)

To prepare this compound, a mixture of Compound 156 (EXAMPLE 56), zinc dust (72 mg, 1.1 mmol) and calcium chloride dihydrate (79 mg, 0.54 mmol) in 3 ml 95% EtOH/water was heated at reflux overnight. The solution was filtered through celite and the solvent was removed under reduced pressure. The resulting oil was dissolved in EtOAc and saturated ammonium chloride. The organic layer was dried over magnesium sulfate, filtered, and concentrated. Flash chromatography (20% EtOAc/hexanes) afforded 75 mg (81%) of Compound 198. ¹H NMR (500 MHz, acetone-d₆) δ 6.80-6.83 (m, 3H), 6.73 (dd, J=1.5, 7.8 Hz, 1H), 6.42 (br s, 1H), 3.36 (s, 3H), 3.28-3.37 (m, 2H), 2.10 (s, 3H), 1.97 (dd, J=6.8, 13.2 Hz, 1H), 1.78-1.82 (m, 1H), 1.41 (d, J=6.8 Hz, 3H), 1.38 (s, 3H), 1.25 (s, 3H).

### Example 99

### (±)-5-Chloro-1,2,3,4-tetrahydro-6-[2-methoxy-3-(methoxycarbonylamino)phenyl]-2,2,4,8-tetramethylquinoline (Compound 199, Structure 45 of Scheme XI, where R¹=Me, R²=H, R⁴=Cl, R⁵=Me, R⁶=H, R⁹=H, R¹⁷=methoxycarbonylamino)

To prepare this compound, a solution of Compound 198 (EXAMPLE 98) (15 mg, 0.043 mmol), methyl chloroformate (5 μliter, 0.065 mmol), DMAP (1 mg) and pyridine (35 μliter, 0.43 mmol) in 1 mL dichloromethane was stirred overnight at room temperature. The mixture was quenched with water and extracted with EtOAc. The organic layer was dried over magnesium sulfate, filtered, and concentrated. Flash chromatography (20% EtOAc/hexanes) afforded 7 mg (40%) of Compound 199. ¹H NMR (500 MHz, acetone-d6) δ 8.05 (d, J=7.1 Hz, 1H), 7.82 (br s, 1H), 7.07 (t, J=6.8 Hz, 1H), 6.81-6.90 (m, 2H), 3.73 (s, 3H), 3.24-3.41 (m, 2H), 2.84 (s, 3H), 1.97 (s, 3H), 1.81-1.83 (m, 1H), 2.02-2.08 (m, 1H), 1.41 (d, J=6.8 Hz, 3H), 1.25 (s, 3H), 0.88 (s, 3H).

### Example 100

### (±)-5-Chloro-1,2,3,4-tetrahydro-6-[3-(tert-butoxycarbonylamino)-2-methoxyphenyl]-2,2,4,8-tetramethylquinoline (Compound 200, Structure 45 of Scheme XI, where R¹=Me, R²=H, R⁴=Cl, R⁵=Me, R⁶=H, R⁹=H, R¹⁷=tert-butoxycarbonylamino)

To prepare this compound, a solution of Compound 198 (EXAMPLE 198) (15 mg, 0.043 mmol), pivaloyl chloride (10 μliter, 0.086 mmol), DMAP (1 mg) and pyridine (35 μliter, 0.43 mmol) in 1 mL dichloromethane was stirred overnight at room temperature. The mixture was quenched with water and extracted with EtOAc. The organic layer was dried over magnesium sulfate, filtered, and concentrated. Flash chromatography (20% EtOAc/hexanes) afforded 15 mg (81%) of Compound 200. ¹H NMR (500 MHz, CDCl₃) δ 8.38 (dd, J=8.3, 1.5, 1H), 8.26 (br s, 1H), 7.10 (t, J=6.7 Hz, 1H), 6.92 (d, J=7.1 Hz, 1H), 6.82 (s, 1H), 3.45 (s, 3/2H), 3.42 (s, 3/2H), 3.35-3.38 (m, 1H), 2.09 (s, 3H), 1.97 (dd, J=6.3, 13.2 Hz, 1H), 1.81-1.83 (m, 1H), 1.41-1.44 (m, 3H), 1.38 (s, 3H), 1.33 (s, 9H), 1.24 (s, 3H).

### Example 101

### (±)-5-Chloro-1,2,3,4-tetrahydro-6-[2-methoxy-3-(methylsulfonamido)phenyl]-2,2,4,8-tetramethylquinoline (Compound 201, Structure 45 of Scheme XI, where R¹=Me, R²=H, R⁴=Cl, R⁵=Me, R⁶=H, R⁹=H)

To prepare this compound, a solution of Compound 198 (EXAMPLE 98) (15 mg, 0.043 mmol), methanesulfonyl chloride (7 μliter, 0.064 mmol), DMAP (1 mg) and pyridine (35 μliter, 0.43 mmol) in 1 mL dichloromethane was stirred overnight at room temperature. The mixture was quenched with water and extracted with EtOAc. The organic layer was dried over magnesium sulfate, filtered, and concentrated. Flash chromatography (30% EtOAc/hexanes) afforded 16 mg (84%) of Compound 201. ¹H NMR (500 MHz, CDCl₃) δ 7.51 (d, J=8.3 Hz, 1H), 7.09 (t, J=7.8 Hz, 1H), 6.92-7.08 (m, 2H), 6.86 (s, 1H), 3.58 (s, 1H), 3.42 (s, 3/2H), 3.38 (s, 3/2H), 3.30-3.38 (m, 1H), 2.09 (s, 3H), 1.92-2.01 (m, 1H), 1.78-1.82 (m, 1H), 1.39-1.43 (m, 3H), 1.38 (s, 3H), 1.24 (s, 3H).

### Example 102

### (±)-5-Chloro-1,2,3,4-tetrahydro-6-[(2-t-butyldimethylsilyl)oxy-3-nitrophenyl]-2,2,4,8-tetramethylquinoline (Structure 6 of Scheme I, where R¹=Me, R³=H, R⁴=Cl, R⁵=Me, R⁶ 6H, R⁹=H, Ar=(2-t-butyldimethylsilyl)oxy-3-nitrophenyl)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline (200 mg, 0.57 mmol) and 1-bromo-2-(t-butyldimethylsilyl)oxy-3-nitrobenzene (173 mg, 0.52 mmol) to afford 75 mg (28%) of (±)-5-chloro-1,2,3,4-tetrahydro-6-[(2-t-butyldimethylsilyl)oxy-3-nitrophenyl]-2,2,4,8-tetramethylquinoline after flash chromatography (10% EtOAc/hexanes).

### (±)-5-Chloro-1,2,3,4-tetrahydro-6-(2-hydroxy-3-nitrophenyl)-2,2,4,8-tetramethylquinoline (Compound 202, Structure 47 of Scheme XII, where R¹=Me, R²=H, R⁴=Cl, R⁵=Me, R⁶=H, R⁹=H)

To prepare this compound, a solution of (±)-5-chloro-1,2,3,4-tetrahydro-6-[(2-t-butyldimethylsilyl)oxy-3-nitrophenyl]-2,2,4,8-tetramethylquinoline (75 mg, 0.16 mmol) and TBAF (0.24 mL of a 1 M solution) in 2 mL THF was stirred at 0° C. then allowed to warm to room temperature. After 16 hours at room temperature, the mixture was quenched with water and extracted with EtOAc. The organic layer was dried over magnesium sulfate, filtered, and concentrated. Flash chromatography (10% EtOAc/hexanes) afforded 29 mg (50%) of Compound 202. ¹H NMR (500 MHz, CDCl₃) δ 6.88 (s, 1H), 6.75-6.77 (m, 1H), 6.73 (dd, J=1.5, 7.8 Hz, 1H), 6.58-6.62 (m, 1H), 3.62 (s, 1H), 3.36-3.40 (m, 1H), 2.09 (s, 3H), 1.96 (dd, J=7.3, 13.7 Hz, 1H), 1.78-2.05 (m, 1H), 1.42-1.45 (m, 3H), 1.39 (s, 3H), 1.27 (s, 3H).

### Example 103

### (±)-5-Chloro-1,2,3,4-tetrahydro-2,2,4,8-tetramethyl-6-[2-(methylbut-2-enyloxy)-3-nitrophenyl]quinoline (Compound 203, Structure 47B of Scheme XII, where R¹=Me, R²=H, R⁴=Cl, R⁵=Me, R⁶=H, R⁹=H, R¹⁶=3,3-dimethylallyl)

To prepare this compound, a mixture of Compound 202 (EXAMPLE 102) 10 mg (0.028 mmol), 4-bromo-2-methyl-2-butene (10 liter, 0.084 mmol), potassium carbonate (8 mg, 0.06 mmol) in 2 mL DMF was heated at 40° C. for 4 hour. The mixture was quenched with water and extracted with EtOAc. The organic layer was dried over magnesium sulfate, filtered, and concentrated. Flash chromatography (10% EtOAc/hexanes) afforded Compound 203. ¹H NMR (500 MHz, CDCl₃) δ 7.72 (d, J=8.2 Hz, 1H), 7.43-7.51 (m, 1H), 7.19 (t, J=7.3 Hz, 1H), 6.91 (s, ½H), 6.88 (s, ½H), 5.12-5.18 (m, 1H), 4.13-4.21 (m, 2H), 3.62 (s, 1H), 3.28-3.33 (m, 1H), 2.09 (s, 3H), 1.94-2.00 (m, 1H), 1.79-1.82 (m, 1H), 1.61 (s, 3H), 1.40-1.44 (m, 3H), 1.37 (s, 3H), 1.34-1.38 (m, 3H), 1.24 (s, 3H).

### Example 104

### (±)-6-(2H-1,4-Benzoxazin-3(4H-on-8-yl)-5chloro-1,2,3,4-tetrahydro-2,2,4,8-tetramethylquinoline (Compound 204, Structure 48 of Scheme XII, where R¹=Me, R²=H, R⁴=Cl, R⁵=Me, R⁶=H, R⁹=H, n=1)

To prepare this compound, a mixture of Compound 202 (50 mg, 0.14 mmol), ethyl bromoacetate (23 liter, 0.21 mmol) and potassium carbonate (48 mg, 0.35 mmol) in 1.5 mL DMF was heated to 80° C. for 2 hours. The mixture was quenched with water and extracted with EtOAc. The organic layer was dried over magnesium sulfate, filtered, and concentrated. Flash chromatography (10% EtOAc/hexanes) afforded 28 mg an oil. The oil was treated with zinc dust (16 mg, 0.25 mmol) and calcium chloride dehydrate (18 mg, 0.13 mmol) in 1.5 mL EtOH and heated at reflux for 2 hour. The mixture was filtered through celite and the solvent evaporated. Flash chromatography (30% EtOAc/hexanes) afforded 8 mg (35%) of Compound 204. ¹H NMR (500 MHz, CDCl₃) δ 7.38 (s, ½H), 7.37 (s, ½H), 7.06 (t, J=7.8 Hz, 1H), 6.78-6.84 (m, 2H), 6.61 (dd, J=1.5, 7.8 Hz, 1H), 4.10-4.15 (m, 2H), 4.08 (s, 1H), 3.28-3.39 (m, 1H), 2.08 (s, 3H), 1.92-2.01 (m, 1H), 1.78 (dd, J=4.4, 13.6 Hz, 1H), 1.32-1.41 (m, 3H), 1.38 (s, 3H), 1.25 (s, 3H).

### Example 105

### (±)-5-Chloro-1,2,3,4-tetrahydro-2,2,4,8-tetramethyl-6-(4-methyl-2H-1,4-benzoxazin-3(4H)-on-8-yl)quinoline (Compound 205, Structure 49 of Scheme XII, where R¹=Me, R²=H, R⁴=Cl, R⁵=Me, R⁶=H, R⁹=H, n=1, R³⁵=Me)

To prepare this compound, a mixture of Compound 204 (EXAMPLE 890721) (10 mg, 0.027 mol), sodium hydride (60% in oil, 3 mg, 0.07 mmol) and methyl iodide

(50 μliter) in 1 mL THF was stirred at 0° C., then allowed to warm to room temperature. The mixture was quenched with water and extracted with EtOAc. The organic layer was dried over magnesium sulfate, filtered, and concentrated. Flash chromatography (33% EtOAc/hexanes) afforded Compound 205. ¹H NMR (500 MHz, CDCl₃) δ 7.12 (t, J=7.8 Hz, 1H), 7.02 (dd, J=1.5, 7.8 Hz, 1H), 6.91-7.01 (m, 1H), 6.86 (s, 1H), 4.61-4.72 (m, 2H), 3.44 (s, 3H), 2.12 (s, 3H), 1.93-2.01 (m, 1H), 1.84 (dd, J=3.9, 13.7 Hz, 1H), 1.41-1.49 (m, 3H), 1.42 (s, 3H), 1.29 (s, 3H).

### Example 106

### (±)-6-(2-Benzoxazolinon-7-yl)-5-chloro-1,2,3,4-tetrahydro-2,2,4,8-tetramethylquinoline (Compound 206, Structure 48 of Scheme XII, where R¹=Me, R²=H, R⁴=Cl, R⁵=Me, R⁶=H, R⁹=H, n=0)

To prepare this compound, a mixture of Compound 202 (92 mg, 0.25 mmol), methyl chloroformate (68 μliter, 0.88 mmol), DMAP (1 mg) and pyridine (0.3 mL) in 3 mL dichloromethane was stirred at room temperature for 2 hours. The mixture was quenched with water and extracted with EtOAc. The organic layer was dried over magnesium sulfate, filtered, and concentrated. Flash chromatography (10% EtOAc/hexanes) affords 100 mg an oil. The oil was treated with tin(II) chloride dihydrate (63 mg, 0.25 mmol) in 1.5 mL EtOH and heated at reflux for 3 hours. The mixture was partitioned between EtOAc and water, and the organic layer was dried over magnesium sulfate, filtered, and the solvent evaporated to 15 mg of an oil. This material was treated with potassium carbonate (6 mg, 0.044 mmol) in 1.5 mL DMF and heated to 110° C. for 1 hour. The mixture was quenched with water and extracted with EtOAc. The organic layer was dried over magnesium sulfate, filtered, and concentrated. Flash chromatography (30% EtOAc/hexanes) afforded 10 mg of Compound 206. ¹H NMR (500 MHz, CDCl₃) δ 8.80 (br s, 1H), 7.19 (t, J=7.3 Hz, 1H), 7.16 (d, J=6.8 Hz, 1H), 7.05 (d, J=6.3 Hz, 1H), 6.97 (s, 1H), 3.63 (broad s, 1H), 3.38-3.44 (m, 1H), 2.09 (s, 3H), 2.00 (dd, J=6.8, 13.7 Hz, 1H), 1.86 (dd, J=4.3, 8.7 Hz, 1H), 1.48 (d, J=7.3, 3H), 1.43 (s, 3H), 1.30 (s, 3H).

### Example 107

### (±)-6-(3-Amino-2-hydroxyphenyl)-5-chloro-1,2,3,4-tetrahydro-2,2,4,8-tetramethylquinoline (Compound 207, Structure 47A of Scheme XII, where R¹=Me, R³=H, R⁴=Cl, R⁶=Me, R⁶H, R⁹=H)

To prepare this compound, a mixture of Compound 202 (EXAMPLE 102) (178 mg, 0.49 mmol), zinc dust (128 mg, 1.97 mmol), calcium chloride dehydrate (144 mg, 0.98 mmol) in 15 mL EtOH was heated at reflux for 4 hours. The mixture was filtered through celite and the solvent removed under reduced pressure. Flash chromatography (30% EtOAc/hexanes) afforded 99 mg (61%) of Compound 207. ¹H NMR (500 MHz, CDCl₃) δ 10.92 (br s, 1H), 8.12 (dd, J=1.5, 8.8 Hz, 1H), 7.49-7.58 (m, 1H), 7.01 (t, J=8.8 Hz, 1H), 6.85 (s, 1H), 3.62 (s, 1H), 3.22-3.31 (m, 1H), 2.10 (s, 3H), 1.85-2.01 (m, 1H), 1.78-1.82 (m, 1H), 1.38 (s, 3H), 1.39-1.45 (m, 3H), 1.26 (s, 3H).

### Example 108

### (±)-6-(2-Amino-6-methoxyphenyl)-5-chloro-1,2,3,4-tetrahydro-2,2,4,8-tetramethylquinoline (Compound 208, Structure 51B of Scheme XIII, where R¹=Me, R²=H, R⁴=Cl, R⁵=Me, R⁶=H, R⁹=H, R²⁴=H, R²⁵=OMe)

To prepare this compound, a mixture of Compound 157 (EXAMPLE 57) (21 mg, 0.056 mmol), zinc dust (22 mg. 0.34 mmol) and calcium chloride dehydrate (25 mg, 0.17 mmol) in 2 mL 95% EtOH/water was heated at reflux for 18 hours. The mixture was filtered through celite while hot, and the solvent was evaporated under reduced pressure. The residue was partitioned between EtOAc and water, and HCl was added until the pH was between 3-4. The organic layer washed with brine, dried over magnesium sulfate, filtered, and concentrated. Flash chromatography (25% EtOAc/hexanes) afforded 17 mg (89%) of Compound 208. ¹H NMR (500 MHz, CDCl₃) δ 7.12 (d, J=7.8, 1H), 6.81 (s, ½H), 6.80 (s, ½H), 6.38-6.45 (m, 2H), 3.72 (s, 3/2H), 3.71 (s, 3/2H), 3.52 (broad s, 3H), 3.30-3.40 (m, 1H), 2.07 (s, 3H), 1.92-1.99 (m, 1H), 1.75-1.81 (m, 1H), 1.44 (d, J=6.9, 3/2H), 1.42 (d, J=7.3, 3/2H), 1.37 (s, 3H), 1.25 (s, 3/2H), 1.24 (s, 3/2H).

### Example 109

### (±)-5-Chloro-1,2,3,4-tetrahydro-6-(2-methoxyindol-7-yl)-2,2,4,8-tetramethylquinoline (Compound 209, Structure 51 of Scheme XIII, where R¹=Me, R²=H, R⁴=Cl, R⁵=Me, R⁶=H, R⁹=H, R²⁴H, R²⁵=OMe, R^{A}=H, R^{B}=H)

This compound was prepared by the conversion of an ortho-substituted nitrobenzene to a 7-substituted indole, as follows. Vinyl magnesium bromide (0.45 mL of a 1 M solution in THF) was added to a solution of Compound 157 (EXAMPLE 57) (48 mg, 0.13 mmol; 1 equiv) in THF (0.15 M), at −40° C. That mixture was stirred at −40 for 0.5-2 hours and then poured into saturated ammonium chloride. The resulting mixture was extracted with EtOAc. The organic layer washed with brine, dried over magnesium sulfate, filtered, and concentrated to afford 21 mg (45%) of Compound 209 after flash chromatography (33% EtOAc/hexanes). ¹H NMR (500 MHz, CDCl₃) δ 7.76 (broad s, ½H), 7.75 (broad s, ½H), 7.56 (d, J=8.3, 1H), 7.02-7.08 (m, 1H), 6.90-6.95 (m, 2H), 6.47-6.52 (m, 1H), 3.83 (s, 3/2H), 3.81 (s, 3/2H), 3.58 (broad s, 1H), 3.30-3.40 (m, 1H), 2.09 (s, 3H), 1.98 (dd, J=13.5, 7.1H), 1.78-1.84 (m, 1H), 1.47 (d, J=6.9, 3/2H), 1.44 (d, J=7.3, 3/2H), 1.40 (s, 3/2H), 1.39 (s, 3/2H), 1.28 (s, 3/2H), 1.27 (s, 3/2H).

### Example 110

### (±)-5-Chloro-1,2,3,4-tetrahydro-6-(indolin-7-yl)-2,2,4,8-tetramethylquinoline (Compound 210, Structure 58 of Scheme XV, where R¹=Me, R²=H, R⁴=Cl, R⁵=Me, R⁶=H, R⁹=H)

To prepare this compound, a mixture of Compound 149 (EXAMPLE 49) (15 mg, 0.044 mmol), sodium cyanoborohydride (19 mg, 0.30 mmol) in 1 mL acetic acid was stirred at room temperature for 2 hours. The mixture was partitioned between EtOAc and saturated sodium bicarbonate. The organic layer washed with brine, dried over magnesium sulfate, filtered, and concentrated under reduced pressure. Flash chromatography (70% dichloromethane/hexanes to 100% dichloromethane) afforded 6 mg (40%) of Compound 210. ¹H NMR (400 MHz, CDCl₃) δ 7.11 (d, J=7.2, 1H), 6.90-7.00 (m, 1H), 6.88 (s, 1H), 6.75 (dd, J=7.6, 7.3, 1H), 3.60-3.90 (broad s, 1H), 3.45-3.65 (m, 3H), 3.28-3.40 (m, 1H), 3.05-3.15 (m, 2H), 2.07 (s, 3H), 1.95 (dd, J=13.5, 7.1, 1H), 1.75-1.82 (m, 1H), 1.40-1.45 (m, 3H), 1.37 (s, 3H), 1.23 (s, 3H).

### Examples 111 and 112

### (±)-5-Chloro-1,2,3,4-tetrahydro-2,2,4,8-tetramethyl-6-(2-oxindol-7-yl)quinoline (Compound 211, Structure 55 of Scheme XV, where R¹=Me, R²=H, R⁴=Cl, R⁵=Me, R⁶=H, R⁹=H) and 6-(3-Bromoindol-7-yl)-5-chloro-1,2,3,4-tetrahydro-2,2,4,8-tetramethylquinoline (Compound 212, Structure 56 of Scheme XV, where R¹=Me, R²=H, R⁴=Cl, R⁵=Me, R⁶=H, R⁹=H).

To prepare these compounds, a solution of Compound 149 (EXAMPLE 49) (20 mg, 0.060 mmol) and N-bromosuccinimide (14 mg, 0.078 mmol) in 1.5 mL 90% t-butanol/water was stirred at room temperature for 4 hours. The mixture was partitioned between EtOAc and water, and the organic layer was dried over magnesium sulfate, filtered, and concentrated under reduced pressure. Flash chromatography (33% EtOAc/hexanes) afforded 3.5 mg (17%) of Compound 211 and 4.5 mg (18%) of Compound 212.

Data for Compound 211: ¹H NMR (500 MHz, CDCl₃) δ 8.08 (broad s, ½H), 8.07 (broad s, ½H), 7.57 (d, J=7.8, 1H), 7.14-7.28 (m, 3H), 6.95 (s, 1H), 3.62 (broad s, 1H), 3.32-3.42 (m, 1H), 2.11 (s, 3H), 1.98 (dd, J=13.7, 6.8, 1H), 1.80-1.88 (m, 1H), 1.47 (d, J=6.8, 3/2H), 1.44 (d, J=6.8, 3/2H), 1.41 (s, 3H), 1.28 (s, 3/2H), 1.27 (s, 3/2H).

Data for Compound 212: ¹H NMR (500 MHz, CDCl₃) δ 7.24 (broad s, 1H), 7.17-7.20 (m, 3/2H), 7.12 (d, J=7.8, ½H), 7.03-7.08 (m, 1H), 6.83 (s, ½H), 6.82 (s, ½H), 3.53-3.67 (m, 3H), 3.30-3.38 (m, 1H), 2.09 (s, 3H), 1.93-2.00 (m, 1H), 1.79-1.85 (m, 1H), 1.44 (d, J=7.3, 3/2H), 1.42 (d, J=7.3, 3/2H), 1.39 (s, 3H), 1.26 (s, 3/2H), 1.25 (s, 3/2H).

### Example 113

### (±)-5-Chloro-1,2,3,4-tetrahydro-4-hydroxy-6-(indol-2-yl)-2,2,4,8-tetramethylquinoline (Compound 213, Structure 6 of Scheme I, where R¹=Me, R²=H, R⁴=Cl, R⁵=Me, R⁶=H, R⁹=OH, Ar=indol-7-yl)

This compound was prepared using General Method 5 (EXAMPLE 1) from (±)-6-bromo-5-chloro-1,2,3,4-tetrahydro-4-hydroxy-2,2,4,8-tetramethylquinoline and 7-(4,4,5,5-tetramethyl-1,3,2-dioxaborolan-2-yl)indole to afford Compound 213. ¹H NMR (500 MHz, CD₃OD) δ 7.51-7.49 (m, 1H), 7.15 (d, 1H, J=3.4), 7.04-6.89 (m, 3H), 6.45-6.43 (m, 1H), 2.15-2.12 (m, 4H), 2.04-1.98 (m, 1H), 1.87 (br s, 3H), 1.33 br s, 6H).

### Example 114

### 6-Bromo-5-chloro-1,2-dihydro-2,2,4,8-tetramethylquinoline (Structure 62 of Scheme XVII where R¹=Me, R²=H, R⁴=Cl, R⁵=Me, R⁶=H)

To prepare this compound, (±)-6-Bromo-5-chloro-1,2,3,4-tetrahydro-4-hydroxy-2,2,4,8-tetramethylquinoline was stirred in 30% trifluoroacetic acid in dichloromethane at room temperature for 1 hour. The mixture was neutralized with sodium bicarbonate and extracting with EtOAc. The organic layer was dried over magnesium sulfate, filtered, and concentrated to afford 6-bromo-5-chloro-1,2-dihydro-2,2,4,8-tetramethylquinoline.

### 5-Chloro-1,2-dihydro-6-(indol-2-yl)-2,2,4,8-tetramethylquinoline (Compound 214, Structure 63 of Scheme XVII, where R¹=Me, R²=H, R⁴=Cl, R⁵=Me, R⁶=H, Ar=indol-7-yl)

This compound was prepared using General Method 5 (EXAMPLE 1) from 6-bromo-5-chloro-1,2-dihydro-2,2,4,8-tetramethylquinoline and 7-(4,4,5,5-tetramethyl-1,3,2-dioxaborolan-2-yl)indole to afford Compound 214. ¹H NMR (500 MHz, CDCl₃) δ 8.06 (br s, 1H), 7.67 (d, 1H, J=7.8), 7.23-7.19 (m, 2H), 7.13 (dd, 1H, J=1.0, J=7.3), 5.55 (br s, 1H), 3.88 (br s, 1H), 2.39 (s, 3H), 2.16 (s, 3H), 1.37 (s, 3H), 1.35 (s, 3H).

### Example 115

### 5-Chloro-1,2,3,4-tetrahydro-4-hydroxy-2,2,4,8-tetramethyl-6-(naphthal-1-yl)quinoline (Compound 215, Structure 6 of Scheme I, where R¹=Me, R²=H, R⁴=Cl, R⁵=Me, R⁶=H, R⁹=OH, Ar=naphthal-1-yl)

This compound was prepared using General Method 5 (EXAMPLE 1) from (±)-6-bromo-5-chloro-1,2,3,4-tetrahydro-4-hydroxy-2,2,4,8-tetramethylquinoline and 1-naphthaleneboronic acid to afford Compound 215. ¹H NMR (500 MHz, CD₃OD) δ 7.81-7.75 (m, 2H), 7.42-7.19 (m, 5H), 6.81 (br s, 1H), 2.10-2.06 (m, 4H), 1.94 (d, 0.5H, J=13.6), 1.92 (d, 0.5H, J=13.6), 1.77 (s, 1.5H), 1.75 (s, 1.5H), 1.28 (s, 1.5H), 1.27 (s, 1.5H), 1.26 (s, 3H).

### Example 116

### (±)-1,2,3,4-Tetrahydro-3β-hydroxy-6-(indol-7-yl)-2,2,4α,5,8-pentamethylquinoline (Compound 216, Structure 6 of Scheme I, where R¹=Me, R²=H, R⁴=Me, R⁵=α-Me, R⁶=β-OH, R⁹=H, Ar=indol-7-yl)

To prepare this compound, first 1,2-dihydro-2,2,4,5,8-pentamethylquinoline was prepared using General Method 1 (EXAMPLE 1) from 2,5-Dimethylaniline. That 1,2-Dihydro-2,2,4,5,8-pentamethylquinoline was treated according to General Method 7 (EXAMPLE 59) to afford 1,2,3,4-tetrahydro-3β-hydroxy-2,2,4α,5,8-pentamethylquinoline. That 1,2,3,4-tetrahydro-3β-hydroxy-2,2,4α,5,8-pentamethylquinoline was treated according to General Method 3 (EXAMPLE 1) to afford 6-bromo-1,2,3,4-tetrahydro-3β-hydroxy-2,2,4α,5,8-pentamethylquinoline. Finally, Compound 216 was prepared using General Method 5 (EXAMPLE 1) from the 6-Bromo-1,2,3,4-tetrahydro-3β-hydroxy-2,2,4α, 5,8-pentamethylquinoline and 7-(4,4,5,5-tetramethyl-1,3,2-dioxaborolan-2-yl)indole (EXAMPLE 71) to afford Compound 216. ¹H NMR (500 MHz, CD₃OD) δ 7.52 (d, 0.5H, J=7.8) 7.51 (d, 0.5H, J=7.8), 7.19 (d, 0.5H, J=2.9), 7.16, (d, 0.5H, J=3.1), 7.06 (t, 1H, J=7.3), 6.91 (d, 0.5H, J=6.8), 6.86-6.85 (m, 1.5H), 6.49 (d, 0.5H, J=2.9), 6.48 (d, 0.5H, J=2.9), 3.51 (d, 0.5H, J=7.3), 3.49 (d, 0.5H, J=7.3), 2.89 (dq, 0.5H, J=6.8, 6.3), 2.84 (dq, 0.5H, J=6.8, 6.3), 2.18 (s, 3H), 2.07 s, 1.5H), 2.02 (s, 1.5H), 1.46 (d, 1.5H, J=6.8), 1.41 (d, 1.5H, J=6.8), 1.39 (s, 3H), 1.09 (s, 1.5H), 1.06 (s, 1.5H).

### Example 117

### (±)-6-(3,5-Dimethylisoxazol-4-yl)-1,2,3,4-tetrahydro-3β-hydroxy-2,2,4α,5,8-pentamethylquinoline (Compound 217, Structure 6 of Scheme I, where R¹=Me, R²=H, R⁴=Me, R⁵=α-Me, R⁶=β-OH, R⁹=H, Ar=3,5-dimethylisoxazol-4-yl)

This compound was prepared using General Method 5 (EXAMPLE 1) from 6-bromo-1,2,3,4-tetrahydro-3β-hydroxy-2,2,4α,5,8-pentamethylquinoline (EXAMPLE 116) and 3,5-dimethyl-4-isoxazolylboronic acid to afford Compound 217. ¹H NMR (500 MHz, CDCl₃) δ 6.68 (s, 0.5H), 6.67 (s, 0.5H), 3.60 (dd, 0.5H, J=5.0, 8.1 Hz), 3.59 (dd, 0.5H J=5.0, 8.1 Hz), 3.52 (s, 1H), 2.99 (dq, 0.5H J=4.7, 7.0 Hz), 2.98 (dq, 0.5H, J=4.7, 7.0 Hz), 2.25 (s, 1.5H), 2.25 (s, 1.5H), 2.14 (s, 1.5H), 2.14 (s, 1.5H), 2.12 (s, 1.5H), 2.11 (s, 1.5H), 2.05 (s, 1.5H), 2.04 (s, 1.5H), 1.95 (d, 1H J=8.1 Hz), 1.43 (d, 1.5H, J=7.1 Hz), 1.42 (d, 1.5H, J=7.0 Hz), 1.37 (s, 1.5H), 1.37 (s, 1.5H), 1.22 (s, 3H).

### Example 118

### (±)-5-Fluoro-1,2,3,4-tetrahydro-3β-hydroxy-2,2,4α,8-tetramethyl-6-(naphthal-1-yl)quinoline (Compound 218, Structure 6 of Scheme I, where R¹=Me, R²=H, R⁴=F, R⁵=α-Me, R⁶=β-OH, R⁹=H, Ar=naphthal-1-yl)

To prepare this compound, first 5-fluoro-1,2-dihydro-2,2,4,8-tetramethylquinoline was prepared using General Method 1 (EXAMPLE 1) from 5-Fluoro-2-methylaniline. That 5-fluoro-1,2-dihydro-2,2,4,8-tetramethylquinoline was treated according to General Method 7 (EXAMPLE 59) to afford 5-fluoro-1,2,3,4-tetrahydro-3β-hydroxy-2,2,4α,8-tetramethylquinoline. That 5-fluoro-1,2,3,4-tetrahydro-3β-hydroxy-2,2,4α,8-tetramethylquinoline was treated according to General Method 3 (EXAMPLE 1) to afford 6-bromo-5-fluoro-1,2,3,4-tetrahydro-3β-hydroxy-2,2,4α,8-tetramethylquinoline. Finally, Compound 218 was prepared using General Method 5 (EXAMPLE 1) from the 6-bromo-5-fluoro-1,2,3,4-tetrahydro-3β-hydroxy-2,2,4α,8-tetramethylquinoline and 1-naphthaleneboronic acid to afford Compound 218. ¹H NMR (500 MHz, CDCl₃) δ 7.88 (d, 1H, J=8.1), 7.84 (d, 1H, J=8.3), 7.78 (d, 0.5H, J=8.1), 7.72 (d, 0.5H, J=8.2), 7.53-7.39 (m, 4H), 6.95 (s, 0.5H), 6.93 (s, 0.5H), 3.62 (s, 1H), 3.49 (dd, 0.5H, J=6.8, 7.2), 3.47 (dd, 0.5H, J=6.8, 7.3), 2.98 (qn, 0.5H, J=6.8), 2.91 (qn, 0.5H, J=6.8), 2.13 (s, 3H), 1.87 (d, 0.5H, J=7.2, 1.84 (d, 0.5H, J=7.2), 1.53 (dd, 1.5H, J=6.7, 1.3), 1.48 (dd, 1.5H, J=6.8, 1.3), 1.38 (s, 1.5H), 1.37 (s, 1.5H), 1.22 (s, 1.5H), 1.18 (s, 1.5H).

### Example 119

### (±)-6-(3,5-Dimethylisoxazol-4-yl)-5-fluoro-1,2,3,4-tetrahydro-3β-hydroxy-2,2,4α,8-tetramethylquinoline (Compound 219, Structure 6 of Scheme I, where R¹=Me, R²=H, R⁴=F, R⁵=α-Me, R⁶=β-OH, R⁹=H, Ar=3,5-dimethylisoxazol-4-yl)

This compound was prepared using General Method 5 (EXAMPLE 1) from 6-bromo-5-fluoro-1,2,3,4-tetrahydro-3β-hydroxy-2,2,4α,8-tetramethylquinoline (EXAMPLE 118) and 3,5-dimethyl-4-isoxazolylboronic acid to afford Compound 219. ¹H NMR (500 MHz, CD₃OD) δ 6.79 (m, 0.5H), 6.78 (m, 0.5H), 3.32 (d, 1H, J=8.8), 2.81 (dq, 1H, J=8.5, 6.6), 2.33 (s, 1.5H), 2.33 (s, 1.5H), 2.18 (s, 1.5H), 2.18 (s, 1.5H), 2.14 (s, 3H), 1.47 (d, 1.5H, J=6.6), 1.46 (d, 1.5H, J=6.6), 1.37 (s, 3H), 1.05 (s, 3H).

### Example 120

### (±)-5-Fluoro-1,2,3,4-tetrahydro-3β-hydroxy-6-(indol-7-yl)-2,2,4α,8-tetramethylquinoline (Compound 220, Structure 6 of Scheme I, where R¹=Me, R²=H, R⁴=F, R⁵=α-Me, R⁶=β-OH, R⁹=H, Ar=indol-7-yl)

This compound was prepared using General Method 5 (EXAMPLE 1) from from 6-bromo-5-fluoro-1,2,3,4-tetrahydro-3β-hydroxy-2,2,4α,8-tetramethylquinoline (EXAMPLE 118) and 7-(4,4,5,5-tetramethyl-1,3,2-dioxaborolan-2-yl)indole (EXAMPLE 71) to afford Compound 220. ¹H NMR (500 MHz, CD₃OD) δ 9.95 (s, 1H), 7.48 (dd, 1H, J=7.6, 1.3), 7.18 (d, 0.5H, J=2.9), 7.17 (d, 0.5H, J=2.9), 7.02 (t, 1H, J=7.4), 7.00-6.97 (m, 2H), 6.45 (d, 1H, J=3.1), 3.34 (d, 1H, J=6.7), 2.82 (dq, 1H, J=8.5, 6.7), 2.15 (s, 3H), 1.48 (d, 1.5H, J=6.6), 1.46 (d, 1.5H, J=6.6), 1.34 (s, 3H), 1.06 (s, 3H).

### Example 121

### (±)-5-Chloro-1,2,3,4-tetrahydro-3β-hydroxy-6-(indolin-7-yl)-2,2,4α,8-tetramethylquinoline (Compound 221, Structure 58 of Scheme XV, where R¹=Me, R²=H, R⁴=Cl, R⁵=α-Me, R⁶=β-OH, R⁹=H)

This compound was prepared using the procedure described in Example 110 to prepare Compound 210, except that (±)-5-chloro-1,2,3,4-tetrahydro-3β-hydroxy-6-(indol-7-yl)-2,2,4α,8-tetramethylquinoline (Compound 161, EXAMPLE 61) was used as the starting material in place of Compound 149 described in Example 110, to afford Compound 221. ¹H NMR (500 MHz, CDCl₃) δ 7.11 (dd, 1H, J=7.2, 1.1 Hz), 6.971-6.890 (m, 2H), 6.75 (t, 1H, J=7.4 Hz), 3.59-3.50 (m, 4H), 3.12-3.05 (m, 3H), 2.10 (s, 1.5H), 2.10 (s, 1.5H), 1.88 (s, 1.5H), 1.52 (d, 1.5H, J=7.2), 1.51 (d, 1.5H, J=7.1), 1.33 (s, 3H), 1.21 (s, 3H).

### Example 122

### (±)-5-Fluoro-1,2,3,4-tetrahydro-3β-hydroxy-6-(indolin-7-yl)-2,2,4α,8-tetramethylquinoline (Compound 222, Structure 58 of Scheme XV, where R¹=Me, R²=H, R⁴=F, R⁵=α-Me, R⁶=β-OH, R⁹=H)

This compound was prepared using the procedure described in Example 110 to prepare Compound 210, except that (±)-5-fluoro-1,2,3,4-tetrahydro-3β-hydroxy-6-(indol-7-yl)-2,2,4α,8-tetramethylquinoline (Compound 220, EXAMPLE 120) was used as the starting material, to afford Compound 222. ¹H NMR (500 MHz, CDCl₃) δ 7.16 (m, 0.5H), 7.14 (m, 0.5H), 7.07 (d, 1H, J=7.7 Hz), 6.98 (d, 1H J=8.1 Hz), 6.87 (t, 1H J=7.4 Hz), 3.61 (t, 1H J=8.4 Hz), 3.60 (t, 1H J=8.2 Hz), 3.47 (d, 1H J=6.8 Hz), 3.15 (t, 2H, J=8.2 Hz), 2.95 (qn, 1H J=6.8 Hz), 2.13 (s, 3H), 1.52 (d, 1.5H J=6.8 Hz), 1.52 (d, 1.5H J=6.8 Hz), 1.37 (s, 3H), 1.18 (s, 3H).

### Example 123

### (±)-5-Chloro-1,2,3,4-tetrahydro-3β-hydroxy-6-[3-(butan-3-on-1-yl)indol-7-yl]-2,2,4α,8-tetramethylquinoline (Compound 223, Structure 53 of Scheme XIV, where R¹=Me, R²=H, R⁴=Cl, R⁵=α-Me, R⁶=β-OH, R⁹=H)

To prepare this compound, freshly distilled methyl vinyl ketone (30 μL, 0.372 mmol) was added to a solution of Compound 161 (EXAMPLE 61) (22 mg, 0.062 mmol) in 2 mL of dichloromethane at room temperature. Then, indium trichloride (7 mg, 0.031 mmol) was added. After stirring for 1 hour, that mixture was poured into saturated aqueous sodium bicarbonate (15 mL) and the aqueous phase was extracted twice with ethyl acetate (2×10 mL). The combined organic phases from those two extractions were washed with brine, dried over sodium sulfate, filtered, and concentrated in vacuo. The crude residue was chromatographed over silica gel, eluting with hexanes-ethyl acetate (3:1) to afford 8 mg (31%) of Compound 123 as a white solid. ¹H NMR (500 MHz, CDCl₃) δ 7.82 (s, 1H), 7.57 (d, 1H, J=7.8), 7.17 (t, 1H, J=7.6), 7.13 (d, 0.5H, J=7.3), 7.08 (d, 0.5H, J=7.3), 6.99 (s, 1H), 6.97 (s, 0.5H), 6.95 (s, 0.5H), 3.64-3.50 (m, 1H), 3.20-3.09 (m, 1H), 3.07 (t, 2H, J=7.5), 2.87 (t, 2H, J=7.5), 2.16 (s, 3H), 2.13 (s, 3H), 1.91 (d, 0.5H, j=7.6), 1.85 (d, 0.5H, J=7.6), 1.56 (d, 1.5H, J=6.8), 1.52 (d, 1.5H, J=6.8), 1.36 (s, 3H), 1.28 (s, 1.5H), 1.23 (s, 1.5H).

### Example 124

### 5-Chloro-6-(3-cyanophenyl)-1,2-dihydro-2,2,4-trimethylquinoline (Compound 224, Structure 11 of Scheme II)

To prepare this compound, first General Method 5 was performed using 4-bromo-3-chloroaniline and 3-Cyanophenylboronic acid. The product of that process was then used as the starting material for General Method 1 (EXAMPLE 1) to afford Compound 224. ¹H NMR (400 MHz, CDCl₃) δ 7.58-7.63 (m, 3H), 7.47 (t, J=7.6, 1H), 6.87 (d, J=8.0, 1H), 6.49 (d, J=8.0, 1H), 5.51 (broad s, 1H), 3.96 (broad s, 1H), 2.32 (d, J=1.6, 3H), 1.28 (s, 6H).

### Example 125

### (±)-5-Chloro-6-(3-cyanophenyl)-1,2,3,4-tetrahydro-2,2,4-trimethylquinoline (Compound 225, Structure 6 of Scheme II)

This compound was prepared using General Method 2 from Compound 224 to afford Compound 225. ¹H NMR (400 MHz, CDCl₃) δ 7.69-7.70 (m, 1H), 7.61-7.63 (m, 1H), 7.55-7.56 (m, 1H), 7.46 (t, J=7.7, 1H), 6.89 (d, J=8.1, 1H), 6.43 (d, J=8.2, 1H), 3.81 (broad s, 1H), 3.31-3.35 (m, 1H), 1.78-1.94 (m, 2H), 1.43 (d, J=7.2), 1.35 (s, 3H), 1.23 (s, 3H).

### Example 125A

### (+)-5-Chloro-6-(3-cyanophenyl)-1,2,3,4-tetrahydro-2,2,4-trimethylquinoline (Compound 225A, Structure (+)-6 of Scheme II) and (−)-5-Chloro-6-(3-cyanophenyl)-1,2,3,4-tetrahydro-2,2,4-trimethylquinoline (Compound 225B, Structure (−)-6 of Scheme II)

These compounds were isolated from the racemic compound of Example 125 using General Method 6 (EXAMPLE 1) on a Chiracel AS column (20×250 mm, 3% isopropanol/hexanes, 6 ml/min, to afford Compounds 225A and 225B. Data for Compound 225A: HPLC (Chiralcel AS, 3% isopropanol/hexanes, 6 ml/min) t_{R} 23.0 min; [α]_{D}=+36. Data for Compound 225B: HPLC (Chiralcel AS, 3% isopropanol/hexanes, 6 ml/min) t_{R} 27.2 min; [α]_{D}=−28.

### Example 126

### 5-Chloro-6-(3-cyanophenyl)-1,2-dihydro-1,2,2,4-tetramethylquinoline (Compound 226)

To prepare this compound, Compound 225 was treatment with NaH (1.5 equiv) and MeI (1.5 equiv) in THF/DMF and heated to 80-90° C. Preparative TLC (9:1 hexanes:EtOAc) afforded Compound 226. ¹H NMR (400 MHz, CDCl₃) δ 7.58-7.70 (m, 3H), 7.49 (t, J=7.6, 1H), 7.02 (d, J=8.2, 1H), 6.62 (d, J=8.2, 1H), 5.55 (d, J=1.6, 1H), 2.85 (s, 3H), 2.31 (d, J=1.6, 3H), 1.28 (s, 6H).

### Example 127

### 5-Chloro-8-fluoro-1,2-dihydro-2,2,4-trimethyl-6-(3-nitrophenyl)quinoline (Compound 227, Structure 11 of Scheme II)

This compound was prepared using the method described in Example 124 except that 4-bromo-2-fluoro-5-chloroaniline and 3-nitrophenylboronic acid were used as starting materials to afford Compound 227. ¹H NMR (400 MHz, CDCl₃) δ 8.24 (t, J=1.9, 1H), 8.20 (dd, J=1.2, J=7.5, 1H), 7.69 (dd, J=1.3, J=7.7, 1H), 7.55 (t, J=7.9, 1H), 6.85 (d, J=10.7, 1H), 5.55 (s, 1H), 4.24 (broad s, 1H), 2.33 (d, J=1.5, 3H), 1.32 (s, 6H).

### Example 128

### 5-Chloro-1,2-dihydro-2,2,4,8-tetramethyl-6-(3-nitrophenyl)quinoline (Compound 228, Structure 11 of Scheme II)

This compound was prepared using the method described in Example 124 except that 4-bromo-5-chloro-2-methylaniline and 3-nitrophenylboronic acid were used as starting materials to afford Compound 228. ¹H NMR (400 MHz, CDCl₃) δ 8.25 (t, J=1.8, 1H), 8.16 (dd, J=1.2, J=7.6, 1H), 7.72 (dd, J=1.3, J=7.7, 1H), 7.52 (t, J=8.0, 1H), 6.87 (s, 1H), 5.53 (d, J=1.5, 1H), 3.87 (s, 1H), 2.33 (d, J=1.3, 3H), 2.13 (s, 3H), 1.30 (s, 6H).

### Example 129

### 6-[3,5-Bis(trifluoromethyl)phenyl]-5-chloro-1,2-dihydro-2,2,4-trimethylquinoline (Compound 229, Structure 11 of Scheme II)

This compound was prepared using the method described in Example 124 except that 4-bromo-3-chloroaniline and 3,5-bis(trifluoromethyl)phenylboronic acid were used as starting materials to afford Compound 229. ¹H NMR (400 MHz, CDCl₃) δ 7.80-7.83 (m, 3H), 6.91 (d, J=8.2, 1H), 6.51 (d, J=8.2, 1H), 5.52 (broad s, 1H), 4.00 (broad s, 1H), 2.32 (s, 3H), 1.28 (s, 6H).

### Example 130

### 5-Chloro-1,2-dihydro-2,2,4-trimethyl-6-[3-(trifluoromethyl)phenyl]quinoline (Compound 230, Structure 11 of Scheme II)

This compound was prepared using the method described in Example 124 except that 4-bromo-3-chloroaniline and 3-(trifluoromethyl)phenylboronic acid were used as starting materials to afford Compound 230. ¹H NMR (400 MHz, CDCl₃) δ 7.55-7.63 (m, 3H), 7.47 (t, J=7.5, 1H), 6.91 (d, J=8.2, 1H), 6.49 (d, J=8.1, 1H), 5.50 (s, 1H), 3.93 (broad s, 1H), 2.32 (s, 3H), 1.27 (s, 6H).

### Example 131

### 5-Chloro-6-(3-cyanophenyl)-1,2-dihydro-2,2,4,8-tetramethylquinoline (Compound 231, Structure 11 of Scheme II)

This compound was prepared using the method described in Example 124 except that 4-bromo-5-chloro-2-methylaniline and 3-cyanophenylboronic acid were used as starting materials to afford Compound 231. ¹H NMR (500 MHz, CDCl₃) δ 7.68 (s, 1H), 7.63 (d, J=7.9, 1H), 7.59 (d, J=7.8, 1H), 7.47 (t, J=7.8, 1H), 6.83 (s, 1H), 5.53 (s, 1H), 3.86 (broad s, 1H), 2.32 (d, J=0.9, 3H), 2.12 (s, 3H), 1.30 (s, 6H).

### Example 132

### 5-Chloro-6-(3-cyano-4-fluorophenyl)-1,2-dihydro-2,2,4,8-tetramethylquinoline (Compound 232 Structure 11 of Scheme II)

This compound was prepared using the method described in Example 124 except that 5-chloro-2-methyl-4-(4,4,5,5-tetramethyl-1,3,2-dioxaborolan-2-yl)aniline and 5-bromo-2-fluorobenzonitrile were used as starting materials to afford Compound 232. ¹H NMR (500 MHz, CDCl₃) δ 7.56-7.58 (m, 2H), 7.18-7.21 (m, 1H), 6.80 (s, 1H), 5.51 (s, 1H), 3.84 (br s, 1H), 2.29 (s, 3H), 2.10 (s, 3H), 1.27 (s, 6H).

### Example 133

### 6-(3-Acetylphenyl)-5-chloro-1,2-dihydro-2,2,4,8-tetramethylquinoline (Compound 233, Structure 11 of Scheme II)

This compound was prepared using the method described in Example 124 except that 4-bromo-5-chloro-2-methylaniline and 3-acetylphenylboronic acid were used as starting materials to afford Compound 233. ¹H NMR (500 MHz, CDCl₃) δ 7.95 (s, 1H), 7.89 (d, J=7.7, 1H), 7.58 (d, J=7.4 Hz, 1H), 7.45 (t, J=7.7, 1H), 6.86 (s, 1H), 5.51 (s, 1H), 3.82 (br s, 1H), 2.61 (s, 3H), 2.31 (s, 3H), 2.11 (s, 3H), 1.28 (s, 6H).

### Example 134

### 5-Chloro-1,2-dihydro-2,2,4,8-tetramethyl-6-(3-methylphenyl)quinoline (Compound 234, Structure 11 of Scheme II)

This compound was prepared using the method described in Example 124 except that 4-bromo-5-chloro-2-methylaniline and 3-methylphenylboronic acid were used as starting materials to afford Compound 234. ¹H NMR (500 MHz, CDCl₃) δ 7.25-7.27 (m, 1H), 7.16-7.18 (m, 2H), 7.11 (d, J=7.5, 1H), 6.85 (s, 1H), 5.49 (s, 1H), 3.76 (br s, 1H), 2.37 (s, 3H), 2.31 (s, 3H), 2.09 (s, 3H), 1.27 (s, 6H).

### Example 135

### 5-Chloro-6-[4-chloro-3-(trifluoromethyl)phenyl]-1,2-dihydro-2,2,4,8-tetramethylquinoline (Compound 235, Structure 11 of Scheme II)

This compound was prepared using the method described in Example 124 except that 5-chloro-2-methyl-4-(4,4,5,5-tetramethyl-1,3,2-dioxaborolan-2-yl)aniline and 3-bromo-2-methylbenzonitrile were used as starting materials to afford Compound 235 ¹H NMR (500 MHz, CDCl₃) δ 7.67 (s, 1H), 7.47-7.48 (m, 2H), 6.81 (s, 1H), 5.50 (s, 1H), 3.82 (br s, 1H), 2.30 (s, 3H), 2.10 (s, 3H), 1.27 (s, 6H).

### Example 136

### 5-Chloro-6-(3-cyano-2-methylphenyl)-1,2-dihydro-2,2,4,8-tetramethylquinoline (Compound 236, Structure 11 of Scheme II)

This compound was prepared using the method described in Example 124 except that 5-chloro-2-methyl-4-(4,4,5,5-tetramethyl-1,3,2-dioxaborolan-2-yl)aniline and 3-bromo-2-methylbenzonitrile were used as starting materials to afford Compound 236. ¹H NMR (500 MHz, CDCl₃) δ 7.57 (d, J=7.4 Hz, 1H), 7.33 (d, J=7.5 Hz, 1H), 7.27 (t, J=7.6 Hz, 1H), 6.67 (s, 1H), 5.50 (s, 1H), 3.81 (br s, 1H), 2.31 (s, 3H), 2.29 (s, 3H), 2.09 (s, 3H), 1.29 (s, 3H), 1.27 (s, 3H).

### Example 137

### 5-Chloro-6-(3-fluoro-2-methylphenyl)-1,2-dihydro-2,2,4,8-tetramethylquinoline (Compound 237, Structure 11 of Scheme II)

This compound was prepared using the method described in Example 124 except that 4-bromo-5-chloro-2-methylaniline and 2-fluoro-3-methylphenylboronic acid were used as starting materials to afford Compound 237. ¹H NMR (400 MHz, CDCl₃) δ 7.10-7.18 (m, 1H), 6.98 (dd, J=8.6, 8.3, 1H), 6.91 (d, J=7.5, 1H), 6.72 (s, 1H), 5.49 (broad s, 1H), 3.78 (broad s, 1H), 2.30 (d, J=1.2, 1H), 2.09 (s, 3H), 2.02 (d, J=2.4, 3H), 1.29 (s, 3H), 1.26 (s, 3H).

### Example 138

### 5-Chloro-1,2-dihydro-2,2,4,8-tetramethyl-6-[3-(propionyl)phenyl]quinoline (Compound 238, Structure 11 of Scheme II)

This compound was prepared using the method described in Example 124 except that 5-chloro-2-methyl-4-(4,4,5,5-tetramethyl-1,3,2-dioxaborolan-2-yl)aniline and 3′-bromopropiophenone were used as starting materials to afford Compound 238. ¹H NMR (500 MHz, CDCl₃) δ 7.95 (s, 1H), 7.90 (d, J=7.9 Hz, 1H), 7.56 (d, J=7.6 Hz, 1H), 7.45 (t, J=7.7 Hz, 1H), 6.86 (s, 1H), 5.50 (s, 1H), 3.81 (br s, 1H), 3.18 (q, J=7.3 Hz, 2H), 2.31 (s, 3H), 2.11 (s, 3H), 1.27 (s, 6H), 1.23 (t, J=7.2 Hz, 3H).

### Example 139

### 6-(3-Carbamoylphenyl)-5-chloro-1,2-dihydro-2,2,4-trimethylquinoline (Compound 239)

To prepare this compound, Compound 224 (EXAMPLE 124) was mixed with 2N KOH in isopropanol and heated at reflux for several hours. The mixture was cooled and partitioned between EtOAc and saturated ammonium chloride. The organic layer was dried over magnesium sulfate, filtered, and concentrated to afford Compound 239. ¹H NMR (400 MHz, CDCl₃) δ 7.80 (s, 1H), 7.78 (d, 1H), 7.55 (d, 1H), 7.48 (t, 1H), 6.93 (d, 1H), 6.50 (d, 1H), 6.00-6.20 (broad s, 1H), 5.50-5.70 (broad s, 1H), 5.50 (s, 1H), 2.32 (s, 3H), 1.28 (s, 6H).

### Example 140

### 6-(3-Carboxymethylphenyl)-5-chloro-1,2-dihydro-2,2,4,8-tetramethylquinoline (Compound 240)

This compound was prepared using the method described in Example 124 except that 5-chloro-2-methyl-4-(4,4,5,5-tetramethyl-1,3,2-dioxaborolan-2-yl)aniline and ethyl 3-bromobenzoate were used as starting materials to afford Compound 240. ¹H NMR (500 MHz, CDCl₃) δ 8.03 (s, 1H), 7.92 (d, J=7.4 Hz, 1H), 7.57 (d, J=7.5 Hz, 1H), 7.42 (t, J=7.6 Hz, 1H), 6.86 (s, 1H), 5.50 (s, 1H), 3.90 (s, 3H), 3.86 (br s, 1H), 2.31 (s, 3H), 2.10 (s, 3H), 1.27 (s, 6H).

### Example 141

### 5-Chloro-6-(5-cyanothiophen-3-yl)-1,2-dihydro-2,2,4,8-tetramethylquinoline (Compound 241)

This compound was prepared using the method described in Example 124 except that 5-chloro-2-methyl-4-(4,4,5,5-tetramethyl-1,3,2-dioxaborolan-2-yl)aniline and 4-bromo-2-cyanothiophene were used as starting materials to afford Compound 241. ¹H NMR (400 MHz, CDCl₃) δ 7.73 (d, J=1.6, 1H), 7.47 (d, J=1.5, 1H), 6.88 (s, 1H), 5.52 (s, 1H), 3.85 (broad s, 1H), 2.31 (d, J=1.2, 3H), 2.11 (s, 3H), 1.28 (s, 6H).

### Example 142

### 5-Chloro-6-(5-cyanopyrid-3-yl)-1,2-dihydro-2,2,4,8-tetramethylquinoline (Compound 242)

This compound was prepared using the method described in Example 124 except that 5-chloro-2-methyl-4-(4,4,5,5-tetramethyl-1,3,2-dioxaborolan-2-yl)aniline and 5-bromonicotinonitrile were used as starting materials to afford Compound 242. ¹H NMR (400 MHz, CDCl₃) δ 8.82 (d, J=2.1, 1H), 8.80 (d, J=1.9, 1H), 8.00 (s, 1H), 5.54 (s, 1H), 3.93 (broad s, 1H), 2.32 (s, 3H), 2.14 (s, 3H), 1.31 (s, 6H).

### Example 143

### (±)-6-(3-Acetylphenyl)-5-chloro-1,2,3,4-tetrahydro-2,2,4,8-tetramethylquinoline (Compound 243, Structure 6 of Scheme I)

This compound was prepared using General Method 5 (EXAMPLE 1) from (±)-6-Bromo-5-chloro-1,2,3,4-tetrahydro-2,2,4,8-tetramethylquinoline and 3-acetylphenylboronic acid to afford Compound 243. ¹H NMR (400 MHz, CDCl₃) δ 7.99 (dd, J=1.6, 1.5, 1H), 7.90 (m, 1H), 7.63 (m, 1H), 7.47 (dd, J=7.7, 7.7, 1H), 6.90 (s, 1H), 3.59 (broad s, 1H), 3.30-3.40 (m, 1H), 2.62 (s, 3H), 2.11 (s, 3H), 1.97 (dd, J=13.6, 7.0, 1H), 1.81 (dd, J=13.6, 4.3, 1H), 1.44 (d, J=6.9, 3H), 1.39 (s, 3H), 1.25 (s, 3H).

### Example 143A

### (+)-6-(3-Acetylphenyl)-5-chloro-1,2,3,4-tetrahydro-2,2,4,8-tetramethylquinoline (Compound 243A) and (−)-6-(3-Acetylphenyl)-5-chloro-1,2,3,4-tetrahydro-2,2,4,8-tetramethylquinoline (Compound 243B)

These compounds were isolated from the racemic compound of Example 125 using General Method 6 (EXAMPLE 1) on a Chiracel AD column (20×250 mm, 5% isopropanol/hexanes, 6.5 ml/min, to afford Compounds 217A and 217B. Data for Compound 243A: HPLC (Chiralcel AD, 5% isopropanol/hexanes, 6 ml/min) t_{R} 14.6 min; [α]_{D}=+17.5. Data for Compound 243B: HPLC (Chiralcel AD, 5% isopropanol/hexanes, 6 ml/min) t_{R} 15.3 min; [α]_{D}=−19.2.

### Example 144

### (±)-5-Chloro-6-(5-cyanothiophen-3-yl)-1,2,3,4-tetrahydro-2,2,4,8-tetramethylquinoline (Compound 244)

This compound was prepared using General Method 2 (EXAMPLE 1) from Compound 241 (EXAMPLE 141) to afford Compound 244. ¹H NMR (400 MHz, CDCl₃) δ 7.77 (d, J=1.1, 1H), 7.49 (d, J=1.1, 1H), 6.90 (s, 1H), 3.63 (broad s, 1H), 3.32-3.37 (m, 1H), 2.09 (s, 3H), 1.95 (dd, J=13.6, 6.9, 1H), 1.81 (dd, J=13.6, 4.3, 1H), 1.41 (d, J=7.2, 3H), 1.37 (s, 3H), 1.24 (s, 3H).

### Example 145

### (±)-5-Acetoxy-6-(3-cyanophenyl)-1,2,3,4-tetrahydro-2,2,4,8-tetramethylquinoline (Compound 245, Structure 6 of Scheme I)

To prepare this compound, first 5-acetoxy-1,2-dihydro-2,2,4,8-tetramethylquinoline was prepared using General Method 1 (EXAMPLE 1) from 5-Acetoxy-2-methylaniline. That 5-Acetoxy-1,2-dihydro-2,2,4,8-tetramethylquinoline was treated according to General Method 2 (EXAMPLE 1) to afford 5-acetoxy-1,2,3,4-tetrahydro-2,2,4,8-tetramethylquinoline. That 5-acetoxy-1,2,3,4-tetrahydro-2,2,4,8-tetramethylquinoline was treated according to General Method 3 (EXAMPLE 1) to afford 5-acetoxy-6-bromo-1,2,3,4-tetrahydro-2,2,4,8-tetramethylquinoline. Finally, Compound 245 was prepared using General Method 5 (EXAMPLE 1) from the 5-acetoxy-6-bromo-1,2,3,4-tetrahydro-2,2,4,8-tetramethylquinoline and 3-cyanophenylboronic acid to afford Compound 245. ¹H NMR (500 MHz, CDCl₃) δ 7.68 (s, 1H), 7.61 (d, J=7.8 Hz, 1H), 7.51 (d, 7.8 Hz, 1H), 7.42 (t, J=7.8 Hz, 1H), 6.90 (s, 1H), 3.57 (br s, 1H), 2.85-2.91 (m, 1H), 2.09 (s, 3H), 2.04 (s, 3H), 1.87 (dd, J=7.1, 13.3 Hz, 1H), 1.56-1.61 (m, 1H), 1.31-1.34 (m, 6H), 1.16 (s, 3H).

### Example 146

### 6-[3-(N-Methoxy-N-methylcarbamoyl)phenyl]-5-chloro-1,2-dihydro-2,2,4-trimethylquinoline (Compound 246)

To prepare this compound, Compound 240 (EXAMPLE 140) was mixed with methoxymethylamine hydrochloride (1.5 equiv) and isopropylmagnesium chloride (2 M in THF, equiv) in THF at −10° C. for 30 minutes. That mixture was quenched with 2M sodium bisulfate and extracted with EtOAc. The organic layer was dried with MgSO₄, filtered and concentrated to afford Compound 246 after column chromatography (2:1 hexanes:EtOAc). ¹H NMR (500 MHz, CDCl₃) δ 7.66 (s, 1H), 7.58 (d, J=7.5 Hz, 1H), 7.45 (d, J=7.7 Hz, 1H), 7.39 (t, J=7.7 Hz, 1H), 6.86 (s, 1H), 5.49 (s, 1H), 3.79 (br s, 1H), 3.58 (s, 3H), 3.34 (s, 3H), 2.30 (s, 3H), 2.09 (s, 3H), 1.27 (s, 6H).

### Example 147

### 5-Chloro-1,2-dihydro-2,2,4,8-tetramethyl-6-[3-(2-methylpropionyl)phenyl]quinoline (Compound 247)

To prepare this compound, Compound 246 (EXAMPLE 146) was mixed with isopropylmagnesium chloride (2M in THF, 3 equiv) in THF (0.13 M) at −78° C. then allowed to warm to room temperature. That mixture was then quenched with 2N sodium bisulfate and extracted with EtOAc. The organic layer was dried over magnesium sulfate, filtered and concentrated to afford Compound 247 after flash chromatography (9:1 hexanes:EtOAc). ¹H NMR (500 MHz, CDCl₃) δ 7.95 (s, 1H), 7.89 (d, J=7.7 Hz, 1H), 7.56 (d, J=7.6 Hz, 1H), 7.45 (t, J=7.6 Hz, 1H), 6.87 (s, 1H), 5.50 (s, 1H), 3.81 (br s, 1H), 3.56 (sept., J=6.8 Hz, 1H), 2.32 (s, 3H), 2.07 (s, 3H), 1.27 (s, 6H), 1.21 (d, J=6.8, 6H).

### Example 148

### (±)-5-Chloro-6-(3-cyano-2-hydroxyphenyl)-1,2,3,4-tetrahydro-2,2,4,8-tetramethylquinoline (Compound 248)

To prepare this compound, Compound 105 (EXAMPLE 5) was mixed with boron tribromide (1 M in heptane, 1.1 equiv) in dichloromethane (0.08 M) at −78° C. That mixture was then stirred overnight at room temperature. The mixture was neutralized with aqueous sodium carbonate and extracted with dichloromethane. The organic layer was dried over sodium sulfate, filtered, and concentrated to afford Compound 248 after prep TLC (9:1 hexanes:EtOAc). ¹H NMR (400 MHz, CDCl₃) δ 7.52 (d, J=7.9, 1H), 7.40 (d, J=7.9, 0.5H), 7.39 (d, J=7.9, 0.5H), 7.00 (t, J=7.6, 1H), 6.81 (s, 1H), 5.69 (s, 0.5H), 5.59 (s, 0.5H), 3.70 (broad s, 1H), 3.34-3.40 (m, 1H), 2.09 (s, 3H), 1.97 (dd, J=13.8, 7.1, 1H), 1.77-1.87 (m, 1H), 1.42-1.43 (m, 3H), 1.39 (s, 3H), 1.25-1.27 (m, 3H).

### Example 149

### (±)-6-(3-Cyanophenyl)-1,2,3,4-tetrahydro-5-hydroxy-2,2,4,8-tetramethylquinoline (Compound 249)

To prepare this compound, Compound 245 (EXAMPLE 145) was mixed with lithium borohydride (2 M in THF, 1 equiv) in 2:1 THF:toluene (0.1 M) and the mixture was heated to 100° C. The mixture was allowed to cool and was partitioned between water and EtOAc. The organic layer was dried over magnesium sulfate, filtered, and concentrated to afford Compound 249 after flash chromatography (4:1 hexanes EtOAc). ¹H NMR (400 MHz, CDCl₃) δ 7.74 (s, 1H), 7.65-7.72 (m, 1H), 7.50-7.55 (m, 1H), 7.49 (dd, J=7.7, 7.7, 1H), 6.76 (s, 1H), 4.88 (s, 1H), 3.53 (broad s, 1H), 3.05-3.15 (m, 1H), 2.05 (s, 3H), 1.93 (dd, J=13.4, 7.4, 1H), 1.65 (dd, J=13.4, 7.4, 1H), 1.42 (d, J=6.9, 3H), 1.32 (s, 3H), 1.19 (s, 3H).

### Example 150

### (±)-6-(3-Cyanophenyl)-1,2,3,4-tetrahydro-5-methoxy-2,2,4,8-tetramethylquinoline (Compound 250)

To prepare this compound, Compound 249 (EXAMPLE 149) was mixed with iodomethane (2.5 equiv) and cesium fluoride (2.5 equiv) in DMF (0.1 M). After 10 minutes, the mixture was diluted with water and extracted with EtOAc. The organic layer was dried over magnesium sulfate, filtered, and concentrated to afford Compound 250 after flash chromatography (4:1 hexanes:EtOAc). ¹H NMR (500 MHz, CDCl₃) δ 7.85 (s, 1H), 7.78 (d, J=7.8 Hz, 1H), 7.49 (d, J=7.7 Hz, 1H), 7.42 (t, J=7.7 Hz, 1H), 6.86 (s, 1H), 3.52 (br s, 1H), 3.33 (s, 3H), 3.23-3.27 (m, 1H), 2.07 (s, 3H), 1.88 (dd, J=7.1, 13.3 Hz, 1H), 1.63 (dd, J=7.8, 13.4 Hz, 1H), 1.43 (d, J=6.8 Hz, 3H), 1.31 (s, 3H), 1.24 (s, 3H).

### Example 151

### (±)-6-(5-Carbamoylpyrid-3-yl)-5-chloro-1,2,3,4-tetrahydro-2,2,4,8-tetramethylquinoline (Compound 251, Structure 6 of Scheme I)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline and 5-bromonicotinamide to afford Compound 251 after flash chromatography (15% EtOAc/hexanes with trace of methanol). ¹H NMR (400 MHz, CDCl₃) δ 8.94 (s, 1H), 8.79 (s, 1H), 8.18 (t, J=2.0, 1H), 6.88 (s, 1H), 6.20-6.40 (v broad s, 1H), 5.95-6.15 (v broad s, 1H), 3.65 (s, 1H), 3.35-3.40 (m, 1H), 2.10 (s, 3H), 1.96 (dd, J=13.6, 6.9, 1H), 1.82 (dd, J=13.7, 4.3, 1H), 1.42 (d, 3H, J=7.1, 3H), 1.39 (s, 3H), 1.25 (s, 3H).

### Example 152

### (±)-5-Chloro-6-(2-cyanothiophen-3-yl)-1,2,3,4-tetrahydro-2,2,4,8-tetramethylquinoline (Compound 252, Structure 6 of Scheme 1)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline and 3-bromo-2-cyanothiophene to afford Compound 252 after prep TLC (95:5 hexanes:EtOAc) ¹H NMR (400 MHz, CDCl₃) δ 7.50 (d, J=5.0, 1H), 7.23 (d, J=5.0, 1H), 6.99 (s, 1H), 3.70 (broad s, 1H), 3.33-3.37 (m, 1H), 2.09 (s, 3H), 1.94 (dd, J=13.6, 6.9, 1H), 1.81 (dd, J=13.5, 4.2, 1H), 1.42 (d, J=7.0, 3H), 1.37 (s, 3H), 1.24 (s, 3H).

### Example 153

### (±)-5-Chloro-6-[3-(cyanomethyl)phenyl]-1,2,3,4-tetrahydro-2,2,4,8-tetramethylquinoline (Compound 253, Structure 6 of Scheme I)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline and 3-bromophenylacetonitrile to afford Compound 253 after flash chromatography (95:5 hexanes:EtOAc). ¹H NMR (400 MHz, CDCl₃) δ 7.36-7.39 (m, 3H), 7.25-7.28 (m, 1H), 6.87 (s, 1H), 3.78 (s, 2H), 3.58 (broad s, 1H), 3.35-3.36 (m, 1H), 2.10 (s, 3H), 1.97 (dd, J=13.5, 7.0, 1H), 1.81 (dd, J=13.5, 4.3, 1H), 1.44 (d, J=7.2, 3H), 1.39 (s, 3H), 1.25 (s, 3H).

### Example 154

### (±)-6-(3-Cyanophenyl)-5-(2,2-dimethylpropionyloxy)-1,2,3,4-tetrahydro-2,2,4,8-tetramethylquinoline (Compound 254, Structure 6 of Scheme I)

To prepare this compound, first 5-(2,2-dimethylpropionyloxy)-1,2-dihydro-2,2,4,8-tetramethylquinoline was prepared using General Method 1 (EXAMPLE 1) from 5-(2,2-dimethylpropionyloxy)-2-methylaniline (Structure 1, R¹=Me, R², R¹=H, R⁴=2,2-dimethylpropionyloxy). That 5-(2,2-dimethylpropionyloxy)-1,2-dihydro-2,2,4,8-tetramethylquinoline was treated according to General Method 2 (EXAMPLE 1) to afford 5-(2,2-dimethylpropionyloxy)-1,2,3,4-tetrahydro-2,2,4,8-tetramethylquinoline. That 5-(2,2-dimethylpropionyloxy)-1,2,3,4-tetrahydro-2,2,4,8-tetramethylquinoline was treated according to General Method 3 (EXAMPLE 1) to afford 6-bromo-5-(2,2-dimethylpropionyloxy)-1,2,3,4-tetrahydro-2,2,4,8-tetramethylquinoline. Finally, Compound 254 was prepared using General Method 5 (EXAMPLE 1) from the 6-bromo-5-(2,2-dimethylpropionyloxy)-1,2,3,4-tetrahydro-2,2,4,8-tetramethylquinoline and 3-cyanophenylboronic acid to afford Compound 254. ¹H NMR (500 MHz, CDCl₃) δ 7.62 (s, 1H), 7.56 (d, J=7.7 Hz, 1H), 7.52 (d, J=7.6 Hz, 1H), 7.41 (t, J=7.7 Hz, 1H), 6.84 (s, 1H), 3.52 (br s, 1H), 2.61-2.64 (m, 1H), 2.09 (s, 3H), 1.88-1.91 (m, 1H), 1.56-1.59 (m, 1H), 1.27 (s, 3H), 1.16 (s, 6H), 1.07 (s, 9H).

### Example 155

### (±)-5-Chloro-1,2,3,4-tetrahydro-2,2,4,8-tetramethyl-6-(5-nitrothiophen-2-yl)quinoline (Compound 255)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline and 2-bromo-5-nitrothiophene to afford Compound 255 after prep TLC (95:5 hexanes:EtOAc). ¹H NMR (400 MHz, CDCl₃) δ 7.87 (d, J=4.4, 1H), 7.16 (d, J=4.4, 1H), 7.11 (s, 1H), 3.82 (broad s, 1H), 3.37-3.39 (m, 1H), 2.10 (s, 3H), 1.90-2.00 (m, 1H), 1.80-1.90 (m, 1H), 1.41 (d, J=7.0, 3H), 1.39 (s, 3H), 1.26 (s, 3H).

### Example 156

### (±)-5-Chloro-1,2,3,4-tetrahydro-2,2,4,8-tetramethyl-6-(pyrimidin-5-yl)quinoline (Compound 256)

This compound was prepared using General Method 5 (EXAMPLE 1) from (±)-5-chloro-1,2,3,4-tetrahydro-2,2,4,8-tetramethyl-6-(4,4,5,5-tetramethyl-1,3,2-dioxaborolan-2-yl)quinoline and 5-bromopyrimidine to afford Compound 256 after prep TLC (95:5 hexanes:EtOAc). ¹H NMR (400 MHz, CDCl₃) δ 9.13 (s, 1H), 8.79 (s, 2H), 6.86 (s, 1H), 3.69 (broad s, 1H), 3.34-3.39 (m, 1H), 2.11 (s, 3H), 1.96 (dd, J=13.6, 6.9, 1H), 1.83 (dd, J=13.6, 4.2, 1H), 1.43 (d, J=7.2, 3H), 1.39 (s, 3H), 1.25 (s, 3H).

### Example 157

### 6-(3-Acetylphenyl)-5,7-dichloro-1,2-dihydro-2,2,4-trimethylquinoline (Compound 257, Structure 11 of Scheme II)

To prepare this compound, first 2-(3-acetylphenyl)-1,3-dichloro-5-nitrobenzene (Structure 10, Scheme II) was prepared using General Method 5 (EXAMPLE 1) from 2-bromo-1,3-dichloro-5-nitrobenzene (Structure 9, Scheme II, where R¹=H, R², R⁴=Cl, X=Br) and 3-acetylphenyboronic acid. That 2-(3-acetylphenyl)-1,3-dichloro-5-nitrobenzene was then mixed with zinc dust (4 equiv) and calcium chloride dehydrate (2 equiv) in 95% ethanol/water heated at reflux. After 24 hours, the mixture was treated with hot ethanol and filtered through Celite. The filtrate was concentrated and dissolved in EtOAc and the pH was adjusted ot 2-4 with 2% HCl (aqueous). The aqueous layer was extracted with EtOAc, and the combined organic layers were washed sequentially with water, saturated sodium bicarbonate, and brine, and then dried over magnesium sulfate, filtered and concentrated. Flash chromatography afforded 4-(3-acetylphenyl)-3,5-dichloroaniline. That compound was then treated according to General Method 1 (EXAMPLE 1) to afford Compound 257 after flash chromatography (2:1 hexanes:EtOAc). ¹H NMR (400 MHz, CDCl₃) δ 7.97 (d, J=7.8, 1H), 7.83 (s, 1H), 7.52 (dd, J=7.7, 7.7, 1H), 7.44 (d, J=7.6, 1H), 6.60 (s, 1H), 5.50 (s, 1H), 4.00 (broad s, 1H), 2.63 (s, 3H), 2.28 (s, 3H), 1.29 (s, 6H).

### Example 158

### (±)-1,2,3,4-Tetrahydro-3β-hydroxy-6-(indol-7-yl)-2,2,4α,8-tetramethylquinoline (Compound 258, Structure 6 of Scheme I, where R¹=Me, R²=H, R⁴=H, R⁵=α-Me, R⁶=β-OH, R⁹=H, Ar=indol-7-yl)

This compound was prepared from o-toluidine using General Method 1 (EXAMPLE 1), General Method 7 (EXAMPLE 59), and General Method 3 (EXAMPLE 1) to afford (±)-6-bromo-1,2,3,4-tetrahydro-3β-hydroxy-2,2,4α,8-tetramethylquinoline. (±)-6-Bromo-1,2,3,4-tetrahydro-3β-hydroxy-2,2,4α,8-tetramethylquinoline and 7-(4,4,5,5-tetramethyl-1,3,2-dioxaborolan-2-yl)indole were treated according to General Method 5 (EXAMPLE 1) to afford compound 258. ¹H NMR (500 MHz, CDCl₃) δ 8.42 (s, 1H), 7.57 (m, 1H), 7.35 (s, 1H), 7.23-7.20 (m, 2H), 7.18-7.15 (m, 2H), 6.60 (dd, J=3.2, 2.1 Hz, 1H), 3.62 (s, 1H), 3.41 (dd, J=9.5, 6.0 Hz, 1H), 2.82 (m, 1H), 2.19 (s, 3H), 1.76 (d, J=6.0 Hz, 1H), 1.48 (d, J=6.8 Hz, 3H), 1.38 (s, 3H), 1.16 (s, 3H).

### Example 159

### (±)-6-(3,5-Dimethylisoxazol-4-yl)-1,2,3,4-tetrahydro-3β-hydroxy-2,2,4α,8-tetramethylquinoline (Compound 259, Structure 6 of Scheme I, where R¹=Me, R²=H, R⁴=H, R⁵=α-Me, R⁶=β-OH, R⁹=H, Ar=3,5-dimethylisoxazol-4-yl)

This compound was prepared according to General Method 5 (EXAMPLE 1) from (±)-6-bromo-1,2,3,4-tetrahydro-3β-hydroxy-2,2,4α,8-tetramethylquinoline and 3,5-dimethyl-4-isoxazolylboronic acid to afford compound 259. ¹H NMR (500 MHz, CDCl₃) δ 6.94 (s, 1H), 6.79 (m, 1H), 3.60 (s, 1H), 3.37 (m, 1H), 2.77 (dq, J=9.1, 6.8 Hz, 1H), 2.39 (s, 3H), 2.26 (s, 3H), 2.13 (s, 3H), 1.86 (m, 1H), 1.44 (d, J=6.6 Hz, 3H), 1.36 (s, 3H), 1.13 (s, 3H).

### Example 160

### (±)-1,2,3,4-Tetrahydro-3β-hydroxy-2,2,4α,8-tetramethyl-6-(quinolin-8-yl)quinoline (Compound 260, Structure 6 of Scheme I, where R¹=Me, R²=H, R⁴=H, R⁵=α-Me, R⁶=β-OH, R⁹=H, Ar=quinolin-8-yl)

This compound was prepared according to General Method 5 (EXAMPLE 1) from (±)-6-bromo-1,2,3,4-tetrahydro-3β-hydroxy-2,2,4α,8-tetramethylquinoline and 8-quinolineboronic acid to afford compound 260. ¹H NMR (500 MHz, CDCl₃) δ 8.94 (dd, J=4.0, 1.5 Hz, 1H), 8.18 (dd, J=8.2, 1.5 Hz, 1H), 7.74 (dd, J=7.5, 1.0 Hz, 1H), 7.70 (dd, J=7.5, 1.0 Hz, 1H), 7.56 (t, J=7.5 Hz, 1H), 7.42 (s, 1H), 7.39 (dd, J=8.2, 4.0 Hz, 1H), 7.33 (s, 1H), 3.62 (s, 1H), 3.40 (dd, J=8.8, 3.7 Hz, 1H), 2.85 (dq, J=8.8, 6.8 Hz, 1H), 2.18 (s, 3H), 1.74 (m, 1H), 1.49 (d, J=6.8 Hz, 3H), 1.37 (s, 3H), 1.16 (s, 3H).

### Example 161

### (±)-5-Chloro-1,2,3,4-tetrahydro-6-(indol-7-yl)-2,2,4,8-tetramethylquinoline (Compound 261, Structure 73 of Scheme XXI, where R¹=Me, R²=H, R⁴=Cl, R⁵=Me, R⁶=H, R⁹=H, R^{E}=allyl)

To a solution of compound 149 (EXAMPLE 49) (20 mg, 0.06 mmol) in 1.2 mL ether was added 40 microliters of ethylmagnesium bromide (3 M) at 0° C. Allyl bromide (14 mg, 0.12 mmol) was added, and the solution was allowed to warm to rt. After 18 h, the mixture was quenched with saturated ammonium chloride and extracted with EtOAc. The organic layer washed with brine, dried over magnesium sulfate, and filtered. Compound 261 (2 mg, 9%) was isolated after purification by semi-prep HPLC (85:15 MeOH:water, ODS column, 10×250, 3 mL/min). ¹H NMR (500 MHz, CDCl₃) δ 7.85 (broad s, ½H), 7.84 (broad s, ½H), 7.58 (d, J=7.3, 1H), 7.06-7.18 (m, 2H), 6.98 (s, 1H), 6.94-6.98 (,. 1H), 6.05-6.15 (m, 1H), 5.16-5.22 (m, 1H), 5.04-5.10 (m, 1H), 3.59 (broad s, 1H), 3.55 (d, J=6.8, 2H), 3.33-3.43 (m, 1H), 2.10 (s, 3H), 2.00 (dd, J=13.7, 6.8, 1H), 1.79-1.86 (m, 1H), 1.47 (d, J=7.3, 3/2H), 1.44 (d, J=7.3, 3/2H), 1.40 (s, 3H), 1.28 (s, 3/2H), 1.26 (s, 3/2H).

### Example 162

### (±)-5-Chloro-1,2,3,4-tetrahydro-3β-hydroxy-2,2,4α,8-tetramethyl-6-(6-fluoro-2-nitrophenyl)-quinoline (Compound 262, Structure 6 of Scheme I, where R¹=Me, R²=H, R⁴=Cl, R⁵=Me, R⁶=β-OH, R⁹=H, Ar=6-fluoro-2-nitrophenyl)

General Method 12: Palladium-catalyzed cross-coupling of an aryl halide and an aryl boronic acid. In a sealed tube or Schlenck flask, a mixture of an aryl bromide (1 equiv); an aryl boronic acid or aryl pinacol boronate (1.0-1.2 equiv), tetrakis(triphenylphosphine)palladium(0) (10 mol %), and barium hydroxide (2 equiv) is flushed with nitrogen. A 90% dioxane/water solution is added to form a 0.1 M solution, and the mixture is heated at 100° C. for 16-24 h. The mixture is distributed between ethyl acetate and saturated ammonium chloride, and the aqueous layer is extracted with ethyl acetate. The organic layers are washed with brine, dried over magnesium sulfate, filtered, and concentrated. Flash chromatography (ethyl acetate:hexanes) affords the product.

### (±)-5-Chloro-1,2,3,4-tetrahydro-3β-hydroxy-2,2,4α,8-tetramethyl-6-(2-nitro-6-fluorophenyl)-quinoline (Compound 262, Structure 6 of Scheme I, where R¹=Me, R²=H, R⁴=Cl, R⁵=Me, R⁶=β-OH, R⁹=H, Ar=2-nitro-6-fluorophenyl)

This compound was prepared according to General Method 12 from (±)-5-chloro-1,2,3,4-tetrahydro-3β-hydroxy-2,2,4α,8-tetramethyl-6-(4,4,5,5-tetramethyl-1,3,2-dioxaborolan-2-yl)quinoline (EXAMPLE 63) and 3-fluoro-2-bromonitrobenzene to afford compound 262. ¹H NMR (500 MHz, CDCl₃) δ 7.81 (m, 1H), 7.52 (dd, J=8.2, 5.3 Hz, 1H), 7.43 (td, J=8.2, 1.0 Hz, 0.5H), 7.42 (td, J=8.2, 1.0 Hz, 0.5H), 6.83 (s, 0.5H), 6.82 (s, 0.5H), 3.68 (s, 1H), 3.61 (m, 1H), 3.12 (dq, J=4.6, 7.0 Hz, 0.5H), 3.10 (dq, J=4.8, 7.0 Hz, 0.5H), 2.14 (s, 3H), 1.92 (d, J=8.2 Hz, 0.5H), 1.89 (d, J=8.2 Hz, 0.5H), 1.54 (d, J=7.0 Hz, 1.5H), 1.53 (d, J=7.0 Hz, 1.5H), 1.38 (s, 3H), 1.26 (s, 1.5H), 1.24 (s, 1.5H).

### Example 163

### (±)-5-Chloro-1,2,3,4-tetrahydro-3β-hydroxy-6-(6-fluoroindol-7-yl)-2,2,4α,8-tetramethylquinoline (Compound 263, Structure 51 of Scheme XIII, where R¹=Me, R²=H, R⁴=Cl, R⁵=α-Me, R⁶=β-OH, R⁹=H, R^{A}=H, R^{B}=H, R²⁴=H, R²⁵=F)

General method 13: Formation of an indole from a 2-substituted nitrobenzene. To a solution of a 2-substituted nitrobenzene (1 equiv) in THF (0.02-0.15 M) is added a vinylmagnesium bromide (ether or THF solution, 5 equiv) at −40° C. Additional vinylmagnesium bromide reagent is added as needed to convert the reaction to completion. After 1-2 h, the reaction is quenched with saturated ammonium chloride. The mixture is extracted with ethyl acetate, washed with brine, dried over magnesium sulfate and filtered. Flash chromatography (ethyl acetate:hexanes) affords the desired product.

### (±)-5-Chloro-1,2,3,4-tetrahydro-3β-hydroxy-6-(6-fluoroindol-7-yl)-2,2,4α,8-tetramethylquinoline (Compound 263, Structure 51 of Scheme XIII, where R¹=Me, R²=H, R⁴=Cl, R⁵=α-Me, R⁶=β-OH, R⁹=H, R^{A}=H, R^{B}=H, R²⁴=H, R²⁵=F)

This compound was prepared according to General Method 13 from compound 262 and vinylmagnesium bromide. ¹H NMR (500 MHz, CDCl₃) δ 8.01 (s, 1H), 7.59 (dd, J=8.6, 4.9 Hz, 1H), 7.20 (dd, J=3.0, 2.3 Hz, 1H), 7.05 (s, 1H), 7.01 (dd, J=10.1, 8.6 Hz, 1H), 6.60 (dd, J=3.0, 2.3 Hz, 1H) 3.69 (s, 1H), 3.64 (dd, J=8.2, 5.0 Hz, 1H), 3.18 (dq, J=5.0, 6.9 Hz, 1H), 2.18 (s, 3H), 1.86 (d, J=8.2 Hz, 1H), 1.59 (d, J=6.9 Hz, 3H), 1.41 (s, 3H), 1.28 (s, 3H).

### Example 164

### (±)-5-Chloro-1,2,3,4-tetrahydro-3β-hydroxy-2,2,4α,8-tetramethyl-6-(4,6-difluoro-2-nitrophenyl)quinoline (Compound 264, Structure 6 of Scheme I, where R¹=Me, R²=H, R⁴=Cl, R⁵=α-Me, R⁶=β-OH, R⁹=H, Ar=4,6-difluoro-2-nitrophenyl)

This compound was prepared according to General Method 12 (EXAMPLE 162) from (±)-5-chloro-1,2,3,4-tetrahydro-3β-hydroxy-2,2,4α,8-tetramethyl-6-(4,4,5,5-tetramethyl-1,3,2-dioxaborolan-2-yl)quinoline (EXAMPLE 63) and 2-bromo-3,5-difluoro-1-nitrobenzene to afford compound 264.

### Example 165

### (±)-5-Chloro-1,2,3,4-tetrahydro-3β-hydroxy-6-(4,6-difluoroindol-7-yl)-2,2,4α,8-tetramethylquinoline (Compound 265, Structure 51 of Scheme XIII, where R¹=Me, R²=H, R⁴=Cl, R⁵=α-Me, R⁶=β-OH, R⁹=H, R^{A}=H, R^{B}=H, R²⁴=4-fluoro, R²⁵=F)

This compound was prepared according to General Method 13 (EXAMPLE 163) from compound 264 (EXAMPLE 164) and vinylmagnesium bromide to afford compound 265. ¹H NMR (500 MHz, CDCl₃) δ 8.10 (s, 1H), 7.16 (dd, J=3.2, 2.3 Hz, 1H), 7.01 (s, 1H), 6.74 (t, J=10.1 Hz, 1H), 6.67 (dd, J=3.2, 2.3 Hz, 1H), 3.70 (s, 1H), 3.64 (m, 1H), 3.17 (dq, J=4.8, 7.1 Hz, 1H), 2.17 (s, 3H), 1.87 (m, 1H), 1.58 (d, J=7.1 Hz, 3H), 1.41 (s, 3H), 1.27 (s, 3H).

### Example 166

### (±)-5-Chloro-1,2,3,4-tetrahydro-3β-hydroxy-6-(5-fluoroindol-7-yl)-2,2,4α,8-tetramethylquinoline (Compound 266, Structure 6 of Scheme I, where R¹=Me, R²=H, R⁴=Cl, R⁵=α-Me, R⁶=β-OH, R⁹=H, Ar=5-fluoroindol-7-yl)

This compound was prepared using General Method 5 (EXAMPLE 1) from (±)-5-chloro-1,2,3,4-tetrahydro-3β-hydroxy-2,2,4α,8-tetramethyl-6-(4,4,5,5-tetramethyl-1,3,2-dioxaborolan-2-yl)quinoline and 7-bromo-5-fluoroindole to afford Compound 266. ¹H NMR (500 MHz, CDCl₃) δ 8.02 (s, 1H), 7.28 (d, J=2.5 Hz, 0.5H), 7.26 (d, J=2.5 Hz, 0.5H), 7.22 (d, J=11.5 Hz, 1H), 7.00 (s, 1H), 6.92 (d, J=9.7 Hz, 0.5H), 6.85 (d, J=9.9 Hz, 0.5H), 6.55 (m, 1H), 3.65 (s, 1H), 3.62 (m, 1H), 3.15 (m, 1H), 2.14 (s, 3H), 1.92 (d, J=7.6 Hz, 0.5H), 1.86 (d, J=7.6 Hz, 0.5H), 1.56 (d, J=6.9 Hz, 1.5H), 1.53 (d, J=7.0 Hz, 1.5H), 1.37 (s, 3H), 1.27 (s, 1.5H), 1.23 (s, 1.5H).

### Example 167

### (±)-1,2,3,4-Tetrahydro-3β-hydroxy-2,2,4α,8-tetramethyl-6-(6-methoxy-2-nitrophenyl)-quinoline (Compound 267, Structure 6 of Scheme I, where R¹=Me, R²=H, R⁴=H, R⁵=α-Me, R⁶=β-OH, R⁹=H, Ar=6-methoxy-2-nitrophenyl)

This compound was prepared according to General Method 12 (EXAMPLE 162) from (±)-1,2,3,4-tetrahydro-3β-hydroxy-2,2,4α,8-tetramethyl-6-(4,4,5,5-tetramethyl-1,3,2-dioxaborolan-2-yl)quinoline (EXAMPLE 63) and 2-bromo-3-nitroanisole to afford compound 267.

### Example 168

### (±)-1,2,3,4-Tetrahydro-3β-hydroxy-6-(6-methoxy-indol-7-yl)-2,2,4α,8-tetramethylquinoline (Compound 268, Structure 51 of Scheme XIII, where R¹=Me, R²=H, R⁴=H, R⁵=α-Me, R⁶=β-OH, R⁹=H, R^{A}=H, R^{B}=H, R²⁴=H, R²⁵=OMe)

This compound was prepared according to General Method 13 (EXAMPLE 163) from compound 267 (EXAMPLE 167) and vinylmagnesium bromide to afford compound 268. ¹H NMR (500 MHz, CDCl₃) δ 8.12 (s, 1H), 7.55 (d, J=8.6 Hz, 1H), 7.32 (m, 1H), 7.15 (m, 1H), 7.12 (m, 1H), 6.97 (d, J=8.6 Hz, 1H), 6.55 (m, 1H), 3.86 (s, 3H), 3.64 (s, 1H), 3.43 (dd, J=9.3, 5.9 Hz, 1H), 2.85 (dq, J=9.3, 6.8 Hz, 1H), 2.20 (s, 3H), 1.87 (d, J=5.9 Hz, 1H), 1.49 (d, J=6.8 Hz, 3H), 1.41 (s, 3H), 1.20 (s, 3H).

### Example 169

### (±)-7-Fluoro-1,2,3,4-tetrahydro-3β-hydroxy-6-(indol-7-yl)-2,2,4α,8-tetramethylquinoline (Compound 269, Structure 6 of Scheme I, where R¹=Me, R²=F, R⁴=H, R⁵=α-Me, R⁶=β-OH, R⁹=H, Ar=indol-7-yl)

This compound was prepared from 3-fluoro-2-methylaniline using General Method 1 (EXAMPLE 1), General Method 7 (EXAMPLE 59), and General Method 3 (EXAMPLE 1) to afford (±)-6-bromo-7-fluoro-1,2,3,4-tetrahydro-3β-hydroxy-2,2,4α,8-tetramethylquinoline. (±)-6-Bromo-7-fluoro-1,2,3,4-tetrahydro-3β-hydroxy-2,2,4α,8-tetramethylquinoline and 7-(4,4,5,5-tetramethyl-1,3,2-dioxaborolan-2-yl)indole were treated according to General Method 5 (EXAMPLE 1) to afford compound 269. ¹H NMR (300 MHz, CDCl₃) δ 8.22 (s, 1H), 7.62 (m, 1H), 7.23-7.16 (m, 4H), 6.60 (dd, J=3.2, 2.1 Hz, 1H), 3.70 (s, 1H), 3.37 (dd, J=9.6, 6.0 Hz, 1H), 2.76 (m, 1H), 2.10 (d, J=1.8 Hz, 3H), 1.77 (d, J=6.0 Hz, 1H), 1.43 (d, J=6.7 Hz, 3H), 1.38 (s, 3H), 1.15 (s, 3H).

### Example 170

### (±)-6-(3,5-Dimethylisoxazol-4-yl)-1,2,3,4-tetrahydro-3β-hydroxy-5-methoxy-2,2,4α,8-tetramethylquinoline (Compound 270, Structure 6 of Scheme I, where R¹=Me, R²=H, R⁴=OMe, R⁵=α-Me, R⁶=β-OH, R⁹=H, Ar=3,5-dimethylisoxazol-4-yl)

This compound was prepared from 5-methoxy-2-methylaniline using General Method 1 (EXAMPLE 1), General Method 7 (EXAMPLE 59), and General Method 3 (EXAMPLE 1) to afford (±)-6-bromo-1,2,3,4-tetrahydro-3β-hydroxy-5-methoxy-2,2,4α,8-tetramethylquinoline. (±)-6-Bromo-1,2,3,4-tetrahydro-3β-hydroxy-5-methoxy-2,2,4α,8-tetramethylquinoline and 3,5-dimethyl-4-isoxazolylboronic acid were treated according to General Method 5 (EXAMPLE 1) to afford compound 270. ¹H NMR (500 MHz, CDCl₃) δ 6.74 (m, 1H), 3.38 (s, 3H), 3.33 (d, J=8.1 Hz, 1H), 2.80 (dq, J=8.1, 6.6 Hz, 1H), 2.34 (s, 3H), 2.19 (s, 3H), 2.14 (s, 1.5H), 2.14 (s, 1.5H), 1.51 (d, J=6.6 Hz, 3H), 1.36 (s, 3H), 1.06 (s, 3H).

### Example 171

### (±)-1,2,3,4-tetrahydro-3β-hydroxy-5-methoxy-2,2,4α,8-tetramethyl-6-(naphth-1-yl)quinoline (Compound 271, Structure 6 of Scheme I, where R¹=Me, R²=H, R⁴=OMe, R⁵=α-Me, R⁶=β-OH, R⁹=H, Ar=naphth-1-yl)

This compound was prepared according to General Method 5 (EXAMPLE 1) from (±)-6-Bromo-1,2,3,4-tetrahydro-3β-hydroxy-5-methoxy-2,2,4α,8-tetramethylquinoline and 1-naphthaleneboronic acid to afford compound 271. ¹H NMR (500 MHz, CDCl₃) δ 7.87 (t, J=7.4 Hz, 1H), 7.82 (m, 1H), 7.79 (t, J=8.5 Hz, 1H), 7.53-7.39 (m, 4H), 6.89 (s, 0.5H), 6.88 (s, 0.5H), 3.55 (s, 1H), 3.51 (t, J=6.8 Hz, 0.5H), 3.51 (dd, J=6.8, 5.8 Hz, 0.5H), 3.18 (s, 1.5H), 3.11 (s, 1.5H), 2.99 (dq, J=5.8, 6.8 Hz, 0.5H), 2.92 (qn, J=6.8 Hz, 0.5H), 2.13 (s, 3H), 1.91 (d, J=7.7 Hz, 0.5H), 1.89 (d, J=7.3 Hz, 0.5H), 1.57 (d, J=6.8 Hz, 1.5H), 1.53 (d, J=6.8 Hz, 1.5H), 1.37 (s, 1.5H), 1.36 (s, 1.5H), 1.24 (s, 1.5H), 1.20 (s, 1.5H).

### Example 172

### (±)-1,2,3,4-tetrahydro-3β-hydroxy-6-(indol-7-yl)-5-methoxy-2,2,4α,8-tetramethylquinoline (Compound 272, Structure 6 of Scheme I, where R¹=Me, R²=H, R⁴=OMe, R⁵=α-Me, R⁶=β-OH, R⁹=H, Ar=indol-7-yl)

This compound was prepared according to General Method 5 (EXAMPLE 1) from (±)-6-bromo-1,2,3,4-tetrahydro-3β-hydroxy-5-methoxy-2,2,4α,8-tetramethylquinoline and 7-(4,4,5,5-tetramethyl-1,3,2-dioxaborolan-2-yl)indole to afford compound 172. ¹H NMR (500 MHz, CDCl₃) δ 9.68 (s, 1H), 7.53 (dd, J=7.4, 1.4 Hz, 0.5H), 7.53 (dd, J=7.4, 1.4 Hz, 0.5H), 7.24 (d, J=3.1 Hz, 0.5H), 7.25 (d, J=3.1 Hz, 0.5H), 7.12 (dd, J=7.4, 1.4 Hz, 1H), 7.08 (t, J=7.4 Hz, 1H), 6.99 (m, 1H), 6.50 (d, J=3.1 Hz, 0.5H), 6.50 (d, J=3.1 Hz, 0.5H), 3.40 (d, J=8.0 Hz, 1H), 3.23 (s, 3H), 2.87 (dq, J=8.0, 6.6 Hz, 1H), 2.18 (s, 1.5H), 2.18 (s, 1.5H), 1.57 (d, J=6.6 Hz, 3H), 1.38 (s, 3H), 1.09 (s, 3H).

### Example 173

### (±)-5-Chloro-6-(2-fluoropyrid-3-yl)-1,2,3,4-tetrahydro-3β-hydroxy-2,2,4α,8-tetramethylquinoline (Compound 273, Structure 6 of Scheme I, where R¹=Me, R²=H, R⁴=Cl, R⁵=α-Me, R⁶=β-OH, R⁹=H, Ar=2-fluoropyrid-3-yl)

This compound was prepared using General Method 5 (EXAMPLE 1) from (±)-6-bromo-5-chloro-1,2,3,4-tetrahydro-3β-hydroxy-2,2,4α,8-tetramethylquinoline and 2-fluoropyrid-3-ylboronic acid to afford Compound 273. ¹H NMR (300 MHz, CDCl₃) δ 8.19 (ddd, J=4.9, 2.0, 1.1 Hz, 1H), 7.73 (ddd, J=9.4, 7.3, 2.0 Hz, 1H), 7.23 (ddd, J=7.3, 4.9, 1.9 Hz, 1H), 6.89 (s, 1H), 3.65 (s, 1H), 3.59 (dd, J=7.8, 4.7 Hz, 1H), 3.11 (dq, J=4.7, 7.0 Hz, 1H), 2.13 (s, 3H), 1.98 (d, J=7.8 Hz, 1H), 1.52 (d, J=7.0 Hz, 3H), 1.35 (s, 3H), 1.22 (s, 3H).

### Example 174

### (±)-5-Chloro-1,2,3,4-tetrahydro-3β-hydroxy-6-(2-methoxypyrid-3-yl)-2,2,4α,8-tetramethylquinoline (Compound 274, Structure 6 of Scheme I, where R¹=Me, R²=H, R⁴=Cl, R⁵=α-Me, R⁶=β-OH, R⁹=H, Ar=2-methoxypyrid-3-yl)

This compound was prepared using General Method 5 (EXAMPLE 1) from (±)-6-bromo-5-chloro-1,2,3,4-tetrahydro-3β-hydroxy-2,2,4α,8-tetramethylquinoline and 2-methoxypyrid-3-ylboronic acid to afford Compound 274. ¹H NMR (300 MHz, CDCl₃) δ 8.17 (dd, J=5.0, 1.9 Hz, 1H), 7.46 (dd, J=7.2, 1.9 Hz, 1H), 6.93 (dd, J=7.2, 5.0 Hz, 1H), 6.86 (q, J=0.6 Hz, 1H), 3.93 (s, 3H), 3.61-3.55 (m, 2H), 3.12 (m, 1H), 2.12 (d, J=0.6 Hz, 3H), 1.88 (d, J=8.4 Hz, 1H), 1.52 (d, J=7.1 Hz, 3H), 1.34 (s, 3H), 1.23 (s, 3H).

### Example 175

### (±)-5-Chloro-1,2,3,4-tetrahydro-8-fluoro-3β-hydroxy-6-(indol-7-yl)-2,2,4α-trimethylquinoline (Compound 275, Structure 6 of Scheme I, where R¹=F, R²=H, R⁴=Cl, R⁵=α-Me, R⁶=β-OH, R⁹=H, Ar=indol-7-yl)

This compound was prepared from 5-chloro-2-fluoroaniline using General Method 1 (EXAMPLE 1), General Method 7 (EXAMPLE 59), and General Method 3 (EXAMPLE 1) to afford (±)-6-bromo-5-chloro-8-fluoro-1,2,3,4-tetrahydro-3β-hydroxy-2,2,4α,-trimethylquinoline. That (±)-6-Bromo-5-chloro-8-fluoro-1,2,3,4-tetrahydro-3β-hydroxy-2,2,4α-trimethylquinoline and 7-(4,4,5,5-tetramethyl-1,3,2-dioxaborolan-2-yl)indole were treated according to General Method 5 (EXAMPLE 1) to afford compound 275. ¹H NMR (300 MHz, CDCl₃) δ 8.04 (s, 1H), 7.66 (d, J=7.6 Hz, 1H), 7.20 (m, 1H), 7.17 (dd, J=7.6, 7.3 Hz, 1H), 7.09 (m, 1H), 6.99 (d, J=11.0 Hz, 1H), 6.60 (m, 1H), 4.06 (m, 1H), 3.64 (m, 1H), 3.15 (m, 1H), 2.02 (d, J=7.2 Hz, 0.5H), 1.95 (d, J=7.2 Hz, 0.5H), 1.58 (d, J=7.1 Hz, 1.5H), 1.54 (d, J=7.1 Hz, 1.5H), 1.37 (s, 3H), 1.30 (s, 1.5H), 1.26 (s, 1.5H).

### Example 176

### (±)-5-Cyano-1,2,3,4-tetrahydro-3β-hydroxy-6-(indol-7-yl)-2,2,4α,8-tetramethylquinoline (Compound 276, Structure 6 of Scheme I, where R¹=Me, R²=H, R⁴=CN, R⁵=α-Me, R⁶=β-OH, R⁹=H, Ar=indol-7-yl)

### 5-Cyano-1,2-dihydro-2,2,4,8-tetramethylquinoline

To prepare this compound, Pd₂(dba)₃ (990 mg, 1.08 mmol), dppf (1.2 g, 2.2 mmol), zinc powder (420 mg, 6.5 mmol) and zinc cyamide (1.92 g, 16.2 mmol) were added to a solution of 5-chloro-1,2-dihydro-2,2,4,8-tetramethylquinoline (6.0 g, 27 mmol) in N,N-dimethylacetamide (120 ml). The reaction vessel was evacuated-purged with nitrogen twice and then heated at 150° C. for 48 h. The reaction was allowed to cool to room temperature, poured into water (500 ml) and extracted with ethyl acetate (3×100 ml). The combined organic extracts were washed with a saturated solution of ammonium chloride (300 ml), dried (Na₂SO₄) and concentrated under reduced pressure. Purification by flash chromatography, eluting with ethyl acetate:hexanes gave 5-cyano-1,2-dihydro-2,2,4,8-tetramethylquinoline (2.45 g, 42%).

### (±)-5-Cyano-1,2,3,4-tetrahydro-3β-hydroxy-2,2,4α,8-tetramethylquinoline

This compound was prepared according to General Method 7 (EXAMPLE 59) to afford (±)-5-cyano-1,2,3,4-tetrahydro-3β-hydroxy-2,2,4α,8-tetramethylquinoline.

### (±)-6-Bromo-5-cyano-1,2,3,4-tetrahydro-3β-hydroxy-2,2,4α,8-tetramethylquinoline

This compound was prepared according to General Method 2 (EXAMPLE 1) from (±)-5-cyano-1,2,3,4-tetrahydro-3β-hydroxy-2,2,4α,8-tetramethylquinoline to afford (±)-6-bromo-5-cyano-1,2,3,4-tetrahydro-3β-hydroxy-2,2,4α,8-tetramethylquinoline.

### (±)-5-Cyano-1,2,3,4-tetrahydro-3β-hydroxy-6-(indol-7-yl)-2,2,4α,8-tetramethylquinoline (Compound 276, Structure 6 of Scheme I, where R¹=Me, R²=H, R⁴=CN, R⁵=α-Me, R⁶=β-OH, R⁹=H, Ar=indol-7-yl)

This compound was prepared according to General Method 5 (EXAMPLE 1) from (±)-6-bromo-5-cyano-1,2,3,4-tetrahydro-3β-hydroxy-2,2,4α,8-tetramethylquinoline and 7-(4,4,5,5-tetramethyl-1,3,2-dioxaborolan-2-yl)indole to afford compound 276. ¹H NMR (500 MHz, CDCl₃) δ 8.12 (br s, 1H), 7.66 (ddd, J=7.6, 1.2, 0.7 Hz, 1H), 7.23 (dd, J=7.2, 1.2 Hz, 1H), 7.20 (dd, J=7.6, 7.2 Hz, 1H), 7.20 (dd, J=3.2, 2.6 Hz, 1H), 7.18 (m, 1H), 6.62 (dd, J=3.2, 2.1 Hz, 1H), 3.71 (br s, 1H), 3.57 (dd, J=7.0, 5.8 Hz, 1H), 3.16 (dq, J=5.8, 7.0 Hz, 1H), 2.22 (d, J=0.6 Hz, 3H), 1.88 (d, J=7.0 Hz, 1H), 1.65 (d, J=7.0 Hz, 3H), 1.38 (s, 3H), 1.21 (s, 3H).

### Example 177

### (±)-5-Ethynyl-1,2,3,4-tetrahydro-3β-hydroxy-6-(indol-7-yl)-2,2,4α,8-tetramethylquinoline (Compound 277, Structure 70 of Scheme XIX, where R¹=Me, R²=H, R⁵=α-Me, R⁹=H, Ar=indol-7-yl)

### (±)-5-Formyl-1,2,3,4-tetrahydro-3β-hydroxy-2,2,4α,8-tetramethylquinoline

To prepare this compound, 1M DIBAL in hexanes (16 ml, 16 mmol) was added dropwise to a solution of (±)-5-cyano-1,2,3,4-tetrahydro-3β-hydroxy-2,2,4α,8-tetramethylquinoline (EXAMPLE 176) (1.0 g, 4.3 mmol) in dichloromethane (200 ml) at 0° C. The solution was stirred at 0° C. for 0.25 h then quenched with the dropwise addition of a saturated solution of Rochelle's salt (100 ml). The layers were separated and the aqueous layer extracted with dichloromethane (3×100 ml). The combined organic extracts were washed with a 1M hydrochloric acid solution (300 ml), a saturated solution of ammonium chloride (300 ml), dried (Na₂SO₄) and concentrated under reduced pressure to give (±)-5-formyl-1,2,3,4-tetrahydro-3β-hydroxy-2,2,4α,8-tetramethylquinoline (770 mg, 76%).

### (±)-6-Bromo-5-formyl-1,2,3,4-tetrahydro-3β-hydroxy-2,2,4α,8-tetramethylquinoline

This compound was prepared according to General Method 3 (EXAMPLE 1) from (±)-5-formyl-1,2,3,4-tetrahydro-3β-hydroxy-2,2,4α,8-tetramethylquinoline to afford (±)-6-bromo-5-formyl-1,2,3,4-tetrahydro-3β-hydroxy-2,2,4α,8-tetramethylquinoline.

### (±)-6-Bromo-5-ethynyl-1,2,3,4-tetrahydro-2,2,4α,8-tetramethyl-3β-(trimethylsilyloxy)quinoline

To prepare this compound, 2.5 M n-butyl lithium in hexanes (0.27 ml, 0.68 mmol) was added dropwise to a solution of diisopropylamine (0.10 ml, 0.68 mmol) in THF (6 ml) at 0° C. The solution was stirred for 0.1 h at 0° C., cooled to −78° C. before the dropwise addition of 2 M (trimethylsilyl)diazomethane in hexanes (0.34 ml, 0.68 mmol). The reaction was stirred at −78° C. for 0.25 h, (±)-6-bromo-5-formyl-1,2,3,4-tetrahydro-3β-hydroxy-2,2,4α,8-tetramethylquinoline (90 mg, 0.29 mmol) added dropwise at this temperature, the reaction allowed to warm to room temperature and stirred for 15 h. A saturated solution of ammonium chloride (30 ml) was added, the layers separated and the aqueous layer extracted with ethyl acetate (3×10 ml). The combined organic extracts were washed with a saturated solution of ammonium chloride (300 ml), dried (Na₂SO₄) and concentrated under reduced pressure. Purification by flash chromatography, eluting with ethyl acetate:hexanes gave (±)-6-bromo-5-ethynyl-1,2,3,4-tetrahydro-2,2,4α,8-tetramethyl-3β-(trimethylsilyloxy)quinoline (27 mg, 25%).

### (±)-5-Ethynyl-1,2,3,4-tetrahydro-3β-hydroxy-6-(indol-7-yl)-2,2,4α,8-tetramethylquinoline (Compound 277, Structure 70 of Scheme XIX, where R¹=Me, R²=H, R⁵=α-Me, R⁶=β-OH, R⁹=H, Ar=indol-7-yl)

This compound was prepared according to General Method 5 (EXAMPLE 1) from (±)-6-bromo-5-ethynyl-1,2,3,4-tetrahydro-2,2,4α,8-tetramethyl-3β-(trimethylsilyloxy) quinoline and 7-(4,4,5,5-tetramethyl-1,3,2-dioxaborolan-2-yl)indole to afford (±)-5-ethynyl-1,2,3,4-tetrahydro-6-(indol-7-yl)-2,2,4α,8-tetramethyl-3β-(trimethylsilyloxy)quinoline, which was dissolved in THF. This was subsequently treated with 1M tetrabutylammonium fluoride in tetrahydrofuran (20 equiv) at 0° C. The reaction solution was stirred for 0.2 h at this temperature, a saturated solution of ammonium chloride was added, then ethyl acetate was added, and the layers separated. The aqueous layer was extracted with ethyl acetate, the combined organic extracts washed with a saturated solution of ammonium chloride, dried (Na₂SO₄) and concentrated under reduced pressure. Purification by flash chromatography, eluting with ethyl acetate:hexanes gave Compound 277 (58%). ¹H NMR (500 MHz, CDCl₃) δ 8.16 (s, 1H), 7.62 (dt, J=7.6, 0.8 Hz, 1H), 7.21-7.14 (m, 3H), 7.05 (s, 1H), 6.59 (dd, J=3.2, 2.1 Hz, 1H), 3.58 (dd, J=7.0, 5.2 Hz, 1H), 3.57 (s, 1H), 3.19 (m, 1H), 3.07 (s, 1H), 2.18 (d, J=0.4 Hz, 3H), 1.86 (d, J=7.1 Hz, 1H), 1.63 (d, J=7.0 Hz, 3H), 1.37 (s, 3H), 1.21 (s, 3H).

### Example 178

### (±)-1,2,3,4-Tetrahydro-3β-hydroxy-6-(indol-7-yl)-2,2,4α,8-tetramethyl-E-(2-phenylethenyl)quinoline (Compound 278, Structure 72 of Scheme XX, where R¹=Me, R²=H, R⁵=α-Me, R⁶=β-OH, R⁹=H, Ar=indol-7-yl, R^{D}=phenyl)

### (±)-6-Bromo-1,2,3,4-tetrahydro-3β-hydroxy-2,2,4α,8-tetramethyl-E-(2-phenylethenyl yl)quinoline

Diethyl benzylphosphonate (0.079 ml, 0.38 mmol) was added dropwise to a suspension of 60% sodium hydride dispersion in mineral oil (31 mg, 0.77 mmol) in tetrahydrofuran (10 ml) at 0° C. A solution of (±)-6-bromo-5-formyl-1,2,3,4-tetrahydro-3β-hydroxy-2,2,4α,8-tetramethylquinoline (EXAMPLE 177) (40 mg, 0.13 mmol) in tetrahydrofuran (3 ml) was added and the reaction stirred at room temperature for 15 h. A saturated solution of ammonium chloride (30 ml) was added, the layers separated and the aqueous layer extracted with ethyl acetate (3×10 ml). The combined organic extracts were washed with a saturated solution of ammonium chloride (300 ml), dried (Na₂SO₄) and concentrated under reduced pressure. Purification by flash chromatography, eluting with ethyl acetate:hexanes gave (±)-6-bromo-1,2,3,4-tetrahydro-3β-hydroxy-2,2,4α,8-tetramethyl-E-(2-phenylethenyl)quinoline-(23 mg, 46%).

### (±)-1,2,3,4-Tetrahydro-3β-hydroxy-6-(indol-7-yl)-2,2,4α,8-tetramethyl-E-(2-phenylethenyl)quinoline (Compound 278, Structure 72 of Scheme XX, where R¹=Me, R²=H, R⁵=α-Me, R⁶=β-OH, R⁹=H, Ar=indol-7-yl, R^{D}=phenyl)

This compound was prepared according to General Method 5 (EXAMPLE 1) from (±)-6-bromo-1,2,3,4-tetrahydro-3β-hydroxy-2,2,4α,8-tetramethyl-E-(2-phenylethenyl)quinoline and 7-(4,4,5,5-tetramethyl-1,3,2-dioxaborolan-2-yl)indole to afford compound 278. ¹H NMR (500 MHz, CDCl₃) δ 8.19 (s, 0.5H), 7.88 (s, 0.5H), 7.55 (m, 1H), 7.23-6.87 (m, 9H), 6.62-6.20 (m, 2H), 3.59 (s, 1H), 3.54 (m, 1H), 3.23 (m, 1H), 2.17 (s, 3H), 1.99 (br s, 0.5H), 1.90 (br s, 0.5H), 1.39 (d, J=6.7 Hz, 3H), 1.38 (s, 3H), 1.25 (s, 3H).

### Example 179

### (±)-5-Carbomethoxy-1,2,3,4-tetrahydro-6-(indol-7-yl)-2,2,4,8-tetramethylquinoline (Compound 279, Structure 6 of Scheme I, where R¹=Me, R²=H, R⁴=CO₂Me, R⁵=Me, R⁶=H, R⁹=H, Ar=indol-7-yl)

This compound was prepared from methyl 3-amino-4-methylbenzoate using General Method 1 (EXAMPLE 1), General Method 2 (EXAMPLE 1), and General Method 3 (EXAMPLE 1) to afford (±)-6-bromo-5-carbomethoxy-1,2,3,4-tetrahydro-2,2,4,8-tetramethylquinoline. (±)-6-Bromo-5-carbomethoxy-1,2,3,4-tetrahydro-2,2,4,8-tetramethylquinoline and 7-(4,4,5,5-tetramethyl-1,3,2-dioxaborolan-2-yl)indole were treated according to General Method 5 (EXAMPLE 1) to afford compound 279. MS (EI) 362 (M⁺).

### Example 180

### (±)-5-Carboxy-1,2,3,4-tetrahydro-6-(indol-7-yl)-2,2,4,8-tetramethylquinoline (Compound 280, Structure 6 of Scheme I, where R¹=Me, R²=H, R⁴=CO₂H, R⁵=Me, R⁶=H, R⁹=H, Ar=indol-7-yl)

This compound was prepared by stirring in a 2M potassium hydroxide solution. The reaction was neutralized with saturated ammonium chloride, then extracted with ethyl acetate. The organic layer washed with brine, dried over magnesium sulfate, filtered, and concentrated. Flash chromatography (6:1 hexanes:ethyl acetate) afforded compound 280. ¹H NMR (500 MHz, CD₃OD) δ 8.02-8.06 (m, 3H), 7.66 (d, J=7.8, 1H), 7.45 (t, J=7.8, 1H), 6.95 (d, J=3.4, 1H), 4.72-4.80 (m, 1H), 2.38 (s, 3H), 2.03 (dd, J=13.2, 7.3, 1H), 1.80 (dd, J=13.2, 6.3, 1H), 1.46 (s, 3H), 1.39 (d, J=6.8, 3H), 1.29 (s, 3H).

### Example 181

### (±)-5-Chloro-1,2,3,4-tetrahydro-6-(6-methoxy-3-methylindol-7-yl)-2,2,4,8-tetramethylquinoline (Compound 281, Structure 51 of Scheme XIII, where R¹=Me, R²=H, R⁴=Cl, R⁵=Me, R⁶=H, R⁹=H, R²=H, R²⁵=OMe, R^{A}=H, R^{B}=Me)

This compound was prepared according to General Method 13 (EXAMPLE 163) from Compound 157 (EXAMPLE 57) and 1-propenyl magnesium bromide to afford compound 281. ¹H NMR (500 MHz, CDCl₃) δ 7.48 (d, J=8.6 Hz, 1H), 6.93 (d, J=8.6 Hz, 0.5H), 6.92 (d, J=8.6 Hz, 0.5H), 6.92 (s, 1H) 6.83 (q, J=1.1 Hz, 0.5H), 6.81 (q, J=1.1 Hz, 0.5H), 3.82 (s, 1.5H), 3.81 (s, 1.5H), 3.56 (m, 1H), 3.37 (m, 1H), 2.32 (d, J=1.1 Hz, 1.5H), 2.32 (d, J=1.1 Hz, 1.5H), 2.08 (s, 3H), 1.98 (dd, J=13.5, 7.0 Hz, 0.5H), 1.97 (dd, J=13.5, 7.0 Hz, 0.5H), 1.81 (dd, J=13.5, 4.2 Hz, 0.5H), 1.80 (dd, J=13.5, 4.2 Hz, 0.5H), 1.46 (d, J=7.0 Hz, 1.5H), 1.44 (d, J=7.0 Hz, 1.5H), 1.40 (s, 1.5H), 1.39 (s, 1.5H), 1.27 (s, 1.5H), 1.26 (s, 1.5H).

### Example 182

### (±)-5-Chloro-1,2,3,4-tetrahydro-3β-hydroxy-2,2,4α,8-tetramethyl-6-(oxazol-5-yl)quinoline (Compound 282, Structure 76)

This compound was prepared from (±)-5-chloro-1,2,3,4-tetrahydro-3β-hydroxy-2,2,4α,8-tetramethylquinoline, triisopropylsilyl triflate and 2,6-lutidine to afford (±)-5-chloro-1,2,3,4-tetrahydro-2,2,4α,8-tetramethyl-3β-(triisopropylsilyloxy)quinoline. Then this compound was combined with a mixture of POCl₃ in DMF at −10° C. to afford (±)-5-chloro-6-formyl-1,2,3,4-tetrahydro-2,2,4α,8-tetramethyl-3β-(triisopropylsilyloxy)quinoline. This compound was heated with a mixture of tosylmethylisocyamide, potassium carbonate, and methanol to afford compound 282. ¹H NMR (300 MHz, CDCl₃) δ 7.88 (s, 1H), 7.54 (s, 1H), 7.36 (m, 1H), 3.70 (s, 1H), 3.59 (m, 1H), 3.13 (dq, J=4.9, 7.0 Hz, 1H), 2.15 (d, J=0.6 Hz, 3H), 1.94 (d, J=7.0 Hz, 1H), 1.51 (d, J=7.0 Hz, 3H), 1.35 (s, 3H), 1.20 (s, 3H).

### Example 183

### (±)-5-Chloro-1,2,3,4-tetrahydro-3β-hydroxy-6-(5-methoxyindol-7-yl)-2,2,4α,8-tetramethylquinoline (Compound 283, Structure 6 of Scheme I, where R¹=Me, R²=H, R⁴=Cl, R⁵=α-Me, R⁶=β-OH, R⁹=H, Ar=5-methoxyindol-7-yl)

This compound was prepared according to General Method 12 (EXAMPLE 162) from (±)-5-chloro-1,2,3,4-tetrahydro-3β-hydroxy-2,2,4α,8-tetramethyl-6-(4,4,5,5-tetramethyl-1,3,2-dioxaborolan-2-yl)quinoline (EXAMPLE 63) and 2-bromo-3-nitroanisole to form atropisomers of (±)-5-chloro-1,2,3,4-tetrahydro-3β-hydroxy-2,2,4α,8-tetramethyl-6-(6-methoxy-2-nitrophenyl)quinoline after purification on silica gel chromatography (EtOAc:hexanes). The product that runs faster on silica gel was treated according to General Method 13 (EXAMPLE 163) and vinyl magnesium bromide from (±)-5-chloro-1,2,3,4-tetrahydro-3β-hydroxy-2,2,4α,8-tetramethyl-6-(6-methoxy-2-nitrophenyl)quinoline to afford one of the atropisomers of compound 283. ¹H NMR (500 MHz, CDCl₃) δ 7.80 (s, 1H), 7.61 (d, J=8.5 Hz, 1H), 7.10 (dd, J=3.0, 2.3 Hz, 1H), 7.01 (s, 1H), 6.97 (d, J=8.5 Hz), 6.54 (dd, J=3.0, 2.3 Hz, 1H), 3.87 (s, 3H), 3.68-3.62 (m, 2H), 3.19 (dq, J=4.3, 7.0 Hz, 1H), 2.16 (s, 3H), 1.94 (m, 1H), 1.59 (d, J=7.0 Hz, 3H), 1.40 (s, 3H), 1.30 (s, 3H).

### Example 184

### (±)-5-Chloro-1,2,3,4-tetrahydro-3β-hydroxy-2,2,4α,8-tetramethyl-6-(pyrid-4-yl)quinoline (Compound 284, Structure 6 of Scheme I, where R¹=Me, R²=H, R⁴=Cl, R⁵=α-Me, R⁶=β-OH, R⁹=H, Ar=pyrid-4-yl)

This compound was prepared according to General Method 5 (EXAMPLE 1) from (±)-6-bromo-5-chloro-1,2,3,4-tetrahydro-3β-hydroxy-2,2,4α,8-tetramethylquinoline and 4-pyridineboronic acid to afford compound 284. ¹H NMR (500 MHz, CDCl₃) δ 8.58-8.62 (m, 1H), 7.32-7.38 (m, 1H), 6.90 (s, 1H), 3.65 (broad s, 1H), 3.59-3.62 (m, 1H), 3.10-3.18 (m, 1H), 2.13 (s, 3H), 1.92 (d, J=7.8, 1H), 1.52 (d, J=7.3, 3H), 1.36 (s, 3H), 1.22 (s, 3H).

### Example 185

### (±)-5-Cyano-1,2,3,4-tetrahydro-3β-hydroxy-6-(indolin-7-yl)-2,2,4α,8-tetramethylquinoline (Compound 285, Structure 58 of Scheme XV, where R¹=Me, R²=H, R⁴=CN, R⁵=α-Me, R⁶=β-OH, R⁹=H)

This compound was prepared from Compound 276 (EXAMPLE 176) and sodium cyanoborohydride (>10 equiv) in acetic acid was stirred at room temperature for 2 hours. The mixture was partitioned between ethyl acetate and aqueous sodium bicarbonate. The solution was dried over magnesium sulfate, filtered, and concentrated. The compound was purified by silica gel chromatography to afford compound 285. ¹H NMR (500 MHz, CDCl₃) δ 7.12 (m, 1H), 7.09 (q, J=0.6 Hz, 1H), 7.03 (d, J=7.4 Hz, 1H), 6.78 (t, J=7.4 Hz, 1H), 3.79 (s, 1H), 3.63 (s, 1H), 3.60-3.51 (m, 3H), 3.15-3.07 (m, 3H), 2.17 (d, J=0.6 Hz, 3H), 1.83 (d, J=7.2 Hz, 1H), 1.63 (d, J=7.1 Hz, 3H), 1.35 (s, 3H), 1.17 (s, 3H).

### Example 186

### (±)-5-Chloro-1,2,3,4-tetrahydro-3α-methoxy-2,2,4α,8-tetramethyl-6-(naphthal-1-yl)quinoline (Compound 286, Structure 77 of Scheme XXIII, where R¹=Me, R²=H, R⁴=Cl, R⁵=cis-Me, Ar=naphthal-1-yl R=Me)

To prepare this compound, compound 195 (EXAMPLE 95) (6 mg) was dissolved in 2 mL THF. NaHMDS (1 M, 66 microliters) and MeI (50 microliters) was added and stirred for 30 min. The reaction was partitioned with water and ethyl acetate, washed with brine, dried over magnesium sulfate, and filtered. Flash chromatography (EtOAc:hexanes, 1:4) afforded Compound 286. ¹H NMR (500 MHz, CDCl₃) δ 7.88 (d, J=7.8, 1H), 7.85 (d, J=8.3, 1H), 7.62 (d, J=8.2, 0.5H), 7.57 (d, J=8.3, 0.5H), 7.53-7.33 (m, 4H), 6.90 (s, 1H), 3.71-3.65 (m, 1H), 3.60 (broad s, 1H), 3.51 (s, 1.5H), 3.50 (s, 1.5H), 3.46 (d, J=6.3, 0.5H), 3.43 (d, J₁=6.3, 0.5H), 2.10 (s, 3H), 1.40-1.34 (m, 9H).

### Example 187

### (±)-1,2,3,4-Tetrahydro-3β-hydroxy-6-(indolin-7-yl)-5-(methoxyimino)-2,2,4α,8-tetramethylquinoline (Compound 287, Structure 79 of Scheme XXIV, where R¹=Me, R²=H, R⁵=α-Me, R⁶=β-OH, R⁹=H, R=Me)

To prepare this compound, a mixture of (±)-5-formyl-1,2,3,4-tetrahydro-3β-hydroxy-2,2,4α,8-tetramethylquinoline (EXAMPLE 177), methoxyamine hydrochloride (3 equiv) in ethanol was heated at 45° C. for 1 h. The mixture was extracted with water and ethyl acetate. The organic layer washed with saturated ammonium chloride, dried over sodium sulfate, filtered and concentrated. The resultant oil was subjected to the bromination conditions of General Method 3 (EXAMPLE 1) and the aryl coupling conditions of General Method 5 (EXAMPLE 1) to afford compound 287. MS (electrospray): 378.2 (M+H).

### Example 188

### (±)-1,2,3,4-Tetrahydro-5-(hydroxymethyl)-6-(indol-7-yl)-2,2,4,8-tetramethylquinoline (Compound 288, Structure 84 of Scheme XXV, where Ar=indol-7-yl)

To prepare this compound, (±)-6-Bromo-5-carbomethoxy-1,2,3,4-tetrahydro-2,2,4,8-tetramethylquinoline (EXAMPLE 179) (470 mg) was dissolved in 15 mL ether. Lithium aluminum hydride (148 mg) was added in 2 portions at ° C. The reaction was allowed to warm to rt and was stirred for 3 h. The reaction was quenched with a saturated solution of Rochelle's salt, and extracted with EtOAc. The reaction was dried over sodium sulfate, filtered, and concentrated to afford 275 mg of (±)-6-bromo-1,2,3,4-tetrahydro-5-(hydroxymethyl)-2,2,4,8-tetramethylquinoline. (±)-6-Bromo-1,2,3,4-tetrahydro-5-(hydroxymethyl)-2,2,4,8-tetramethylquinoline (60 mg) was treated according to General Method 5 (EXAMPLE 1) with 7-(4,4,5,5-tetramethyl-1,3,2-dioxaborolan-2-yl)indole (60 mg) to afford compound 288. ¹H NMR (500 MHz, CDCl₃) δ 8.51 (broad s, ½H), 8.29 (broad s, ½H), 7.59 (d, J=7.6, 1H), 7.05-7.20 (m, 3H), 6.92 (d, J=5.5, 1H), 6.57 (m, 1H), 4.35-4.60 (m, 3H), 3.51 (broad s, 1H), 3.40-3.50 (m, 1H), 2.12 (s, 3/2H), 2.11 (s, 3/2H), 1.80-2.00 (m, 2H), 1.46 (t, J=5.5, ½H), 1.41 (s, 3H), 1.35-1.45 (6H), 1.36 (t, J=5.6, ½H), 1.25 (s, 3/2H), 1.24 (3/2H).

### Example 189

### (±)-5-(3-(2-Fluoroethoxy)benzyloxymethyl)-1,2,3,4-tetrahydro-6-(indol-7-yl)-2,2,4,8-tetramethylquinoline (Compound 289, Structure 83 of Scheme XXV, where Ar=indol-7-yl R=3-(2-fluoroethoxy)benzyl)

To prepare this compound, a solution of (±)-6-bromo-1,2,3,4-tetrahydro-5-(hydroxymethyl)-2,2,4,8-tetramethylquinoline (EXAMPLE 188) (30 mg), 3-(2-fluoroethoxy)benzyl bromide (50 mg), NaH (60% mineral oil dispersion, 10 mg) in 1 mL DMF was stirred at room temperature for 2 h. The reaction was quenched with water, extracted with ethyl acetate, and the organic layer washed with brine. The organic layer was dried over sodium sulfate, filtered, and concentrated. Flash chromatography (4:1 hexanes:ethyl acetate afforded (±)-6-bromo-1,2,3,4-tetrahydro-5-(3-(2-fluoroethoxy)benzyloxymethyl)-2,2,4,8-tetramethylquinoline. (±)-6-Bromo-1,2,3,4-tetrahydro-5-(3-(2-fluoroethoxy)benzyloxymethyl)-2,2,4,8-tetramethylquinoline was treated according to General Method 5 (EXAMPLE 1) with 7-(4,4,5,5-tetramethyl-1,3,2-dioxaborolan-2-yl)indole to afford compound 289. ¹H NMR (500 MHz, CD₃OD) δ 9.8 (m, 1H), 7.54 (d, J=7.8, 1H), 7.12-7.21 (m, 2H), 7.04-7.08 (m, 1H), 6.96-7.01 (m, 1H), 6.81-6.87 (m, 2H), 6.66-6.77 (m, 2H), 6.48-6.52 (m, 1H), 4.76-4.80 (m, 1H), 4.66-4.70 (m, 1H), 4.10-4.45 (m, 6H), 3.38-3.48 (m, 1H), 2.20 (s, 3H), 1.84-1.98 (m, 4H), 1.45 (s, 3H), 1.38 (s, 3/2H), 1.37 (s, 3/2H).

### Example 190

### (±)-5-((6-Fluoro-4H-benzo[1,3]dioxin-8-yl)methoxymethyl)-1,2,3,4-tetrahydro-6-(indol-7-yl)-2,2,4,8-tetramethylquinoline (Compound 289, Structure 83 of Scheme XXV, where Ar=indol-7-yl R=(6-fluoro-4H-benzo[1,3]dioxin-8-yl)methyl)

To prepare this compound, a solution of (±)-6-bromo-1,2,3,4-tetrahydro-5-(hydroxymethyl)-2,2,4,8-tetramethylquinoline (EXAMPLE 188) (30 mg), 8-chloromethyl-6-fluoro-4H-[1,3]-benzodioxine (50 mg), tetra-n-butylammonium iodide (10 mg) and sodium bis(trimethylsilyl)amide (1M in THF, excess) in 2 mL THF was stirred at RT. The reaction was quenched with water, extracted with ethyl acetate, and the organic layer washed with brine. The organic layer was dried over sodium sulfate, filtered, and concentrated. Flash chromatography (EtOAc:hexanes) afforded 16 mg of (±)-6-bromo-5-((6-fluoro-4H-benzo[1,3]dioxin-8-yl)methoxymethyl)-1,2,3,4-tetrahydro-6-(indol-7-yl)-2,2,4,8-tetramethylquinoline. (±)-6-Bromo-5-((6-fluoro-4H-benzo[1,3]dioxin-8-yl)methoxymethyl)-1,2,3,4-tetrahydro-6-(indol-7-yl)-2,2,4,8-tetramethylquinoline (15 mg) was treated according to General Method 5 (EXAMPLE 1) with 7-(4,4,5,5-tetramethyl-1,3,2-dioxaborolan-2-yl)indole to afford compound 290. ¹H NMR (500 MHz, CDCl₃) δ 8.96 (s, ½H), 8.72 (s, ½H), 7.59 (d, J=7.8, 1H), 7.05-7.16 (m, 2H), 7.01 (t, J=2.6, 1H), 6.96 (d, J=7.8, 1H), 6.82-6.90 (m, 1H), 6.56-6.64 (m, 1H), 6.52-6.56 (m, 1H), 5.18 (s, 1H), 5.15 (s, 1H), 4.87 (1H), 4.10-4.50 (m, 4H), 3.3-3.5 (m, 2H), 2.15 (s, 3/2H), 2.14 (s, 3/2H), 1.85-2.0 (m, 2H), 1.32-1.45 (6H), 1.27 (s, 3/2H), 1.25 (s, 3/2H).

### Example 191

### (±)-5-(2-Fluoro-3-methylbenzyloxymethyl)-1,2,3,4-tetrahydro-6-(indol-7-yl)-2,2,4,8-tetramethylquinoline (Compound 289, Structure 83 of Scheme XXV, where Ar=indol-7-yl R=2-fluoro-3-methylbenzyl)

To prepare this compound, a solution of (±)-6-bromo-1,2,3,4-tetrahydro-5-(hydroxymethyl)-2,2,4,8-tetramethylquinoline (EXAMPLE 188) (30 mg), 2-fluoro-3-methylbenzyl bromide (38 mg), NaH (60% mineral oil dispersion) in 1 mL DMF was stirred at rt for 2 h. The reaction was quenched with water, extracted with ethyl acetate, and the organic layer washed with brine. The organic layer was dried over sodium sulfate, filtered, and concentrated. Flash chromatography (EtOAc:hexanes) afforded (±)-6-bromo-5-(2-fluoro-3-methylbenzyloxymethyl)-1,2,3,4-tetrahydro-2,2,4,8-tetramethylquinoline. (±)-6-Bromo-5-(2-fluoro-3-methylbenzyloxymethyl)-1,2,3,4-tetrahydro-2,2,4,8-tetramethylquinoline was treated according to General Method 5 (EXAMPLE 1) with 7-(4,4,5,5-tetramethyl-1,3,2-dioxaborolan-2-yl)indole to afford compound 291. ¹H NMR (500 MHz, CDCl₃) δ 9.11 (s, ½H), 8.88 (s, ½H), 7.64 (d, J=7.3, 1H), 6.94-7.20 (m, 8H), 6.59-6.62 (m, 1H), 4.20-4.55 (m, 4H), 3.52-3.56 (m, 1H), 3.34-3.54 (m, 1H), 2.28-2.34 (3H), 2.18 (s, 3/2H), 2.17 (s, 3/2H), 1.90-2.02 (m, 2H), 1.44-1.50 (4.5H), 1.37 (d, J=6.8, 3/2H), 1.29 (s, 3H).

### Example 192

Glucocorticoid Binding Assays

Preparation of GR

A baculovirus expression plasmid comprising cDNA encoding the human glucocorticoid receptor protein (GR) was prepared using standard techniques. See e.g. E. A. Allegretto et. al. 268 J. Biol. Chem., 26625 (1993); G. Srinivasan and B. Thompson, 4 Mol. Endo., 209 (1990); and D. R. O'Reilly et. al., in “Baculovirus Expression Vectors”, D. R. O'Reilly et. al., eds., W. H. Freeman, New York, N.Y., pp. 139-179 (1992). That expression plasmid was co-transfected together with wild type Autographa californica multiple nuclear polyhedrosis virus DNA into Spodopter frugiperda-21 (Sf-21) cells to generate recombinant virus comprising GR cDNA. See e.g. O'Reilly, D. R., Miller, L. K., Luckow, V. A., Regulation of expression of a baculovirus ecdysteroid UDP glucosyltransferase gene. “Baculovirus Expression Vectors.” WH Freeman, NY, 139-179 (1992). That recombinant virus comprising GR cDNA was collected.

A suspension culture of uninfected Sf21 cells was grown to a density of 1.2×10⁶ cells/ml and then infected with the recombinant virus comprising GR cDNA at a multiplicity of infection of 2. Those infected Sf21 cells were incubated for 48 hours and then collected by centrifugation at 1000×g for 10 minutes at 4° C. The resulting cell pellets were resuspended in lysis buffer (50 mM Potassium Phosphate buffer, pH 7.0, 10 mM Monothioglycerol, 5 mM DTT, 20 mM Sodium Molybdate, 1 mM PMSF, 1 μg/mL aprotinin, and 10 μg/mL leupeptin) and incubated for 15 minutes on ice. Those resuspended cell pellets were homogenized using a Dounce homogenizer and a B pestle. A volume of 2 M KCl was added to the homogenized cell pellets to a final concentration of 0.4 M. The resulting GR lysates were centrifuged at 100,000×g for 60 min at 4° C. and stored for use in binding assays.

Binding Assays

Binding assay samples were prepared in separate mini-tubes in a 96-well format at 4° C. Each binding assay sample was prepared in a volume of 250 μl of GR-Assay Buffer (10% glycerol, 25 mM sodium phosphate, 10 mM potassium fluoride, 10 mM sodium molybdate, 0.25 mM CHAPS, 2 mM DTT and 1 mM EDTA, (adjusted to pH 7.5)) containing 50 μg of GR lysate; 2-4 nM of [³H]dexamethasone at 84 Ci/mmol; and either a reference compound or a test compound. Test compounds included selective glucocorticoid binding compounds of the present invention. Reference compounds were unlabeled dexamethasone and prednisone, which have been previously shown to bind to glucocorticoid receptors. Each reference compound and test compound was assayed at varying concentrations, ranging from 0 to 10⁻⁵ M. Each concentration of each reference compound and each test compound was assayed in triplicate. The assay samples were incubated for 16 hours at 4° C.

After incubation, 200 μl of 6.25% hydroxylapatite in assay buffer was added to each assay sample to precipitate the protein. The assay samples were then centrifuged and the supernatants were discarded. The resulting pellets were washed twice with assay buffer lacking DTT. Radioactivity in counts per minute (CPM) of each washed pellet was determined by liquid scintillation counter (MicroBeta™, Wallach).

Specific binding for a particular sample was calculated using the equation:
(Sample CPM)−(Average Non-specific CPM)
Average Non-specific CPM was defined as the amount of radioactivity from samples comprising an excess (i.e. 1000 nM) of unlabeled dexamethasone. IC₅₀ values (the concentration of test compound required to decrease specific binding by 50%) were determined using the log-logit (Hill) method. Kᵢ values were determined using the Cheng-Prusoff equation using a previously determined K_{d} value for dexamethasone:
Kᵢ=IC₅₀/(1+[L]/K_{d})
[L]=concentration of labeled dexamethasone
K_{d}=dissociation constant of labeled dexamethasone

For a discussion of the calculation of Kᵢ, see e.g. Cheng, Y. C. and Prusoff, W. H. Biochem. Pharmacol. 22:3099 (1973). Kᵢ values for certain glucocorticoid binding compounds are shown in Table 1.

TABLE 1
GR Binding Data

| Compound | Kᵢ | Compound | Kᵢ |
|---|---|---|---|
| 104 | 34 | 160 | 130 |
| 149 | 0.6 | 161 | 1.8 |
| 109 | 4 | 213 | 2.5 |
| 116 | 420 | 215 | 5.9 |
| 118 | 22 | 165 | 2.7 |
| 119 | 97 | 185 | 2.5 |
| 121 | 14 | 179 | 2.6 |
| 122 | 78 | 192 | 9.4 |
| 130 | 81 | 193 | 10 |
| 134 | 24 | 194 | 13 |
| 135 | 8.8 | 186 | 5.8 |
| 139 | 11 | 189 | 6 |
| 141 | 1.3 | 196 | 18 |
| 147 | 2 | 203 | 56 |
| 210 | 5.7 | 204 | 26 |
| 151 | 3.6 | 205 | 87 |
| 154 | 0.4 | 191 | 4.8 |
| 155 | 1.4 | 209 | 1.6 |
| 156 | 2.9 | Dex | 1.9 |
| 164 | 3.3 | Pred | 5.3 |

### Example 159

Mineralocorticoid Binding Assays

Preparation of MR

Human mineralocorticoid receptor protein was prepared from a baculovirus expression plasmid comprising cDNA encoding human mineralocorticoid Receptor-α (MRα), as described for GR (Example 158).

Binding Assays

Binding assay samples were prepared in separate mini-tubes in a 96-well format at 4° C. Each binding assay sample was prepared in a volume of 250 μl of MR-Assay Buffer (10% glycerol, 10 mM sodium phosphate, 10 mM potassium fluoride, 20 mM sodium molybdate, 0.25 mM CHAPS, 2 mM DTT, (adjusted to pH 7.35)) containing 5-10 μg of MR lysate; 2-4 nM of [³H]aldosterone; unlabeled aldosterone; and a test compound. Each test compound was assayed at several different concentrations, ranging from 0 to 10⁻⁵ M and each was tested in the presence and in the absence of several different concentrations of unlabeled aldosterone. Each concentration of each test compound was assayed in triplicate. The assay samples were incubated for 16 hours at 4° C.

After incubation, protein was precipitated with hydroxylapatite, collected, and counted as described in Example 158 for GR. Specific binding for a particular sample was calculated using the same equation as was used for GR:
(Sample CPM)−(Average Non-specific CPM)
Average Non-specific CPM was defined as the amount of radioactivity from samples comprising an excess (i.e. 1000 nM) of unlabeled aldosterone. IC₅₀ values (the concentration of test compound required to decrease specific binding by 50%) were determined using the log-logit (Hill) method. Kᵢ values were determined using the Cheng-Prusoff equation using a previously determined K_{d} value for aldosterone:
Kᵢ=IC₅₀/(1+[L]/K_{d})
[L]=concentration of labeled aldosterone
K_{d}=dissociation constant of labeled aldosterone

Kᵢ values for certain mineralocorticoid receptor binding compounds are shown in Table 2.

TABLE 2
MR Binding Data

| | |
|---|---|
| Compound | Kᵢ (nM) |

| | |
|---|---|
| 230 | 12 |
| 234 | 85 |
| 238 | 18 |
| 239 | 22 |
| 244 | 6 |
| 245 | 47 |
| 256 | 60 |

1. A compound of Formula I, II, or III: 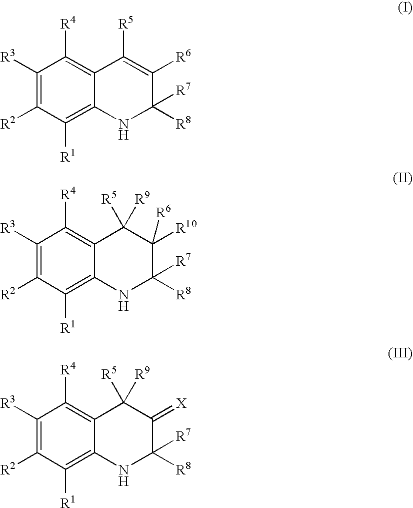
or a pharmaceutically acceptable salt, ester, amide, or prodrug thereof,
wherein:
R¹ and R² are each independently selected from the group consisting of hydrogen, a halogen, —CN, —OR¹⁶, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R³ is selected from the group consisting of (a), (b), (c), (d), (e), (f), (g), (h), (i), (j), (k), (l), (m), and (n) 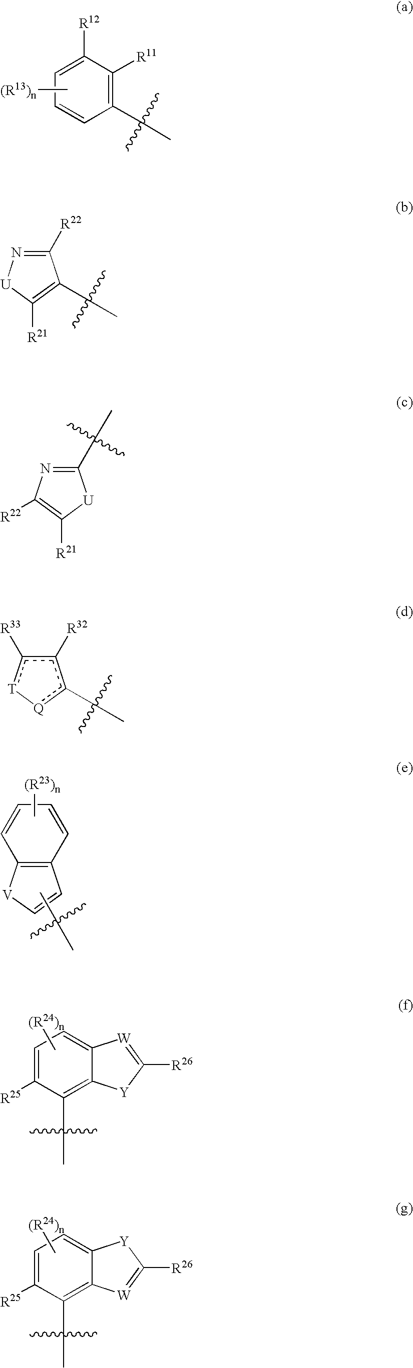 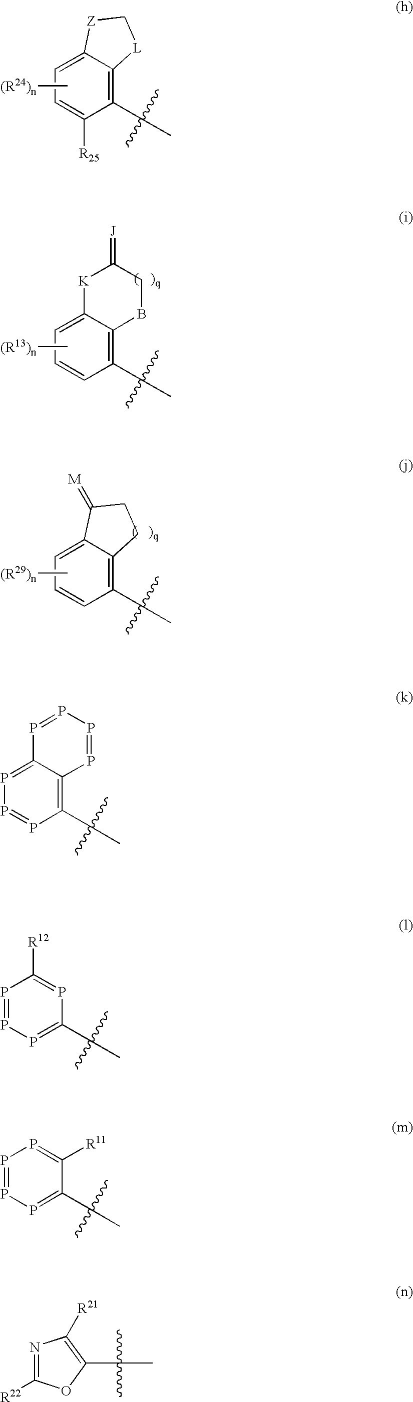
wherein,
R¹¹ is selected from the group consisting of hydrogen, a halogen, —CN, —OR¹⁶, —NR¹⁷R¹⁸, —CH₂R¹⁶, —COR²⁰, —CO₂R²⁰, —CONR²⁰R³⁷, —SOR²⁰, —SO₂R²⁰—NO₂, NR¹⁷ (OR¹⁶), an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R¹² is selected from the group consisting of hydrogen, a halogen, —CN, —COR²⁰, —CO₂R²⁰, —CONR²⁰R³⁷, —NR¹⁷SO₂R²⁰, —NR¹⁷CO₂R²⁰, —NO₂, —OR¹⁶, —NR¹⁷R¹⁸, NR¹⁷(OR¹⁶) an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl or R¹² taken together with R¹ form a 3-7 membered ring;
each R¹³ is independently selected from the group consisting of hydrogen, a halogen, CN, —NO₂, OR¹⁶, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl or R¹³ taken together with R¹² form a 3-7 membered ring;
R²¹ is selected from the group consisting of hydrogen, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R²² is selected from the group consisting of hydrogen, a halogen, —NR¹⁷R¹⁸ an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R³² and R³³ are each independently selected from the group consisting of hydrogen, a halogen, —OR¹⁶, —CN, COR²⁰, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
each R²³ is independently selected from the group consisting of hydrogen, a halogen, OR¹⁶, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
each R²⁴ is independently selected from the group consisting of hydrogen, a halogen, an optionally substituted C₁-C₆alkyl, and —OR¹⁶;
R²⁵ is selected from the group consisting of hydrogen, a halogen, —OR¹⁶, —CN, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R²⁶ is selected from the group consisting of hydrogen, a halogen, —OR¹⁶, —CN, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
each R²⁹ is independently selected from the group consisting of hydrogen, a halogen, and —OR¹⁶;
U is selected from the group consisting of oxygen, sulfur, and —NR¹⁷;
Q and T are each selected from the group consisting of S, O, —NR¹⁷ and CR³⁴ wherein
either Q is —CR³⁴ and T is selected from the group consisting of S, O, and —NR¹⁷,
or T is CR³⁴ and Q is selected from the group consisting of S, O, and —NR¹⁷;
V is selected from the group consisting of O, S, and —NR¹⁷;
W is selected from the group consisting of —CR²⁷ and N;
Y is selected from the group consisting of —NR³⁶, S, and O;
Z and L are each selected from the group consisting of CH₂, —NR²⁸, and O, wherein
either Z is CH₂ and L is selected from the group consisting of —NR²⁸ and O.
or L is CH₂ and Z is selected from the group consisting of —NR²⁸ and O;
K is selected from the group consisting of O and —NR³⁵;
J is selected from the group consisting of O and S;
B is selected from the group consisting of O and CR²⁷;
M is selected from the group consisting of O and —NOR³⁰;
each P is independently selected from the group consisting of N and CR³¹, provided that no more than two of the Ps are N;
n is selected from 0, 1, 2, 3, and 4; and
q is selected from 0, 1, and 2;
R⁴ is selected from the group consisting of hydrogen, a halogen, NO₂, OR¹⁶, NR¹⁷R¹⁸, CN, C═N(OR¹⁶), CO₂R²⁰, CONR²⁰R³⁷, NR¹⁷(OR¹⁶) CR³(OR¹⁶), an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R⁵ is selected from the group consisting of hydrogen, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R⁶ is selected from the group consisting of hydrogen and OR¹⁶;
R⁷ and R⁸ are each independently selected from the group consisting of hydrogen, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R⁹ is selected from the group consisting of hydrogen, OR¹⁶, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R¹⁰ is selected from the group consisting of hydrogen and OR¹⁶; and
X is selected from the group consisting of O, S, and NOR¹⁶;
wherein:
R¹⁶ is selected from the group consisting of hydrogen, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R¹⁷ and R¹⁸ are each independently selected from the group consisting of hydrogen, COR²⁰, CO₂R²⁰, SO₂R²⁰, S(O)R²⁰, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl; or R¹⁷ and R¹⁸ together form a 3 to 7 membered ring;
R²⁰ and R³⁷ are each independently selected from the group consisting of hydrogen, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl; or R³⁷ and R²⁰ together form a 3-7 membered ring;
R³⁴ is selected from the group consisting of hydrogen, a halogen, —NO₂, —OR¹⁶, —NR¹⁷R¹⁸, —CN, —COR²⁰, NR¹⁷(OR¹⁶), an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R³⁶ is selected from the group consisting of hydrogen, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R²⁷ is selected from the group consisting of hydrogen, a halogen, CO₂R²⁰, COR²⁰, CONR²⁰R³⁷, C═N(OR¹⁶), an optionally substituted C₁-C₈ alkyl, an optionally substituted C₁-C₈ heteroalkyl, an optionally substituted C₁-C₈ haloalkyl, an optionally substituted C—C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl or R²⁷ taken together with R²⁶ form a 3-7 membered ring;
R²⁸ is selected from the group consisting of hydrogen, —COR²⁰, —CO₂R²⁰, —CONR²⁰R³⁷, SO₂R²⁰, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R³⁵ is selected from the group consisting of hydrogen, —COR²⁰, —CO₂R²⁰, CONR²⁰R³⁷, SO₂R²⁰, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R³⁰ is selected from the group consisting of hydrogen an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl; and
R³¹ is selected from the group consisting of hydrogen, a halogen, an optionally substituted C₁-C₆ alkyl, and —OR¹⁶;
wherein,
at least one of R¹, R² and R⁴ is not hydrogen; and
at least one of R¹¹, R¹², and one R¹³ is not hydrogen.

2. The compound of claim 1, wherein
R³ is selected from the group consisting of (a), (b), (c), (d), (e), (f), (g), (h), (i), (j) (k), (l), and (m): 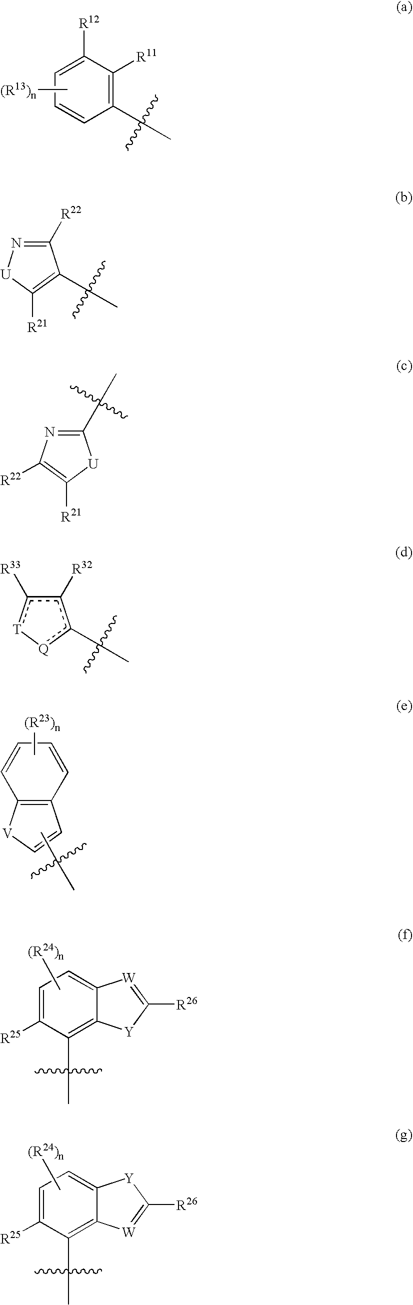 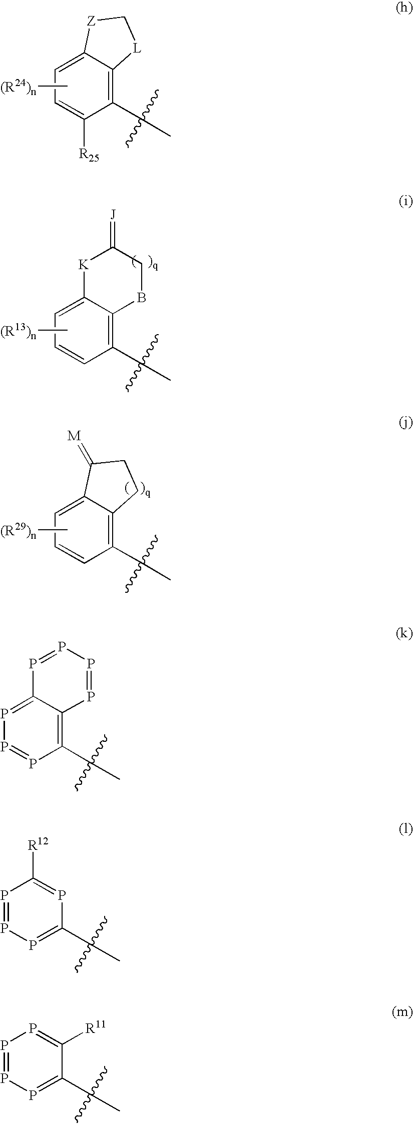
R⁴ is selected from hydrogen, a halogen, and an optionally substituted C₁-C₈ alkyl;
R⁷ and R⁸ are each independently selected from the group consisting of hydrogen and methyl;
R¹¹ is selected from the group consisting of hydrogen, halogen, —CN, —OR¹⁶, —NR¹⁷R¹⁸, —COR²⁰, CO₂R²⁰, —CONR²⁰R³⁷, —SOR²⁰, SO₂R²⁰, —NO₂, an optionally substituted C₁-C₈ alkyl, an optionally substituted C₁-C₈ heteroalkyl, an optionally substituted C₁-C₈ haloalkyl;
R¹² is selected from the group consisting of hydrogen, halogen, —CN, —COR²⁰, —NR¹⁷SO₂R²⁰, —NR¹⁷CO₂R²⁰, —NO₂, —OR¹⁶, —NR¹⁷R¹⁸, NR¹⁷(OR¹⁶), an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, or R¹² taken together with R¹¹ form a 3-7 membered ring;
each R¹³ is independently selected from the group consisting of hydrogen, halogen, CN, OR¹⁶, C₁₋₈ alkyl, a C₁-C₈ haloalkyl, or R¹³ taken together with R¹² form a 3-7 membered ring;
R¹⁷ and R¹⁸ are each independently selected from the group consisting of hydrogen, C₁-C₈ alkyl, C₁-C₈ heteroalkyl, and C₁-C₈ haloalkyl, or R¹⁷ and R¹⁸ together form a 3 to 7 membered ring;
R²⁰ and R³⁷ are each independently selected from the group consisting of hydrogen, C₁-C₈ alkyl, C₁-C₈ heteroalkyl, and C₁-C₈ haloalkyl; and
R²¹ is selected from the group consisting of hydrogen, a C₁-C₈ alkyl, a C₁-C₈ heteroalkyl, and a C₁-C₈ haloalkyl
R²² is selected from the group consisting of hydrogen, halogen, —NR¹⁷R¹⁸, a C₁-C₈ alkyl, a C₁-C₈ heteroalkyl, and a C₁-C₈ haloalkyl;
R²⁷ is selected from the group consisting of hydrogen, a halogen, C₁-C₈ alkyl, C₁-C₈ heteroalkyl, and C₁-C₈ haloalkyl;
each R²⁹ is independently selected from the group consisting of hydrogen, halogen, and —OR¹⁶;
U is selected from the group consisting of oxygen, sulfur, and —NR¹⁷;
n is selected from 1 and 2; and
q is selected from 1 and 2.

3. The compound of claim 1, wherein
R¹ and R² are independently selected from the group consisting of hydrogen, F, Cl, methyl, ethyl, and methoxy;
R⁴ is selected from the group consisting of hydrogen, a halogen, and an optionally substituted C₁-C₆ alkyl;
R⁵ is selected from the group consisting of hydrogen, methyl, ethyl, and methoxy;
R⁶ is hydrogen or hydroxy;
R⁷ and R⁸ are each independently hydrogen, methyl, and ethyl;
R⁹ is selected from the group consisting of hydrogen, OR¹⁶, and methyl;
R¹⁰ is selected from the group consisting of hydrogen and OR¹⁶; and
R¹⁶ is hydrogen, an optionally substituted C₁-C₆ alkyl, or an optionally substituted C₁-C₆ heteroalkyl.

4-78. (canceled)

79. The compound of claim 1, wherein R³ is Formula (b), 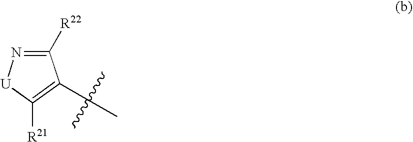
wherein
U is oxygen or —NR¹⁷;
R¹⁷ is selected from the group consisting of hydrogen and a C₁-C₆ alkyl;
R²¹ is selected from the group consisting of hydrogen, a C₁-C₆ alkyl, and a C₁-C₆ haloalkyl; and
R²² is selected from the group consisting of hydrogen, F, Cl, a C₁-C₆ alkyl, a C₁-C₆ a heteroalkyl, and —NR¹⁷R¹⁸.

80-112. (canceled)

113. The compound of claim 1, wherein R³ has the structure of Formula (f) or (g), 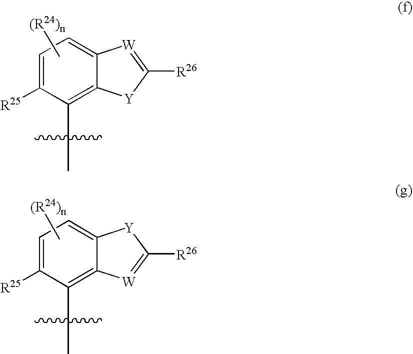
wherein
W is selected from the group consisting of —CR²⁷ and nitrogen;
Y is selected from the group consisting of —NR²⁶, sulfur, and oxygen;
R²⁴ is selected from the group consisting of hydrogen, a halogen, methyl, and methoxy;
R²⁵ is selected from the group consisting of hydrogen, a halogen, —OMe, —CN, and an optionally substituted C₁-C₆ alkyl;
R²⁶ is selected from the group consisting of hydrogen, a halogen, methyl, and methoxy; and
n is 0, 1, or 2.

114-181. (canceled)

182. A compound selected from the group consisting of
(±)-5-Chloro-1,2,3,4-tetrahydro-2,2,4,8-tetramethyl-6-(thiazol-2-yl)quinoline (compound 101),
(±)-6-(4-Acetylthiophen-2-yl)-5-chloro-1,2,3,4-tetrahydro-2,2,4,8-tetramethylquinoline (compound 102),
(±)-5-Chloro-1,2,3,4-tetrahydro-6-(indol-2-yl)-2,2,4,8-tetramethylquinoline (compound 103),
(±)-5-Chloro-6-(2,6-dimethoxyphenyl)-1,2,3,4-tetrahydro-2,2,4,8-tetramethylquinoline (compound 104),
(±)-5-Chloro-6-(3-cyano-2-methoxyphenyl)-1,2,3,4-tetrahydro-2,2,4,8-tetramethylquinoline (compound 105),
(+)-5-Chloro-6-(3-cyano-2-methoxyphenyl)-1,2,3,4-tetrahydro-2,2,4,8-tetramethylquinoline (compound 105A),
(−)-5-Chloro-6-(3-cyano-2-methoxyphenyl)-1,2,3,4-tetrahydro-2,2,4,8-tetramethylquinoline (compound 105B),
(±)-6-(3-Amino-5-methylisoxazol-4-yl)-5-chloro-1,2,3,4-tetrahydro-2,2,4,8-tetramethylquinoline (compound 106),
(±)-5-Chloro-1,2,3,4-tetrahydro-6-(2-methoxyphenyl)-2,2,4,8-tetramethylquinoline (compound 107),
(±)-5-Chloro-1,2,3,4-tetrahydro-2,2,4,8-tetramethyl-6-(quinolin-8-yl)quinoline (compound 108),
(±)-6-(Benzothiophen-3-yl)-5-chloro-1,2,3,4-tetrahydro-2,2,4,8-tetramethylquinoline (compound 109),
(±)-5-Chloro-1,2,3,4-tetrahydro-2,2,4,8-tetramethyl-6-(5-methyl-3-phenylisoxazol-4-yl)quinoline (compound 110),
(±)-5-Chloro-1,2,3,4-tetrahydro-2,2,4,8-tetramethyl-6-(1,3,5-trimethylpyrazol-4-yl)quinoline (compound 111),
(±)-5-Chloro-6-(2,4-dimethoxyphenyl)-1,2,3,4-tetrahydro-2,2,4,8-tetramethylquinoline (compound 112),
(±)-6-(2-Aminophenyl)-5-chloro-1,2,3,4-tetrahydro-2,2,4,8-tetramethylquinoline (compound 113),
(±)-5-Chloro-6-(3,5-dimethylisoxazol-4-yl)-1,2,3,4-tetrahydro-2,2,4,8-tetramethylquinoline (compound 114),
(−)-5-Chloro-6-(3,5-dimethylisoxazol-4-yl)-1,2,3,4-tetrahydro-2,2,4,8-tetramethylquinoline (compound 114B),
(+)-5-Chloro-6-(3,5-dimethylisoxazol-4-yl)-1,2,3,4-tetrahydro-2,2,4,8-tetramethylquinoline (compound 114A),
(±)-6-(5-Acetylthiophen-2-yl)-5-chloro-1,2,3,4-tetrahydro-2,2,4,8-tetramethylquinoline (compound 115),
(±)-6-(Benzothiophen-2-yl)-5-chloro-1,2,3,4-tetrahydro-2,2,4,8-tetramethylquinoline (compound 116),
(±)-5-Chloro-6-(2-fluorophenyl)-1,2,3,4-tetrahydro-2,2,4,8-tetramethylquinoline (compound 117),
(±)-5-Chloro-6-(2-chlorophenyl)-1,2,3,4-tetrahydro-2,2,4,8-tetramethylquinoline (compound 118),
(±)-6-(2-Acetylphenyl)-5-chloro-1,2,3,4-tetrahydro-2,2,4,8-tetramethylquinoline (compound 119),
(±)-5-Chloro-1,2,3,4-tetrahydro-6-(indol-4-yl)-2,2,4,8-tetramethylquinoline (compound 120),
(±)-5-Chloro-6-(5-chloro-2-methoxyphenyl)-1,2,3,4-tetrahydro-2,2,4,8-tetramethylquinoline (compound 121),
(±)-5-Chloro-1,2,3,4-tetrahydro-2,2,4,8-tetramethyl-6-(2-nitrophenyl)quinoline (compound 122),
(±)-5-Chloro-6-(2,3-dichlorophenyl)-1,2,3,4-tetrahydro-2,2,4,8-tetramethylquinoline (compound 123),
(±)-5-Chloro-1,2,3,4-tetrahydro-2,2,4,8-tetramethyl-6-[2-(trifluoromethyl)phenyl]quinoline (compound 124),
(±)-5-Chloro-1,2,3,4-tetrahydro-2,2,4,8-tetramethyl-6-(2-methyl-3-nitrophenyl)quinoline (compound 125),
(±)-6-(2-Biphenyl)-5-chloro-1,2,3,4-tetrahydro-2,2,4,8-tetramethylquinoline (compound 126),
(±)-5-Chloro-6-(dibenzofuran-1-yl)-1,2,3,4-tetrahydro-2,2,4,8-tetramethylquinoline (compound 127),
(±)-5-Chloro-1,2,3,4-tetrahydro-6-(indol-6-yl)-2,2,4,8-tetramethylquinoline (compound 128),
(±)-5-Chloro-6-(2,3-dihydro-1,4-benzodioxin-6-yl)-1,2,3,4-tetrahydro-2,2,4,8-tetramethylquinoline (compound 129),
(±)-5-Chloro-6-[2-fluoro-3-(trifluoromethyl)phenyl]-1,2,3,4-tetrahydro-2,2,4,8-tetramethylquinoline (compound 130),
(±)-5-Chloro-1,2,3,4-tetrahydro-2,2,4,8-tetramethyl-6-[2-(trifluoromethoxy)phenyl]quinoline (compound 131),
(±)-5-Chloro-6-(5-cyano-2-methoxyphenyl)-1,2,3,4-tetrahydro-2,2,4,8-tetramethylquinoline (compound 132),
(±)-6-(1-Acetyl-3,5-dimethylpyrazol-4-yl)-5-chloro-1,2,3,4-tetrahydro-2,2,4,8-tetramethylquinoline (compound 133),
(±)-5-Chloro-1,2,3,4-tetrahydro-6-(indol-3-yl)-2,2,4,8-tetramethylquinoline (compound 134),
(±)-5-Chloro-1,2,3,4-tetrahydro-2,2,4,8-tetramethyl-6-(naphthal-1-yl)quinoline (compound 135),
(±)-5-Chloro-1,2,3,4-tetrahydro-2,2,4,8-tetramethyl-6-(3-methylpyrid-2-yl)quinoline (compound 136),
(±)-5-Chloro-6-(5-fluoroindol-7-yl)-1,2,3,4-tetrahydro-2,2,4,8-tetramethylquinoline (compound 137),
(±)-5-Chloro-1,2,3,4-tetrahydro-2,2,4,8-tetramethyl-6-(2-methylindol-7-yl)quinoline (compound 138),
(±)-5-Chloro-1,2,3,4-tetrahydro-2,2,4,8-tetramethyl-6-(3-methylindol-7-yl)quinoline (compound 139),
(±)-5-Chloro-6-(5-chloroindol-7-yl)-1,2,3,4-tetrahydro-2,2,4,8-tetramethylquinoline (compound 140),
(±)-5-Chloro-6-(4-fluoroindol-7-yl)-1,2,3,4-tetrahydro-2,2,4,8-tetramethylquinoline (compound 141),
(±)-5-Chloro-6-(4-chloroindol-7-yl)-1,2,3,4-tetrahydro-2,2,4,8-tetramethylquinoline (compound 142),
(±)-5-Chloro-6-(4,5-difluoroindol-7-yl)-1,2,3,4-tetrahydro-2,2,4,8-tetramethylquinoline (compound 143),
(±)-5-Chloro-1,2,3,4-tetrahydro-6-(4-methoxyindol-7-yl)-2,2,4,8-tetramethylquinoline (compound 144),
(±)-5-Chloro-6-(4-chloro-3-methylindol-7-yl)-1,2,3,4-tetrahydro-2,2,4,8-tetramethylquinoline (compound 145),
(±)-5-Chloro-6-(2,3-dimethylindol-7-yl)-1,2,3,4-tetrahydro-2,2,4,8-tetramethylquinoline (compound 146),
(±)-5-Chloro-6-(4-fluoro-3-methylindol-7-yl)-1,2,3,4-tetrahydro-2,2,4,8-tetramethylquinoline (compound 147),
(±)-5-Chloro-1,2,3,4-tetrahydro-2,2,4,8-tetramethyl-6-(1-methylindol-7-yl)quinoline (compound 148),
(±)-5-Chloro-1,2,3,4-tetrahydro-6-(indol-7-yl)-2,2,4,8-tetramethylquinoline (compound 149),
(−)-5-Chloro-1,2,3,4-tetrahydro-6-(indol-7-yl)-2,2,4,8-tetramethylquinoline (compound 149B),
(+)-5-Chloro-1,2,3,4-tetrahydro-6-(indol-7-yl)-2,2,4,8-tetramethylquinoline (compound 149A),
(±)-5-Chloro-6-(3-cyano-2,6-dimethoxyphenyl)-1,2,3,4-tetrahydro-2,2,4,8-tetramethylquinoline (compound 150),
(±)-5-Chloro-1,2,3,4-tetrahydro-6-(3-hydroxy-2-methoxyphenyl)-2,2,4,8-tetramethylquinoline (compound 151),
(±)-5-Chloro-6-(1-tetralon-5-yl)-1,2,3,4-tetrahydro-2,2,4,8-tetramethylquinoline (compound 152),
(±)-5-Chloro-6-(1-indanon-4-yl)-1,2,3,4-tetrahydro-2,2,4,8-tetramethylquinoline (compound 153),
(±)-5-Chloro-6-(1-hydroxyiminoindan-4-yl)-1,2,3,4-tetrahydro-2,2,4,8-tetramethylquinoline (compound 154),
(±)-5-Chloro-6-(3-cyano-2-methylphenyl)-1,2,3,4-tetrahydro-2,2,4,8-tetramethylquinoline (compound 155),
(±)-5-Chloro-1,2,3,4-tetrahydro-6-(2-methoxy-3-nitrophenyl)-2,2,4,8-tetramethylquinoline (compound 156),
(±)-5-Chloro-1,2,3,4-tetrahydro-6-(2-methoxy-6-nitrophenyl)-2,2,4,8-tetramethylquinoline (compound 157),
(±)-6-(2-Benzyloxy-3-nitrophenyl)-5-chloro-1,2,3,4-tetrahydro-2,2,4,8-tetramethylquinoline (compound 158),
(±)-6-(Benzothiophen-3-yl)-5-chloro-1,2,3,4-tetrahydro-3β-hydroxy-2,2,4α,8-tetramethylquinoline (compound 159),
(±)-5-Chloro-1,2,3,4-tetrahydro-3β-hydroxy-2,2,4α,8-tetramethyl-6-(thiophen-3-yl)quinoline (compound 160),
(±)-5-Chloro-1,2,3,4-tetrahydro-3β-hydroxy-6-(indol-7-yl)-2,2,4α,8-tetramethylquinoline (compound 161),
(+)-5-Chloro-1,2,3,4-tetrahydro-3β-hydroxy-6-(indol-7-yl)-2,2,4α,8-tetramethylquinoline (compound 161A),
(−)-5-Chloro-1,2,3,4-tetrahydro-3β-hydroxy-6-(indol-7-yl)-2,2,4α,8-tetramethylquinoline (compound 161B),
(±)-5-Chloro-1,2,3,4-tetrahydro-3β-hydroxy-2,2,4α,8-tetramethyl-6-(naphthal-1-yl)quinoline (compound 162),
(±)-5-Chloro-6-(4-fluoroindol-7-yl)-1,2,3,4-tetrahydro-3β-hydroxy-2,2,4α,8-tetramethylquinoline (compound 163),
(±)-5-Chloro-6-(3,5-dimethylisoxazol-4-yl)-1,2,3,4-tetrahydro-3β-hydroxy-2,2,4α,8-tetramethylquinoline (compound 164),
(±)-5-Chloro-6-(3-cyano-2-methoxyphenyl)-1,2,3,4-tetrahydro-3β-hydroxy-2,2,4α,8-tetramethylquinoline (compound 165),
(±)-5-Chloro-1,2,3,4-tetrahydro-3β-hydroxy-6-(4-fluoro-3-methylindol-7-yl)-2,2,4α,8-tetramethylquinoline (compound 166),
(±)-5-Chloro-1,2,3,4-tetrahydro-3β-hydroxy-6-(5-fluoroindol-7-yl)-2,2,4α,8-tetramethylquinoline (compound 167),
(±)-5-Chloro-1,2,3,4-tetrahydro-3β-hydroxy-6-(3-methylindol-7-yl)-2,2,4α,8-tetramethylquinoline (compound 168),
(±)-7-Chloro-6-(3-cyano-2-methoxyphenyl)-1,2,3,4-tetrahydro-2,2,4,8-tetramethylquinoline (compound 169),
(±)-7-Chloro-6-(3-cyanophenyl)-1,2,3,4-tetrahydro-2,2,4,8-tetramethylquinoline (compound 170),
(±)-7-Chloro-1,2,3,4-tetrahydro-6-(indol-7-yl)-2,2,4,8-tetramethylquinoline (compound 171),
(±)-7-Chloro-6-(3,5-dimethylisoxazol-4-yl)-1,2,3,4-tetrahydro-3β-hydroxy-2,2,4a,8-tetramethylquinoline (compound 172),
(±)-7-Chloro-1,2,3,4-tetrahydro-3β-hydroxy-6-(indol-7-yl)-2,2,4α,8-tetramethylquinoline (compound 173),
5-Chloro-6-(3-cyano-2-methoxyphenyl)-1,2-dihydro-2,2,4-trimethylquinoline (compound 174),
7-Chloro-6-(3-cyano-2-methoxyphenyl)-1,2-dihydro-2,2,4-trimethylquinoline (compound 175),
(±)-5-Chloro-6-(3-cyano-2-methoxyphenyl)-1,2,3,4-tetrahydro-2,2,4-trimethylquinoline (compound 176),
(±)-7-Chloro-6-(3-cyano-2-methoxyphenyl)-1,2,3,4-tetrahydro-2,2,4-trimethylquinoline (compound 177),
5-Chloro-6-(3,5-dimethylisoxazol-4-yl)-1,2-dihydro-2,2,4,8-tetramethylquinoline (compound 178),
(±)-5-Chloro-6-(3-cyano-2-methoxyphenyl)-1,4-dihydro-2,2,4,8-tetramethyl-2H-quinolin-3-one (compound 179),
(±)-4-Benzyl-5-chloro-6-(3-cyano-2-methoxyphenyl)-1,4-dihydro-2,2,4,8-tetramethyl-2H-quinolin-3-one (compound 180),
5-Chloro-6-(3-cyano-2-methoxyphenyl)-1,4-dihydro-2,2,4,4,8-pentamethyl-2H-quinolin-3-one (compound 181),
(±)-5-Chloro-6-(3,5-dimethylisoxazol-4-yl)-1,4-dihydro-2,2,4,8-tetramethyl-2H-quinolin-3-one (compound 182),
5-Chloro-6-(3,5-dimethylisoxazol-4-yl)-1,4-dihydro-2,2,4,4,8-pentamethyl-2H-quinolin-3-one (compound 183),
(±)-4-Benzyl-5-chloro-6-(3,5-dimethylisoxazol-4-yl)-1,4-dihydro-2,2,4,8-tetramethyl-2H-quinolin-3-one (compound 184),
(±)-5-Chloro-4-(3,3-dimethylallyl)-6-(3,5-dimethylisoxazol-4-yl)-1,4-dihydro-2,2,4,8-tetramethyl-2H-quinolin-3-one (compound 185),
(±)-5-Chloro-1,4-dihydro-6-(indol-7-yl)-2,2,4,8-tetramethyl-2H-quinolin-3-one (compound 186),
5-Chloro-1,4-dihydro-6-(indol-7-yl)-2,2,4,4,8-pentamethyl-2H-quinolin-3-one (compound 187),
(±)-4-Benzyl-5-chloro-1,4-dihydro-6-(indol-7-yl)-2,2,4,8-tetramethyl-2H-quinolin-3-one (compound 188),
(±)-5-Chloro-4-(3,3-dimethylallyl)-1,4-dihydro-6-(indol-7-yl)-2,2,4,8-tetramethyl-2H-quinolin-3-one (compound 189),
(±)-4-Allyl-5-chloro-1,4-dihydro-6-(indol-7-yl)-2,2,4,8-tetramethyl-2H-quinolin-3-one (compound 190),
(±)-5-Chloro-6-(3-cyano-2-methoxyphenyl)-1,2,3,4-tetrahydro-3α-hydroxy-2,2,4α,8-tetramethylquinoline (compound 191),
(±)-5-Chloro-6-(3,5-dimethylisoxazol-4-yl)-1,2,3,4-tetrahydro-3α-hydroxy-2,2,4α,8-tetramethylquinoline (compound 192),
(±)-5-Chloro-1,2,3,4-tetrahydro-3α-hydroxy-6-(indol-7-yl)-2,2,4α,8-tetramethylquinoline (compound 193),
(±)-6-(Benzothiophen-3-yl)-5-chloro-1,2,3,4-tetrahydro-3α-hydroxy-2,2,4α,8-tetramethylquinoline (compound 194),
(±)-5-Chloro-1,2,3,4-tetrahydro-3α-hydroxy-2,2,4α,8-tetramethyl-6-(naphthal-1-yl)quinoline (compound 195),
(±)-5-Chloro-1,2,3,4-tetrahydro-3-hydroxy-6-(indol-7-yl)-2,2,4,4,8-pentamethylquinoline (compound 196),
(±)-5-Chloro-6-(3,5-dimethylisoxazol-4-yl)-1,2,3,4-tetrahydro-3-hydroxy-2,2,4,4,8-pentamethylquinoline (compound 197),
(±)-6-(3-Amino-2-methoxyphenyl)-5-chloro-1,2,3,4-tetrahydro-2,2,4,8-tetramethylquinoline (compound 198),
(±)-5-Chloro-1,2,3,4-tetrahydro-6-[2-methoxy-3-(methoxycarbonylamino)phenyl]-2,2,4,8-tetramethylquinoline (compound 199),
(±)-5-Chloro-1,2,3,4-tetrahydro-6-[3-(tert-butoxycarbonylamino)-2-methoxyphenyl]-2,2,4,8-tetramethylquinoline (compound 200),
(±)-5-Chloro-1,2,3,4-tetrahydro-6-[2-methoxy-3-(methylsulfonamido)phenyl]-2,2,4,8-tetramethylquinoline (compound 201),
(±)-5-Chloro-1,2,3,4-tetrahydro-6-(2-hydroxy-3-nitrophenyl)-2,2,4,8-tetramethylquinoline (compound 202),
(±)-5-Chloro-1,2,3,4-tetrahydro-2,2,4,8-tetramethyl-6-[2-(methylbut-2-enyloxy)-3-nitrophenyl]quinoline (compound 203),
(±)-6-(2H-1,4-Benzoxazin-3(4H)-on-8-yl)-5-chloro-1,2,3,4-tetrahydro-2,2,4,8-tetramethylquinoline (compound 204),
(±)-5-Chloro-1,2,3,4-tetrahydro-2,2,4,8-tetramethyl-6-(4-methyl-2H-1,4-benzoxazin-3(4H)-on-8-yl)quinoline (compound 205),
(±)-6-(2-Benzoxazolinon-7-yl)-5-chloro-1,2,3,4-tetrahydro-2,2,4,8-tetramethylquinoline (compound 206),
(±)-6-(3-Amino-2-hydroxyphenyl)-5-chloro-1,2,3,4-tetrahydro-2,2,4,8-tetramethylquinoline (compound 207),
(±)-6-(2-Amino-6-methoxyphenyl)-5-chloro-1,2,3,4-tetrahydro-2,2,4,8-tetramethylquinoline (compound 208),
(±)-5-Chloro-1,2,3,4-tetrahydro-6-(6-methoxyindol-7-yl)-2,2,4,8-tetramethylquinoline (compound 209),
(±)-5-Chloro-1,2,3,4-tetrahydro-6-(indolin-7-yl)-2,2,4,8-tetramethylquinoline (compound 210),
(±)-6-(3-Bromoindol-7-yl)-5-chloro-1,2,3,4-tetrahydro-2,2,4,8-tetramethylquinoline (compound 211),
(±)-5-Chloro-1,2,3,4-tetrahydro-2,2,4,8-tetramethyl-6-(2-oxindol-7-yl)quinoline (compound 212),
(±)-5-Chloro-1,2,3,4-tetrahydro-4-hydroxy-6-(indol-2-yl)-2,2,4,8-tetramethylquinoline (compound 213),
5-Chloro-1,2-dihydro-6-(indol-2-yl)-2,2,4,8-tetramethylquinoline (compound 214),
(±)-5-Chloro-1,2,3,4-tetrahydro-4-hydroxy-2,2,4,8-tetramethyl-6-(naphthal-1-yl)quinoline (compound 215),
(±)-1,2,3,4-Tetrahydro-3β-hydroxy-6-(indol-7-yl)-2,2,4α,5,8-pentamethylquinoline (compound 216),
(±)-6-(3,5-Dimethylisoxazol-4-yl)-1,2,3,4-tetrahydro-3β-hydroxy-2,2,4α,5,8-pentamethylquinoline (compound 217),
(±)-5-Fluoro-1,2,3,4-tetrahydro-3β-hydroxy-2,2,4α,8-tetramethyl-6-(naphthal-1-yl)quinoline (compound 218),
(±)-6-(3,5-Dimethylisoxazol-4-yl)-5-fluoro-1,2,3,4-tetrahydro-3β-hydroxy-2,2,4α,8-tetramethylquinoline (compound 219),
(±)-5-Fluoro-1,2,3,4-tetrahydro-3β-hydroxy-6-(indol-7-yl)-2,2,4α,8-tetramethylquinoline (compound 220),
(±)-5-Chloro-1,2,3,4-tetrahydro-3β-hydroxy-6-(indolin-7-yl)-2,2,4α,8-tetramethylquinoline (compound 221),
(±)-5-Fluoro-1,2,3,4-tetrahydro-3β-hydroxy-6-(indolin-7-yl)-2,2,4α,8-tetramethylquinoline (compound 222),
(±)-5-Chloro-1,2,3,4-tetrahydro-3β-hydroxy-6-[3-(butan-3-on-1-yl)indol-7-yl]-2,2,4α,8-tetramethylquinoline (compound 223);
5-Chloro-6-(3-cyanophenyl)-1,2-dihydro-2,2,4-trimethylquinoline (compound 224);
(±)-5-Chloro-6-(3-cyanophenyl)-1,2,3,4-tetrahydro-2,2,4-trimethylquinoline (compound 225);
(+)-5-Chloro-6-(3-cyanophenyl)-1,2,3,4-tetrahydro-2,2,4-trimethylquinoline (compound 225A);
(−)-5-Chloro-6-(3-cyanophenyl)-1,2,3,4-tetrahydro-2,2,4-trimethylquinoline (compound 225B);
5-Chloro-6-(3-cyanophenyl)-1,2-dihydro-1,2,2,4-tetramethylquinoline (compound 226);
5-Chloro-8-fluoro-1,2-dihydro-2,2,4-trimethyl-6-(3-nitrophenyl)quinoline (compound 227);
5-Chloro-1,2-dihydro-2,2,4,8-tetramethyl-6-(3-nitrophenyl)quinoline (compound 228);
6-[3,5-Bis(trifluoromethyl)phenyl]-5-chloro-1,2-dihydro-2,2,4-trimethylquinoline (compound 229);
5-Chloro-1,2-dihydro-2,2,4-trimethyl-6-[3-(trifluoromethyl)phenyl]quinoline (compound 230);
5-Chloro-6-(3-cyanophenyl)-1,2-dihydro-2,2,4,8-tetramethylquinoline (compound 231);
5-Chloro-6-(3-cyano-4-fluorophenyl)-1,2-dihydro-2,2,4,8-tetramethylquinoline (compound 232);
6-(3-Acetylphenyl)-5-chloro-1,2-dihydro-2,2,4,8-tetramethylquinoline (compound 233);
5-Chloro-1,2-dihydro-2,2,4,8-tetramethyl-6-(3-methylphenyl)quinoline (compound 234);
5-Chloro-6-[4-chloro-3-(trifluoromethyl)phenyl]-1,2-dihydro-2,2,4,8-tetramethylquinoline (compound 235);
5-Chloro-6-(3-cyano-2-methylphenyl)-1,2-dihydro-2,2,4,8-tetramethylquinoline (compound 236);
5-Chloro-6-(3-fluoro-2-methylphenyl)-1,2-dihydro-2,2,4,8-tetramethylquinoline (compound 237);
5-Chloro-1,2-dihydro-2,2,4,8-tetramethyl-6-[3-(propionyl)phenyl]quinoline (compound 238);
6-(3-Carbamoylphenyl)-5-chloro-1,2-dihydro-2,2,4-trimethylquinoline (compound 239);
6-(3-Carboxymethylphenyl)-5-chloro-1,2-dihydro-2,2,4,8-tetramethylquinoline (compound 240);
5-Chloro-6-(5-cyanothiophen-3-yl)-1,2-dihydro-2,2,4,8-tetramethylquinoline (compound 241);
5-Chloro-6-(5-cyanopyrid-3-yl)-1,2-dihydro-2,2,4,8-tetramethylquinoline (compound 242);
(±)-6-(3-Acetylphenyl)-5-chloro-1,2,3,4-tetrahydro-2,2,4,8-tetramethylquinoline (compound 243);
(+)-6-(3-Acetylphenyl)-5-chloro-1,2,3,4-tetrahydro-2,2,4,8-tetramethylquinoline (compound 243A);
(−)-6-(3-Acetylphenyl)-5-chloro-1,2,3,4-tetrahydro-2,2,4,8-tetramethylquinoline (compound 243B);
(±)-5-Chloro-6-(5-cyanothiophen-3-yl)-1,2,3,4-tetrahydro-2,2,4,8-tetramethylquinoline (compound 244);
(±)-5-Acetoxy-6-(3-cyanophenyl)-1,2,3,4-tetrahydro-2,2,4,8-tetramethylquinoline (compound 245);
6-[3-(N-Methoxy-N-methylcarbamoyl)phenyl]-5-chloro-1,2-dihydro-2,2,4-trimethylquinoline (compound 246);
5-Chloro-1,2-dihydro-2,2,4,8-tetramethyl-6-[3-(2-methylpropionyl)phenyl]quinoline (compound 247);
(±)-5-Chloro-6-(3-cyano-2-hydroxyphenyl)-1,2,3,4-tetrahydro-2,2,4,8-tetramethylquinoline (compound 248);
(±)-6-(3-Cyanophenyl)-1,2,3,4-tetrahydro-5-hydroxy-2,2,4,8-tetramethylquinoline (compound 249);
(±)-6-(3-Cyanophenyl)-1,2,3,4-tetrahydro-5-methoxy-2,2,4,8-tetramethylquinoline (compound 250);
(±)-6-(5-Carbamoylpyrid-3-yl)-5-chloro-1,2,3,4-tetrahydro-2,2,4,8-tetramethylquinoline (compound 251);
(±)-5-Chloro-6-(2-cyanothiophen-3-yl)-1,2,3,4-tetrahydro-2,2,4,8-tetramethylquinoline (compound 252);
(±)-5-Chloro-6-[3-(cyanomethyl)phenyl]-1,2,3,4-tetrahydro-2,2,4,8-tetramethylquinoline (compound 253);
(±)-6-(3-Cyanophenyl)-5-(2,2-dimethylpropionyloxy)-1,2,3,4-tetrahydro-2,2,4,8-tetramethylquinoline (compound 254);
(±)-5-Chloro-1,2,3,4-tetrahydro-2,2,4,8-tetramethyl-6-(5-nitrothiophen-2-yl)quinoline (compound 255);
(±)-5-Chloro-1,2,3,4-tetrahydro-2,2,4,8-tetramethyl-6-(pyrimidin-5-yl)quinoline (compound 256);
6-(3-Acetylphenyl)-5,7-dichloro-1,2-dihydro-2,2,4-trimethylquinoline (compound 257);
(±)-1,2,3,4-Tetrahydro-3β-hydroxy-6-(indol-7-yl)-2,2,4α,8-tetramethylquinoline (compound 258);
(±)-6-(3,5-Dimethylisoxazol-4-yl)-1,2,3,4-tetrahydro-3β-hydroxy-2,2,4α,8-tetramethylquinoline (compound 259);
(±)-1,2,3,4-Tetrahydro-3β-hydroxy-2,2,4α,8-tetramethyl-6-(quinolin-8-yl)quinoline (compound 260);
(±)-5-Chloro-1,2,3,4-tetrahydro-6-(indol-7-yl)-2,2,4,8-tetramethylquinoline (compound 261);
(±)-5-Chloro-1,2,3,4-tetrahydro-3β-hydroxy-2,2,4α,8-tetramethyl-6-(6-fluoro-2-nitrophenyl)-quinoline (compound 262);
(±)-5-Chloro-1,2,3,4-tetrahydro-3β-hydroxy-6-(6-fluoroindol-7-yl)-2,2,4α,8-tetramethylquinoline (compound 263);
(±)-5-Chloro-1,2,3,4-tetrahydro-3β-hydroxy-2,2,4α,8-tetramethyl-6-(4,6-difluoro-2-nitrophenyl)quinoline (compound 264);
(±)-5-Chloro-1,2,3,4-tetrahydro-3β-hydroxy-6-(4,6-difluoroindol-7-yl)-2,2,4α,8-tetramethylquinoline (compound 265);
(±)-5-Chloro-1,2,3,4-tetrahydro-3β-hydroxy-6-(5-fluoroindol-7-yl)-2,2,4α,8-tetramethylquinoline (compound 266);
(±)-1,2,3,4-Tetrahydro-3β-hydroxy-2,2,4α,8-tetramethyl-6-(6-methoxy-2-nitrophenyl)-quinoline (compound 267);
(±)-1,2,3,4-Tetrahydro-3β-hydroxy-6-(6-methoxy-indol-7-yl)-2,2,4α,8-tetramethylquinoline (compound 268);
(±)-7-Fluoro-1,2,3,4-tetrahydro-3β-hydroxy-6-(indol-7-yl)-2,2,4α,8-tetramethylquinoline (compound 269);
(±)-6-(3,5-Dimethylisoxazol-4-yl)-1,2,3,4-tetrahydro-3β-hydroxy-5-methoxy-2,2,4α,8-tetramethylquinoline (compound 270);
(±)-1,2,3,4-tetrahydro-3β-hydroxy-5-methoxy-2,2,4α,8-tetramethyl-6-(naphth-1-yl)quinoline (compound 271);
(±)-1,2,3,4-tetrahydro-3β-hydroxy-6-(indol-7-yl)-5-methoxy-2,2,4α,8-tetramethylquinoline (compound 272);
(±)-5-Chloro-6-(2-fluoropyrid-3-yl)-1,2,3,4-tetrahydro-3β-hydroxy-2,2,4α,8-tetramethylquinoline (compound 273);
(±)-5-Chloro-1,2,3,4-tetrahydro-3β-hydroxy-6-(2-methoxypyrid-3-yl)-2,2,4α,8-tetramethylquinoline (compound 274);
(±)-5-Chloro-1,2,3,4-tetrahydro-8-fluoro-3β-hydroxy-6-(indol-7-yl)-2,2,4α-trimethylquinoline (compound 275);
(±)-5-Cyano-1,2,3,4-tetrahydro-3β-hydroxy-6-(indol-7-yl)-2,2,4α,8-tetramethylquinoline (compound 276);
(±)-5-Ethynyl-1,2,3,4-tetrahydro-3β-hydroxy-6-(indol-7-yl)-2,2,4α,8-tetramethylquinoline (compound 277);
(±)-1,2,3,4-Tetrahydro-3β-hydroxy-6-(indol-7-yl)-2,2,4α,8-tetramethyl-E-(2-phenylethenyl)quinoline (compound 278);
(±)-5-Carbomethoxy-1,2,3,4-tetrahydro-6-(indol-7-yl)-2,2,4,8-tetramethylquinoline (compound 279);
(±)-5-Carboxy-1,2,3,4-tetrahydro-6-(indol-7-yl)-2,2,4,8-tetramethylquinoline (compound 280);
(±)-5-Chloro-1,2,3,4-tetrahydro-6-(6-methoxy-3-methylindol-7-yl)-2,2,4,8-tetramethylquinoline (compound 281);
(±)-5-Chloro-1,2,3,4-tetrahydro-3β-hydroxy-2,2,4α,8-tetramethyl-6-(oxazol-5-yl)quinoline (compound 282);
(±)-5-Chloro-1,2,3,4-tetrahydro-3β-hydroxy-6-(5-methoxyindol-7-yl)-2,2,4α,8-tetramethylquinoline (compound 283);
(±)-5-Chloro-1,2,3,4-tetrahydro-3β-hydroxy-2,2,4α,8-tetramethyl-6-(pyrid-4-yl)quinoline (compound 284);
(±)-5-Cyano-1,2,3,4-tetrahydro-3β-hydroxy-6-(indolin-7-yl)-2,2,4α,8-tetramethylquinoline (compound 285);
(±)-5-Chloro-1,2,3,4-tetrahydro-3α-methoxy-2,2,4α,8-tetramethyl-6-(naphthal-1-yl)quinoline (compound 286);
(±)-1,2,3,4-Tetrahydro-3β-hydroxy-6-(indolin-7-yl)-5-(methoxyimino)-2,2,4α,8-tetramethylquinoline (compound 287);
(±)-1,2,3,4-Tetrahydro-5-(hydroxymethyl)-6-(indol-7-yl)-2,2,4,8-tetramethylquinoline (compound 288);
(±)-5-(3-(2-Fluoroethoxy)benzyloxymethyl)-1,2,3,4-tetrahydro-6-(indol-7-yl)-2,2,4,8-tetramethylquinoline (compound 289);
(±)-5-((6-Fluoro-4H-benzo[1,3]dioxin-8-yl)methoxymethyl)-1,2,3,4-tetrahydro-6-(indol-7-yl)-2,2,4,8-tetramethylquinoline (compound 290);
(±)-5-(2-Fluoro-3-methylbenzyloxymethyl)-1,2,3,4-tetrahydro-6-(indol-7-yl)-2,2,4,8-tetramethylquinoline (compound 291);
and a pharmaceutically acceptable salt, ester, amide, or prodrug of any of the above.

183. A compound of claim 1 that is a selective glucocorticoid receptor modulator.

184. A compound of claim 1 that is a selective mineralocorticoid receptor modulator.

185. A compound of claim 1 that is a selective glucocorticoid/mineralocorticoid receptor modulator.

186. The compound of claim 183 that is a glucocorticoid receptor agonist.

187. The compound of claim 183 that is a glucocorticoid receptor antagonist.

188. The compound of claim 183 that is a glucocorticoid receptor partial agonist.

189. (canceled)

190. The compound of claim 184 that is a mineralocorticoid receptor antagonist.

191. (canceled)

192. A selective glucocorticoid receptor binding compound of claim 1.

193. A selective mineralocorticoid receptor binding compound of claim 1.

194. A selective glucocorticoid/mineralocorticoid receptor binding compound of claim 1.

195-200. (canceled)

201. A method for modulating an activity of a glucocorticoid receptor comprising contacting the receptor with a compound of claim 1.

202. (canceled)

203. A method for modulating an activity of a glucocorticoid receptor and an activity of a mineralocorticoid receptor comprising contacting the glucocorticoid receptor and the mineralocorticoid receptor with a compound of claim 1.

204. (canceled)

205. (canceled)

206. A method comprising contacting a cell expressing a glucocorticoid receptor and a mineralocorticoid receptor with a compound of claim 1 and monitoring an effect on the cell.

207. A method of treating a patient suffering from a mineralocorticoid receptor related disorder or a glucocorticoid receptor related disorder, comprising identifying a patient in need thereof and contacting said patient with a compound of claim 1.

208. The method of claim 207 wherein the patient suffers form a condition selected from the group consisting of: inflammation, transplant rejection, psoriasis, dermatitis, autoimmune disorder, malignancy, adrenal insufficiency, congenital adrenal hyperplasia, rheumatic fever, granulomatous disease, immune proliferation/apoptosis, conditions of the HPA axis, hypercortisolemia, cytokine imbalance, kidney disease, liver disease, stroke, spinal cord injury, hypercalcemia, hyperglycemia, cerebral edema, thrombocytopenia, Little's syndrome, Addison's disease, cystic fibrosis, myasthenia gravis, autoimmune hemolytic anemia, uveitis, pemphigus vulgaris, multiple sclerosis, nasal polyps, sepsis, infections, type II diabetes, obesity, metabolic syndrome, depression, schizophrenia, mood disorders, Cushing's syndrome, anxiety, sleep disorders, poor memory, glaucoma, wasting, heart disease, fibrosis, hypertension, hyperaldosteronism, and sodium and/or potassium imbalance.

209. A pharmaceutical agent comprising a physiologically acceptable carrier, diluent, or excipient; and a compound of claim 1.

210. A pharmaceutical agent comprising a physiologically acceptable carrier, diluent, or excipient; and a compound of claim 182.

211. The pharmaceutical agent of claim 209 or claim 210 for use in treating a condition selected from the group consisting of: inflammation, transplant rejection, psoriasis, dermatitis, autoimmune disorder, malignancy, adrenal insufficiency, congenital adrenal hyperplasia, rheumatic fever, granulomatous disease, immune proliferation/apoptosis, conditions of the HPA axis, hypercortisolemia, cytokine imbalance, kidney disease, liver disease, stroke, spinal cord injury, hypercalcemia, hyperglycemia, cerebral edema, thrombocytopenia, Little's syndrome, Addison's disease, cystic fibrosis, myasthenia gravis, autoimmune hemolytic anemia, uveitis, pemphigus vulgaris, multiple sclerosis, nasal polyps, sepsis, infections, type II diabetes, obesity, metabolic syndrome, depression, schizophrenia, mood disorders, Cushing's syndrome, anxiety, sleep disorders, poor memory, glaucoma, wasting, heart disease, fibrosis, hypertension, hyperaldosteronism, and sodium and/or potassium imbalance.

212. The compound of claim 1, wherein R³ has the structure of formula (a), (l), or (m): 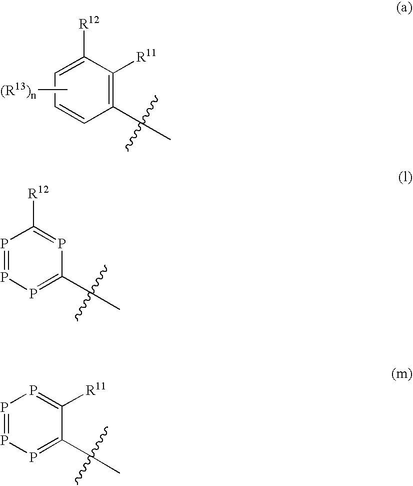
wherein,
R¹¹ is selected from the group consisting of hydrogen, halogen, CN, OMe, an optionally substituted C₁-C₅ alkyl, and an optionally substituted C₁-C₅ heteroalkyl;
R¹² is selected from the group consisting of hydrogen, halogen, CN, OMe, an optionally substituted C₁-C₅ alkyl, a C₁-C₅ heteroalkyl, a C₁-C₅ haloalkyl, or R¹² taken together with R¹¹ form a 5-6 membered ring;
each R¹³ is independently selected from the group consisting of hydrogen, F, Cl, methyl, ethyl, methoxy, CF₃; or R¹³ taken together with R¹² form a 5-6 membered ring; and
R³¹ is selected from the group consisting of hydrogen, halogen, methyl, and OMe.

213. A compound of Formula II: 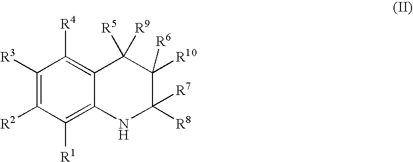
or a pharmaceutically acceptable salt, ester, amide, or prodrug thereof,
wherein:
R¹ and R² are each independently selected from the group consisting of hydrogen, halogen, —CN, —OR¹⁶, an optionally substituted C₁-C₈ alkyl, an optionally substituted C₁-C₈ heteroalkyl, an optionally substituted C₁-C₈ haloalkyl, and a C₁-C₈ heterohaloalkyl;
R³ is selected from the group consisting of (a), (b), (c), (d), (e), (f), (g), (h), (i), (j), (k), (l), (m), and (n); 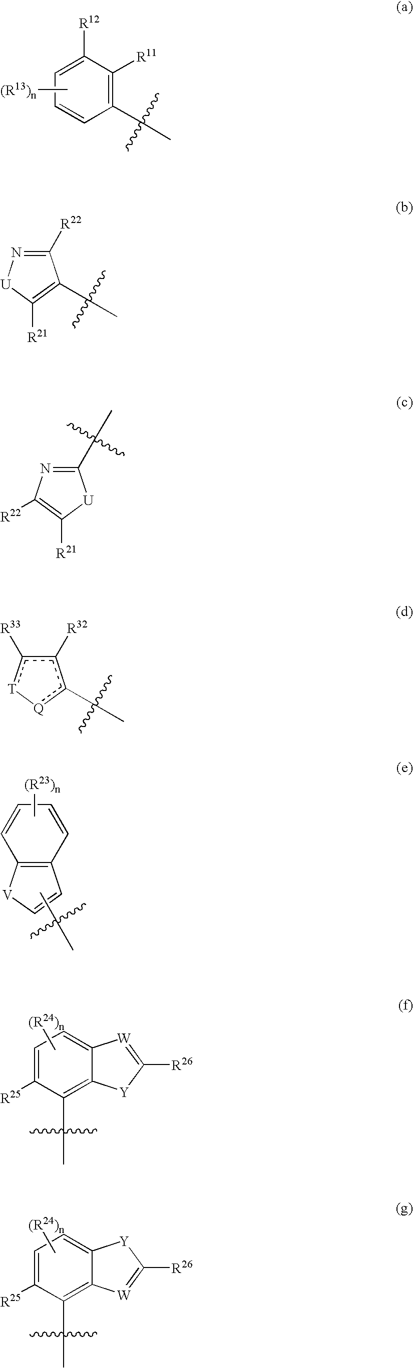 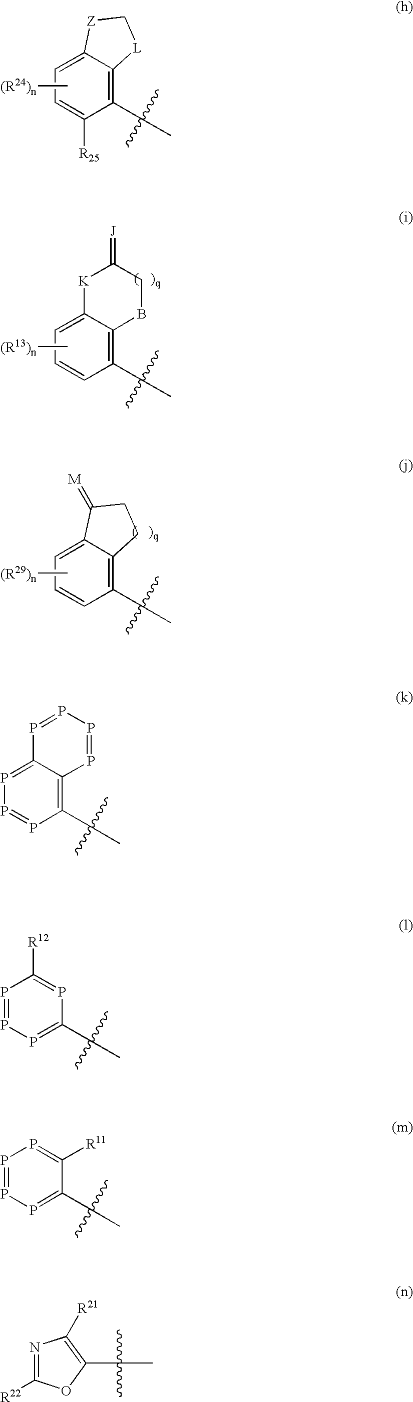
wherein,
R¹¹ is selected from the group consisting of hydrogen, halogen, —CN, —OR¹⁶, —NR¹⁷R¹⁸, —CH₂R¹⁶, —COR²⁰, —SOR²⁰, —SO₂R²⁰, —NO₂, NR¹⁷(OR¹⁶), an optionally substituted C₁-C₈ alkyl, an optionally substituted C₁-C₈ heteroalkyl, an optionally substituted C₁-C₈ haloalkyl, and C₁-C₈ heterohaloalkyl;
R¹² is selected from the group consisting of hydrogen, a halogen, —CN, —COR²⁰, —NR¹⁷SO₂R²⁰, —NR¹⁷CO₂R²⁰, —NO₂, —OR¹⁶, —NR¹⁷R¹⁸, NR¹⁷(OR¹⁶), an optionally substituted C₁-C₈ alkyl, an optionally substituted C₁-C₈ heteroalkyl, an optionally substituted C₁-C₈ haloalkyl, C₁-C₈ heterohaloalkyl, or R¹² taken together with R¹¹ form a 3-7 membered ring;
each R¹³ is independently selected from the group consisting of hydrogen, halogen, CN, —NO₂, OR¹⁶, an optionally substituted C₁-C₈ alkyl, an optionally substituted C₁-C₈ heteroalkyl, an optionally substituted C₁-C₈ haloalkyl, or R¹³ taken together with R¹² form a 3-7 membered ring;
R²¹ is selected from the group consisting of hydrogen, an optionally substituted C₁-C₈ alkyl, a C₁-C₈ heteroalkyl, a C₁-C₈ haloalkyl, and a C₁-C₈ heterohaloalkyl;
R²² is selected from the group consisting of hydrogen, halogen, —NR¹⁷R¹⁸, an optionally substituted C₁-C₈ alkyl, a C₁-C₈ heteroalkyl, a C₁-C₈ haloalkyl, and a C₁-C₈ heterohaloalkyl;
R³² and R³³ are each independently selected from the group consisting of hydrogen, halogen, OR¹⁶, —CN, COR²⁰, an optionally substituted C₁-C₈ alkyl, a C₁-C₈ heteroalkyl, C₁-C₈ haloalkyl, and a C₁-C₈ heterohaloalkyl;
each R²³ is independently selected from the group consisting of hydrogen, halogen, OR¹⁶, an optionally substituted C₁-C₈ alkyl, a C₁-C₈ heteroalkyl, a C₁-C₈ haloalkyl, and a C₁-C₈ heterohaloalkyl;
each R²⁴ is independently selected from the group consisting of hydrogen, halogen, and OR¹⁶;
R²⁵ is selected from the group consisting of hydrogen, halogen, OR¹⁶, —CN, an optionally substituted C₁-C₈ alkyl, a C₁-C₈ heteroalkyl, a C₁-C₈ haloalkyl, and a C₁-C₈ heterohaloalkyl;
R²⁶ is selected from the group consisting of hydrogen, halogen, OR¹⁶, —CN, an optionally substituted C₁-C₈ alkyl, a C₁-C₈ heteroalkyl, a C₁-C₈ haloalkyl, and a C₁-C₈ heterohaloalkyl;
each R²⁹ is independently selected from the group consisting of hydrogen, halogen, and ORE;
U is selected from the group consisting of O, S, and —NR¹⁷;
Q and T are each selected from the group consisting of S, O, and CR³⁴ wherein
either Q is —CR³⁴ and T is selected from the group consisting of S, O, and —NR¹⁷,
or T is CR³⁴ and Q is selected from the group consisting of S, O, and —NR¹⁷;
V is selected from the group consisting of O, S, and —NR¹⁷;
W is selected from the group consisting of —CR²⁷ and N;
Y is selected from the group consisting of NR³⁶, S, and O;
Z and L are each selected from the group consisting of CH₂, —NR²⁸, and O, provided that Z and L can not be both CH₂;
K is selected from the group consisting of O and —NR³⁵;
J is selected from the group consisting of O and S;
B is selected from the group consisting of O and CR²⁷;
M is selected from the group consisting of O and —NOR³⁰;
each P is independently selected from the group consisting of N and CR³¹, provided that no more than two of the Ps are N;
n is selected from 1, 2, 3, and 4; and
q is selected from 0, 1, and 2;
R⁴ is selected from the group consisting of hydrogen, halogen, NO₂, OR¹⁶, NR¹⁷R¹⁸, CN, C═N(OR¹⁶), CO₂R²⁰, CONR²⁰R³⁷, NR¹⁷(OR¹⁶), CR³(OR¹⁶), an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₃-C₈ cycloalkyl, an optionally substituted C₂-C₈ heterocycle, an optionally substituted C₅-C₈ aryl, and an optionally substituted C₃-C₈ heteroaryl;
R⁵ is selected from the group consisting of hydrogen, an optionally substituted C₁-C₈ alkyl, a C₁-C₈ heteroalkyl, a C₁-C₈ haloalkyl, and a C₁-C₈ heterohaloalkyl;
R⁶ is selected from the group consisting of hydrogen and OR¹⁶; R⁷ and R⁸ are each independently selected from the group consisting of hydrogen and C₁-C₈ alkyl;
R⁹ is selected from the group consisting of hydrogen, OR¹⁶, and a C₁-C₈ alkyl;
R¹⁰ is selected from the group consisting of hydrogen and OR¹⁶; and
X is selected from the group consisting of O, S, and NOR¹⁶;
wherein:
R¹⁶ is selected from the group consisting of hydrogen, an optionally substituted C₁-C₈ alkyl, an optionally substituted C₁-C₈ heteroalkyl, an optionally substituted C₁-C₈ haloalkyl, an optionally substituted C₁-C₈ heterohaloalkyl, an optionally substituted C₅-C₈ aryl, and an optionally substituted C₃-C₈ heteroaryl;
R¹⁷ and R¹⁸ are each independently selected from the group consisting of hydrogen, C₁-C₈ alkyl, C₁-C₈ heteroalkyl, C₁-C₈ haloalkyl; or R¹⁷ and R¹⁸ together form a 3 to 7 membered ring;
R²⁰ and R³⁷ are each independently selected from the group consisting of hydrogen, a C₁-C₈ alkyl, a C₁-C₈ heteroalkyl, a C₁-C₈ haloalkyl, a C₁-C₈ heterohaloalkyl; or R³⁷ and R²⁰ together form a 3-7 membered ring;
R³⁴ is selected from the group consisting of hydrogen, a halogen, NO₂, OR¹⁶, NR¹⁷R¹⁸, —CN, COR²⁰, NR¹⁷(OR¹⁶), an optionally substituted C₁-C₈ alkyl, a C₁-C₈ heteroalkyl, a C₁-C₈ haloalkyl, and a C₁-C₈ heterohaloalkyl;
R³⁶ is selected from the group consisting of hydrogen, an optionally substituted C₁-C₈ alkyl, a C₁-C₈ heteroalkyl, a C₁-C₈ haloalkyl, a C₁-C₈ heterohaloalkyl;
R²⁷ is selected from the group consisting of hydrogen, a halogen, CO₂R²⁰, COR²⁰, CONR²⁰R³⁷, C═N(OR¹⁶), an optionally substituted C₁-C₈ alkyl, a C₁-C₈ heteroalkyl, a C₁-C₈ haloalkyl, a C₁-C₈ heterohaloalkyl or R²⁷ taken together with R²⁶ form a 3-7 membered ring;
R²⁸ is selected from the group consisting of hydrogen, —COR²⁰, —CO₂R²⁰, —CONR²⁰R³⁷, SO₂R²⁰, an optionally substituted C₁-C₈ alkyl, a C₁-C₈ heteroalkyl, a C₁-C₈ haloalkyl, and a C₁-C₈ heterohaloalkyl;
R³⁵ is selected from the group consisting of hydrogen, —COR²⁰, —CO₂R²⁰, CONR²⁰R³⁷, SO₂R²⁰, an optionally substituted C₁-C₈ alkyl, a C₁-C₈ heteroalkyl, a C₁-C₈ haloalkyl, a C₁-C₈ heterohaloalkyl;
R³⁰ is selected from the group consisting of hydrogen an optionally substituted C₁-C₈ alkyl, a C₁-C₈ heteroalkyl, a C₁-C₈ haloalkyl, a C₁-C₈ heterohaloalkyl; and
R³¹ is selected from the group consisting of hydrogen, halogen, and OR¹⁶;
wherein,
at least one of R¹, R² and R⁴ is not hydrogen; and
at least one of R¹¹, R¹², and one R¹³ is not hydrogen.

214. The compound of claim 213, wherein:
R¹ and R² are each independently selected from the group consisting of hydrogen, halogen, —CN, —OR¹⁶, C₁-C₈ alkyl;
R³ is selected from the group consisting of (a), (b), (f), (g), (h), (k), (l), and (m): 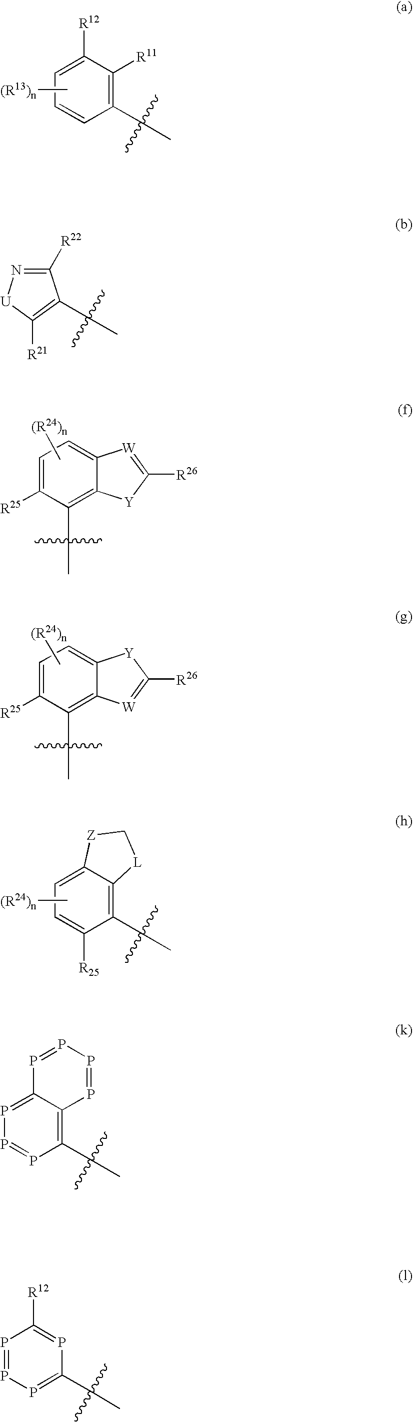 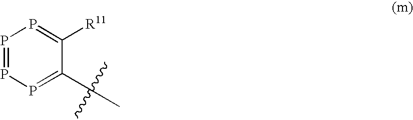
wherein,
R¹¹ is selected from the group consisting of hydrogen, halogen, —CN, —OR¹⁶, —NR¹⁷R¹⁸, —CH₂R¹⁶, —COR²⁰, —SOR²⁰, —SO₂R²⁰, NR¹⁷(OR¹⁶), an optionally substituted C₁-C₈ alkyl, a C₁-C₈ heteroalkyl, a C₁-C₈ haloalkyl, and a C₁-C₈ heterohaloalkyl;
R¹² is selected from the group consisting of hydrogen, halogen, —CN, —COR²⁰, OR¹⁶, NR¹⁷R¹⁸, an optionally substituted C₁-C₈ alkyl, a C₁-C₈ heteroalkyl, a C₁-C₈ haloalkyl, a C₁-C₈ heterohaloalkyl, or R¹² taken together with R¹¹ form a 3-7 membered ring;
each R¹³ is independently selected from the group consisting of hydrogen, halogen, CN, OR¹⁶, an optionally substituted C₁-C₈ alkyl, a C₁-C₈ heteroalkyl, a C₁-C₈ haloalkyl, a C₁-C₈ heterohaloalkyl, or R¹³ taken together with R¹² form a 3-7 membered ring;
R²¹ is selected from the group consisting of hydrogen, a C₁-C₈ alkyl, a C₁-C₈ heteroalkyl, and a C₁-C₈ haloalkyl;
R²² is selected from the group consisting of hydrogen, halogen, —NR¹⁷R¹⁸, a C₁-C₈ alkyl, a C₁-C₈ heteroalkyl, and a C₁-C₈ haloalkyl;
R²⁵ is selected from the group consisting of hydrogen, halogen, OR¹⁶, —CN, a C₁-C₈ alkyl, C₁-C₈ heteroalkyl, and C₁-C₈ haloalkyl;
R²⁶ is selected from the group consisting of hydrogen, halogen, OR¹⁶, —CN, a C₁-C₈ alkyl, a C₁-C₈ heteroalkyl, and a C₁-C₈ haloalkyl;
n is selected from 1, 2, and 3;
R⁵ is selected from the group consisting of hydrogen, a C₁-C₈ alkyl, a C₁-C₈ heteroalkyl, and a C₁-C₈ haloalkyl;
R⁷ and R⁸ are each C₁-C₈ alkyl;
R¹⁶ is selected from the group consisting of hydrogen, an optionally substituted C₁-C₈ alkyl, an optionally substituted C₁-C₈ heteroalkyl, and an optionally substituted C₁-C₈ haloalkyl;
R¹⁷ and R¹⁸ are each independently selected from the group consisting of hydrogen, a C₁-C₈ alkyl, or R¹⁷ and R¹⁸ together form a 3 to 7 membered ring;
R²⁰ and R³⁷ are each independently selected from the group consisting of hydrogen, a C₁-C₈ alkyl, or R³⁷ and R²⁰ together form a 3-7 membered ring;
R³⁶ is selected from the group consisting of hydrogen and a C₁-C₈ alkyl;
R²⁷ is selected from the group consisting of hydrogen, halogen, and a C₁-C₈ alkyl; and
R²⁸ is selected from the group consisting of hydrogen, —COR²⁰, SO₂R²⁰, a C₁-C₈ alkyl, a C₁-C₈ heteroalkyl, and a C₁-C₈ haloalkyl.

215. The compound of claim 213 wherein:
R¹ and R² are each independently selected from the group consisting of hydrogen, F, Br, Cl, CN, OMe, methyl, and ethyl;
R³ is selected from the group consisting of (a), (b), (f), (g), (l), and (m): 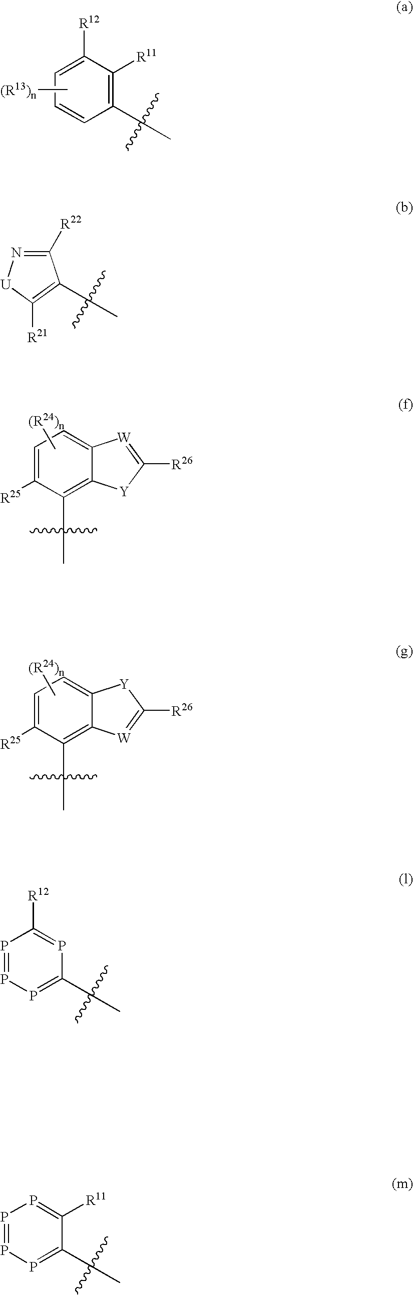
wherein,
R¹¹ is selected from the group consisting of hydrogen, halogen, CN, OMe, and an optionally substituted C₁-C₅ alkyl;
R¹² is selected from the group consisting of hydrogen, halogen, CN, OMe, an optionally substituted C₁-C₅ alkyl, a C₁-C₅ heteroalkyl, a C₁-C₅ haloalkyl, or R¹² taken together with R¹¹ form a 5-6 membered ring;
each R¹³ is independently selected from the group consisting of hydrogen, F, Cl, a C₁-C₅ alkyl, a C₁-C₅ heteroalkyl, a C₁-C₅ haloalkyl, or R¹³ taken together with R¹² form a 5-6 membered ring;
R²¹ is selected from the group consisting of hydrogen, methyl, ethyl, propyl, methoxy, and CF₃;
R²² is selected from the group consisting of hydrogen, F, Cl, NH₂, methyl, ethyl, propyl, methoxy, and CF₃;
each R²⁴ is independently selected from the group consisting of hydrogen, F, Br, Cl, methyl, ethyl, methoxy, and CF₃;
R²⁵ is selected from the group consisting of hydrogen, F, Cl, OMe, CN, a C₁-C₃ alkyl, a C₁-C₃ heteroalkyl, and a C₁-C₃ haloalkyl;
R²⁶ is selected from the group consisting of hydrogen, F, Cl, OMe, CN, a C₁-C₃ alkyl, and a C₁-C₃ haloalkyl;
n is selected from 1 and 2;
R⁴ is selected from the group consisting of hydrogen, F, Br, Cl, OR¹⁶, CN, C═N(OR¹⁶), CR³(OR¹⁶), an optionally substituted C₁-C₅ alkyl, an optionally substituted C₁-C₅ heteroalkyl, and an optionally substituted C₁-C₅ haloalkyl;
R⁵ is selected from the group consisting of hydrogen, methyl, ethyl, propyl, methoxy, and CF₃;
R⁷ and R⁸ are each independently selected from the group consisting of methyl and ethyl;
R⁹ is selected from the group consisting of hydrogen, OR¹⁶, and C₁-C₅ alkyl;
R¹⁰ is selected from the group consisting of hydrogen and OR¹⁶;
R¹⁶ is selected from the group consisting of hydrogen, an optionally substituted C₁-C₅ alkyl, an optionally substituted C₁-C₅ heteroalkyl, and an optionally substituted C₁-C₅ haloalkyl;
R¹⁷ and R²⁰ are independently selected from the group consisting of hydrogen and C₁-C₈ alkyl;
R³⁶ is selected from the group consisting of hydrogen and C₁-C₈ alkyl;
R²⁷ is selected from the group consisting of hydrogen, F, Cl, and C₁-C₈ alkyl;
R²⁸ is selected from the group consisting of hydrogen, —COR²⁰, SO₂R²⁰, C₁-C₈ alkyl, C₁-C₈ heteroalkyl, and C₁-C₈ haloalkyl; and
R³¹ is selected from the group consisting of hydrogen, F, Cl, methyl, and OMe.
